(19) United States
(12) Patent Application Publication
(10) Pub. No.: US 2023/0115640 A1
(43) Pub. Date: Apr. 13, 2023

(54) BLASTOCYST-LIKE STRUCTURES FROM EXTENDED PLURIPOTENT STEM CELLS

(71) Applicant: Salk Institute for Biological Studies, La Jolla, CA (US)

(72) Inventors: Juan Carlos Izpisua Belmonte, La Jolla, CA (US); Ronghui Li, La Jolla, CA (US); Cuiqing Zhong, San Diego, CA (US); Jun Wu, Farmers Branch, TX (US)

(73) Assignee: Salk Institute for Biological Studies, La Jolla, CA (US)

(21) Appl. No.: 17/766,176

(22) PCT Filed: Oct. 2, 2020
(86) PCT No.: PCT/US2020/054128

§ 371 Date: Apr. 1, 2022

Related U.S. Application Data
(60) Provisional application No. 62/910,335, filed on Oct. 3, 2019.

(51) Int. Cl. C12N 5/071; C12N 5/0735

(52) U.S. Cl. CPC C12N 5/0697; C12N 5/0606; C12N 2501/115; C12N 2501/155; C12N 2501/91; C12N 2506/02; C12N 2513/00; C12N 2501/40

(57) ABSTRACT

Provided herein are blastoids and methods for producing the same that are obtained from an extended pluripotent stem (EPS) cell. The herein-disclosed methods provide a unique and highly malleable in vitro system for studying early preimplantation development. Also provided are EPS-blastoids derived from a somatic cell.

### CROSS-REFERENCE

This application claims the benefit of U.S. Provisional Application No. 62/910,335, filed Oct. 3, 2019, which is incorporated herein by reference in its entirety.

### BACKGROUND

Current methods for generating mouse blastocyst-like structures, termed blastoids, require the sequential aggregation of embryonic stem cells (ESCs) and trophoblast stem cells (TSCs) in microwells. However, these blastoids are unable to model post-implantation development since they poorly develop into post-implantation embryo-like structures in vitro and only generate trophoblast cell types in vivo. These blastoids also are unable to model the pre-implantation because of the nature of the assembly method. Thus, there remains an unmet need for blastoids that are derived from one cell type and which develop to include all three founder tissues of a blastocysts: pluripotent epiblast (EPI) cells, trophectoderm (TE), and primitive endoderm (PE).

SUMMARY

In an aspect, there are provided methods of producing a blastoid, the method comprising: (a) obtaining or providing an extended pluripotent stem (EPS) cell; (b) culturing the EPS cell in a medium comprising one or more factors selected from the group consisting of a ROCK inhibitor, a FGF, Heparin, a Wnt agonist, a BMP, and a TGF-β signaling inhibitor; and (c) isolating the resulting blastoid. In some cases, the medium comprises two or more factors selected from the group consisting of the ROCK inhibitor, the FGF, Heparin, the Wnt agonist, the BMP, and the TGF-β signaling inhibitor. In some cases, the medium comprises three or more factors selected from the group consisting of the ROCK inhibitor, the FGF, Heparin, the Wnt agonist, the BMP, and the TGF-β signaling inhibitor. In some cases, the medium comprises four or more factors selected from the group consisting of the ROCK inhibitor, the FGF, Heparin, the Wnt agonist, the BMP, and the TGF-β signaling inhibitor. In some cases, the medium comprises five or more factors selected from the group consisting of the ROCK inhibitor, the FGF, Heparin, the Wnt agonist, the BMP, and a TGF-β signaling inhibitor. In some cases, the medium comprises the ROCK inhibitor, the FGF, Heparin, the Wnt agonist, the BMP, and the TGF-β signaling inhibitor. In some cases, the ROCK inhibitor is Y-27632, the FGF is FGF4, the Wnt agonist is Wnt-3a or CHIR99021, the BMP is BMP4, and/or the TGF-β signaling inhibitor is A83-01, SB431543, OR REPSOX. In some cases, the EPS cell is cultured in a v-bottomed microwell plate. In some cases, the v-bottomed microwell plate is an AggreWell plate. In some cases, the v-bottomed plate is centrifuged at about 300×g after the cell and medium is added to the plate. In some cases, after about 24 hours, the medium is replaced with a medium without the ROCK inhibitor. In some cases, the culturing is conducted for about 5 days. In some cases, the method further comprises at step (b) culturing the EPS cell with a trophectoderm (TE) cell.

In another aspect, there are provided methods of assisted reproduction of an individual, the method comprising: (a) obtaining or providing an extended pluripotent stem (EPS) cell derived from the individual; (b) culturing the EPS cell in a medium comprising one or more of factors selected from the group consisting of a ROCK inhibitor, a FGF, Heparin, a Wnt agonist, a BMP, and a TGF-β signaling inhibitor; (c) isolating a resulting blastoid; (d) transferring the resulting blastoid to a uterus. In some cases, the medium comprises two or more factors selected from the group consisting of the ROCK inhibitor, the FGF, Heparin, the Wnt agonist, the BMP, and the TGF-β signaling inhibitor. In some cases, the medium comprises three or more factors selected from the group consisting of the ROCK inhibitor, the FGF, Heparin, the Wnt agonist, the BMP, and the TGF-β signaling inhibitor. In some cases, the medium comprises four or more factors selected from the group consisting of the ROCK inhibitor, the FGF, Heparin, the Wnt agonist, the BMP, and the TGF-β signaling inhibitor. In some cases, the medium comprises five or more factors selected from the group consisting of the ROCK inhibitor, the FGF, Heparin, the Wnt agonist, the BMP, and the TGF-β signaling inhibitor. In some cases, the medium comprises the ROCK inhibitor, the FGF, Heparin, the Wnt agonist, the BMP, and the TGF-β signaling inhibitor. In some cases, the ROCK inhibitor is Y-27632, the FGF is FGF4, the Wnt agonist is Wnt-3a or CHIR99021, the BMP is BMP4, and/or the TGF-β signaling inhibitor is A83-01, SB431543, OR REPSOX. In some cases, the EPS cell is cultured in a v-bottomed microwell plate. In some cases, the v-bottomed microwell plate is an AggreWell plate. In some cases, the v-bottomed plate is centrifuged at about 300×g after the cell and medium is added to the plate. In some cases, after about 24 hours, the medium is replaced with a medium without Y-27632. In some cases, the culturing is conducted for about 5 days. In some cases, the individual is a mammal selected from a mouse, a rat, a rabbit, a horse, a sheep, a cow, a dog, a cat, an elephant, a whale, a rhinoceros, a non-human primate, or a human. In some cases, the EPS cell is an induced EPS cell derived from a somatic cell. In some cases, the method further comprises at step (b) culturing the EPS cell with a trophectoderm (TE) cell. In some cases, the uterus is receptive to implantation.

In another aspect, there are provided methods of determining a drug toxicity, the method comprising: (a) obtaining or providing a blastoid produced by a method according to any method provided herein; (b) contacting the blastoid to the drug; and (c) detecting signs of toxicity. In some cases, the signs of toxicity comprise cell death, loss of blastoid cell organization, arrest in blastoid growth or development.

In various embodiments of methods herein, the EPS cell is cultured in a medium comprising a KSOM or comprising an M16 medium.

In another aspect, there are provided blastoids, produced or producible by a method comprising: (a) obtaining an extended pluripotent stem (EPS) cell; (b) culturing the EPS cell in a medium comprising one or more of factors selected from the group consisting of a ROCK inhibitor, a FGF, Heparin, a Wnt agonist, a BMP, and a TGF-β signaling inhibitor; and (c) isolating the resulting blastoid. In some cases, the medium comprises two or more factors selected from the group consisting of the ROCK inhibitor, the FGF, Heparin, the Wnt agonist, the BMP, and the TGF-β signaling inhibitor. In some cases, the medium comprises three or more factors selected from the group consisting of the ROCK inhibitor, the FGF, Heparin, the Wnt agonist, the BMP, and the TGF-β signaling inhibitor. In some cases, the medium comprises four or more factors selected from the group consisting of the ROCK inhibitor, the FGF, Heparin, the Wnt agonist, the BMP, and the TGF-β signaling inhibitor. In some cases, the medium comprises five or more factors selected from the group consisting of the ROCK inhibitor, the FGF, Heparin, the Wnt agonist, the BMP, and the TGF-β signaling inhibitor. In some cases, the medium comprises the ROCK inhibitor, the FGF, Heparin, the Wnt agonist, the BMP, and the TGF-β signaling inhibitor. In some cases, the ROCK inhibitor is Y-27632, the FGF is FGF4, the Wnt agonist is Wnt-3a or CHIR99021, the BMP is BMP4, and/or the TGF-β signaling inhibitor is A83-01, SB431543, OR REPSOX. In some cases, the EPS cell is cultured in a v-bottomed microwell plate. In some cases, the v-bottomed microwell plate is an AggreWell plate. In some cases, the v-bottomed plate is centrifuged at about 300×g after the cell and medium is added to the plate. In some cases, after about 24 hours, the medium is replaced with a medium without the ROCK inhibitor. In some cases, the culturing is conducted for about 5 days. In some cases, the EPS cell is cultured in a medium comprising a KSOM or comprising an M16 medium. In some cases, the Wnt agonist is CHIR99021. In some cases, the TGF-β signaling inhibitor comprises A83-01, SB431543, OR REPSOX. In some cases, the EPS cell is an induced EPS cell derived from a somatic cell. In some cases, the blastoid is produced by a method where at step (b) culturing the EPS cell with a trophectoderm (TE) cell. In some cases, the EPS is derived from a mammal selected from a mouse, a rat, a rabbit, a horse, a sheep, a cow, a dog, a cat, an elephant, a whale, a rhinoceros, a non-human primate, or a human.

Provided herein are 3D differentiation systems that, in some embodiments, enable generation of blastocyst-like structures (EPS-blastoids) which are derived from a single stem cell type, the extended pluripotent stem (EPS) cell. When cultured, the EPS-blastoids generate structures characteristic of an E5.0-E5.5 post-implantation embryo. EPS-blastoids are transcriptionally similar to natural E3.5 blastocysts and contain all three blastocyst cell lineages. In utero EPS-blastoids are capable of implantation, triggering decidualization, and give rise to structures containing live tissues of EPI, TE, and PE origins. Also, EPS-blastoids can be generated from mouse fibroblasts; thus, embryo-like structures are produced from somatic cells. Accordingly, the herein disclosed EPS-blastoids serve as a model of early embryogenesis (both preimplantation and postimplantation) for testing candidate gene mutations, drug screening, and understanding the basic principles of embryo development. Further, the 3D differentiation system also serves as a framework for advancing the development of fully functional synthetic blastocysts in mice or other mammalian species.

In certain aspects, there are provided methods of producing a blastoid. The method comprising steps of (a) obtaining or providing an extended pluripotent stem (EPS) cell; (b) culturing the EPS cell in a medium comprising one or more factors selected from the group consisting of Y-27632, FGF4, Heparin, a Wnt agonist, BMP4, and a TGF-β signaling inhibitor; and (c) isolating the resulting blastoid.

In embodiments, the medium comprises two or more factors selected from the group consisting of Y-27632, FGF4, Heparin, a Wnt agonist, BMP4, and a TGF-β signaling inhibitor. In embodiments, the medium comprises three or more factors selected from the group consisting of Y-27632, FGF4, Heparin, a Wnt agonist, BMP4, and a TGF-β signaling inhibitor.

In embodiments, the medium comprises four or more factors selected from the group consisting of Y-27632, FGF4, Heparin, a Wnt agonist, BMP4, and a TGF-β signaling inhibitor. In embodiments, the medium comprises five or more factors selected from the group consisting of Y-27632, FGF4, Heparin, a Wnt agonist, BMP4, and a TGF-β signaling inhibitor. In embodiments, the medium comprises Y-27632, FGF4, Heparin, a Wnt agonist, BMP4, and a TGF-β signaling inhibitor.

In embodiments, the EPS cell is cultured in a v-bottomed microwell plate. In embodiments, the v-bottomed microwell plate is an AggreWell plate. In embodiments, the v-bottomed plate is centrifuged at about 300×g after the cell and medium is added to the plate.

In embodiments, after about 24 hours, the medium is replaced with a medium without a ROCK inhibitor, such as Y-27632.

In embodiments, the culturing is conducted for about 5 days.

In additional aspects, there are provided, methods of assisted reproduction of an individual. The method comprising steps of: (a) obtaining or providing an extended pluripotent stem (EPS) cell derived from the individual; (b) culturing the EPS cell in a medium comprising one or more of factors selected from the group consisting of Y-27632, FGF4, Heparin, a Wnt agonist, BMP4, and a TGF-β signaling inhibitor; (c) isolating a resulting blastoid; (d) transferring the resulting blastoid to a uterus.

In embodiments, the medium comprises two or more factors selected from the group consisting of Y-27632, FGF4, Heparin, a Wnt agonist, BMP4, and a TGF-β signaling inhibitor. In embodiments, the medium comprises three or more factors selected from the group consisting of Y-27632, FGF4, Heparin, a Wnt agonist, BMP4, and a TGF-β signaling inhibitor. In embodiments, the medium comprises four or more factors selected from the group consisting of Y-27632, FGF4, Heparin, a Wnt agonist, BMP4, and a TGF-β signaling inhibitor. In embodiments, the medium comprises five or more factors selected from the group consisting of Y-27632, FGF4, Heparin, a Wnt agonist, BMP4, and a TGF-β signaling inhibitor. In embodiments, the medium comprises Y-27632, FGF4, Heparin, a Wnt agonist, BMP4, and a TGF-β signaling inhibitor.

In embodiments, the EPS cell is cultured in a v-bottomed microwell plate. In embodiments, the v-bottomed microwell plate is an AggreWell plate. In embodiments, the v-bottomed plate is centrifuged at about 300×g after the cell and medium is added to the plate.

In embodiments, after about 24 hours, the medium is replaced with a medium without a ROCK inhibitor, such as Y-27632.

In embodiments, the culturing is conducted for about 5 days.

In embodiments, the individual is a mammal. In embodiments, the individual is a mouse, a rat, a rabbit, a horse, a sheep, a cow, a dog, a cat, an elephant, a whale, a rhinoceros, a non-human primate, or a human. In embodiments, the uterus is receptive to implantation.

In embodiments, the EPS cell is an induced EPS cell derived (e.g., reprogrammed) from a somatic cell.

In embodiments, the EPS cell is cultured in a medium comprising a KSOM or comprising an M16 medium.

In embodiments, the Wnt agonist comprises CHIR99021 or Wnt-3a.

In embodiments, the TGF-β signaling inhibitor comprises A83-01, SB431543, OR REPSOX.

In additional aspects, there are provided methods of determining a drug toxicity. The method comprising steps of: (a) obtaining or providing a blastoid produced by a method according to any herein-described method (b) contacting the blastoid to the drug; and (c) detecting signs of toxicity.

In embodiments, the signs of toxicity comprise cell death, loss of blastoid cell organization, arrest in blastoid growth or development.

In embodiments, the EPS cell is cultured in a medium comprising a KSOM or comprising an M16 medium.

In embodiments, the Wnt agonist comprises CHIR99021 or Wnt-3a.

In embodiments, the TGF-β signaling inhibitor comprises A83-01, SB431543, OR REPSOX.

In certain aspects, there are provided, blastoids, e.g., produced or producible by a method comprising steps of: (a) obtaining an extended pluripotent stem (EPS) cell; (b) culturing the EPS cell in a medium comprising one or more of factors selected from the group consisting of Y-27632, FGF4, Heparin, a Wnt agonist, BMP4, and a TGF-β signaling inhibitor; and (c) isolating the resulting blastoid.

In embodiments, the medium comprises two or more factors selected from the group consisting of Y-27632, FGF4, Heparin, a Wnt agonist, BMP4, and a TGF-β signaling inhibitor. In embodiments, the medium comprises three or more factors selected from the group consisting of Y-27632, FGF4, Heparin, a Wnt agonist, BMP4, and a TGF-β signaling inhibitor. In embodiments, the medium comprises four or more factors selected from the group consisting of Y-27632, FGF4, Heparin, a Wnt agonist, BMP4, and a TGF-β signaling inhibitor. In embodiments, the medium comprises five or more factors selected from the group consisting of Y-27632, FGF4, Heparin, a Wnt agonist, BMP4, and a TGF-β signaling inhibitor. In embodiments, the medium comprises Y-27632, FGF4, Heparin, a Wnt agonist, BMP4, and a TGF-β signaling inhibitor.

In embodiments, the EPS cell is cultured in a v-bottomed microwell plate. In embodiments, the v-bottomed microwell plate is an AggreWell plate. In embodiments, the v-bottomed plate is centrifuged at about 300×g after the cell and medium is added to the plate.

In embodiments, after about 24 hours, the medium is replaced with a medium without a ROCK inhibitor, such as Y-27632.

In embodiments, the culturing is conducted for about 5 days.

In embodiments, the EPS cell is cultured in a medium comprising a KSOM or comprising an M16 medium.

In embodiments, the Wnt agonist comprises CHIR99021 or Wnt-3a.

In embodiments, the TGF-β signaling inhibitor comprises A83-01, SB431543, OR REPSOX.

In embodiments, the EPS cell is an induced EPS cell derived (e.g., reprogrammed) from a somatic cell.

In additional aspects, there are provided blastoids in which at least ⅛ of its cells are derived from a common progenitor. As examples, at least ¼, at least ½, or at least ¾ of the cells in a blastoid are derived from a common progenitor.

Any herein described aspect or embodiment may be combined with any other herein described aspect or embodiment.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

An understanding of the features and advantages of the present invention will be obtained by reference to the following detailed description that sets forth illustrative embodiments, in which the principles of the invention are utilized, and the accompanying drawings of which:

FIG. 1A is a diagram showing a 3D Differentiation System for Generating Blastocyst-like Structures from EPS Cells.

FIG. 1B shows phase contrast images of EPS cell aggregates.

FIG. 1C shows phase contrast images of multicellular structures in microwells.

FIG. 1D shows representative phase contrast (upper panel) and fluorescence images (lower panel) of EPS cell aggregates.

FIG. 1E shows quantification of EPS-blastoids formation efficiency.

FIG. 1F shows phase contrast images of E3.5 blastocysts (upper panel) and EPS-blastoids (lower panel).

FIG. 1G shows histograms showing the distribution of the diameters of E3.5 blastocysts (upper panel) and EPS-blastoids (lower panel).

FIG. 1H shows phase contrast images of mouse embryos.

FIG. 1I shows quantification of the cavity area in the mouse embryos shown in FIG. 1H.

FIG. 1J shows phase contrast images of multicellular structures in microwells.

FIG. 1K shows quantification of EPS-blastoids formation efficiency.

FIG. 1L is a diagram showing the strategy for a single EPS cell to generate a clonal EPS-blastoid.

FIG. 1M shows phase contrast (left) and fluorescent (right) images of an EPS-blastoid generated using the strategy shown in FIG. 1L.

FIG. 1N shows quantification of EPS-blastoids formation efficiency.

FIG. 1O shows phase contrast images of multicellular structures in microwells.

FIG. 1P shows quantification of EPS-blastoids formation efficiency for ES-converted EPS or ES cells.

FIG. 1Q shows a phase contrast image of blastoids generated from Liu-EPS cells.

FIG. 1R shows quantification of EPS-blastoids formation efficiency from Liu-EPS cells.

FIG. 1S shows quantification of the diameter of blastocyst or Liu-EPS-blastoids.

FIG. 2A shows phase contrast (upper panel) and fluorescent (lower panel) images of EPS cells.

FIG. 2B shows immunofluorescence staining of an EPS aggregate at day 1 (left), day 2 (middle), and a compacted 8-cell embryo (right) for E-cadherin (E-CAD).

FIG. 2C shows quantification of the percentage of cell aggregates showing E-CAD+ staining.

FIG. 2D shows immunofluorescence staining of an EPS aggregate at day 1 (left) and a compacted 8-cell embryo (right) for ZO1.

FIG. 2E shows quantification of the percentage of cell aggregates showing ZO1+ staining at day 1 or day 2.

FIG. 2F shows a heatmap showing the FPKM values of the indicated genes in two EPS and ES cell lines.

FIG. 2G shows immunofluorescence staining of 2D EPS cells for ZO1 and OCT4.

FIG. 2H shows immunofluorescence staining of EPS aggregates.

FIG. 2I shows quantification of the percentage of cell aggregates.

FIG. 2J shows immunofluorescence staining of 2D EPS cells for YAP (FIG. 2I).

FIG. 2K shows immunofluorescence staining for active YAP in EPS aggregates.

FIG. 2L shows immunofluorescence staining for active YAP in an EPS-blastoid and an E4.5 blastocyst.

FIG. 2M shows quantification of the percentage of structures showing active YAP+.

FIG. 2N shows phase contrast images of mouse embryos.

FIG. 2O shows quantification of the cavity area in the mouse embryos shown in (FIG. 2K).

FIG. 2P shows phase contrast images of multicellular structures in microwells.

FIG. 2Q shows quantification of EPS-blastoids formation efficiency.

FIG. 2R shows immunostaining of an EPS-blastoid from a paternal X-GFP cell line.

FIG. 2S shows quantification of the frequency of different EPS-blastoid categories based on paternal X-GFP expression pattern.

FIG. 3A shows Immunofluorescence staining of EPS-blastoids for CDX2.

FIG. 3B shows immunofluorescence staining of EPS-blastoids for EOMES and OCT4.

FIG. 3C shows immunofluorescence staining of EPS-blastoids for CK8.

FIG. 3D, FIG. 3E, and FIG. 3F show immunofluorescence staining of EPS-blastoids for SOX2 (FIG. 3D), NANOG (FIG. 3E), and OCT4 (FIG. 3F).

FIG. 3G shows immunofluorescence staining of EPS-blastoids for CDX2 and NANOG.

FIG. 3H shows quantification of the frequency of different EPS-blastoid categories based on CDX2 and SOX2.

FIG. 3I shows quantification of the number of cells with SOX2+ or CDX2+ staining in the ICM or TE compartment.

FIG. 3J shows immunofluorescence staining of EPS aggregates at the indicated day for SOX2 and CDX2 expression.

FIG. 3K shows quantification of different patterns of SOX2 and CDX2 expression in EPS cell aggregates.

FIG. 3L shows immunofluorescence staining of an EPS-blastoid.

FIG. 3M shows quantification of the frequency of EPS-blastoids with or without GATA4+ PE-like cells.

FIG. 3N shows quantification of the number of cells with NANOG+ or GATA4+ staining in the EPI- or PE-like compartment, respectively, of blastocysts or EPS-blastoids.

FIG. 3O shows immunofluorescence staining of ES-converted EPS-blastoids.

FIG. 3P shows immunofluorescence staining of ES-converted EPS-blastoids.

FIG. 3Q shows immunofluorescence staining of a Liu-EPS-blastoid.

FIG. 3R shows immunofluorescence staining of an EPS-blastoid.

FIG. 4A show a principle component analysis (PCA) of bulk RNA-Seq data from individual EPS-blastoid, blastocyst, and morula. The number of biological replicates in each group was shown inside the parenthesis.

FIG. 4B shows unsupervised average clustering analysis of RNA-Seq data from individual EPS-blastoid , blastocyst, and morula.

FIG. 4C shows summary of differential gene expression analysis between EPS-blastoids and blastocysts.

FIG. 4D shows a volcano plot showing the differentially expressed genes (DEGs) between EPS-blastoids and blastocysts.

FIG. 4E shows a summary of differential gene expression analysis between EPS-blastoids and morulae.

FIG. 4F shows pathways enrichment analysis of DEGs between EPS-blastoids and blastocysts.

FIG. 4G shows a Uniform Manifold Approximation and Projection (UMAP) plot of 2702 cells from blastocysts and EPS-blastoids after alignment using the Seurat package.

FIG. 4H shows a UMAP plot showing the clustering of all cells.

FIG. 4I shows a UMAP plot showing the clustering of cells from blastocysts (left) or EPS-blastoids (right), respectively.

FIG. 4J shows the expression of lineage-specific genes shown in UMAP plots.

FIG. 4K shows an Unsupervised clustering analysis showing the cells of similar lineage identities cluster to each other regardless of sample type.

FIG. 4L, FIG. 4N, and FIG. 4P show Dot plots showing the differentially expressed genes (DEGs) in the ICM/EPI lineage (FIG. 4L), PE lineage (FIG. 4N), and TE lineage (FIG. 4P) between blastocysts and EPS-blastoids.

FIG. 4M and FIG. 4O show Gene ontology analysis of biological functions for DEGs in the ICM/EPI lineage (FIG. 4M) and PE lineage (FIG. 4O) between blastocyst and EPS-blastoids.

FIG. 5A shows a phase contrast image of de novo derived ES cell lines from EPS-blastoids.

FIG. 5B shows immunofluorescence staining of ES cells derived from EPS-blastoids.

FIG. 5C shows a brightfield image of two littermates generated from blastocyst injected with EPS-blastoid-derived ES cells.

FIG. 5D shows phase contrast image of de novo derived TS cell lines from EPS-blastoids.

FIG. 5E shows immunofluorescence staining of TS cells derived from EPS-blastoids.

FIG. 5F shows immunofluorescence staining of a placental section.

FIG. 5G shows phase contrast image of de novo derived XEN cell lines from EPS-blastoids.

FIG. 5H and FIG. 5I show immunofluorescence staining of EPS-blastoids-derived XEN cells for GATA6 (FIG. 5H) and GATA4 (FIG. 5I).

FIG. 5J shows a brightfield image of a yolk sac overlaid with tdTomato epifluorescence image.

FIG. 5K shows immunofluorescence staining of blastocyst-derived postimplantation embryo-like structures.

FIG. 5L shows immunofluorescence staining of EPS-blastoid-derived postimplantation embryo-like structures.

FIG. 5M shows quantification of the percentage of postimplantation embryo-like and malformed structures formed after in vitro culture of blastocysts and EPS -blastoids.

FIG. 5N shows immunofluorescence staining of an EPS-blastoid-derived peri-implantation embryo-like structure.

FIG. 5O shows immunofluorescence staining of an EPS-blastoid-derived postimplantation embryo-like structure.

FIG. 5P and FIG. 5Q show immunofluorescence staining of a blastocyst—(FIG. 5P) or an EPS-blastoid—(FIG. 5Q) derived postimplantation embryo-like structure for PCX and SOX2 (FIG. 5P) or PCX and OCT4 (FIG. 5Q).

FIG. 5R and FIG. 5S show immunofluorescence staining of postimplantation embryo-like structures.

Data above are represented as mean±SEM. Scale bar, 500 μm (FIG. 5F, upper), 100 μm (FIG. 5A, FIG. 5D, FIG. 5G, and FIG. 5J), 50 μm (FIG. 5K, FIG. 5I, FIG. 5O, FIG. 5P, and FIG. 5Q), 20 μm (FIG. 5F, bottom; and FIG. 5N). Ho, Hoechst.

FIG. 6A shows a brightfield image showing the formation of decidua in the mouse uterus.

FIG. 6B shows a brightfield image of the uterus of a control mouse at 7.5 dpc.

FIG. 6C shows a brightfield image of a mouse uterus 5 days after EPS-blastoids transfer.

FIG. 6D shows a brightfield image of a decidua (circled with dotted line) after removing the uterus wall.

FIG. 6E shows quantification of the efficiency of decidua formation per EPS-blastoid transferred into the mouse uterus.

FIG. 6F shows a brightfield images of deciduae recovered from control 7.5 dpc mice (left) or surrogate mice.

FIG. 6G shows PCR analysis of genomic DNA for the tdTomato gene.

FIG. 6H shows immunohistochemistry analysis of decidua sections.

FIG. 6I shows immunofluorescence staining of a section from control decidua (left) or EPS-blastoid-induced decidua (right) for CK8.

FIG. 6J and FIG. 6K show brightfield images of a control E7.5 embryo (FIG. 6J) or an in vivo EPS-blastoid-derived structure recovered from decidua at 7.5 dpc (5 days after EPS-blastoids transfer) (FIG. 6K).

FIG. 6L shows a brightfield images of control embryos.

FIG. 6M shows a brightfield (left) and fluorescent (right) images of EPS-blastoid-derived structures recovered from decidua.

FIG. 6N to FIG. 6P show Immunofluorescence staining of sections from an in vivo EPS-blastoid-derived structure recovered from decidua at 7.5 dpc (5 days after EPS-blastoids transfer) for OCT4 (FIG. 6N), EOMES (FIG. 60), and GATA4 (FIG. 6P).

FIG. 6Q to FIG. 6S show immunofluorescence staining for OCT4 (FIG. 6Q), EOMES (FIG. 6R), and GATA4 (FIG. 6S) in tissue sections.

FIG. 7A shows a phase contrast image of iEPS-blastoids.

FIG. 7B shows quantification of iEPS-blastoids formation efficiency.

FIG. 7C shows a histogram showing the distribution of diameters of iEPS-blastoids.

FIG. 7D shows immunofluorescence staining of iEPS aggregates.

FIG. 7E shows immunofluorescence staining of an iEPS aggregate.

FIG. 7F shows immunofluorescence staining of an iEPS-blastoid.

FIG. 7G shows immunofluorescence staining of an iEPS-blastoid.

FIG. 7H shows immunofluorescence staining of iEPS-blastoids.

FIG. 7I shows immunofluorescence staining of a postimplantation embryo-like structure.

FIG. 7J and FIG. 7K shows immunofluorescence staining of postimplantation embryo-like structure.

FIG. 7L shows a brightfield image showing the formation of decidua in the mouse uterus.

FIG. 8 shows a diagram summarizing the major findings of the herein disclosed data.

FIG. 9A shows a diagram showing the strategy of human EPS-blastoid formation.

FIG. 9B shows phase-contrast images of human EPS aggregates.

FIG. 9C shows quantification of human EPS-blastoid formation efficiency.

FIG. 9D shows immunofluorescence staining of human EPS-blastoid for CDX2 and SOX2.

FIG. 10A shows phase-contrast images of human EPSC aggregates.

FIG. 10B shows quantification of human EPSC-blastoid formation efficiency.

FIG. 10C shows immunofluorescence staining of human EPSC-blastoid for CDX2, OCT4, and SOX2.

### DETAILED DESCRIPTION

Provided herein are 3D differentiation systems that enable generation of blastocyst-like structures (EPS-blastoids) through lineage segregation and self-organization and which are derived from a single stem cell type, extended pluripotent stem (EPS) cell.

Additionally provided herein are EPS-blastoids that resemble blastocysts in morphology and cell lineage allocation and recapitulate key morphogenetic events during preimplantation and early postimplantation development in vitro. Upon transfer, EPS-blastoids undergo implantation, induce decidualization, and generate live tissues in utero. EPS-blastoids contain all three blastocyst cell lineages and share transcriptional similarity with natural blastocysts. EPS-blastoids can be generated from adult cells which have acquired stem-cell like characteristics via cellular reprogramming. EPS-blastoids provide a unique platform for studying early embryogenesis and pave the way to create viable synthetic embryos using cultured cells.

Indeed, since the EPS-blastoids undergo morphogenetic events characteristic of post-implantation development upon further culturing, the EPS-blastoid model provided herein allows a unique platform for studying the peri- and post-implantation in a high throughput manner.

Moreover, the EPS-blastoid model provided herein offers a unique platform for studying the effects of genetic variants on early embryogenesis. Compared to the use of genetically modified mouse, the herein-described EPS-blastoid model bypasses the time-consuming process of establishing mouse model and serves as a screening process at the beginning of an animal study. Similarly, the EPS-blastoid model offers a unique platform for studying the effects of de novo mutations (DNMs) on early embryogenesis.

Also, the EPS-blastoid model serves as a platform to test drug toxicity on early embryo development, and in a high throughput manner. Compared to normal mouse embryos, the herein-disclosed model has an advantage of integrating genetic variants and drug toxicology, hence providing a chance to look into how genetic variants affect the response to drugs. Further, since the EPS-blastoids may be derived from a single cell, or at least one cell type, each or a majority of cells in the resulting blastoid may similarly respond to a drug and/or express the same genetic variant. In other words, the present disclosure enables more straightforward interpretation of the readouts after introducing genetic and/or epigenetic changes.

The EPS-blastoids as disclosed herein are useful to produce specific lineage progenitors in a 3D setting, which has the advantages of mimicking the natural environment; this is a significant advantage over methods employing 2D cultures.

Additionally, the EPS-blastoid model is useful for the development of ways to preserve the endangered species, by creating adult animals that are derived from somatic cells, e.g., from a male or from an infertile female.

### Methods of Producing a Mammalian Blastoid

Provided herein are methods of producing a blastoid, such as a mammalian blastoid. In some cases, the method comprises obtaining or providing an extended pluripotent stem (EPS) cell. In some cases, the method comprises culturing the EPS cell in a medium comprising one or more factors selected from the group consisting of a ROCK inhibitor, a FGF, Heparin, a Wnt agonist, a BMP, and a TGF-β signaling inhibitor. In some cases, the method comprises isolating the resulting blastoid. In some cases, the medium comprises two or more factors selected from the group consisting of the ROCK inhibitor, the FGF, Heparin, the Wnt agonist, the BMP, and the TGF-β signaling inhibitor. In some cases, the medium comprises three or more factors selected from the group consisting of the ROCK inhibitor, the FGF, Heparin, the Wnt agonist, the BMP, and the TGF-β signaling inhibitor. In some cases, the medium comprises four or more factors selected from the group consisting of the ROCK inhibitor, the FGF, Heparin, the Wnt agonist, the BMP, and the TGF-β signaling inhibitor. In some cases, the medium comprises five or more factors selected from the group consisting of the ROCK inhibitor, the FGF, Heparin, the Wnt agonist, the BMP, and a TGF-β signaling inhibitor. In some cases, the medium comprises the ROCK inhibitor, the FGF, Heparin, the Wnt agonist, the BMP, and the TGF-β signaling inhibitor. In some cases, the ROCK inhibitor is Y-27632. In some cases, the FGF is FGF4. In some cases, the Wnt agonist is Wnt-3a or CHIR99021. In some cases, the BMP is BMP4. In some cases, the TGF-β signaling inhibitor is A83-01, SB431543, OR REPSOX.

In additional aspects, there are provided methods of producing a blastoid. The method comprising steps of (a) obtaining or providing an extended pluripotent stem (EPS) cell; (b) culturing the EPS cell in a medium comprising one or more factors selected from the group consisting of Y-27632, FGF4, Heparin, a Wnt agonist, BMP4, and a TGF-β signaling inhibitor; and (c) isolating the resulting blastoid.

Methods herein culture EPS cells in methods of producing a blastoid in any suitable culture vessel. In some cases, the EPS cell is cultured in a v-bottomed microwell plate. In some cases, the v-bottomed microwell plate is an AggreWell plate.

In additional aspects, methods of producing a blastoid herein comprise centrifuging a culture vessel comprising the EPS cells and the culture media. In some cases, the v-bottomed plate is centrifuged at about 50×g after the cell and medium is added to the plate. In some cases, the v-bottomed plate is centrifuged at about 100×g after the cell and medium is added to the plate. In some cases, the v-bottomed plate is centrifuged at about 150×g after the cell and medium is added to the plate. In some cases, the v-bottomed plate is centrifuged at about 200×g after the cell and medium is added to the plate. In some cases, the v-bottomed plate is centrifuged at about 250×g after the cell and medium is added to the plate. In some cases, the v-bottomed plate is centrifuged at about 300×g after the cell and medium is added to the plate. In some cases, the v-bottomed plate is centrifuged at about 350×g after the cell and medium is added to the plate. In some cases, the v-bottomed plate is centrifuged at about 400×g after the cell and medium is added to the plate. In some cases, the v-bottomed plate is centrifuged at about 450×g after the cell and medium is added to the plate. In some cases, the v-bottomed plate is centrifuged at about 500×g after the cell and medium is added to the plate.

In further aspects of methods of producing a blastoid provided herein, the contents of the culture medium are changed after culturing the EPS cell for a period of time. In some cases, the contents of the culture medium are changed after about 8 hours, about 12 hours, about 16 hours, about 24 hours, about 36 hours, or about 48 hours after culturing the EPS cell. In some cases, the medium is replaced with a medium without the ROCK inhibitor. In some cases, the medium is replaced with a medium without the FGF. In some cases, the medium is replaced with a medium without heparin. In some cases the medium is replaced with a medium without the Wnt agonist. In some cases, the medium is replaced with a medium without the BMP. In some cases, the medium is replaced with a medium without the TGF-β signaling inhibitor.

In further aspects of methods of producing a blastoid provided herein, the EPS cell is cultured for an appropriate period of time sufficient to form the blastoid. In some cases, the culturing is conducted for about 1 day, about 2 days, about 3 days, about 4 days, about 5 days, about 6 days, about 7 days, about 8 days, about 9 days, about 10 days, or more as needed. In some cases, the culturing is conducted for about 1-5 days, about 2-6 days, about 3-7 days, about 4-8 days, about 5-9 days, or about 6-10 days.

In additional aspects of methods of producing a blastoid provided herein, the method comprises culturing the EPS cell with additional cell types that facilitate production of the blastoid. For example, in some cases the method comprises culturing the EPS cell with a trophectoderm (TE) cell.

In further aspects provided herein, in some cases, blastoids are derived from a mammalian EPS cell. In some cases, the mammalian EPS cell is an EPS cell from a human, a mouse, a rat, a rabbit, a cat, a dog, a guinea pig, a hamster, a horse, a cow, a sheep, a pig, a goat, an elephant, a rhinoceros, an orangutan, a gorilla, a bonobo, a chimpanzee, a monkey, a panda, a tiger, a whale, a dolphin, a sea lion, a narwhal, a beluga, a fox, a wolf, a pronghorn, a kangaroo, a sloth, a koala, a hippopotamus, a bear, or a leopard.

### Methods of Assisted Reproduction

Further provided herein are methods of assisted reproduction of an individual. In some cases, the method comprises obtaining or providing an extended pluripotent stem (EPS) cell derived from the individual. In some cases, the method comprises culturing the EPS cell in a medium comprising one or more of factors selected from the group consisting of a ROCK inhibitor, a FGF, Heparin, a Wnt agonist, a BMP, and a TGF-β signaling inhibitor. In some cases, the method comprises isolating a resulting blastoid. In some cases, the method comprises transferring the resulting blastoid to a uterus. In some cases, the medium comprises two or more factors selected from the group consisting of the ROCK inhibitor, the FGF, Heparin, the Wnt agonist, the BMP, and the TGF-β signaling inhibitor. In some cases, the medium comprises three or more factors selected from the group consisting of the ROCK inhibitor, the FGF, Heparin, the Wnt agonist, the BMP, and the TGF-β signaling inhibitor. In some cases, the medium comprises four or more factors selected from the group consisting of the ROCK inhibitor, the FGF, Heparin, the Wnt agonist, the BMP, and the TGF-β signaling inhibitor. In some cases, the medium comprises five or more factors selected from the group consisting of the ROCK inhibitor, the FGF, Heparin, the Wnt agonist, the BMP, and a TGF-β signaling inhibitor. In some cases, the medium comprises the ROCK inhibitor, the FGF, Heparin, the Wnt agonist, the BMP, and the TGF-signaling inhibitor. In some cases, the ROCK inhibitor is Y-27632. In some cases, the FGF is FGF4. In some cases, the Wnt agonist is Wnt-3a or CHIR99021. In some cases, the BMP is BMP4. In some cases, the TGF-β signaling inhibitor is A83-01, SB431543, OR REPSOX.

In further aspects, there are provided methods of assisted reproduction of an individual. The method comprising steps of: (a) obtaining or providing an extended pluripotent stem (EPS) cell derived from the individual; (b) culturing the EPS cell in a medium comprising one or more of factors selected from the group consisting of Y-27632, FGF4, Heparin, a Wnt agonist, BMP4, and a TGF-β signaling inhibitor; (c) isolating a resulting blastoid; (d) transferring the resulting blastoid to a uterus.

In further aspects of methods of assisted reproduction provided herein, a blastoid is produced in any suitable culture vessel. In some cases, the EPS cell is cultured in a v-bottomed microwell plate. In some cases, the v-bottomed microwell plate is an AggreWell plate.

In additional aspects, methods of assisted reproduction provided herein comprise centrifuging a culture vessel comprising the EPS cells and the culture media. In some cases, the v-bottomed plate is centrifuged at about 50×g after the cell and medium is added to the plate. In some cases, the v-bottomed plate is centrifuged at about 100×g after the cell and medium is added to the plate. In some cases, the v-bottomed plate is centrifuged at about 150×g after the cell and medium is added to the plate. In some cases, the v-bottomed plate is centrifuged at about 200×g after the cell and medium is added to the plate. In some cases, the v-bottomed plate is centrifuged at about 250×g after the cell and medium is added to the plate. In some cases, the v-bottomed plate is centrifuged at about 300×g after the cell and medium is added to the plate. In some cases, the v-bottomed plate is centrifuged at about 350×g after the cell and medium is added to the plate. In some cases, the v-bottomed plate is centrifuged at about 400×g after the cell and medium is added to the plate. In some cases, the v-bottomed plate is centrifuged at about 450×g after the cell and medium is added to the plate. In some cases, the v-bottomed plate is centrifuged at about 500×g after the cell and medium is added to the plate.

In further aspects of methods of assisted reproduction provided herein, the contents of the culture medium are changed after culturing the EPS cell for a period of time. In some cases, the contents of the culture medium are changed after about 8 hours, about 12 hours, about 16 hours, about 24 hours, about 36 hours, or about 48 hours after culturing the EPS cell. In some cases, the medium is replaced with a medium without the ROCK inhibitor. In some cases, the medium is replaced with a medium without the FGF. In some cases, the medium is replaced with a medium without heparin. In some cases the medium is replaced with a medium without the Wnt agonist. In some cases, the medium is replaced with a medium without the BMP. In some cases, the medium is replaced with a medium without the TGF-β signaling inhibitor.

In further aspects of methods of assisted reproduction provided herein, the EPS cell is cultured for an appropriate period of time sufficient to form the blastoid. In some cases, the culturing is conducted for about 1 day, about 2 days, about 3 days, about 4 days, about 5 days, about 6 days, about 7 days, about 8 days, about 9 days, about 10 days, or more as needed. In some cases, the culturing is conducted for about 1-5 days, about 2-6 days, about 3-7 days, about 4-8 days, about 5-9 days, or about 6-10 days.

In further aspects of assisted reproduction provided herein, in some cases, the individual is a mammal. In some cases, the mammal is a human, a mouse, a rat, a rabbit, a cat, a dog, a guinea pig, a hamster, a horse, a cow, a sheep, a pig, a goat, an elephant, a rhinoceros, an orangutan, a gorilla, a bonobo, a chimpanzee, a monkey, a panda, a tiger, a whale, a dolphin, a sea lion, a narwhal, a beluga, a fox, a wolf, a pronghorn, a kangaroo, a sloth, a koala, a hippopotamus, a bear, or a leopard.

In additional aspects of methods of assisted reproduction provided herein, the EPS cell is an induced EPS cell derived from a somatic cell. In some cases, the somatic cell is any cell derived from an individual that is not a germ cell. Any suitable somatic cell is contemplated to be used in methods herein. A non-limiting list of somatic cells for use in methods herein include an endothelial cell, an epithelial cell, a blood cell, an adipocyte, a neuron, an osteoclast, a chondrocyte, a myocyte, or other cell type.

In additional aspects of methods of assisted reproduction provided herein, the method comprises culturing the EPS cell with additional cell types that facilitate production of the blastoid. For example, in some cases the method comprises culturing the EPS cell with a trophectoderm (TE) cell.

In additional aspects of methods of assisted reproduction provided herein the uterus of the individual is receptive to implantation. In some cases, the individual is treated with a medication in order to prepare the uterus for implantation. In some cases, the menstrual cycle and endometrial thickness of the individual is monitored for receptivity to implantation.

### Mammalian Blastoids

In an aspect there provided, compositions comprising an extended pluripotent stem cell and at least one factor selected from the group consisting of a ROCK inhibitor, a FGF, Heparin, a Wnt agonist, a BMP, and a TGF-β signaling inhibitor. In some cases, isolating the resulting blastoid. In some cases, the medium comprises two or more factors selected from the group consisting of the ROCK inhibitor, the FGF, Heparin, the Wnt agonist, the BMP, and the TGF-β signaling inhibitor. In some cases, the medium comprises three or more factors selected from the group consisting of the ROCK inhibitor, the FGF, Heparin, the Wnt agonist, the BMP, and the TGF-β signaling inhibitor. In some cases, the medium comprises four or more factors selected from the group consisting of the ROCK inhibitor, the FGF, Heparin, the Wnt agonist, the BMP, and the TGF-β signaling inhibitor. In some cases, the medium comprises five or more factors selected from the group consisting of the ROCK inhibitor, the FGF, Heparin, the Wnt agonist, the BMP, and a TGF-β signaling inhibitor. In some cases, the medium comprises the ROCK inhibitor, the FGF, Heparin, the Wnt agonist, the BMP, and the TGF-β signaling inhibitor. In some cases, the ROCK inhibitor is Y-27632. In some cases, the FGF is FGF4. In some cases, the Wnt agonist is Wnt-3a or CHIR99021. In some cases, the BMP is BMP4. In some cases, the TGF-β signaling inhibitor is A83-01, SB431543, OR REPSOX. In some cases, the composition further comprises a trophectoderm cell. In some cases, the composition comprises a blastoid.

In some aspects there are provided blastoids that are produced or producible by a method herein. In some cases, the method comprises obtaining an extended pluripotent stem (EPS) cell. In some cases, the method comprises culturing the EPS cell in a medium comprising one or more of factors selected from the group consisting of a ROCK inhibitor, a FGF, Heparin, a Wnt agonist, a BMP, and a TGF-β signaling inhibitor. In some cases, the method comprises isolating the resulting blastoid. In some cases, the medium comprises two or more factors selected from the group consisting of the ROCK inhibitor, the FGF, Heparin, the Wnt agonist, the BMP, and the TGF-β signaling inhibitor. In some cases, the medium comprises three or more factors selected from the group consisting of the ROCK inhibitor, the FGF, Heparin, the Wnt agonist, the BMP, and the TGF-β signaling inhibitor. In some cases, the medium comprises four or more factors selected from the group consisting of the ROCK inhibitor, the FGF, Heparin, the Wnt agonist, the BMP, and the TGF-β signaling inhibitor. In some cases, the medium comprises five or more factors selected from the group consisting of the ROCK inhibitor, the FGF, Heparin, the Wnt agonist, the BMP, and a TGF-β signaling inhibitor. In some cases, the medium comprises the ROCK inhibitor, the FGF, Heparin, the Wnt agonist, the BMP, and the TGF-β signaling inhibitor. In some cases, the ROCK inhibitor is Y-27632. In some cases, the FGF is FGF4. In some cases, the Wnt agonist is Wnt-3a or CHIR99021. In some cases, the BMP is BMP4. In some cases, the TGF-β signaling inhibitor is A83-01, SB431543, OR REPSOX.

In additional aspects, there are provided blastoids, e.g., produced or producible by a method comprising steps of: (a) obtaining an extended pluripotent stem (EPS) cell; (b) culturing the EPS cell in a medium comprising one or more of factors selected from the group consisting of Y-27632, FGF4, Heparin, a Wnt agonist, BMP4, and a TGF-β signaling inhibitor; and (c) isolating the resulting blastoid.

In additional aspects, blastoids herein are produced or producible by a method comprising centrifuging a culture vessel comprising the EPS cells and the culture media. In some cases, the v-bottomed plate is centrifuged at about 50×g after the cell and medium is added to the plate. In some cases, the v-bottomed plate is centrifuged at about 100×g after the cell and medium is added to the plate. In some cases, the v-bottomed plate is centrifuged at about 150×g after the cell and medium is added to the plate. In some cases, the v-bottomed plate is centrifuged at about 200×g after the cell and medium is added to the plate. In some cases, the v-bottomed plate is centrifuged at about 250×g after the cell and medium is added to the plate. In some cases, the v-bottomed plate is centrifuged at about 300×g after the cell and medium is added to the plate. In some cases, the v-bottomed plate is centrifuged at about 350×g after the cell and medium is added to the plate. In some cases, the v-bottomed plate is centrifuged at about 400×g after the cell and medium is added to the plate. In some cases, the v-bottomed plate is centrifuged at about 450×g after the cell and medium is added to the plate. In some cases, the v-bottomed plate is centrifuged at about 500×g after the cell and medium is added to the plate.

In further aspects, blastoids herein are produced or producible by a method wherein the contents of the culture medium are changed after culturing the EPS cell for a period of time. In some cases, the contents of the culture medium are changed after about 8 hours, about 12 hours, about 16 hours, about 24 hours, about 36 hours, or about 48 hours after culturing the EPS cell. In some cases, the medium is replaced with a medium without the ROCK inhibitor. In some cases, the medium is replaced with a medium without the FGF. In some cases, the medium is replaced with a medium without heparin. In some cases the medium is replaced with a medium without the Wnt agonist. In some cases, the medium is replaced with a medium without the BMP. In some cases, the medium is replaced with a medium without the TGF-β signaling inhibitor.

In further aspects, blastoids herein are produced or producible by a method wherein the EPS cell is cultured for an appropriate period of time sufficient to form the blastoid. In some cases, the culturing is conducted for about 1 day, about 2 days, about 3 days, about 4 days, about 5 days, about 6 days, about 7 days, about 8 days, about 9 days, about 10 days, or more as needed. In some cases, the culturing is conducted for about 1-5 days, about 2-6 days, about 3-7 days, about 4-8 days, about 5-9 days, or about 6-10 days.

In further aspects, blastoids herein are produced or producible by a method wherein the EPS cell is an induced EPS cell derived from a somatic cell. In some cases, the somatic cell is any cell derived from an individual that is not a germ cell. Any suitable somatic cell is contemplated to be used in methods herein. A non-limiting list of somatic cells for use in methods herein include an endothelial cell, an epithelial cell, a blood cell, an adipocyte, a neuron, an osteoclast, a chondrocyte, a myocyte, or other cell type.

In additional aspects, blastoids herein are produced or producible by a method wherein the EPS cell is cultured with additional cell types that facilitate production of the blastoid. For example, in some cases the method comprises culturing the EPS cell with a trophectoderm (TE) cell.

In additional aspects provided herein, in some cases, blastoids are derived from a mammalian EPS cell. In some cases, the mammalian EPS cell is an EPS cell from a human, a mouse, a rat, a rabbit, a cat, a dog, a guinea pig, a hamster, a horse, a cow, a sheep, a pig, a goat, an elephant, a rhinoceros, an orangutan, a gorilla, a bonobo, a chimpanzee, a monkey, a panda, a tiger, a whale, a dolphin, a sea lion, a narwhal, a beluga, a fox, a wolf, a pronghorn, a kangaroo, a sloth, a koala, a hippopotamus, a bear, or a leopard.

### Uses for Mammalian Blastoids

Mammalian blastoids created using methods disclosed herein are contemplated for a variety of uses including drug screening, reproductive medicine, and other research uses and methods. In some cases, uses of mammalian blastoids provided herein is determining a drug toxicity.

In further aspects, there are provided methods of determining a drug toxicity. The method comprising steps of: (a) obtaining or providing a blastoid produced by a method according to any herein-described method (b) contacting the blastoid to the drug; and (c) detecting signs of toxicity. In some cases, the method comprises obtaining or providing an extended pluripotent stem (EPS) cell. In some cases, the method comprises culturing the EPS cell in a medium comprising one or more factors selected from the group consisting of a ROCK inhibitor, a FGF, Heparin, a Wnt agonist, a BMP, and a TGF-β signaling inhibitor. In some cases, the method comprises isolating the resulting blastoid. In some cases, the medium comprises two or more factors selected from the group consisting of the ROCK inhibitor, the FGF, Heparin, the Wnt agonist, the BMP, and the TGF-β signaling inhibitor. In some cases, the medium comprises three or more factors selected from the group consisting of the ROCK inhibitor, the FGF, Heparin, the Wnt agonist, the BMP, and the TGF-β signaling inhibitor. In some cases, the medium comprises four or more factors selected from the group consisting of the ROCK inhibitor, the FGF, Heparin, the Wnt agonist, the BMP, and the TGF-β signaling inhibitor. In some cases, the medium comprises five or more factors selected from the group consisting of the ROCK inhibitor, the FGF, Heparin, the Wnt agonist, the BMP, and a TGF-β signaling inhibitor. In some cases, the medium comprises the ROCK inhibitor, the FGF, Heparin, the Wnt agonist, the BMP, and the TGF-β signaling inhibitor. In some cases, the ROCK inhibitor is Y-27632. In some cases, the FGF is FGF4. In some cases, the Wnt agonist is Wnt-3a or CHIR99021. In some cases, the BMP is BMP4. In some cases, the TGF-β signaling inhibitor is A83-01, SB431543, OR REPSOX.

Methods herein culture EPS cells in methods of producing a blastoid for testing drug toxicity in any suitable culture vessel. In some cases, the EPS cell is cultured in a v-bottomed microwell plate. In some cases, the v-bottomed microwell plate is an AggreWell plate.

In additional aspects, methods of producing a blastoid for testing drug toxicity herein comprise centrifuging a culture vessel comprising the EPS cells and the culture media. In some cases, the v-bottomed plate is centrifuged at about 50×g after the cell and medium is added to the plate. In some cases, the v-bottomed plate is centrifuged at about 100×g after the cell and medium is added to the plate. In some cases, the v-bottomed plate is centrifuged at about 150×g after the cell and medium is added to the plate. In some cases, the v-bottomed plate is centrifuged at about 200×g after the cell and medium is added to the plate. In some cases, the v-bottomed plate is centrifuged at about 250×g after the cell and medium is added to the plate. In some cases, the v-bottomed plate is centrifuged at about 300×g after the cell and medium is added to the plate. In some cases, the v-bottomed plate is centrifuged at about 350×g after the cell and medium is added to the plate. In some cases, the v-bottomed plate is centrifuged at about 400×g after the cell and medium is added to the plate. In some cases, the v-bottomed plate is centrifuged at about 450×g after the cell and medium is added to the plate. In some cases, the v-bottomed plate is centrifuged at about 500×g after the cell and medium is added to the plate.

In further aspects of methods of producing a blastoid for testing drug toxicity provided herein, the contents of the culture medium are changed after culturing the EPS cell for a period of time. In some cases, the contents of the culture medium are changed after about 8 hours, about 12 hours, about 16 hours, about 24 hours, about 36 hours, or about 48 hours after culturing the EPS cell. In some cases, the medium is replaced with a medium without the ROCK inhibitor. In some cases, the medium is replaced with a medium without the FGF. In some cases, the medium is replaced with a medium without heparin. In some cases the medium is replaced with a medium without the Wnt agonist. In some cases, the medium is replaced with a medium without the BMP. In some cases, the medium is replaced with a medium without the TGF-β signaling inhibitor.

In further aspects of methods of producing a blastoid for testing drug toxicity provided herein, the EPS cell is cultured for an appropriate period of time sufficient to form the blastoid. In some cases, the culturing is conducted for about 1 day, about 2 days, about 3 days, about 4 days, about 5 days, about 6 days, about 7 days, about 8 days, about 9 days, about 10 days, or more as needed. In some cases, the culturing is conducted for about 1-5 days, about 2-6 days, about 3-7 days, about 4-8 days, about 5-9 days, or about 6-10 days.

In additional aspects of methods of producing a blastoid for testing drug toxicity provided herein, the method comprises culturing the EPS cell with additional cell types that facilitate production of the blastoid. For example, in some cases the method comprises culturing the EPS cell with a trophectoderm (TE) cell.

In further aspects provided herein, in some cases, blastoids are derived from a mammalian EPS cell. In some cases, the mammalian EPS cell is an EPS cell from a human, a mouse, a rat, a rabbit, a cat, a dog, a guinea pig, a hamster, a horse, a cow, a sheep, a pig, a goat, an elephant, a rhinoceros, an orangutan, a gorilla, a bonobo, a chimpanzee, a monkey, a panda, a tiger, a whale, a dolphin, a sea lion, a narwhal, a beluga, a fox, a wolf, a pronghorn, a kangaroo, a sloth, a koala, a hippopotamus, a bear, or a leopard.

In embodiments, the EPS cell is cultured in a medium comprising a KSOM or comprising an M16 medium.

In embodiments, the EPS cell is cultured in a medium comprising a Wnt agonist, e.g., CHIR99021 or Wnt-3a.

In embodiments, the EPS cell is cultured in a medium comprising a TGF-β signaling inhibitor, e.g., A83-01, SB431543, OR REPSOX.

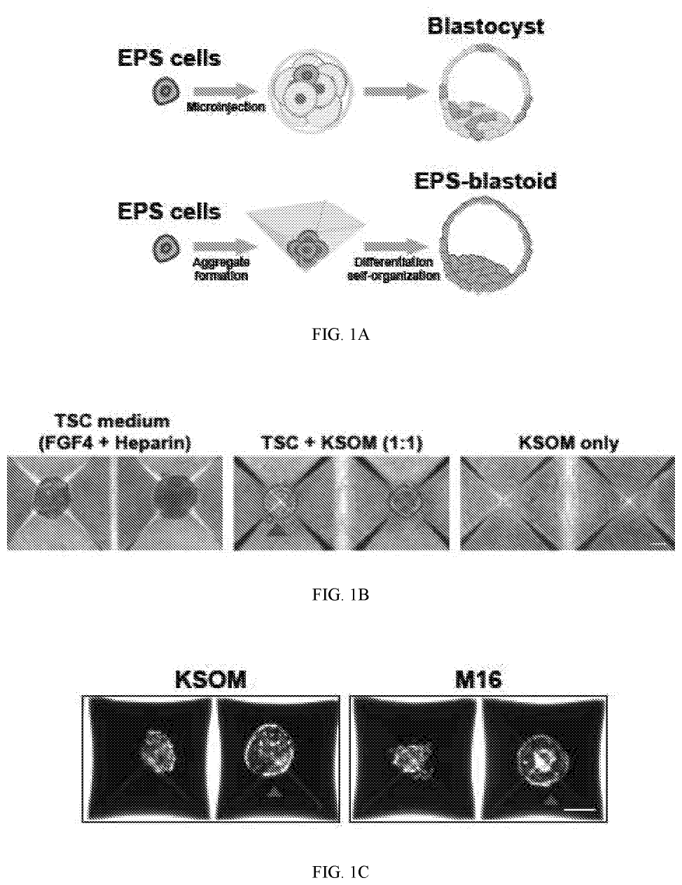

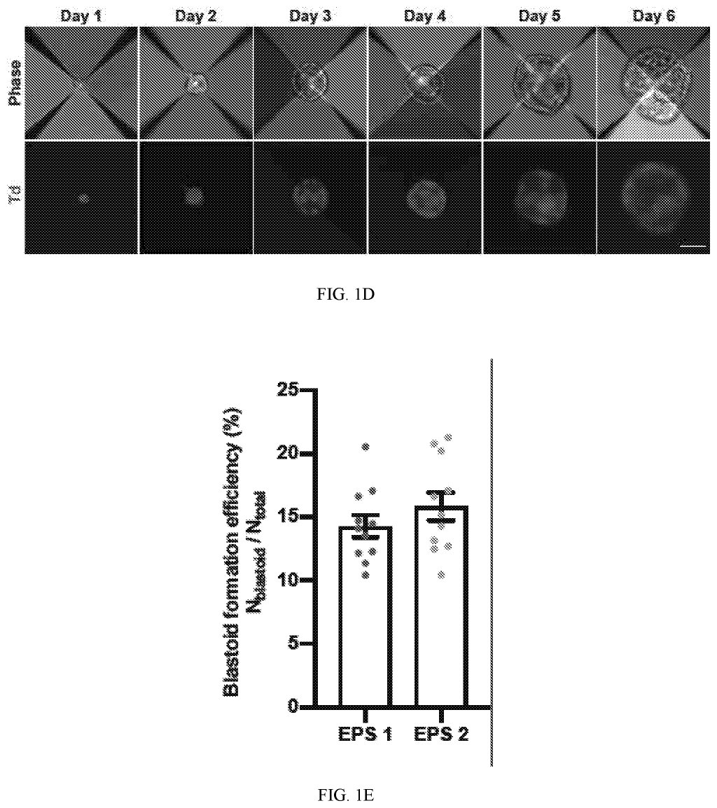

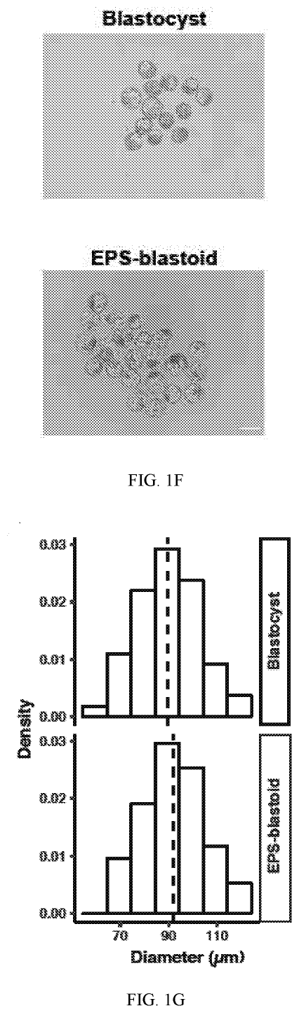

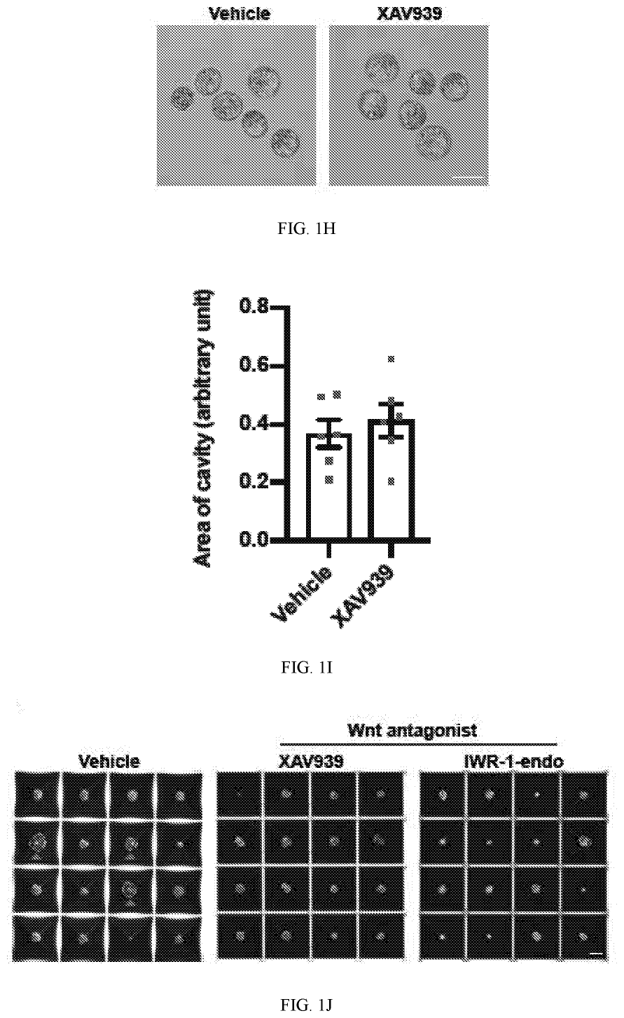

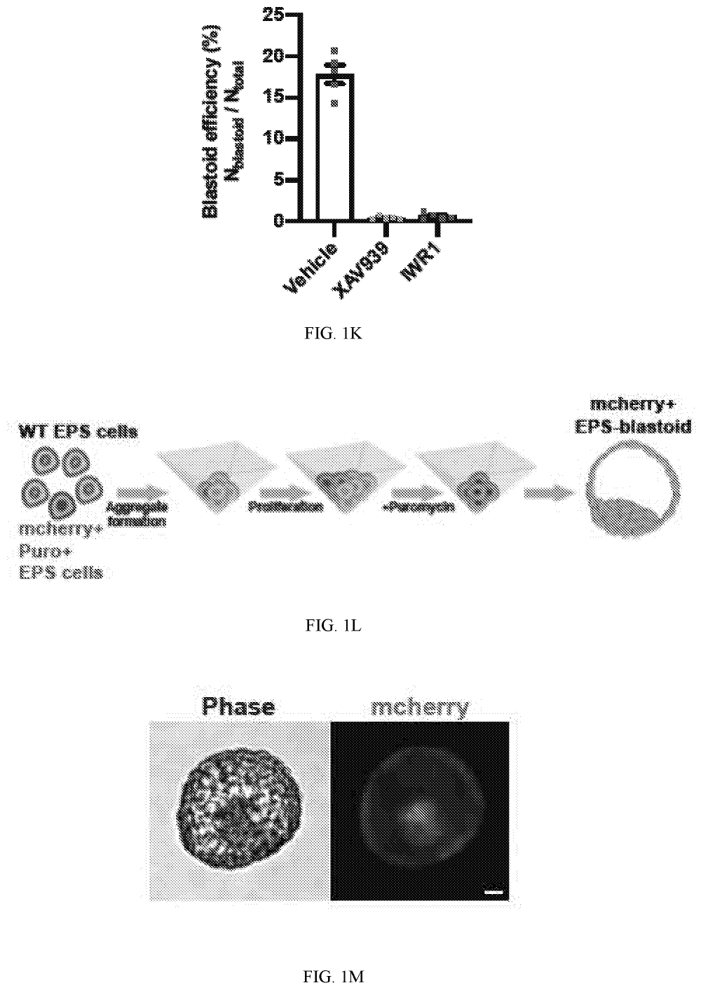

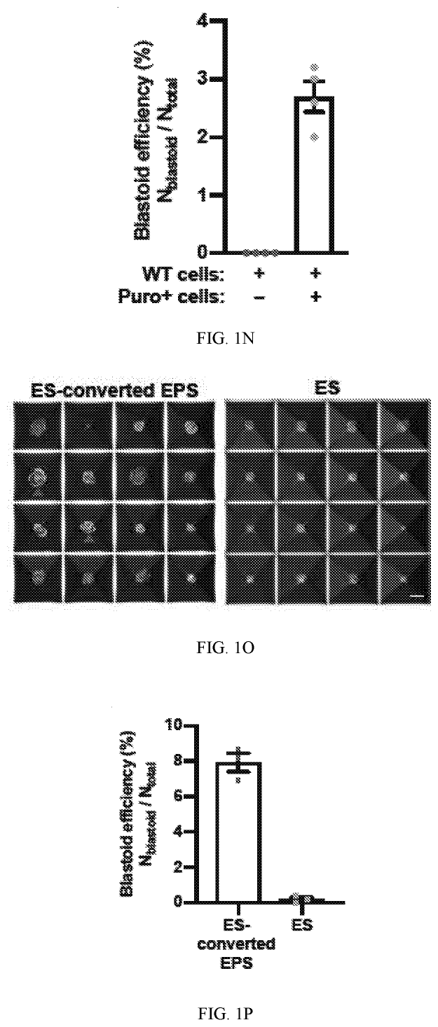

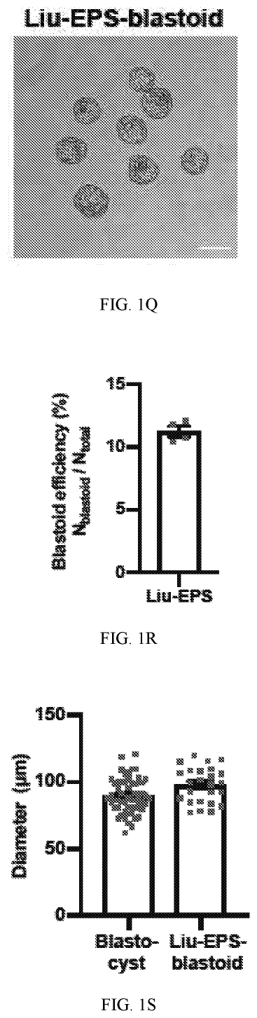

FIG. 1A shows a 3D Differentiation System for Generating Blastocyst-like Structures from EPS Cells. The top panel is a diagram showing that a single EPS cell contributes to both embryonic (Em) and extraembryonic (ExEm) lineages in the blastocyst after injection into an 8-cell embryo. Bottom panel: A diagram showing that EPS cells differentiate and self-organize into an EPS-blastoid.

Indeed, the present disclosure provides for use of single EPS which differentiates and self-organize into blastocyst-like structures which comprises all three blastocyst lineages.

FIG. 1B shows phase contrast images of EPS cell aggregates cultured in the indicated medium conditions for four days. The red triangle indicates an EPS-blastoid.

FIG. 1C shows phase contrast images of multicellular structures in microwells after five days in the blastoid induction medium containing with either KSOM (left) or M16 (right) embryo culture medium. The red triangles indicate EPS-blastoids.

In embodiments, differentiation conditions are modified by addition of, as examples, different growth factors, cytokines, and small molecules. These additions enable the generation of structures mimicking blastocysts (EPS-blastoids), which contain a cavity and an inner cell mass. As described herein, combinations of FGF4, Heparin, BMP4, CHIR99021, and/or A83-01 provide high efficiency EPS-blastoid formation. Each of these additions may be added alone or in any combination thereof. EPS cells treated with Y-27632, a Rho kinase (ROCK) inhibitor, on the day of seeding enhances cell survival. In embodiments, combinations of the additions provide emergence of EPS-blastoids around three days after cell seeding.

FIG. 1D shows representative phase contrast (upper panel) and fluorescence images (lower panel) of EPS cell aggregates at the indicated time point showing the formation of EPS-blastoids. Phase, phase contrast; Td, tdTomato.

In embodiments, EPS-blastoids enlarge and acquire an early blastocyst-like size at around day five or day six.

FIG. 1E shows quantification of EPS-blastoids formation efficiency. n=11 independent assays for each EPS cell line.)

. The average diameter of EPS-blastoids produced according to embodiments provided herein are comparable to that of E3.5 blastocysts.

FIG. 1F shows phase contrast images of E3.5 blastocysts (upper panel) and EPS-blastoids (lower panel).

FIG. 1G shows histograms showing the distribution of the diameters of E3.5 blastocysts (upper panel) and EPS-blastoids (lower panel). n=55 blastocysts and n=95 EPS-blastoids. The vertical dotted line denotes the mean of the group.

WNT/β-catenin signaling pathway is dispensable for blastocyst formation and , the WNT antagonists, XAV939 or IWR-1-endo, significantly inhibited EPS-blastoids formation.

FIG. 1H shows phase contrast images of mouse embryos 48hrs after treating with either vehicle (left) or XAV939 (5 μM) (right) at the 4-cell stage. Scale bar, 100 μm.

FIG. 1I shows quantification of the cavity area in the mouse embryos shown in FIG. 1H. Data are represented as mean±SEM; n=6 embryos in each group. FIG. 1J shows phase contrast images of multicellular structures in microwells after five days in blastoid induction medium supplemented with vehicle (left), XAV939 (middle), or IWR-1-endo (right). The red triangles indicate EPS-blastoids. Scale bar, 100 μm.

FIG. 1K shows quantification of EPS-blastoids formation efficiency with the indicated treatment. Data are represented as mean±SEM; n=5 independent assays for each group.

In embodiments, a single EPS cell gives rise to an entire EPS-blastoid.

FIG. 1L is a diagram showing the strategy for a single EPS cell to generate a clonal EPS-blastoid. FIG. 1M shows phase contrast (left) and fluorescent (right) images of an EPS-blastoid generated using the strategy shown in FIG. 1L.

FIG. 1N shows quantification of EPS-blastoids formation efficiency in the assay described in FIG. 1L. n=4 independent assays. Data are represented as mean±SEM. Scale bars, 50 μm (FIG. 1B and FIG. 1D), 100 μm (FIG. 1F), 20 μm (FIG. 1M).

The extended pluripotency of EPS cells are helpful for generating blastoids, when compared to use of ES cells.

FIG. 1O shows phase contrast images of multicellular structures in microwells after five days of blastoid induction from ES-converted EPS (left) or ES (right) cells. The red triangles indicate EPS-blastoids. Scale bar, 100 μm.

FIG. 1P shows quantification of EPS-blastoids formation efficiency for ES-converted EPS or ES cells. Data are represented as mean±SEM; n=3 independent assays for each group. EPS cells generated and cultured under conditions developed by Pengtao Liu's group (referred to as Liu-EPS; Yang et al., 2017a) also generate EPS-blastoids.

FIG. 1Q shows a phase contrast image of blastoids generated from Liu-EPS cells. Scale bar, 100 μm.

FIG. 1R shows quantification of EPS-blastoids formation efficiency from Liu-EPS cells. Data are represented as mean±SEM; n=4 independent assays.

FIG. 1S shows quantification of the diameter of blastocyst or Liu-EPS-blastoids. n=55 blastocysts; n=25 Liu-EPS-blastoids.

Key cellular and molecular events characteristic of early preimplantation development are recapitulated during EPS-blastoid formation.

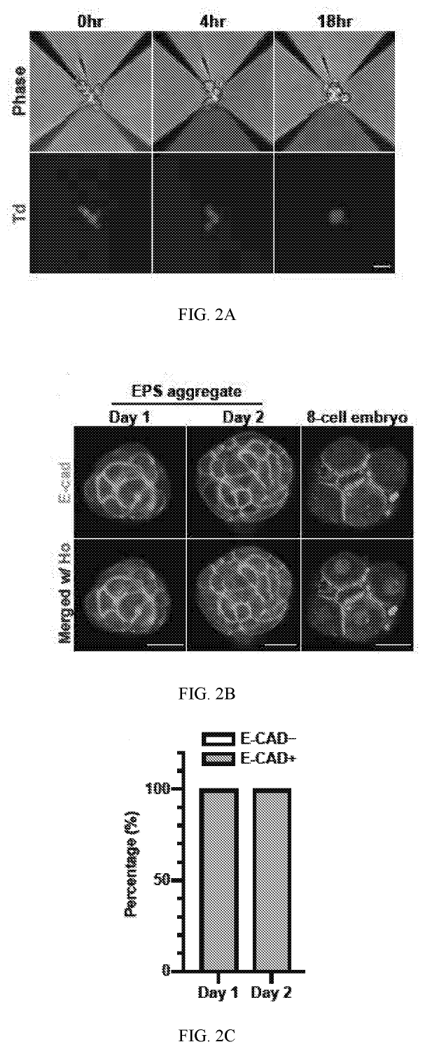

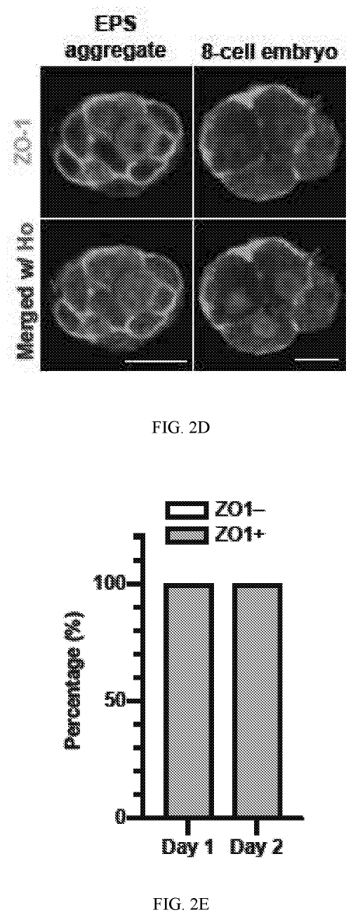

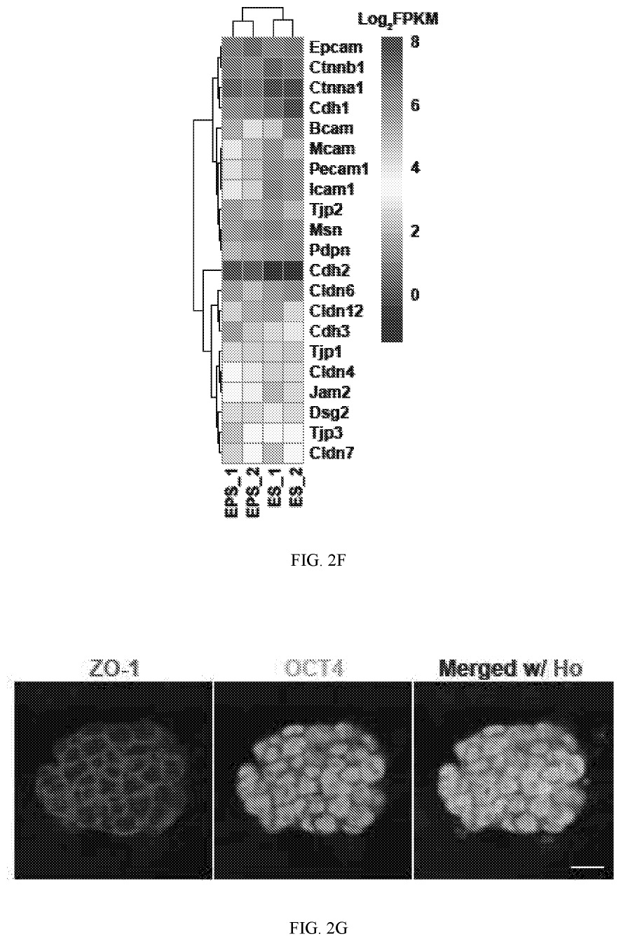

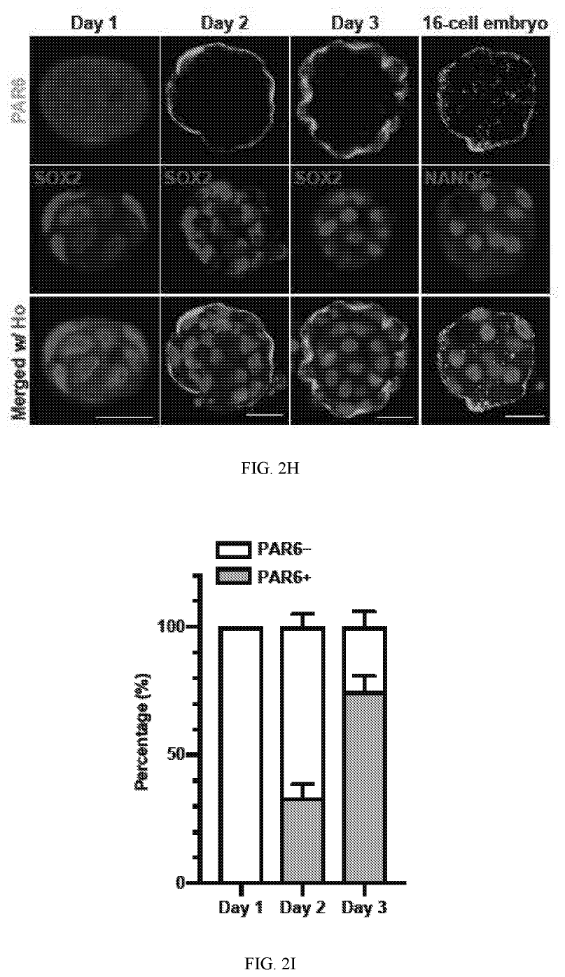

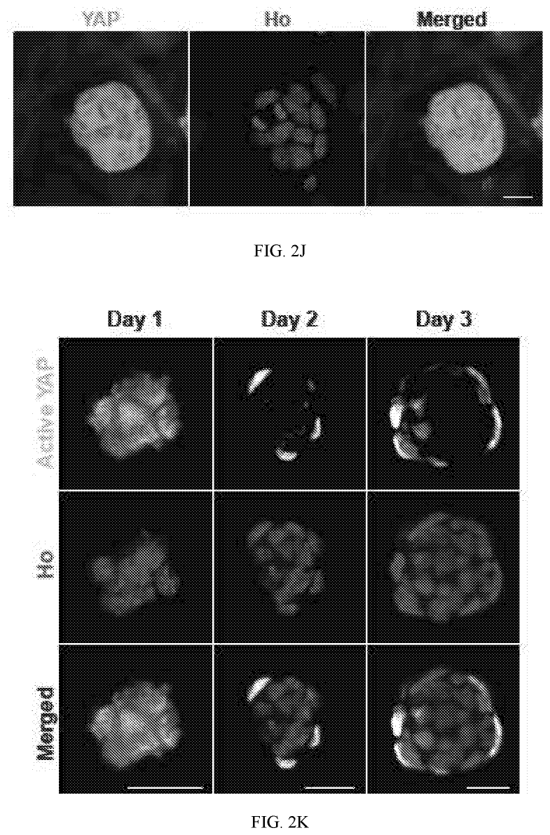

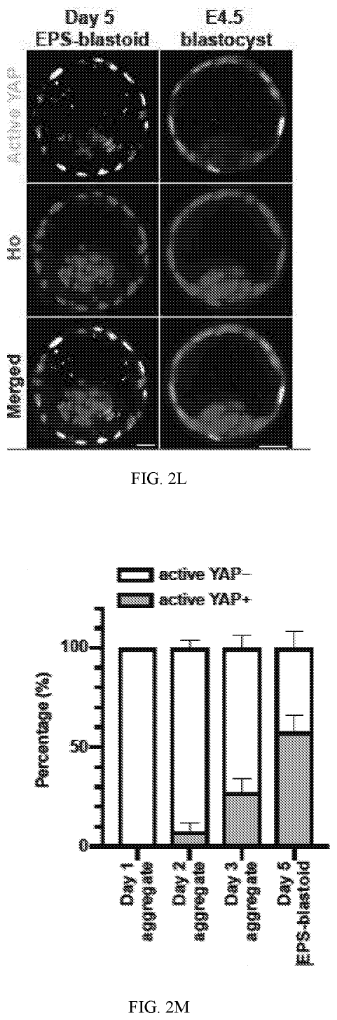

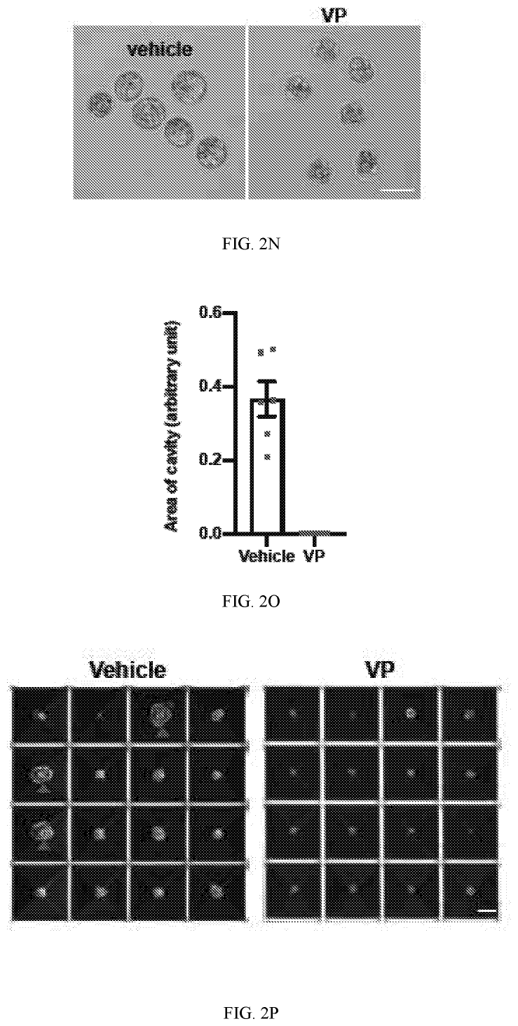

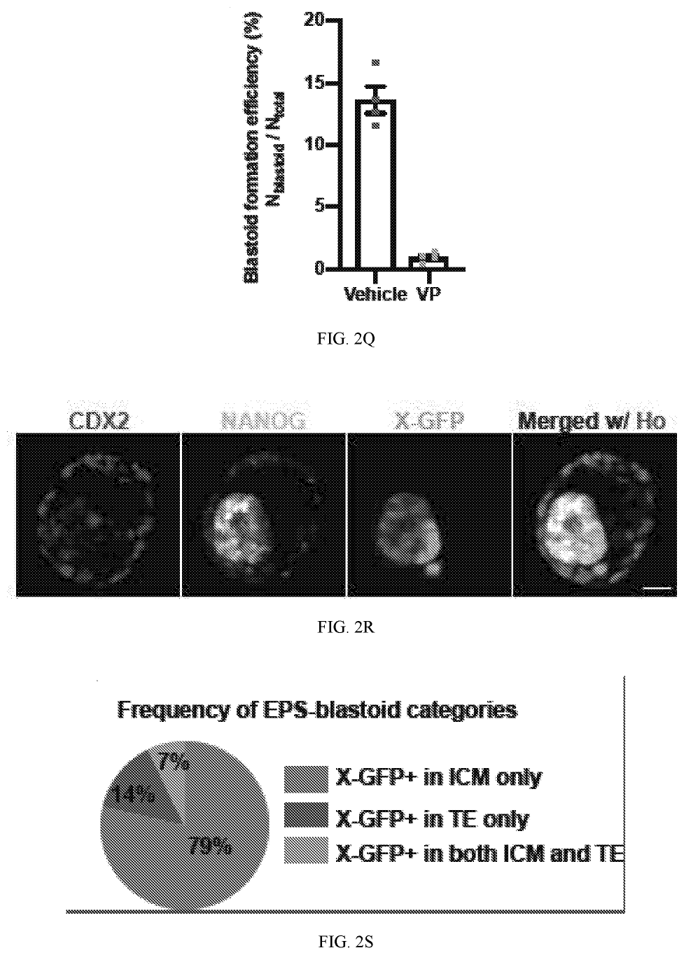

FIG. 2A shows phase contrast (upper panel) and fluorescent (lower panel) images of EPS cells at the indicated time after cell seeding. Td, tdTomato.

FIG. 2B shows immunofluorescence staining of an EPS aggregate at day 1 (left), day 2 (middle), and a compacted 8-cell embryo (right) for E-cadherin (E-CAD).

FIG. 2C shows quantification of the percentage of cell aggregates showing E-CAD+ staining at the indicated day. n=3 biological replicates for each time point.

FIG. 2D shows immunofluorescence staining of an EPS aggregate at day 1 (left) and a compacted 8-cell embryo (right) for ZO1. Ho, Hoechst. Scale bars, 20 μm.

FIG. 2E shows quantification of the percentage of cell aggregates showing ZO1+ staining at day 1 or day 2. Data are represented as mean±SEM; n=3 biological replicates for each time point.

FIG. 2F shows a heatmap showing the FPKM values of the indicated genes in two EPS and ES cell lines. FPKM, Fragments Per Kilobase of transcript per Million mapped reads.

FIG. 2G shows immunofluorescence staining of 2D EPS cells for ZO1 and OCT4. Ho, Hoechst. Scale bar, 50 μm.

Like blastocysts, EPS cell aggregates undergo polarization and recapitulate the process of polarization characteristic of early preimplantation development.

FIG. 2H shows immunofluorescence staining of EPS aggregates at the indicated time points and 16-cell embryos for PAR6 and SOX2 or NANOG.

FIG. 2I shows quantification of the percentage of cell aggregates showing a PAR6 ring at the indicated time points. n=3 biological replicates for each time point.

Like blastocysts, EPS-blastoid formation also requires an intact Hippo/YAP signaling pathway.

FIG. 2J shows immunofluorescence staining of 2D EPS cells for YAP (FIG. 2I). Ho, Hoechst. Scale bar, 50 μm.

FIG. 2K shows immunofluorescence staining for active YAP in EPS aggregates at the indicated time point.

FIG. 2L shows immunofluorescence staining for active YAP in an EPS-blastoid and an E4.5 blastocyst.

FIG. 2M shows quantification of the percentage of structures showing active YAP+ at the indicated time points. n=3 independent assays for day 1, day 2 aggregates, and day 5 EPS-blastoids; n=5 independent assays for day 3 aggregates. Data are represented as mean±SEM. Scale bars, 20 μm. Ho, Hoechst.

FIG. 2N shows phase contrast images of mouse embryos 48 hrs after treating with either vehicle (left) or VP (right) at the 4-cell stage. Scale bar, 100 μm. VP, verteporfin.

FIG. 2O shows quantification of the cavity area in the mouse embryos shown in (FIG. 2K). Data are represented as mean±SEM; n=6 embryos in each group.

FIG. 2P shows phase contrast images of multicellular structures in microwells after five days of blastoid induction in medium supplemented with vehicle (left) or VP (right). The red triangles indicate EPS-blastoids. Scale bar, 100 μm. VP, verteporfin.

FIG. 2Q shows quantification of EPS-blastoids formation efficiency with the indicated treatment. Data are represented as mean±SEM; n=4 independent assays for each group.

EPS-blastoid formation recapitulates key molecular and cellular processes characteristic of early preimplantation development. For example, cells of the EPS-blastoid gradually reactivate inactivated paternal X chromosome.

FIG. 2R shows immunostaining of an EPS-blastoid from a paternal X-GFP cell line for CDX2, NANOG, and X-GFP. Ho, Hoechst. Scale bar, 20 μm.

FIG. 2S shows quantification of the frequency of different EPS-blastoid categories based on paternal X-GFP expression pattern. n=14 X-GFP EPS-blastoids.

The cellular composition of EPS-blastoids resembles early blastocysts. For example, EPS-blastoids possess the three lineages of blastocysts

In particular, like E3.5 mouse blastocysts, EPS blastoids have two cell lineages, the external trophectoderm (TE) layer and the internal inner cell mass (ICM).

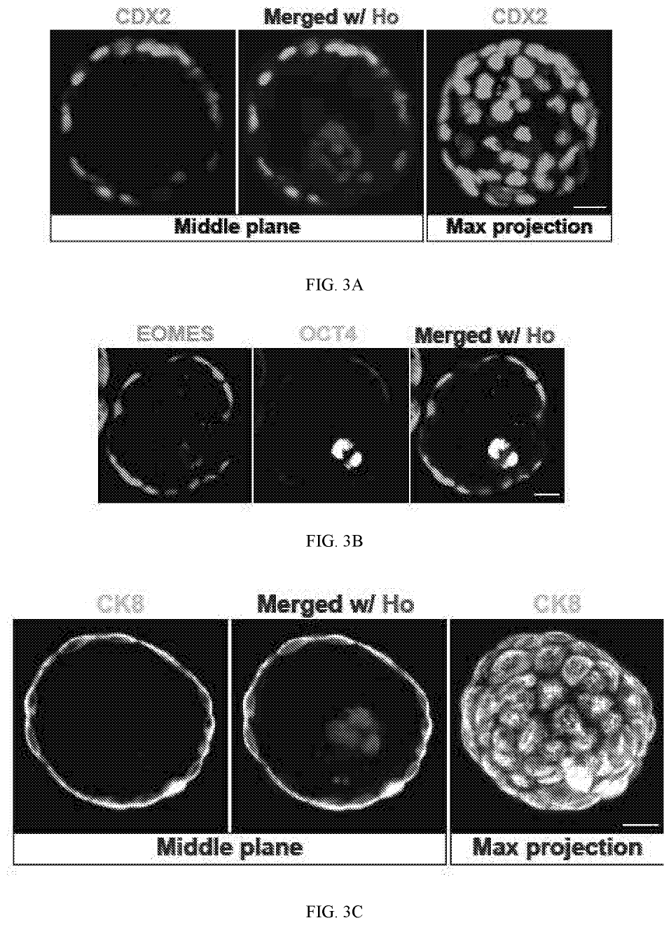

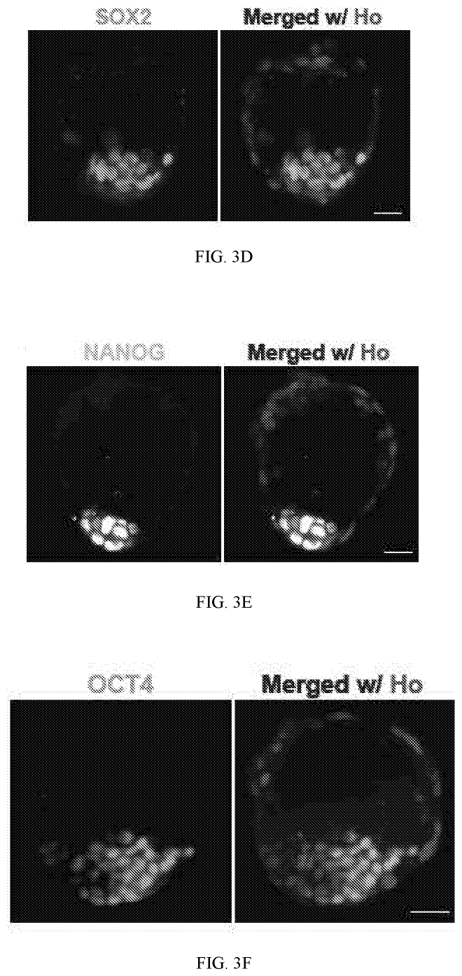

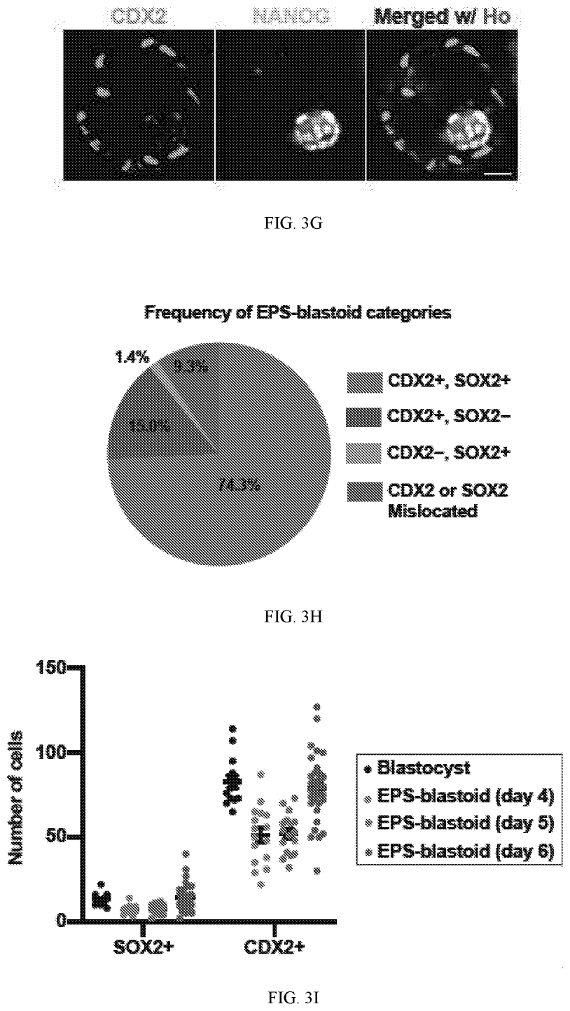

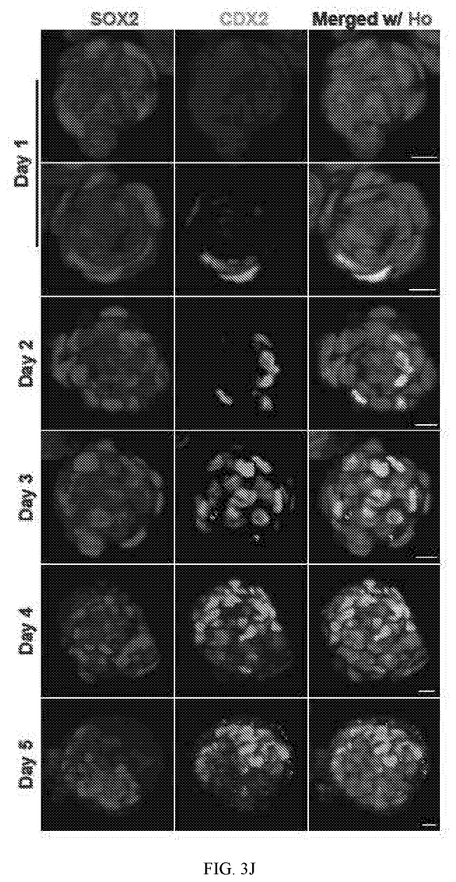

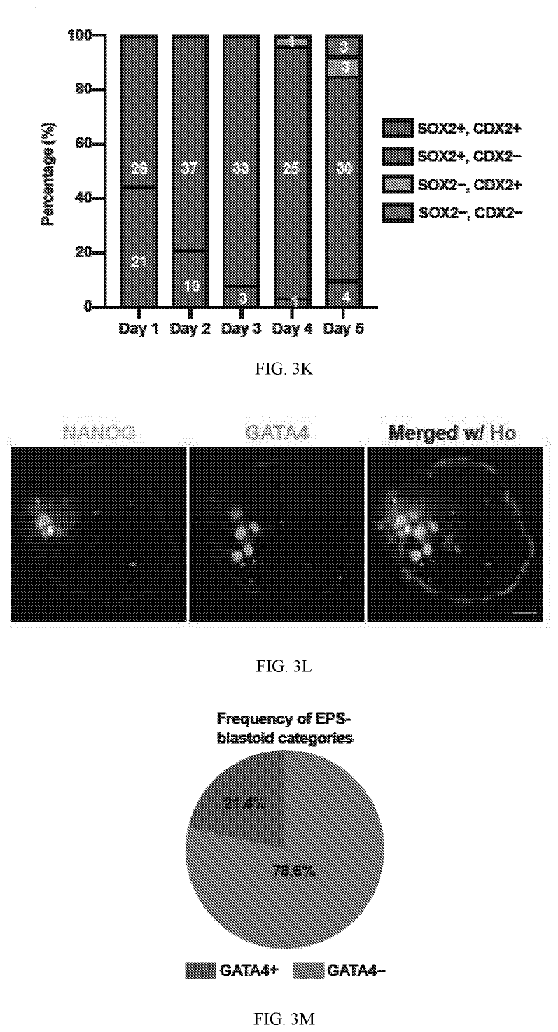

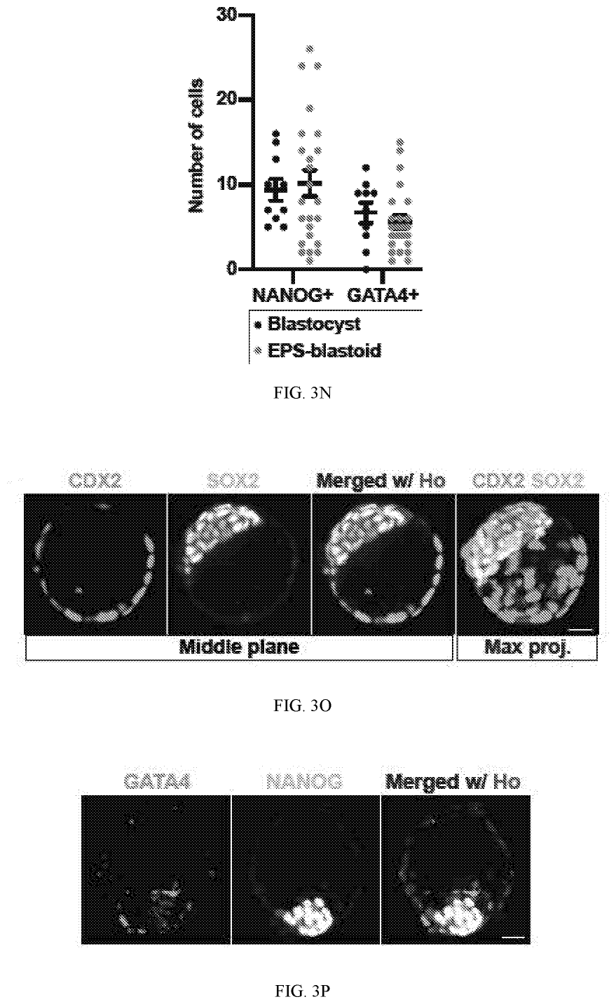

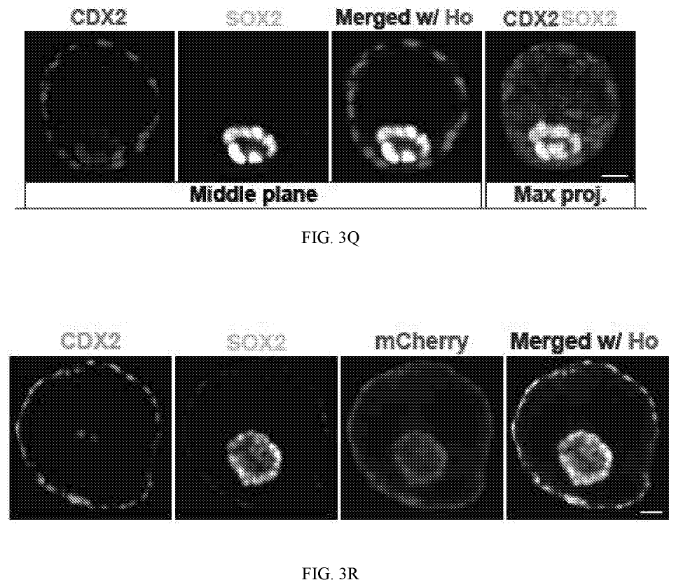

FIG. 3A shows Immunofluorescence staining of EPS-blastoids for CDX2. The rightmost panel is the maximum intensity projection of z-stack images of the indicated protein.

FIG. 3B shows immunofluorescence staining of EPS-blastoids for EOMES and OCT4. Ho, Hoechst. Scale bars, 20 μm.

FIG. 3C shows immunofluorescence staining of EPS-blastoids for CK8. The rightmost panel is the maximum intensity projection of z-stack images of the indicated protein.

FIG. 3D, FIG. 3E, and FIG. 3F show immunofluorescence staining of EPS-blastoids for SOX2 (FIG. 3D), NANOG (FIG. 3E), and OCT4 (FIG. 3F).

FIG. 3G shows immunofluorescence staining of EPS-blastoids for CDX2 and NANOG. Ho, Hoechst. Scale bars, 20 μm.

FIG. 3H shows quantification of the frequency of different EPS-blastoid categories based on CDX2 and SOX2. n=140 EPS-blastoids.

Like blastocysts, the TE and ICM lineages segregate during EPS-blastoid formation.

FIG. 3I shows quantification of the number of cells with SOX2+ or CDX2+ staining in the ICM or TE compartment, respectively, of the indicated samples. n=14 blastocysts, 16 EPS-blastoids at day 4, 17 EPS-blastoids at day 5, and 34 EPS-blastoids at day 6.

FIG. 3J shows immunofluorescence staining of EPS aggregates at the indicated day for SOX2 and CDX2 expression. Ho, Hoechst. Scale bars, 10 μm.

FIG. 3K shows quantification of different patterns of SOX2 and CDX2 expression in EPS cell aggregates at the indicated day. n=47, 47, 36, 27, and 40 for EPS cell aggregates at day 1, 2, 3, 4, and 5, respectively.

Similar to how early blastocysts further develop, the ICM of EPS-blastoids segregates into two lineages: epiblast (EPI) cells and primitive endoderm (PE) cells.

FIG. 3L shows immunofluorescence staining of an EPS-blastoid for NANOG and GATA4.

FIG. 3M shows quantification of the frequency of EPS-blastoids with or without GATA4+ PE-like cells. n=112 EPS-blastoids.

FIG. 3N shows quantification of the number of cells with NANOG+ or GATA4+ staining in the EPI- or PE-like compartment, respectively, of blastocysts or EPS-blastoids. n=15 blastocysts and 24 EPS-blastoids. Data are represented as mean±SEM. Scale bars, 20 μm. Ho, Hoechst.

EPS-blastoids exhibit blastocyst-like allocation of cell lineages. For example, they exhibit CDX2, SOX2/NANOG, and GATA4 staining consistent with blastocysts.

FIG. 3O shows immunofluorescence staining of ES-converted EPS-blastoids for CDX2 and SOX2. Ho, Hoechst. The rightmost panel is the maximum intensity projection of z-stack images of the indicated protein. Scale bars, 20 μm.

FIG. 3P shows immunofluorescence staining of ES-converted EPS-blastoids for GATA4 and NANOG. Ho, Hoechst. Scale bars, 20 μm.

FIG. 3Q shows immunofluorescence staining of a Liu-EPS-blastoid for CDX2 and SOX2. Ho, Hoechst. The rightmost panel shows the maximum intensity projection of z-stack images of the indicated protein. Scale bars, 20 μm.

FIG. 3R shows immunofluorescence staining of an EPS-blastoid generated from a single EPS cell for CDX2, SOX2, and mCherry. Ho, Hoechst. Scale bars, 20 μm.

RNA expression of EPS-blastoids more resembled blastocysts than morulae.

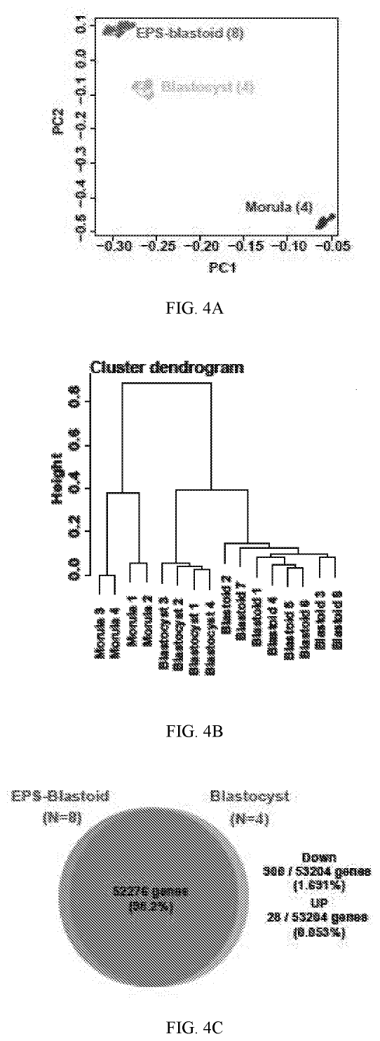

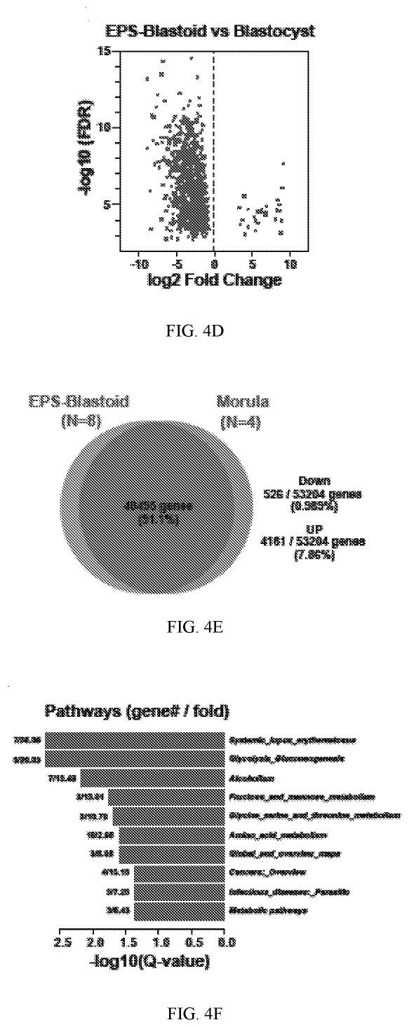

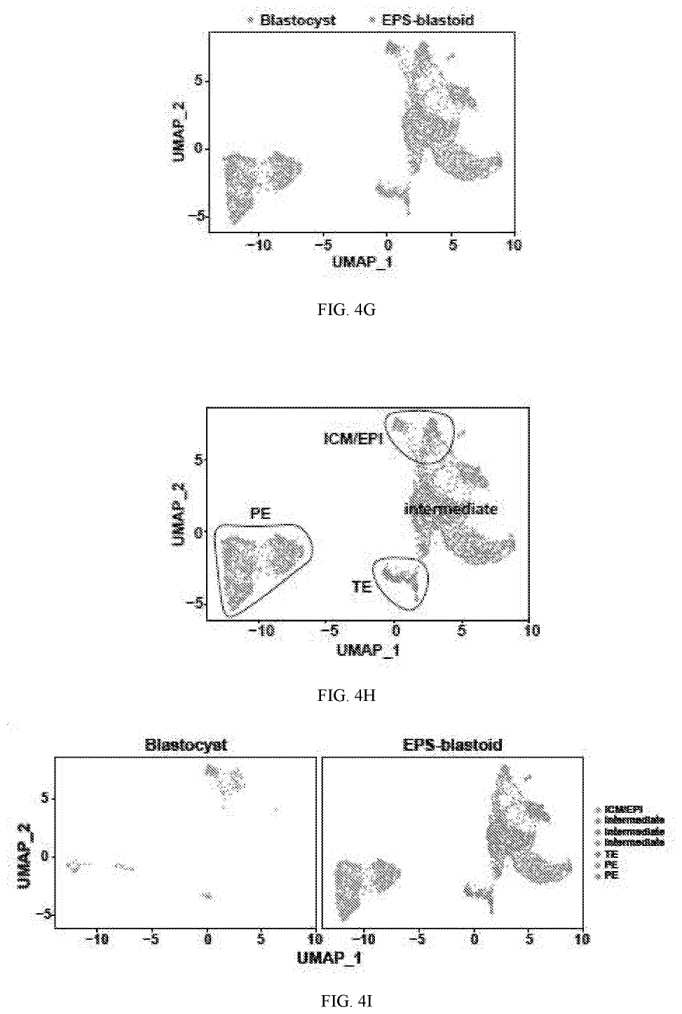

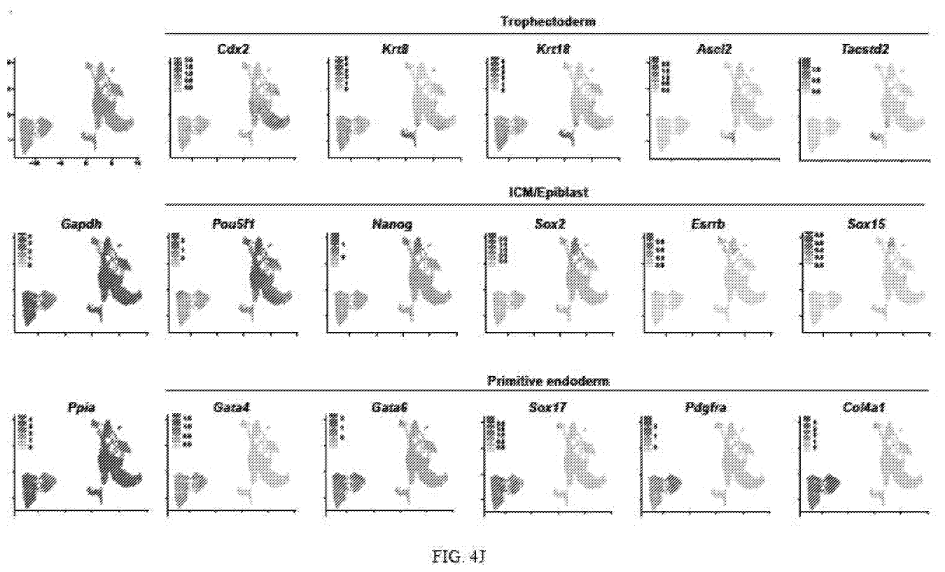

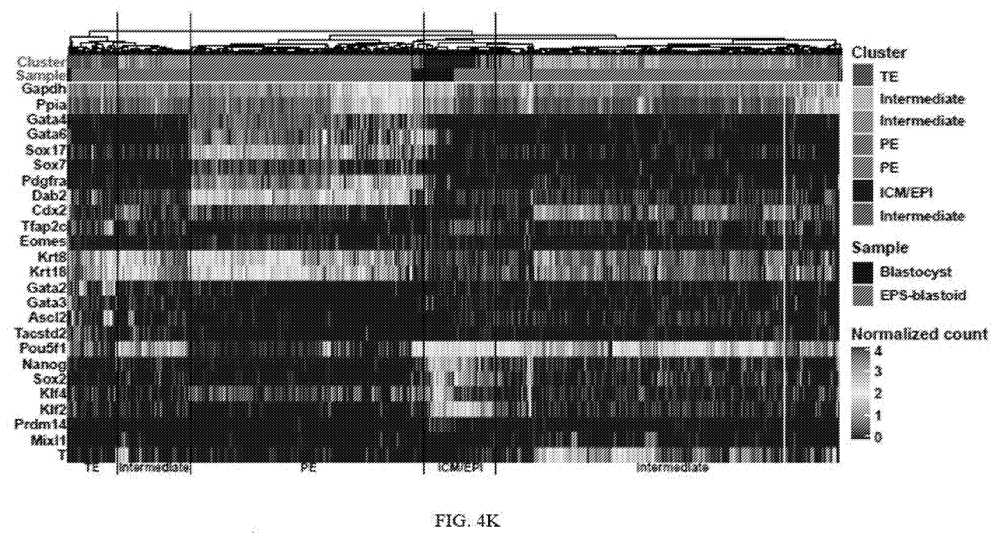

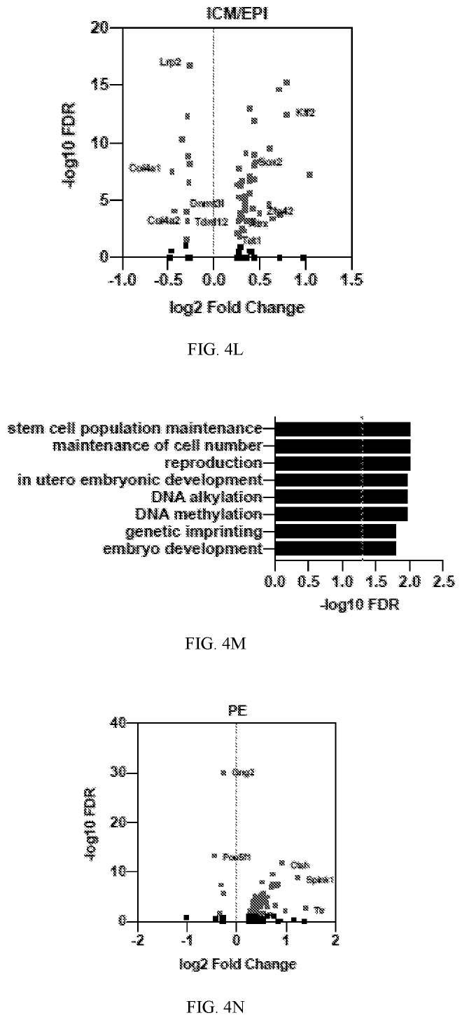

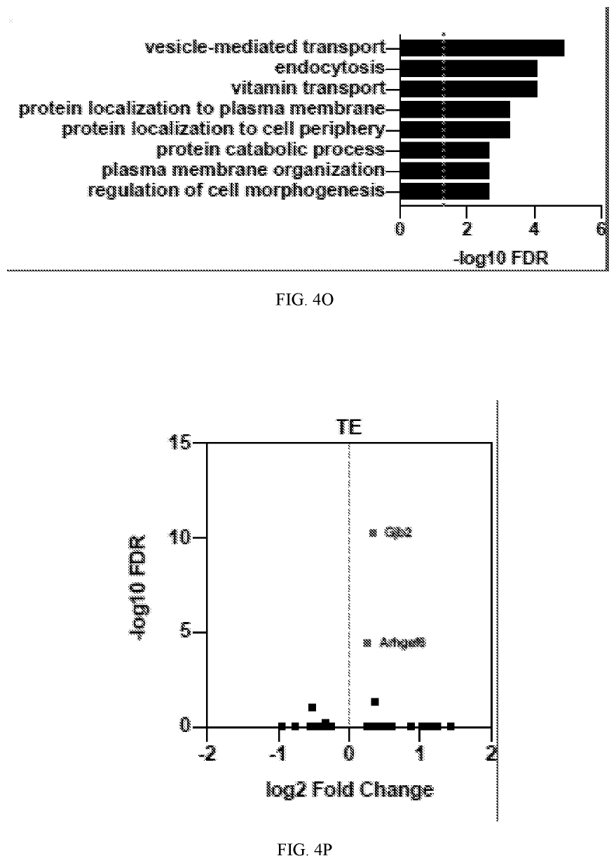

FIG. 4A show a principle component analysis (PCA) of bulk RNA-Seq data from individual EPS-blastoid, blastocyst, and morula. The number of biological replicates in each group was shown inside the parenthesis.

FIG. 4B shows unsupervised average clustering analysis of RNA-Seq data from individual EPS-blastoid, blastocyst, and morula.

FIG. 4C shows summary of differential gene expression analysis between EPS-blastoids and blastocysts.

FIG. 4D shows a volcano plot showing the differentially expressed genes (DEGs) between EPS-blastoids and blastocysts.

FIG. 4E shows a summary of differential gene expression analysis between EPS-blastoids and morulae.

FIG. 4F shows pathways enrichment analysis of DEGs between EPS-blastoids and blastocysts.

FIG. 4G shows a Uniform Manifold Approximation and Projection (UMAP) plot of 2702 cells from blastocysts and EPS-blastoids after alignment using the Seurat package.

FIG. 4H shows a UMAP plot showing the clustering of all cells. The identities of each cluster were determined based on the expression of the lineage markers.

FIG. 4I shows a UMAP plot showing the clustering of cells from blastocysts (left) or EPS-blastoids (right), respectively.

FIG. 4J shows the expression of lineage-specific genes shown in UMAP plots.

EPS-blastoids contain all three blastocyst cell lineages. FIG. 4K shows an Unsupervised clustering analysis showing the cells of similar lineage identities cluster to each other regardless of sample type. The cluster row indicates the subpopulation defined in (FIG. 4H). The sample row indicates the sample type.

FIG. 4L, FIG. 4N, and FIG. 4P show Dot plots showing the differentially expressed genes (DEGs) in the ICM/EPI lineage (FIG. 4L), PE lineage (FIG. 4N), and TE lineage (FIG. 4P) between blastocysts and EPS-blastoids. Genes with FDR exceeding the statistical significance cutoff (FDR <0.05) are labeled with red color.

FIG. 4M and FIG. 4O show Gene ontology analysis of biological functions for DEGs in the ICM/EPI lineage (FIG. 4M) and PE lineage (FIG. 4O) between blastocyst and EPS-blastoids. Red dotted line indicates the cutoff (FDR<0.05).

Like blastocysts, ESCs, TSCs, and XEN cells, which are considered the in vitro counterparts of EPI, TE, and PE lineages are derivable from EPS-blastoids. Also, cells of the EPS-blastoids express genes consistent with natural blastocysts.

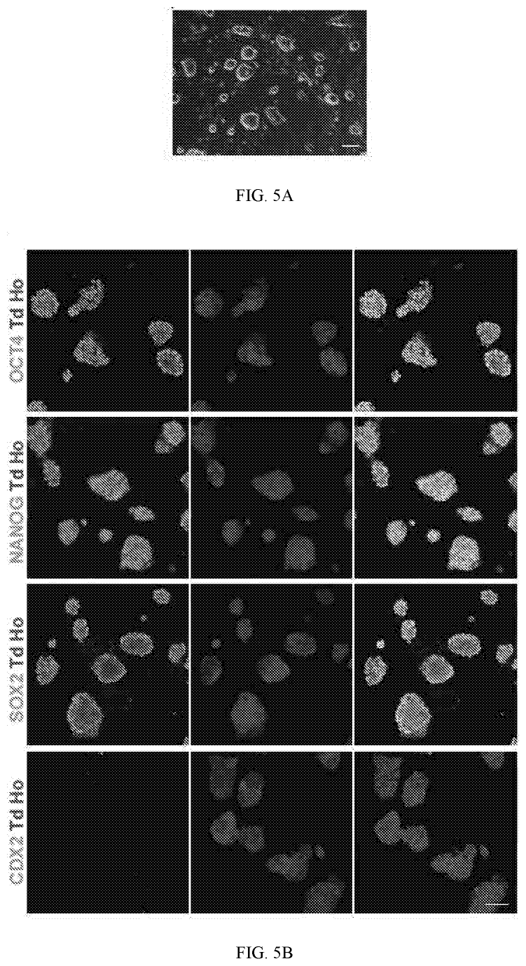

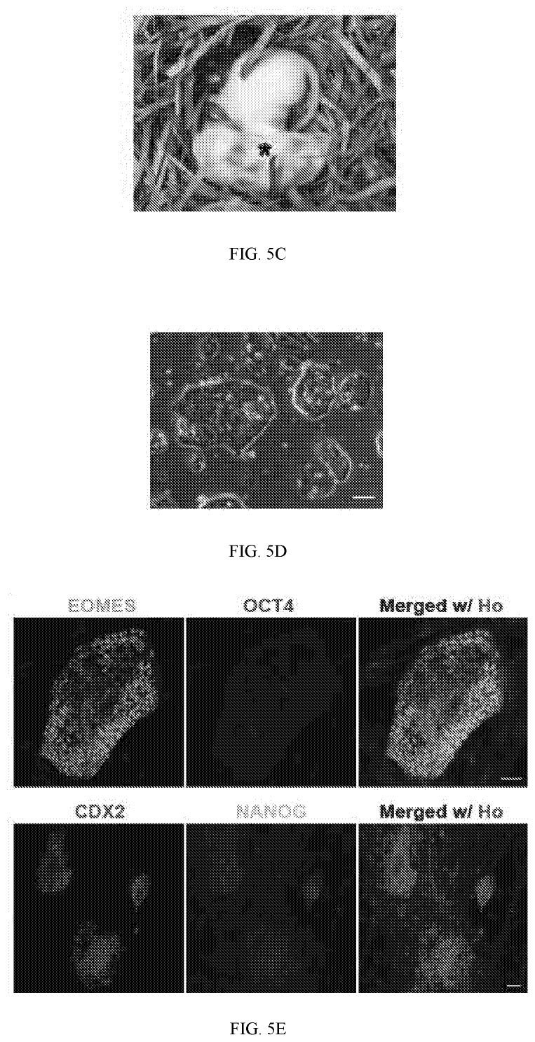

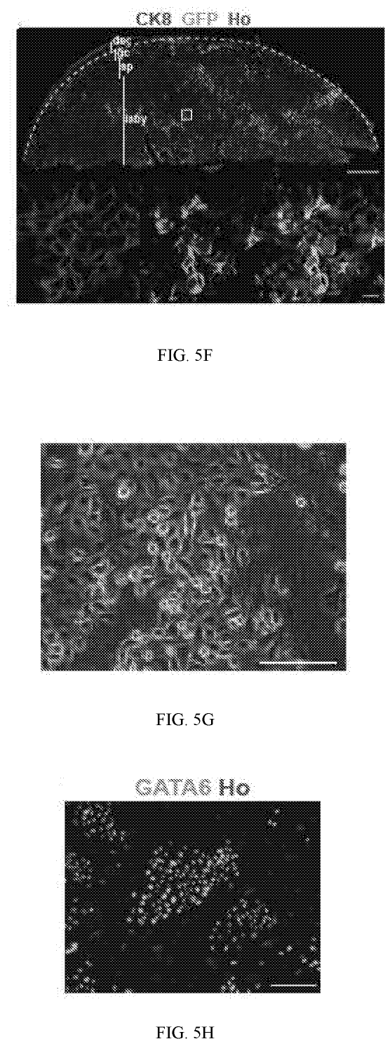

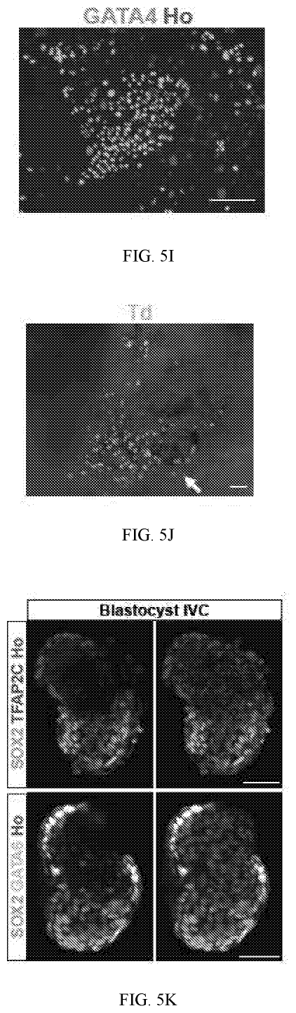

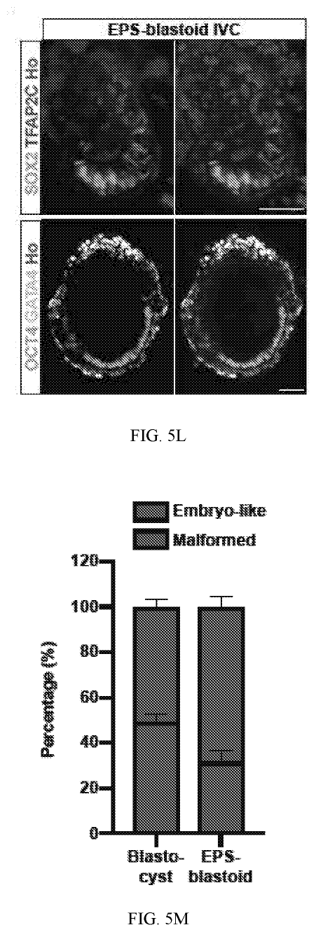

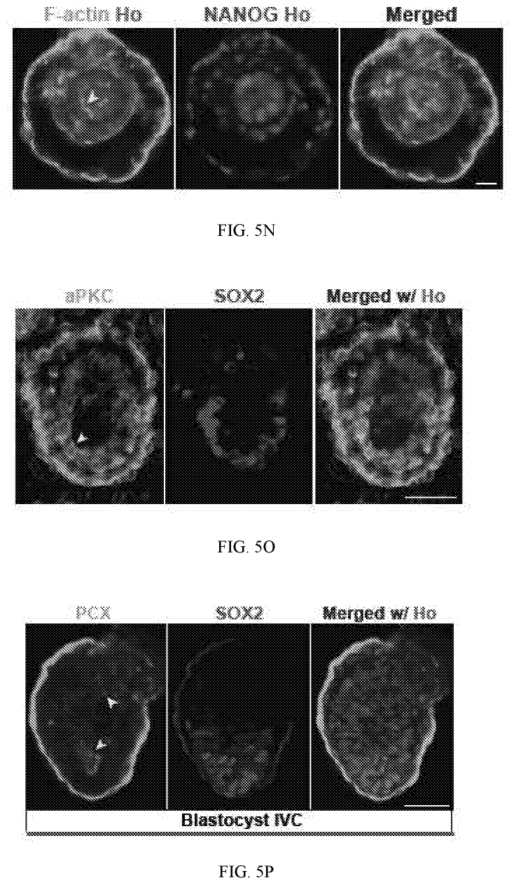

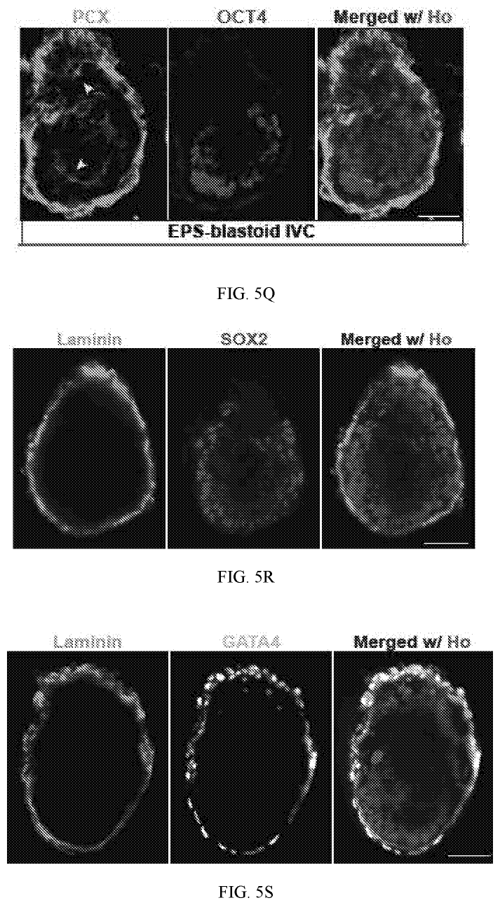

FIG. 5A shows a phase contrast image of de novo derived ES cell lines from EPS-blastoids.

FIG. 5B shows immunofluorescence staining of ES cells derived from EPS-blastoids for OCT4, NANOG, SOX2, and CDX2. Scale bar, 50 μm.

FIG. 5C shows a brightfield image of two littermates generated from blastocyst injected with EPS-blastoid-derived ES cells showing that these cells contribute to a chimeric mouse. The star symbol denotes a chimeric mouse.

FIG. 5D shows phase contrast image of de novo derived TS cell lines from EPS-blastoids.

FIG. 5E shows immunofluorescence staining of TS cells derived from EPS-blastoids for EOMES, CDX2, OCT4, and NANOG. Scale bar, 100 μm.

FIG. 5F shows immunofluorescence staining of a placental section for CK8 and GFP. The panels below are the enlargement of the yellow boxed region. The placenta was delineated by a dotted line; dec, decidua layer; gc, giant cell layer; sp, spongiotrophoblast layer; laby, labyrinth layer.

FIG. 5G shows phase contrast image of de novo derived XEN cell lines from EPS-blastoids.

FIG. 5H and FIG. 5I show immunofluorescence staining of EPS-blastoids-derived XEN cells for GATA6 (FIG. 5H) and GATA4 (FIG. 5I). Ho, Hoechst. Scale bar, 100 μm.

FIG. 5J shows a brightfield image of a yolk sac overlaid with tdTomato epifluorescence image showing EPS-blastoid-derived XEN cells contribute to the developing yolk sac. Td, tdTomato.

FIG. 5K shows immunofluorescence staining of blastocyst-derived postimplantation embryo-like structures for TFAP2C and SOX2 (upper panel) or GATA6 and SOX2 (lower panel).

FIG. 5L shows immunofluorescence staining of EPS-blastoid-derived postimplantation embryo-like structures for TFAP2C and SOX2 (upper panel) or GATA4 and OCT4 (lower panel).

FIG. 5M shows quantification of the percentage of postimplantation embryo-like and malformed structures formed after in vitro culture of blastocysts and EPS-blastoids. n=3 and 4 independent assays for blastocysts and EPS-blastoids, respectively.

Upon further cultivation, EPS-blastoids give develop into postimplantation embryo-like structures.

FIG. 5N shows immunofluorescence staining of an EPS-blastoid-derived pen-implantation embryo-like structure for F-actin and NANOG showing the formation of rosette EPI-like structure.

FIG. 5O shows immunofluorescence staining of an EPS-blastoid-derived postimplantation embryo-like structure for aPKC and SOX2. Yellow arrowhead denotes the apical domain.

FIG. 5P and FIG. 5Q show immunofluorescence staining of a blastocyst—(FIG. 5P) or an EPS-blastoid—(FIG. 5Q) derived postimplantation embryo-like structure for PCX and SOX2 (FIG. 5P) or PCX and OCT4 (FIG. 5Q). Yellow arrowheads denote the enrichment of PCX protein around the lumens in both the EPI and ExE-like structure. PCX, podocalyxin.

FIG. 5R and FIG. 5S show immunofluorescence staining of postimplantation embryo-like structures from in vitro culture of EPS-blastoids for Laminin and SOX2 (FIG. 5R), or Laminin and GATA4 (FIG. 5S). Scale bar, 50 μm.

Data above are represented as mean±SEM. Scale bar, 500 μm (FIG. 5F, upper), 100 μm (FIG. 5A, FIG. 5D, FIG. 5G, and FIG. 5J), 50 μm (FIG. 5K, FIG. 5I, FIG. 5O, FIG. 5P, and FIG. 5Q), 20 um (FIG. 5F, bottom; and FIG. 5N). Ho, Hoechst.

EPS-blastoids implant, trigger decidualization, and continue to grow inside the uterus.

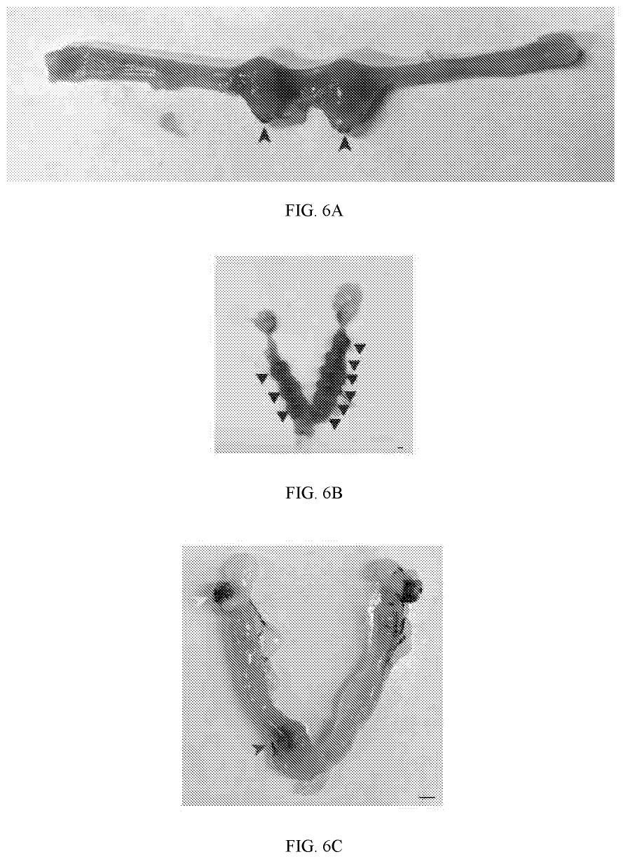

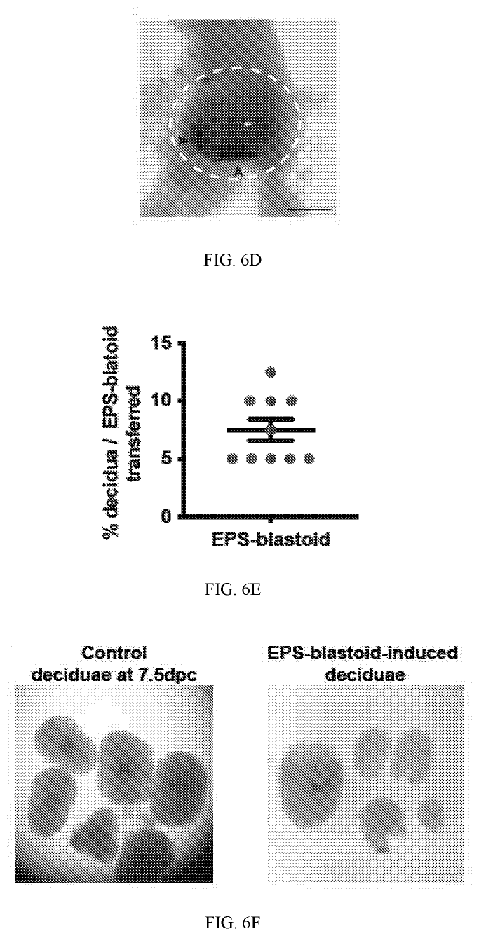

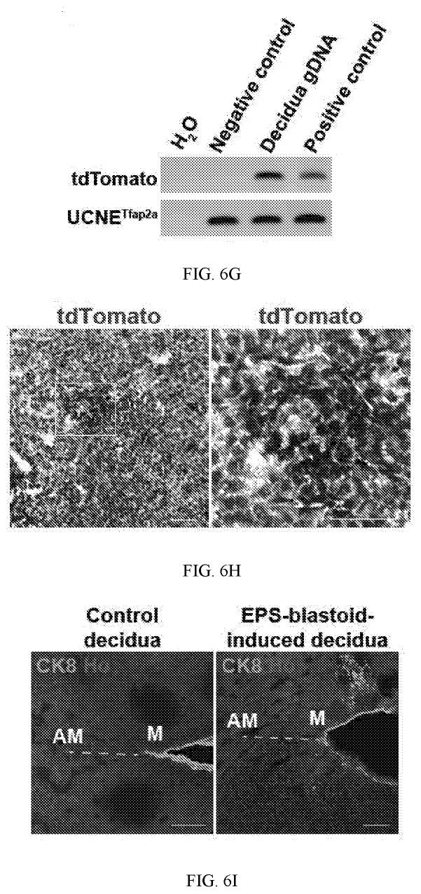

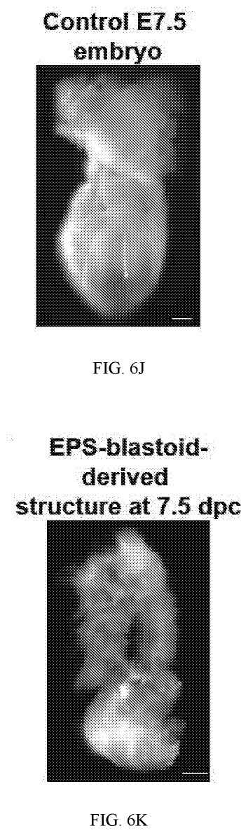

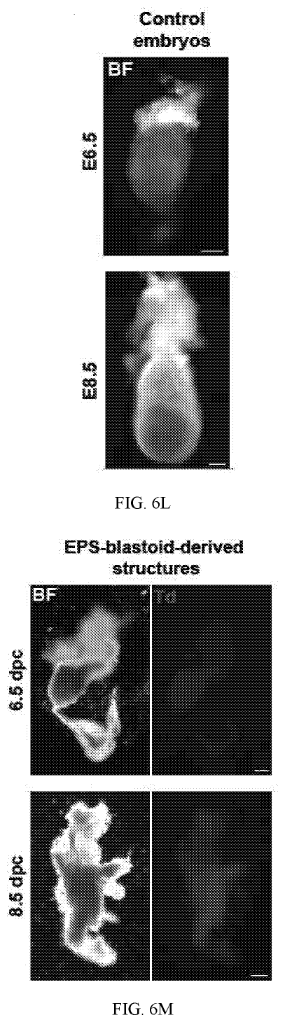

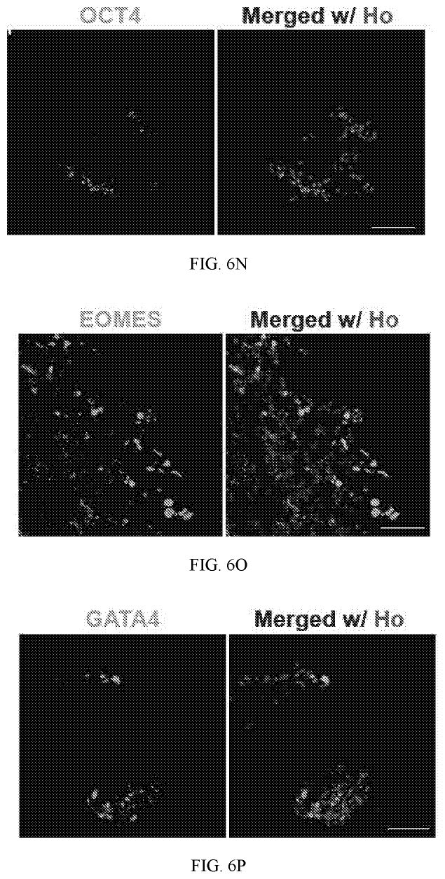

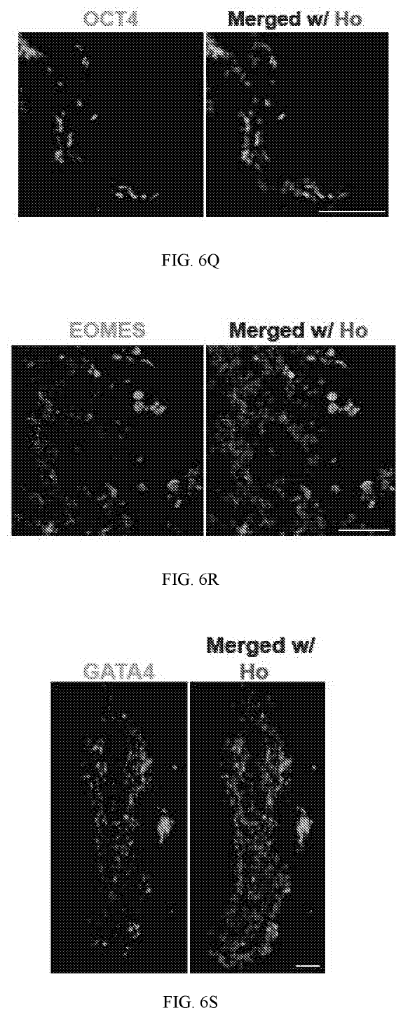

FIG. 6A shows a brightfield image showing the formation of decidua in the mouse uterus 5 days after EPS-blastoids transfer at 2.5 dpc. Black arrowheads indicate deciduae.

FIG. 6B shows a brightfield image of the uterus of a control mouse at 7.5 dpc. Black triangles indicate deciduae. Scale bars, 1 mm.

FIG. 6C shows a brightfield image of a mouse uterus 5 days after EPS-blastoids transfer at 2.5 dpc with Evan's blue staining. The red arrowhead indicates a decidua. Yellow arrowheads denote the ovaries.

FIG. 6D shows a brightfield image of a decidua (circled with dotted line) after removing the uterus wall showing blood infiltration (indicated by black arrowheads). Scale bar, 1 mm.

FIG. 6E shows quantification of the efficiency of decidua formation per EPS-blastoid transferred into the mouse uterus. Data are represented as mean±SEM; n=10 independent assays.

FIG. 6F shows a brightfield images of deciduae recovered from control 7.5 dpc mice (left) or surrogate mice at 7.5 dpc with EPS-blastoids transfer at 2.5 dpc. Scale bar, 1 mm.

FIG. 6G shows PCR analysis of genomic DNA for the tdTomato gene reveals the presence of EPS-blastoid-derived cells in the decidua tissue. UCNETfap2a was used as an internal loading control.

FIG. 6H shows immunohistochemistry analysis of decidua sections showing the decidua contains EPS-blastoid-derived tdTomato+ cells. The image on the right is the enlargement of the yellow box region.

FIG. 6I shows immunofluorescence staining of a section from control decidua (left) or EPS-blastoid-induced decidua (right) for CK8. The dotted line indicates the embryonic axis. AM, antimesometrial pole; M, mesometrial pole.

FIG. 6J and FIG. 6K show brightfield images of a control E7.5 embryo (FIG. 6J) or an in vivo EPS-blastoid-derived structure recovered from decidua at 7.5 dpc (5 days after EPS-blastoids transfer) (FIG. 6K).

FIG. 6L shows a brightfield images of control embryos at the indicated embryonic days. BF, brightfield. Scale bar, 100 μm.

FIG. 6M shows a brightfield (left) and fluorescent (right) images of EPS-blastoid-derived structures recovered from decidua at the indicated time points. EPS-blastoids were transferred at 2.5 dpc. BF, brightfield. Td, tdTomato. Scale bar, 100 μm.

FIG. 6N to FIG. 6P show Immunofluorescence staining of sections from an in vivo EPS-blastoid-derived structure recovered from decidua at 7.5 dpc (5 days after EPS-blastoids transfer) for OCT4 (FIG. 6N), EOMES (FIG. 60), and GATA4 (FIG. 6P).

FIG. 6Q to FIG. 6S show immunofluorescence staining for OCT4 (FIG. 6Q), EOMES (FIG. 6R), and GATA4 (FIG. 6S) in tissue sections of an in vivo EPS-blastoid-derived structure recovered from decidua at 6.5 dpc (4 days after EPS-blastoids transfer). Ho, Hoechst. Scale bar, 50 μm.

Scale bar, 1 mm (FIG. 6A and FIG. 6C), 100 μm (FIG. 6H to FIG. 6K), and 50 μm (FIG. 6N to

Induced EPS (iEPS) cells can be used to generate iEPS-blastoids. iEPS-blastoids generated from adult somatic cells are similar to those from embryo-derived stem cells. Similar to EPS-blastoids, iEPS-blastoids also morphologically resemble natural blastocysts and are of similar size as E3.5 blastocysts.

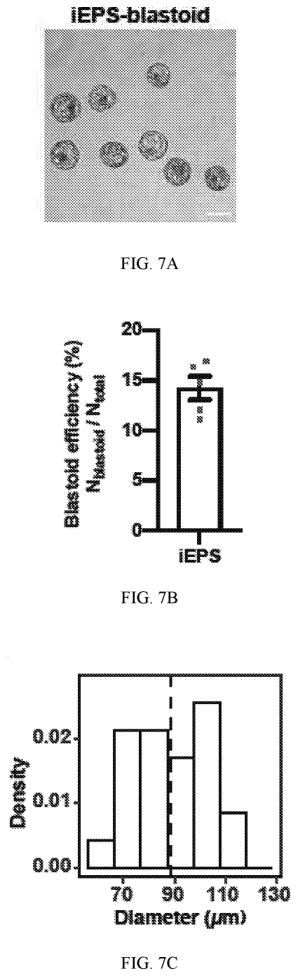

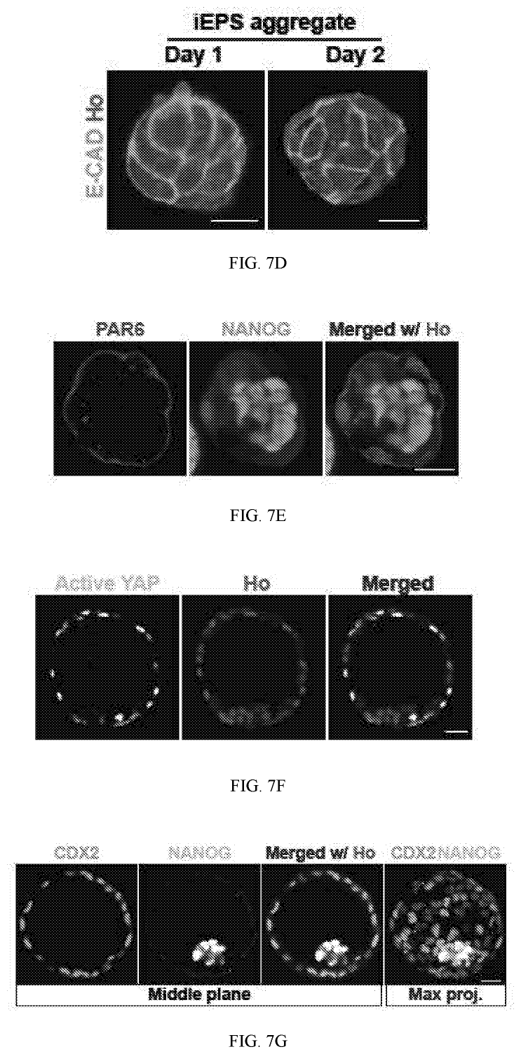

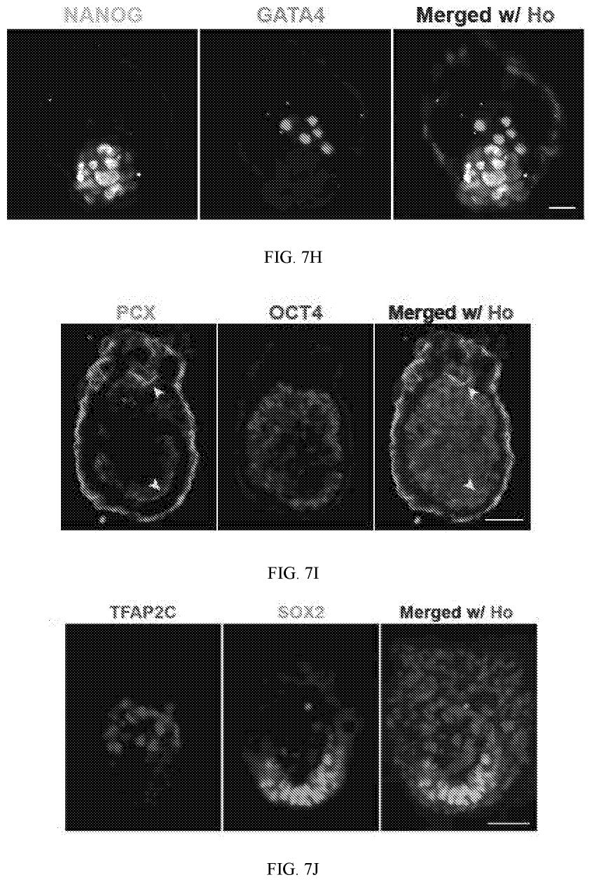

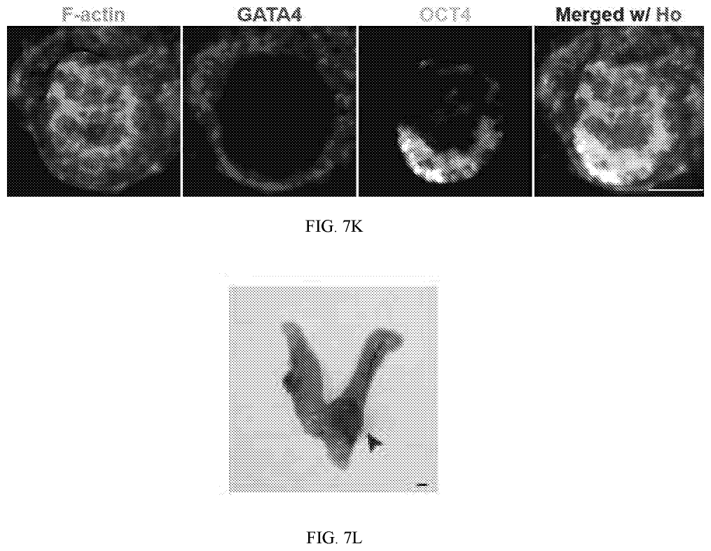

FIG. 7A shows a phase contrast image of iEPS-blastoids.

FIG. 7B shows quantification of iEPS-blastoids formation efficiency. n=5 independent assays.

FIG. 7C shows a histogram showing the distribution of diameters of iEPS-blastoids. n=23 iEPS-blastoids.

The process of the induction of iEPS-blastoids recapitulates the compaction, polarization, and changes in subcellular YAP localization FIG. 7D shows immunofluorescence staining of iEPS aggregates at the indicated day of blastoid induction for E-cadherin.

FIG. 7E shows immunofluorescence staining of an iEPS aggregate at day 2 of blastoid induction for PARE and NANOG.

FIG. 7F shows immunofluorescence staining of an iEPS-blastoid for active YAP.

iEPS-blastoids displayed the correct spatial expression of markers for both embryonic and extraembryonic lineages.

FIG. 7G shows immunofluorescence staining of an iEPS-blastoid for CDX2 and NANOG. The rightmost panel shows the maximum intensity projection of z-stack images of the indicated protein.

FIG. 7H shows immunofluorescence staining of iEPS-blastoids for NANOG and GATA4. Ho, Hoechst. Scale bar, 20 μm.

Further culture of iEPS-blastoids generates egg-cylinder structures containing ExE-, EPI-, and VE-like compartments (marked by TFAP2C, SOX2/OCT4, and GATA4, respectively).

FIG. 7I shows immunofluorescence staining of a postimplantation embryo-like structure from in vitro culture of iEPS-blastoids for PCX and OCT4. The yellow arrowheads indicate the lumens lined with PCX.

FIG. 7J and FIG. 4K shows immunofluorescence staining of postimplantation embryo-like structure from in vitro culture of iEPS-blastoids for TFAP2C and SOX2 (FIG. 7J), or F-actin, GATA4, and OCT4 (FIG. 7K). Ho, Hoechst. Scale bar, 50 μm.

iEPS-blastoids implant into the uterus and induce the formation of decidua. FIG. 7L shows a brightfield image showing the formation of decidua in the mouse uterus 5 days after iEPS-blastoids transfer at 2.5 dpc. Black arrowhead indicates decidua.

Data are represented as mean±SEM. Scale bar, 1 mm (FIG. 7L), 100 μm (FIG. 7A), 50 μm (FIGS. 71), and 20 μm (FIG. 7D to FIG. 7G). Ho, Hoechst.

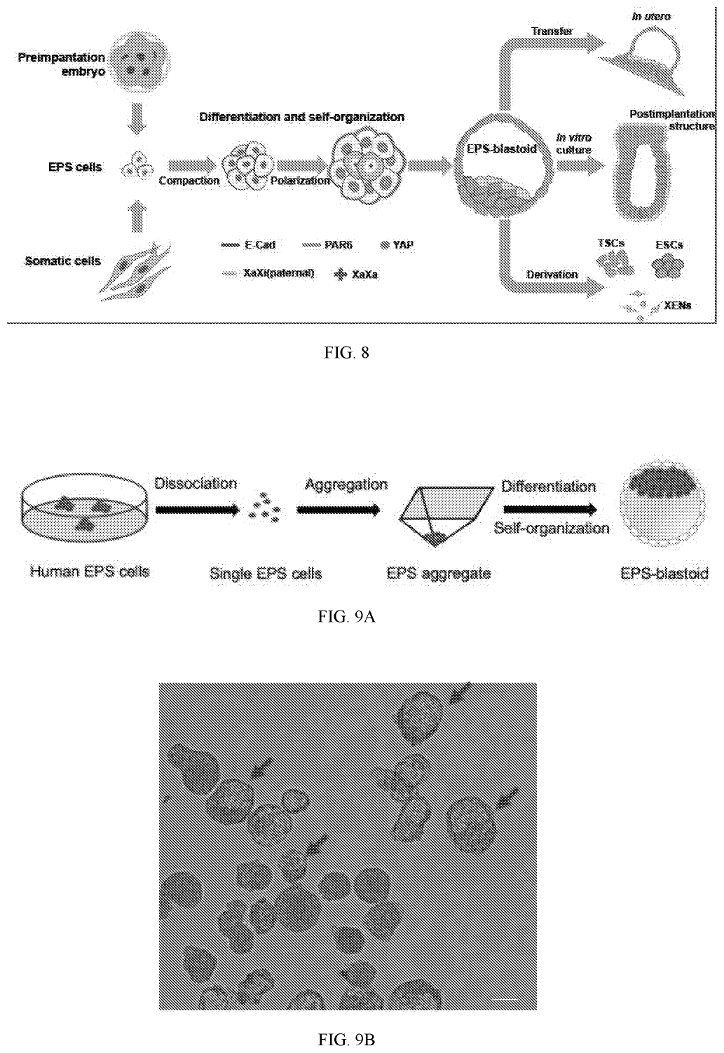

FIG. 8 shows a diagram summarizing the major findings of the herein disclosed data

In the herein-disclosed data, it is shown that EPS cells alone differentiate and self-organize to generate blastocyst-like structures that share several cellular, molecular, and functional features with natural blastocysts (FIG. 8).

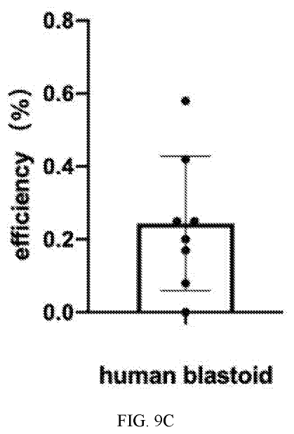

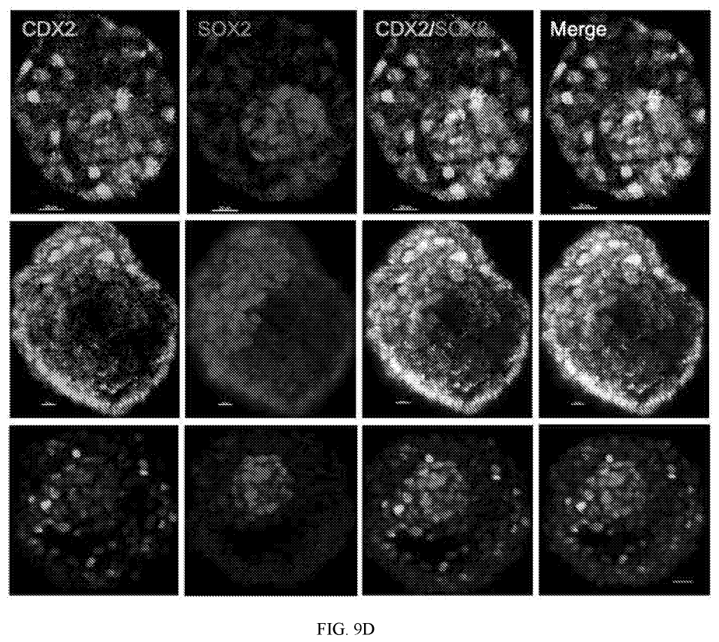

FIG. 9A shows a diagram showing the strategy of human EPS-blastoid formation. In this figure human EPS cells are dissociated to single EPS cells then allowed to aggregate. The cells then self-organize creating an EPS-blastoid.

FIG. 9B shows phase-contrast images of human EPS aggregates. The arrows indicate EPS-blastoid; scale bar=100 μm.

FIG. 9C shows quantification of human EPS-blastoid formation efficiency. N=7 independent replicates.

FIG. 9D shows immunofluorescence staining of human EPS-blastoid for CDX2 and SOX2. The images show the maximum intensity projection of z stack images. Scale bar=μm (top), 15 μm (middle) and 20 μm (bottom).

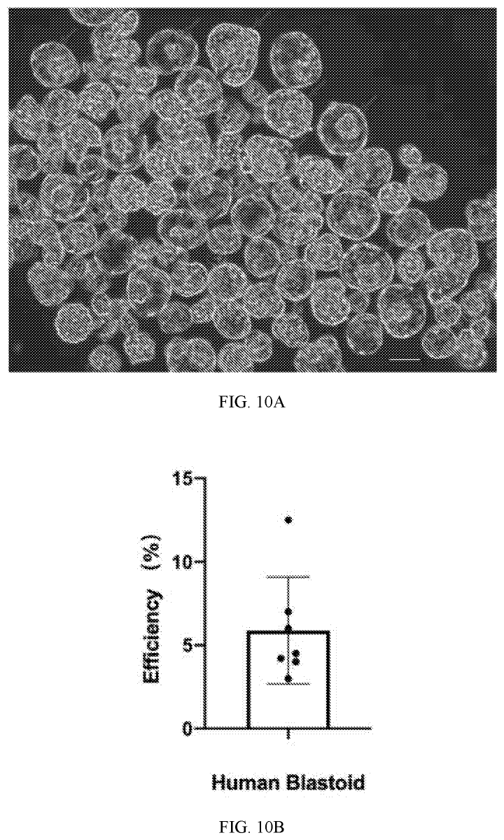

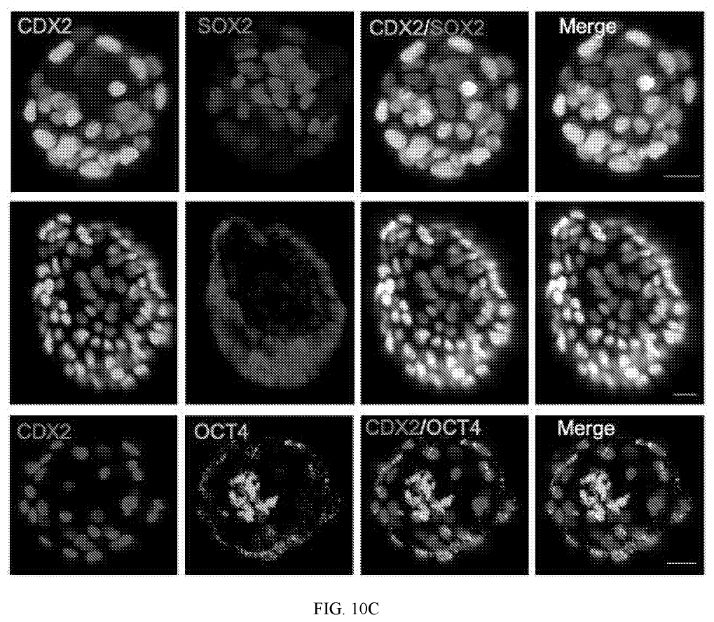

FIG. 10A shows phase-contrast images of human EPSC aggregates. The arrows indicate EPSC-blastoid. Scale bar=100 μm.

FIG. 10B shows quantification of human EPSC-blastoid formation efficiency. N=7 independent replicates.

FIG. 10C shows immunofluorescence staining of human EPSC-blastoid for CDX2, OCT4, and SOX2. The images show the maximum intensity projection of z stack images. Scale bar=20 μm. As disclosed herein, EPS-blastoid formation recapitulates several key early preimplantation developmental processes, including compaction, polarization, changes in subcellular YAP localization, and paternal XCI in TE. Thus, EPS-blastoid formation shares and follows certain similar developmental paths with those that generate a mouse blastocyst. Accordingly, provided herein is a better understanding of the evolutionarily distinct molecular signals that underlie the different forms and patterns that arise during embryogenesis.

In addition, the herein disclosed EPS-only blastoid approach has several additional advantages. First, unlike ETS-blastoids, which are generated by sequential aggregation using multiple cells from two stem cell types (ESCs and TSCs), all cells within EPS-blastoids come from a single cell type (even from a single cell). This enables a more straightforward interpretation of the readouts after introducing genetic or epigenetic changes. Second, EPS-blastoids further cultured in vitro recapitulate several key morphogenetic processes of early postimplantation development, forming an egg cylinder embryo-like structure. Without wishing to be bound by theory, an advantage provided herein is due to EPS cells' superior ability to generate PE cells than ESCs.

Bulk RNA-Seq analysis of individual EPS-blastoids revealed that they were more similar to blastocysts than morulae. Single-cell RNA-Seq analysis confirmed that EPS-blastoids contained all three cell lineages of blastocysts. Several DEGs for each lineage (EPFICM, TE, and PE) were determined between EPS-blastoids and blastocysts. For PE, DEGs seem to be enriched in terms related to vesicle transport and endocytosis. For ICM/EPI, a group of DNA methylation- and genomic imprinting-related genes, namely Tet1, Dnmt3L, Zfp42, Atrx, and Tdrd12, were expressed at lower levels in EPS-blastoids than blastocysts. These data suggest that epigenetic abnormalities, well-known defects for embryonic development (Barton et al., 1991; Surani et al., 1990), likely play a negative role in EPS-blastoids development in utero. Another notable observation from single-cell RNA-Seq analysis is that there are several subpopulations between EPI/ICM and TE. These cells likely represent uncommitted or improperly differentiated cells, likely as a result of suboptimal differentiation condition.

Although mouse EPS cells exhibit bi-directional developmental potency (in vivo chimera formation (Yang et al., 2017b) and in vitro blastoid generation) as well as some molecular features of early preimplantation embryos, they are clearly not equivalent to totipotent blastomeres. Nonetheless, and despite that, the EPS-blastoids provided herein followed evolutionary conserved developmental processes which faithfully recapitulated highlights of the plastic yet remarkably regulatory nature of early mammalian embryos. The transcriptional differences between laboratory created and naturally evolved blastocysts uncovered in the herein disclosed data, reflect distinct molecular trajectories that, nonetheless, lead to the generation of similarly patterned structures.

In summary, the present disclosure provides a 3D differentiation system for generating blastoids from cultured EPS cells derived from embryonic or adult sources. The herein disclosed data serves as a framework for advancing the development of fully functional synthetic blastocysts, not only in mice but also in other mammalian species, including humans. As such this system could be used as an in vitro model for studying fundamental questions in both preimplantation and early postimplantation mammalian embryogenesis, modeling diseases related to early pregnancy, high-throughput pharmacological and toxicological screens, and possibly bioengineered embryogenesis.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: 3D Differentiation System for Generating Blastocyst-Like Structures from EPS Cells

The ability of a single EPS cell to contribute to all three blastocyst lineages suggests that, under certain condition, EPS cells differentiate and self-organize into blastocyst-like structures (FIG. 1A). To test this idea, dissociated mouse EPS cells were seeded into microwells (˜5 cells/microwell) to form small aggregates. During early preimplantation development, trophectoderm (TE) cells are specified before inner cell mass (ICM) cells (Posfai et al., 2017). Thus, optimizing conditions that bias the differentiation of EPS cells toward trophoblasts were an initial focus. A commonly used TSC culture medium (Tanaka et al., 1998) was used; this showed that although cell aggregates grew well in TSC medium alone, they failed to form any sphere-like structures (FIG. 1B). Interestingly, mixing an embryo culture medium (KSOM) with TSC medium at a 1:1 ratio induced cavity formation in a small number of cell aggregates (FIG. 1B). KSOM is a potassium-supplemented SOM (Summers 2013). The effect of the KSOM was replicated with another embryo culture medium M16 (FIG. 1C). Later, instead of KSOM: TSC medium, KSOM: ETS medium (a 2:1:1 mixture of KSOM, N2B27 basal, and TSC basal medium), was used; the KSOM: ETS medium which supports the growth of both ESC and TSC aggregates (Harrison et al., 2017).

To further improve the differentiation conditions, different growth factors, cytokines, and small molecules were included to identify condition(s) that enable the generation of structures mimicking blastocysts (EPS-blastoids), which contain a cavity and an inner cell mass. A combination of FGF4, Heparin, BMP4, CHIR99021, and A83-01 resulted in the highest efficiency of EPS-blastoid formation. EPS cells treated with Y-27632, a Rho kinase (ROCK) inhibitor, on the day of seeding enhanced cell survival. Using this optimized condition, EPS-blastoids consistently emerged three days after cell seeding (FIG. 1D). EPS-blastoids continued to enlarge, reaching an early blastocyst-like size at around day five or day six. To quantify the efficiency of EPS-blastoid formation, the total number of blastocyst-like structures were counted, with this count divided it by the total number of cell aggregates. For this, two independent EPS cell lines were tested; approximately 15% of the cell aggregates exhibited typical blastocyst-like morphology at day five or day six (FIG. 1E and Table 1). The average diameter of these EPS-blastoids was comparable to that of E3.5 blastocysts (FIG. 1F and FIG. 1G).

TABLE 1
Summary of EPS-blastoid efficiency from different EPS cell lines.

| Cell line | Experiment | N_{blastoid} | Nₜₒₜₐₗ | N_{blastoid}/Nₜₒₜₐₗ (%) |
|---|---|---|---|---|
| EPS 1 | 1 | 66 | 633 | 10.43 |
| EPS 1 | 2 | 119 | 809 | 14.71 |
| EPS 1 | 3 | 130 | 782 | 16.62 |
| EPS 1 | 4 | 146 | 711 | 20.53 |
| EPS 1 | 5 | 119 | 697 | 17.07 |
| EPS 1 | 6 | 100 | 815 | 12.27 |
| EPS 1 | 7 | 106 | 871 | 12.17 |
| EPS 1 | 8 | 126 | 870 | 14.48 |
| EPS 1 | 9 | 118 | 837 | 14.1 |
| EPS 1 | 10 | 111 | 821 | 13.52 |
| EPS 1 | 11 | 90 | 793 | 11.35 |
| EPS 2 | 1 | 69 | 524 | 13.17 |
| EPS 2 | 2 | 166 | 799 | 20.78 |
| EPS 2 | 3 | 160 | 752 | 21.28 |
| EPS 2 | 4 | 105 | 735 | 14.29 |
| EPS 2 | 5 | 129 | 755 | 17.09 |
| EPS 2 | 6 | 101 | 809 | 12.48 |
| EPS 2 | 7 | 138 | 830 | 16.63 |
| EPS 2 | 8 | 128 | 844 | 15.17 |
| EPS 2 | 9 | 92 | 881 | 10.44 |
| EPS 2 | 10 | 167 | 826 | 20.22 |
| EPS 2 | 11 | 76 | 599 | 12.69 |
| Liu-EPS | 1 | 66 | 617 | 10.7 |
| Liu-EPS | 2 | 75 | 633 | 11.85 |
| Liu-EPS | 3 | 52 | 500 | 10.4 |
| Liu-EPS | 4 | 46 | 380 | 12.11 |
| iEPS | 1 | 125 | 741 | 16.87 |
| iEPS | 2 | 68 | 613 | 11.09 |
| iEPS | 3 | 101 | 617 | 16.37 |
| iEPS | 4 | 60 | 495 | 12.12 |
| iEPS | 5 | 102 | 689 | 14.8 |

WNT/β-catenin signaling pathway is dispensable for blastocyst formation (FIG. 1H and FIG. 1I) (Biechele et al., 2013; Haegel et al., 1995). However, Wnt-3a transiently up-regulates CDX2 (a trophoblast transcription factor) in mouse ESCs (He et al., 2008), and canonical WNT pathway activation was necessary for the generation of ETS-blastoids (Rivron et al., 2018). Similarly, the WNT antagonists, XAV939 or IWR-1-endo, significantly inhibited EPS-blastoids formation (FIG. 1J, FIG. 1K, and Table 2).

TABLE 2
Summary of EPS-blastoid efficiency
with treatment of Wnt antagonists.

| Experiment | Treatment | N_{blastoid} | Nₜₒₜₐₗ | N_{blastoid}/Nₜₒₜₐₗ (%) |
|---|---|---|---|---|
| 1 | Vehicle | 158 | 764 | 20.68 |
| | XVA939 | 5 | 623 | 0.8 |
| | IWR1 | 3 | 709 | 0.42 |
| 2 | Vehicle | 148 | 811 | 18.25 |
| | XVA939 | 2 | 660 | 0.3 |
| | IWR1 | 2 | 716 | 0.28 |
| 3 | Vehicle | 150 | 782 | 19.18 |
| | XVA939 | 2 | 705 | 0.28 |
| | IWR1 | 3 | 735 | 0.41 |
| 4 | Vehicle | 130 | 782 | 16.62 |
| | XVA939 | 3 | 669 | 0.45 |
| | IWR1 | 9 | 733 | 1.23 |
| 5 | Vehicle | 105 | 735 | 14.29 |
| | XVA939 | 3 | 735 | 0.41 |
| | IWR1 | 6 | 738 | 0.81 |

Next, it was determined that a single EPS cell could give rise to an entire EPS-blastoid. Surprisingly, individual EPS cells did not survive in the herein-disclosed 3D differentiation system. To overcome this problem, puromycin resistant and mCherry+ EPS cells were mixed with helper wild type EPS cells at the ratio of 1:10 for the initial plating. Low concentration of puromycin (0.25 μg/ml) was added to the differentiation medium 24 hours later to eliminate helper cells gradually (FIG. 1L). Using this strategy, clonal EPS-blastoids were generated from a single EPS cell with an efficiency of approximately 2.7% (FIG. 1M, FIG. 1N, and Table 3). These results demonstrate that a single EPS cell retains the capacity to form an entire EPS-blastoid.

TABLE 3
Summary of EPS-blastoid Efficiency from a single EPS cell.

| | WT | Puro+ | | | |
|---|---|---|---|---|---|
| Experiment | cells | cells | N_{blastoid} | Nₜₒₜₐₗ | N_{blastoid}/Nₜₒₜₐₗ (%) |
| 1 | + | − | 0 | 0 | 0 |
| | + | + | 4 | 134 | 2.99 |
| 2 | + | − | 0 | 0 | 0 |
| | + | + | 9 | 280 | 3.21 |
| 3 | + | − | 0 | 0 | 0 |
| | + | + | 3 | 115 | 2.61 |
| 4 | + | − | 0 | 0 | 0 |
| | + | + | 4 | 198 | 2.02 |

Extended pluripotency was then shown to be helpful for generating blastoids. To this end, blastoid formation efficiencies were compared between an ES cell line (Tanimoto et al., 2008) and EPS cells converted from the same ES cell line. While approximately 8% of ES-converted EPS cell aggregates formed EPS-blastoids, little to no (˜0.2%) ES cell aggregates generated blastocyst-like structures (ES-blastoids) (FIG. 1O, FIG. 1P, and Table 4).

TABLE 4
Summary of EPS-blastoid Efficiency
from ES and ES-converted EPS.

| Experiment | cell type | N_{blastoid} | Nₜₒₜₐₗ | N_{blastoid}/Nₜₒₜₐₗ (%) |
|---|---|---|---|---|
| 1 | ES | 1 | 522 | 0.19 |
| | ES-converted EPS | 51 | 738 | 6.91 |
| 2 | ES | 2 | 496 | 0.4 |
| | ES-converted EPS | 54 | 660 | 8.18 |
| 3 | ES | 0 | 605 | 0 |
| | ES-converted EPS | 45 | 518 | 8.69 |

Besides EPS cells cultured in LCDM condition (Yang et al., 2017b), EPS cells generated and cultured under a different culture condition developed by Pengtao Liu's group (referred to as Liu-EPS; Yang et al., 2017a) also generated EPS-blastoids using the herein-disclosed 3D differentiation system in approximately 11% of cell aggregates (FIG. 1Q, FIG. 1R, and Table 1, above). The Liu-EPS-blastoids were of similar size as E3.5 blastocysts (FIG. 1S).

### Example 2: EPS-Blastoid Formation Recapitulates Key Preimplantation Developmental Processes

Key cellular and molecular events characteristic of early preimplantation development were recapitulated during EPS-blastoid formation. Beginning at the 8-cell stage, blastomeres undergo compaction, which is characterized by the formation of intercellular junctions (Rossant and Tam, 2009; Wang et al., 2008). A similar process occurs during EPS-blastoid formation. To assay this, the dynamics of EPS cell aggregation during the first 18 hours was monitored. Four hours after seeding, cells were found loosely connected. At approximately 18 hours, cells started to form compact aggregates (FIG. 2A), with the cell adhesion protein, E-cadherin, and the tight junction protein, ZO1, beginning to accumulate at the cell-cell junctions, reminiscent of a compacted 8-cell embryo (FIG. 2B, FIG. 2C, FIG. 2D, and FIG. 2E). The fast kinetics of EPS cell aggregate formation was likely due to the high levels of the Cdh1 and Tjp1 (genes encoding E-cadherin and ZO1, respectively) mRNA and ZO1 protein in cultured EPS cells (FIG. 2F and FIG. 2G).

Following blastomere compaction, polarization begins. Apical domain proteins, such as atypical protein kinase C (aPKC) and the Par complex proteins (PAR3 and PAR6), start to accumulate at the apical side of the blastomeres (Chazaud and Yamanaka, 2016; Rossant and Tam, 2009). EPS cell aggregates also underwent polarization. At day 1, PAR6 protein was absent from all cell aggregates examined, which homogenously expressed the pluripotency factor SOX2 (FIG. 2H). At day 2, a fraction of cell aggregates (˜33%) began to show PAR6 enrichment on the apical surface, reminiscent of a 16-cell embryo (FIG. 2H and FIG. 2I). At day 3, the majority of cell aggregates (˜75%) exhibited a polarized pattern of PAR6 enrichment towards the apical side (FIG. 2H and FIG. 2I). Thus, EPS-blastoid formation recapitulates the process of polarization characteristic of early preimplantation development. During early embryogenesis, the Hippo/YAP signaling pathway plays an essential role in specifying the TE and ICM lineages (Kaneko and DePamphilis, 2013; Nishioka et al., 2009; Posfai et al., 2017; Rayon et al., 2014; Yagi et al., 2007). The formation of EPS-blastoids depended on YAP activity. To assay this, YAP localization during EPS-blastoid induction was monitored. YAP is predominantly localized in the cytoplasm in cultured EPS cells (FIG. 2J). At day 2, YAP was found in the nucleus of some outside cells of the aggregate (FIG. 2K). This YAP nuclear localization was observed in ˜7% of aggregates (FIG. 2L). At day 3, ˜27% of aggregates contained outside cells with nuclear localization of YAP (FIG. 2K and FIG. 2M). In the EPS-blastoids collected at day 5, nuclear YAP localization was evident in most outside cells, whereas inside cells still exhibited cytoplasmic YAP localization (FIG. 2L). This YAP localization pattern mirrored that in the blastocysts and was found in about 60% of EPS-blastoids (FIG. 2L and FIG. 2M). Furthermore, inhibiting the interaction between YAP and TEAD4 via a small molecule inhibitor prevented cavity formation in mouse embryos and EPS aggregates (FIG. 2N, FIG. 2O, FIG. 2P, FIG. 2Q, and Table 5). Therefore, like blastocysts, EPS-blastoid formation also requires an intact Hippo/YAP signaling pathway.

TABLE 5
Summary of EPS-blastoid efficiency with
treatment of YAP-TEAD inhibitor

| Experiment | Treatment | N_{blastoid} | Nₜₒₜₐₗ | N_{blastoid}/Nₜₒₜₐₗ (%) |
|---|---|---|---|---|
| 1 | Vehicle | 138 | 830 | 16.63 |
| | VP | 6 | 686 | 0.87 |
| 2 | Vehicle | 98 | 849 | 11.54 |
| | VP | 9 | 855 | 1.05 |
| 3 | Vehicle | 112 | 820 | 13.66 |
| | VP | 12 | 841 | 1.43 |
| 4 | Vehicle | 76 | 599 | 12.69 |
| | VP | 2 | 549 | 0.36 |

In an early female blastocyst, while both X-chromosomes are active in the ICM, the paternally-inherited X chromosome is preferentially inactivated in TE (Takagi and Sasaki, 1975; West et al., 1977). The X-chromosome status in different lineages of EPS-blastoids were assayed. Here, an epiblast stem cell (EpiSC) line, which contained a green fluorescent protein (GFP) transgene in the paternal X-chromosome (X-GFP). X-GFP EpiSCs were FACS-sorted to obtain a pure GFP negative population (Paternally X inactivated, or Xpi-GFP) (Bao et al., 2009). Xpi-GFP EpiSCs were converted to EPS cells by culture adaptation. Over passaging, the percentage of GFP+ cells was increased, indicating gradual reactivation of the inactivated paternal X chromosome during conversion. Next, GFP+ EPS cells were FACS-sorted out and used to generate EPS-blastoids. These blastoids were stained with NANOG and CDX2, which showed that a large fraction of them (79%, 11/14) contained GFP+ cells only in the ICM-like compartment (FIG. 2R and FIG. 2S). Thus, upon EPS cell differentiation, paternal X-chromosome was preferentially silenced in TE-like cells while most ICM-like cells contained two active X chromosomes. Collectively, these results demonstrate EPS-blastoid formation recapitulate key molecular and cellular processes characteristic of early preimplantation development.

### Example 3: EPS-Blastoids Possess Three Lineages of Blastocysts

E3.5 mouse blastocysts have two cell lineages, the external trophectoderm (TE) layer and the internal inner cell mass (ICM). Whether EPS-blastoids also had these two early blastocyst lineages was tested. Immunofluorescence analysis revealed that cells in the outer layer of EPS-blastoids expressed the TE transcription factors CDX2 and EOMES (FIG. 3A and FIG. 3B). The outer layer of cells also expressed the trophoblast-specific cytokeratin KRT8 (FIG. 3C). Within the ICM-like compartment of EPS-blastoids, expression of pluripotency factors SOX2, NANOG, and OCT4 were detected (FIG. 3D, FIG. 3E, and FIG. 3F). Co-staining of CDX2 and NANOG confirmed the presence of both TE- and ICM-like lineages within the same EPS-blastoid (FIG. 3G). Of the 140 EPS-blastoids examined, 74.2% had correctly allocated TE-(CDX2+) and ICM-like (SOX2+) lineages, 15% had only the TE-like lineage, 1.4% had only the ICM-like lineage, and 9.3% exhibited mislocalization of the TE- and/or ICM-like lineages (FIG. 3H). The number of cells within both the TE- and ICM-like compartments in EPS-blastoids collected on day 4, 5, and 6 were counted. Day 4 and 5 EPS-blastoids had fewer cells in both lineages than E3.5 blastocysts (FIG. 3I). At day 6, cell numbers in both lineages were comparable to E3.5 blastocysts (FIG. 3I). These results demonstrate that the cellular composition of EPS-blastoids resembles early blastocysts.

The segregation of TE and ICM lineage occurred during EPS-blastoid formation and how the cells of the two lineages were spatially distributed in the aggregates. To this end expression of CDX2 and SOX2 in EPS cell aggregates collected from day 1 to day 5 was analyzed. At day 1, —55% of the aggregates exhibited a composition of mixed CDX2+ and SOX2+ cell. This percentage continued to increase from day 1 to day 3 (FIG. 3J and FIG. 3K). Non-blastoid EPS cell aggregates at day 4 or day 5 were also mostly composed of CDX2+ and SOX2+ cells (FIG. 3J and FIG. 3K). However, CDX2+ cells were randomly distributed, and the number of CDX2+ cells varied among aggregates.

As early blastocysts further develop, ICM segregates into two lineages: epiblast (EPI) cells and primitive endoderm (PE) cells. Therefore, whether EPS-blastoids could develop into a late blastocyst-like structure comprised of all three blastocyst lineages: TE, EPI, and PE, was examined. Indeed, in some EPS-blastoids, GATA4+ PE-like cells enclosing the NANOG+ EPI-like compartment was detected, reminiscent of a peri-implantation E4.5 blastocyst (FIG. 3L). This pattern was observed in 24 out of 112 EPS-blastoids (21.4%) collected on day 5 or 6 (FIG. 3M). The number of EPI- and PE-like cells in these GATA4+ EPS-blastoids were comparable to that in E4.5 blastocysts, although EPS-blastoids exhibited higher variations (FIG. 3N).

The expression of the lineage markers in EPS-blastoids generated from ES-converted EPS, Liu-EPS, as well as a single EPS cell was assayed. Consistently, all EPS-blastoids exhibited blastocyst-like allocation of cell lineages, as revealed by CDX2, SOX2/NANOG, and GATA4 staining (FIG. 3O to FIG. 3R).

### Example 4: Transcriptome Analysis of EPS-Blastoids.

RNA-Seq analysis using individual EPS-blastoids was performed. Their transcriptomes were compared to E3.5 early blastocysts and E3.0 morulae from published datasets (Sampath Kumar et al., 2017; Wang et al., 2018). Principle component analysis revealed that EPS-blastoids were closer to blastocysts than morulae on both PC1 and PC2 axis (FIG. 4A). Unsupervised correlation clustering also showed that EPS-blastoids clustered closer to blastocysts than to morulae (FIG. 4B). These RNA-seq data also revealed differentially expressed genes (DEGs) between EPS-blastoids, blastocysts, and morulae (Table 6 and Table 7).

TABLE 6
List of DEGs Between EPS-blastoids and Blastocysts

| GeneName | Feature | GeneId | FC | pval | qval |
|---|---|---|---|---|---|
| Calcoco2 | gene | MSTRG.25615 | 1411.692516 | 0 | 0 |
| Gm15981 | gene | MSTRG.107116 | 10.60330116 | 3.11E−15 | 9.18E−11 |
| . | gene | MSTRG.143119 | 147.7272067 | 4.66E−15 | 9.18E−11 |
| Pramel4 | gene | MSTRG.101970 | 166.4512166 | 3.18E−14 | 4.69E−10 |
| Gm16166 | gene | MSTRG.18583 | 5.866517892 | 5.42E−14 | 6.40E−10 |
| Fbp2 | gene | MSTRG.37912 | 642.0575405 | 6.64E−14 | 6.54E−10 |
| Gm13740 | gene | MSTRG.78564 | 31.15041552 | 5.76E−13 | 4.86E−09 |
| AA467197 | gene | MSTRG.80472 | 244.3011412 | 7.84E−13 | 5.79E−09 |
| . | gene | MSTRG.17028 | 5.271174142 | 1.21E−12 | 7.96E−09 |
| Gm13039 | gene | MSTRG.101870 | 12.44922929 | 1.82E−12 | 1.04E−08 |
| Rpl5-ps1 | gene | MSTRG.694 | 126.238537 | 1.94E−12 | 1.04E−08 |
| Rps12-ps10 | gene | MSTRG.80441 | 14.04028913 | 3.47E−12 | 1.59E−08 |
| RP23-181P23.2 | gene | MSTRG.124667 | 4.84719641 | 3.51E−12 | 1.59E−08 |
| Rpl39l | gene | MSTRG.53755 | 274.2806684 | 4.56E−12 | 1.92E−08 |
| Gm9057 | gene | MSTRG.103906 | 22.55578786 | 5.81E−12 | 2.29E−08 |
| . | gene | MSTRG.120965 | 6.093912636 | 1.10E−11 | 4.06E−08 |
| . | gene | MSTRG.21764 | 436.8033656 | 1.62E−11 | 5.30E−08 |
| . | gene | MSTRG.3931 | 20.70063296 | 1.85E−11 | 5.37E−08 |
| Clic3 | gene | MSTRG.75028 | 44.43778238 | 1.90E−11 | 5.37E−08 |
| Gm8250 | gene | MSTRG.70797 | 4.924399171 | 1.94E−11 | 5.37E−08 |
| Crxos | gene | MSTRG.121773 | 341.2191274 | 2.04E−11 | 5.37E−08 |
| Trim43c | gene | MSTRG.143079 | 52.42820835 | 2.09E−11 | 5.37E−08 |
| Dppa3-ps | gene | MSTRG.146914 | 20.20210097 | 2.31E−11 | 5.69E−08 |
| Fam107b | gene | MSTRG.73600 | 14.82419282 | 2.93E−11 | 6.66E−08 |
| Gm16471 | gene | MSTRG.149327 | 6.377290781 | 3.39E−11 | 7.41E−08 |
| . | gene | MSTRG.18159 | 59.0671765 | 3.67E−11 | 7.41E−08 |
| Gm17786 | gene | MSTRG.140228 | 17.18183966 | 3.94E−11 | 7.41E−08 |
| . | gene | MSTRG.37937 | 26.60801659 | 3.94E−11 | 7.41E−08 |
| Gm8482 | gene | MSTRG.32977 | 22.6516874 | 3.97E−11 | 7.41E−08 |
| Gm24276 | gene | MSTRG.369 | 14.24849995 | 4.12E−11 | 7.41E−08 |
| Rpl21-ps14 | gene | ENSMUSG00000062152 | 6.581647496 | 4.14E−11 | 7.41E−08 |
| Gm13413 | gene | MSTRG.75676 | 15.85047361 | 4.62E−11 | 8.03E−08 |
| Gm11955 | gene | MSTRG.19277 | 12.83058832 | 5.20E−11 | 8.77E−08 |
| Gm32536 | gene | MSTRG.119301 | 9.721144665 | 5.41E−11 | 8.88E−08 |
| Rpl5-ps2 | gene | MSTRG.82501 | 23.17260448 | 5.90E−11 | 9.42E−08 |
| Gm9083 | gene | MSTRG.146435 | 14.83694674 | 6.43E−11 | 1.00E−07 |
| Gm8425 | gene | MSTRG.55729 | 3.859639548 | 6.80E−11 | 1.03E−07 |
| Gm6425 | gene | MSTRG.70153 | 42.8184629 | 7.34E−11 | 1.08E−07 |
| Apoa1 | gene | MSTRG.139873 | 88.16386749 | 7.81E−11 | 1.11E−07 |
| . | gene | MSTRG.145763 | 3.442317421 | 7.90E−11 | 1.11E−07 |
| . | gene | MSTRG.130919 | 10.82223729 | 8.09E−11 | 1.11E−07 |
| Pramel7 | gene | MSTRG.78549 | 81.93891785 | 8.27E−11 | 1.11E−07 |
| Gm6375 | gene | MSTRG.119819 | 32.03721076 | 9.08E−11 | 1.19E−07 |
| Gm4222 | gene | MSTRG.78653 | 108.3603286 | 9.30E−11 | 1.19E−07 |
| RP23-363E22.1 | gene | MSTRG.126762 | 9.018696869 | 1.18E−10 | 1.47E−07 |
| Gm12447 | gene | MSTRG.96005 | 14.79812918 | 1.20E−10 | 1.47E−07 |
| . | gene | MSTRG.64779 | 9.160686399 | 1.28E−10 | 1.54E−07 |
| Cyp2s1 | gene | MSTRG.122506 | 29.76715914 | 1.34E−10 | 1.59E−07 |
| RP23-412J12.1 | gene | MSTRG.129333 | 3.796346221 | 1.46E−10 | 1.69E−07 |
| Ywhaq-ps2 | gene | MSTRG.109517 | 9.331129762 | 1.65E−10 | 1.87E−07 |
| mt-Th | gene | MSTRG.145686 | 13.11214778 | 1.69E−10 | 1.88E−07 |
| Pramel6 | gene | MSTRG.78552 | 78.80313426 | 1.76E−10 | 1.93E−07 |
| Gm13653 | gene | MSTRG.77943 | 14.48474865 | 2.10E−10 | 2.26E−07 |
| Gm11979 | gene | MSTRG.19533 | 4.519779232 | 2.27E−10 | 2.36E−07 |
| . | gene | MSTRG.94783 | 24.37740756 | 2.28E−10 | 2.36E−07 |
| . | gene | MSTRG.138098 | 31.06088428 | 2.66E−10 | 2.71E−07 |
| . | gene | MSTRG.120793 | 7.740931491 | 2.78E−10 | 2.79E−07 |
| Gm15267 | gene | MSTRG.151082 | 17.68181941 | 2.89E−10 | 2.84E−07 |
| Gm28530 | gene | MSTRG.143521 | 38.90915049 | 3.06E−10 | 2.93E−07 |
| Gm6285 | gene | MSTRG.149492 | 11.59123954 | 3.08E−10 | 2.93E−07 |
| Rpl7a-ps3 | gene | MSTRG.49185 | 11.15301393 | 3.24E−10 | 3.04E−07 |
| Gm44078 | gene | MSTRG.119573 | 5.986091119 | 3.37E−10 | 3.11E−07 |
| . | gene | MSTRG.135989 | 50.90039662 | 3.92E−10 | 3.56E−07 |
| . | gene | MSTRG.69293 | 169.2277649 | 4.58E−10 | 4.10E−07 |
| . | gene | MSTRG.3933 | 111.8313999 | 4.79E−10 | 4.22E−07 |
| Gm6265 | gene | MSTRG.30496 | 55.76095259 | 5.27E−10 | 4.58E−07 |
| Rpl35a-ps7 | gene | MSTRG.114963 | 7.920204075 | 5.39E−10 | 4.61E−07 |
| Rpsa-ps5 | gene | MSTRG.20793 | 15.45109581 | 5.51E−10 | 4.62E−07 |
| Gm14541 | gene | MSTRG.146884 | 14.94008649 | 5.55E−10 | 4.62E−07 |
| Gm12816 | gene | MSTRG.99438 | 33.56278245 | 6.07E−10 | 4.96E−07 |
| Gm7792 | gene | MSTRG.108148 | 90.84364835 | 6.13E−10 | 4.96E−07 |
| Gm15616 | gene | MSTRG.106923 | 43.37479483 | 6.61E−10 | 5.20E−07 |
| Fam151a | gene | MSTRG.99011 | 82.00160661 | 6.66E−10 | 5.20E−07 |
| Gm11971 | gene | MSTRG.19537 | 3.852641269 | 6.69E−10 | 5.20E−07 |
| Gm12891 | gene | MSTRG.100136 | 19.47512136 | 6.85E−10 | 5.26E−07 |
| Gm7589 | gene | MSTRG.141199 | 29.29165227 | 7.01E−10 | 5.31E−07 |
| Gm6051 | gene | MSTRG.107265 | 376.8267244 | 7.14E−10 | 5.34E−07 |
| . | gene | MSTRG.95106 | 49.0017705 | 7.55E−10 | 5.51E−07 |
| RP24-490A22.9 | gene | MSTRG.121830 | 86.50571767 | 8.12E−10 | 5.85E−07 |
| Gm7867 | gene | MSTRG.5443 | 34.26990889 | 8.97E−10 | 6.37E−07 |
| Gm13675 | gene | MSTRG.78278 | 7.638556686 | 9.06E−10 | 6.37E−07 |
| . | gene | MSTRG.79122 | 155.155452 | 9.39E−10 | 6.52E−07 |
| RP23-375O10.1 | gene | MSTRG.124441 | 19.86615409 | 9.61E−10 | 6.60E−07 |
| Mir684-2 | gene | MSTRG.94131 | 6.448624304 | 1.05E−09 | 7.05E−07 |
| Rpl7a-ps10 | gene | MSTRG.143475 | 9.728285801 | 1.05E−09 | 7.05E−07 |
| Gm5218 | gene | MSTRG.51633 | 8.648342802 | 1.08E−09 | 7.15E−07 |
| . | gene | MSTRG.145569 | 9.884276478 | 1.09E−09 | 7.15E−07 |
| Rpl21-ps6 | gene | MSTRG.62345 | 7.384669489 | 1.11E−09 | 7.22E−07 |
| . | gene | MSTRG.93412 | 15.70039338 | 1.13E−09 | 7.22E−07 |
| Gm5981 | gene | MSTRG.91562 | 6.356673852 | 1.19E−09 | 7.48E−07 |
| Gm5529 | gene | MSTRG.4425 | 81.41194686 | 1.21E−09 | 7.52E−07 |
| . | gene | MSTRG.22814 | 254.6099724 | 1.25E−09 | 7.68E−07 |
| Gm15483 | gene | MSTRG.129506 | 43.71464437 | 1.27E−09 | 7.72E−07 |
| Gm5619 | gene | MSTRG.142854 | 17.11934296 | 1.32E−09 | 7.98E−07 |
| Gm6274 | gene | MSTRG.146857 | 52.70030827 | 1.36E−09 | 8.11E−07 |
| Gm5943 | gene | MSTRG.149840 | 14.64506788 | 1.43E−09 | 8.46E−07 |
| Gm7866 | gene | MSTRG.141993 | 43.82172109 | 1.48E−09 | 8.66E−07 |
| Gm13921 | gene | MSTRG.79226 | 21.61130662 | 1.54E−09 | 8.90E−07 |
| Gm42845 | gene | MSTRG.107736 | 19.46000148 | 1.82E−09 | 1.03E−06 |
| Gm37599 | gene | MSTRG.8036 | 7.728541222 | 1.85E−09 | 1.03E−06 |
| . | gene | MSTRG.71972 | 18.15250018 | 1.85E−09 | 1.03E−06 |
| Gm15013 | gene | MSTRG.150582 | 5.759510012 | 1.87E−09 | 1.03E−06 |
| AC125178.1 | gene | MSTRG.145588 | 25.19411142 | 1.96E−09 | 1.07E−06 |
| Slc30a4 | gene | MSTRG.80485 | 4.214866018 | 2.17E−09 | 1.17E−06 |
| Gm8282 | gene | MSTRG.142978 | 8.866420019 | 2.24E−09 | 1.20E−06 |
| Bhmt | gene | MSTRG.39629 | 627.3137472 | 2.28E−09 | 1.21E−06 |
| AC123873.2 | gene | MSTRG.145597 | 24.17372875 | 2.42E−09 | 1.28E−06 |
| Gm5842 | gene | MSTRG.85612 | 8.844373614 | 2.51E−09 | 1.30E−06 |
| Gm12624 | gene | MSTRG.22808 | 96.67762949 | 2.62E−09 | 1.35E−06 |
| Gm5829 | gene | MSTRG.3932 | 12.35757236 | 2.64E−09 | 1.35E−06 |
| Gm38358 | gene | MSTRG.10668 | 3.791828901 | 2.82E−09 | 1.42E−06 |
| Rpsa-ps1 | gene | MSTRG.9329 | 17.22532595 | 2.87E−09 | 1.44E−06 |
| Gm10045 | gene | MSTRG.60459 | 18.37760371 | 2.92E−09 | 1.45E−06 |
| Gm12568 | gene | MSTRG.21720 | 6.882912319 | 3.01E−09 | 1.48E−06 |
| Rps25-ps1 | gene | MSTRG.120496 | 23.335267 | 3.12E−09 | 1.51E−06 |
| Gm7384 | gene | MSTRG.120253 | 9.636234472 | 3.12E−09 | 1.51E−06 |
| Rpl31-ps10 | gene | MSTRG.101416 | 13.37177443 | 3.30E−09 | 1.57E−06 |
| Gm7899 | gene | ENSMUSG00000103823 | 15.37264633 | 3.75E−09 | 1.77E−06 |
| . | gene | MSTRG.9539 | 43.89576775 | 3.90E−09 | 1.81E−06 |
| Gm16367 | gene | MSTRG.145619 | 25.99951195 | 3.93E−09 | 1.81E−06 |
| Gm12013 | gene | MSTRG.20012 | 48.67097349 | 3.94E−09 | 1.81E−06 |
| Rps6-ps2 | gene | MSTRG.135185 | 7.807796851 | 4.03E−09 | 1.83E−06 |
| Rnf34 | gene | MSTRG.110151 | 8.015227991 | 4.07E−09 | 1.83E−06 |
| Bin2 | gene | MSTRG.52987 | 36.40073939 | 4.31E−09 | 1.93E−06 |
| . | gene | MSTRG.68375 | 8.438355532 | 4.39E−09 | 1.95E−06 |
| Gm6794 | gene | MSTRG.115904 | 27.76654328 | 4.43E−09 | 1.95E−06 |
| Rps8-ps1 | gene | MSTRG.14347 | 27.96048404 | 4.51E−09 | 1.97E−06 |
| 1700029P11Rik | gene | MSTRG.51747 | 61.51014865 | 4.67E−09 | 2.03E−06 |
| Foxm1 | gene | MSTRG.119521 | 4.369370331 | 4.96E−09 | 2.13E−06 |
| Gm3183 | gene | MSTRG.108128 | 26.20847273 | 4.97E−09 | 2.13E−06 |
| Gm6223 | gene | MSTRG.143117 | 17.78493033 | 5.00E−09 | 2.13E−06 |
| Gm7299 | gene | MSTRG.9714 | 19.21486957 | 5.04E−09 | 2.13E−06 |
| . | gene | MSTRG.8055 | 4.498433059 | 5.31E−09 | 2.22E−06 |
| E530001F21Rik | gene | MSTRG.149499 | 8.289349975 | 5.65E−09 | 2.35E−06 |
| Gm13215 | gene | MSTRG.100850 | 12.40246845 | 5.78E−09 | 2.39E−06 |
| Gm11478 | gene | MSTRG.25041 | 30.24897067 | 5.83E−09 | 2.39E−06 |
| Slc43a3 | gene | MSTRG.78418 | 38.82877969 | 5.98E−09 | 2.43E−06 |
| Gm12627 | gene | MSTRG.22799 | 65.63738705 | 6.08E−09 | 2.46E−06 |
| Gm12619 | gene | MSTRG.22807 | 59.54391778 | 6.77E−09 | 2.72E−06 |
| Rps12-ps24 | gene | MSTRG.132424 | 16.40211244 | 7.23E−09 | 2.87E−06 |
| . | gene | MSTRG.53937 | 6.012754444 | 7.24E−09 | 2.87E−06 |
| Gm11824 | gene | MSTRG.94282 | 15.12183599 | 7.34E−09 | 2.89E−06 |
| Hmgb1-ps4 | gene | MSTRG.69931 | 10.37451922 | 7.65E−09 | 2.99E−06 |
| . | gene | MSTRG.28990 | 22.51966592 | 7.72E−09 | 3.00E−06 |
| Gm8652 | gene | MSTRG.112776 | 7.316374444 | 7.88E−09 | 3.02E−06 |
| Gm14056 | gene | MSTRG.81365 | 3.515530139 | 7.88E−09 | 3.02E−06 |
| Gm12094 | gene | MSTRG.20920 | 8.080286144 | 8.13E−09 | 3.08E−06 |
| Gm12171 | gene | MSTRG.21746 | 10.20576029 | 8.20E−09 | 3.08E−06 |
| Rpl27a-ps1 | gene | ENSMUSG00000061488 | 13.241642 | 8.51E−09 | 3.18E−06 |
| Gm3160 | gene | MSTRG.131153 | 2.857994751 | 8.62E−09 | 3.20E−06 |
| Gm16427 | gene | MSTRG.108157 | 133.6602608 | 8.80E−09 | 3.25E−06 |
| Gm5883 | gene | MSTRG.119164 | 12.92558535 | 9.17E−09 | 3.35E−06 |
| Gm5851 | gene | MSTRG.89409 | 8.585927544 | 9.18E−09 | 3.35E−06 |
| . | gene | MSTRG.41983 | 11.44865834 | 9.26E−09 | 3.36E−06 |
| Gm5803 | gene | MSTRG.48551 | 14.66675556 | 9.45E−09 | 3.41E−06 |
| Gm12034 | gene | MSTRG.20214 | 26.15463555 | 1.00E−08 | 3.54E−06 |
| Gulo | gene | MSTRG.44961 | 44.73599236 | 1.00E−08 | 3.54E−06 |
| Rps4l-ps | gene | MSTRG.128348 | 9.521225645 | 1.01E−08 | 3.54E−06 |
| Gm13545 | gene | MSTRG.77024 | 4.091343779 | 1.01E−08 | 3.54E−06 |
| Gm14279 | gene | MSTRG.82726 | 33.42541309 | 1.06E−08 | 3.72E−06 |
| Gm42814 | gene | MSTRG.89113 | 3.515755864 | 1.08E−08 | 3.75E−06 |
| . | gene | MSTRG.56455 | 45.93854896 | 1.11E−08 | 3.82E−06 |
| . | gene | MSTRG.82927 | 4.551070847 | 1.12E−08 | 3.84E−06 |
| Gm8062 | gene | MSTRG.38523 | 6.366094152 | 1.15E−08 | 3.93E−06 |
| Gm3106 | gene | MSTRG.108114 | 22.12433121 | 1.17E−08 | 3.98E−06 |
| . | gene | MSTRG.76410 | 29.68212056 | 1.19E−08 | 4.00E−06 |
| Gm14383 | gene | MSTRG.80230 | 33.46685834 | 1.19E−08 | 4.00E−06 |
| . | gene | MSTRG.145635 | 18.26501038 | 1.20E−08 | 4.00E−06 |
| . | gene | MSTRG.29251 | 15.25836502 | 1.23E−08 | 4.08E−06 |
| Gm5850 | gene | MSTRG.89413 | 8.430614086 | 1.35E−08 | 4.47E−06 |
| Gm10224 | gene | MSTRG.119141 | 31.84774522 | 1.38E−08 | 4.52E−06 |
| Gm13226 | gene | MSTRG.102719 | 39.5510744 | 1.41E−08 | 4.61E−06 |
| . | gene | MSTRG.145637 | 17.37468008 | 1.47E−08 | 4.76E−06 |
| Gm8624 | gene | MSTRG.27312 | 6.975324624 | 1.48E−08 | 4.77E−06 |
| Gm23374 | gene | MSTRG.57114 | 120.5661687 | 1.49E−08 | 4.77E−06 |
| Gm6133 | gene | MSTRG.68887 | 6.237221571 | 1.53E−08 | 4.88E−06 |
| Rpl31-ps16 | gene | MSTRG.64064 | 22.62131815 | 1.54E−08 | 4.88E−06 |
| Gm28911 | gene | MSTRG.6813 | 142.2721936 | 1.56E−08 | 4.89E−06 |
| Rpl31-ps13 | gene | MSTRG.40063 | 33.90652677 | 1.56E−08 | 4.89E−06 |
| Slc5a11 | gene | MSTRG.128912 | 87.17259371 | 1.57E−08 | 4.89E−06 |
| Gm17511 | gene | MSTRG.129193 | 5.291499115 | 1.59E−08 | 4.92E−06 |
| Rps12-ps5 | gene | MSTRG.122988 | 7.455403012 | 1.59E−08 | 4.92E−06 |
| Hmgb1-ps9 | gene | MSTRG.40105 | 11.92551043 | 1.62E−08 | 4.97E−06 |
| Gm16513 | gene | MSTRG.108177 | 21.32902392 | 1.64E−08 | 5.02E−06 |
| Gm8696 | gene | MSTRG.61106 | 8.088619265 | 1.69E−08 | 5.15E−06 |
| Gm15464 | gene | MSTRG.3307 | 16.11013968 | 1.71E−08 | 5.17E−06 |
| Tnfrsf9 | gene | MSTRG.102353 | 14.99325282 | 1.73E−08 | 5.22E−06 |
| Ccdc42 | gene | MSTRG.23369 | 77.00676956 | 1.79E−08 | 5.37E−06 |
| . | gene | MSTRG.145761 | 5.098291638 | 1.88E−08 | 5.62E−06 |
| Gm9701 | gene | MSTRG.87708 | 12.03855548 | 1.90E−08 | 5.63E−06 |
| . | gene | MSTRG.43662 | 65.91104294 | 1.91E−08 | 5.63E−06 |
| BC048679 | gene | MSTRG.126200 | 230.3401132 | 1.94E−08 | 5.70E−06 |
| Tab1 | gene | MSTRG.51548 | 5.76333006 | 2.00E−08 | 5.84E−06 |
| Rpl19-ps1 | gene | MSTRG.4873 | 52.82626134 | 2.05E−08 | 5.97E−06 |
| Gm10237 | gene | MSTRG.55654 | 13.76838525 | 2.07E−08 | 5.99E−06 |
| Gm12727 | gene | MSTRG.98964 | 44.28340033 | 2.10E−08 | 6.04E−06 |
| RP24-144C5.3 | gene | MSTRG.129310 | 25.92869444 | 2.24E−08 | 6.40E−06 |
| . | gene | MSTRG.98736 | 78.35758461 | 2.27E−08 | 6.43E−06 |
| Actg-ps1 | gene | MSTRG.132929 | 97.89772726 | 2.28E−08 | 6.45E−06 |
| Gm5445 | gene | MSTRG.34753 | 5.266884358 | 2.36E−08 | 6.64E−06 |
| Rpl10-ps2 | gene | MSTRG.136240 | 13.02117005 | 2.40E−08 | 6.73E−06 |
| Cd81 | gene | MSTRG.130268 | 0.002077654 | 2.49E−08 | 6.90E−06 |
| . | gene | MSTRG.55443 | 9.231770697 | 2.50E−08 | 6.90E−06 |
| Rpl17-ps4 | gene | MSTRG.96939 | 21.03405864 | 2.51E−08 | 6.90E−06 |
| Trim38 | gene | MSTRG.35535 | 25.8397546 | 2.51E−08 | 6.90E−06 |
| Gm12337 | gene | MSTRG.24012 | 25.11963999 | 2.63E−08 | 7.20E−06 |
| . | gene | MSTRG.47212 | 10.48154537 | 2.68E−08 | 7.29E−06 |
| Timd2 | gene | MSTRG.21843 | 316.9369166 | 2.70E−08 | 7.31E−06 |
| . | gene | MSTRG.137500 | 4.077576158 | 2.78E−08 | 7.47E−06 |
| Gm6652 | gene | MSTRG.8646 | 89.04499214 | 2.78E−08 | 7.47E−06 |
| . | gene | MSTRG.28565 | 19.89300469 | 2.81E−08 | 7.51E−06 |
| RP24-471H15.4 | gene | MSTRG.123043 | 23.5004527 | 2.86E−08 | 7.61E−06 |
| Gm14870 | gene | MSTRG.149669 | 3.146426809 | 2.97E−08 | 7.84E−06 |
| Gm11675 | gene | MSTRG.26894 | 7.151188684 | 2.97E−08 | 7.84E−06 |
| Gm3076 | gene | MSTRG.93428 | 11.43884432 | 3.01E−08 | 7.89E−06 |
| Gm14036 | gene | MSTRG.81330 | 37.41937884 | 3.05E−08 | 7.97E−06 |
| . | gene | MSTRG.138135 | 85.2439273 | 3.11E−08 | 8.06E−06 |
| Gm10163 | gene | MSTRG.143042 | 16.26947101 | 3.12E−08 | 8.06E−06 |
| Gm6286 | gene | MSTRG.9536 | 15.18564043 | 3.13E−08 | 8.06E−06 |
| . | gene | MSTRG.28701 | 7.232213192 | 3.28E−08 | 8.40E−06 |
| Gm10250 | gene | MSTRG.47660 | 21.10034626 | 3.54E−08 | 9.02E−06 |
| Rplp0-ps1 | gene | MSTRG.89456 | 5.127509881 | 3.69E−08 | 9.34E−06 |
| Gm5527 | gene | MSTRG.2807 | 10.52281879 | 3.76E−08 | 9.49E−06 |
| Rps19-ps4 | gene | MSTRG.66809 | 46.34237045 | 3.80E−08 | 9.54E−06 |
| Slc35g1 | gene | MSTRG.72023 | 19.94393814 | 3.81E−08 | 9.54E−06 |
| Rpl31-ps12 | gene | MSTRG.54288 | 35.75808386 | 3.84E−08 | 9.58E−06 |
| Alppl2 | gene | MSTRG.4936 | 62.71471227 | 4.08E−08 | 1.01E−05 |
| . | gene | MSTRG.84211 | 6.818934061 | 4.11E−08 | 1.02E−05 |
| . | gene | MSTRG.17163 | 203.0090238 | 4.38E−08 | 1.08E−05 |
| Ube2nl | gene | MSTRG.124885 | 5.038327057 | 4.45E−08 | 1.09E−05 |
| Gm14633 | gene | MSTRG.147532 | 69.11395493 | 4.47E−08 | 1.09E−05 |
| Hmgb1-ps3 | gene | MSTRG.23083 | 11.34839443 | 4.47E−08 | 1.09E−05 |
| . | gene | MSTRG.35050 | 40.244115 | 4.58E−08 | 1.11E−05 |
| Sycp3 | gene | MSTRG.16696 | 38.55433729 | 4.69E−08 | 1.13E−05 |
| Gm12231 | gene | MSTRG.22337 | 62.65548657 | 4.78E−08 | 1.15E−05 |
| Gm8213 | gene | MSTRG.118666 | 12.30297882 | 4.82E−08 | 1.15E−05 |
| . | gene | MSTRG.25510 | 10.4750226 | 4.93E−08 | 1.18E−05 |
| Gm14173 | gene | MSTRG.82634 | 30.48427713 | 4.97E−08 | 1.18E−05 |
| . | gene | MSTRG.121732 | 3.388212175 | 5.06E−08 | 1.19E−05 |
| Gm10288 | gene | MSTRG.93086 | 64.22385525 | 5.19E−08 | 1.22E−05 |
| Gm43097 | gene | MSTRG.90884 | 5.952401444 | 5.33E−08 | 1.25E−05 |
| . | gene | MSTRG.70232 | 22.5671172 | 5.52E−08 | 1.29E−05 |
| Gm3851 | gene | MSTRG.6654 | 14.78564041 | 5.63E−08 | 1.30E−05 |
| Gm9575 | gene | MSTRG.56527 | 6.803160787 | 5.63E−08 | 1.30E−05 |
| . | gene | MSTRG.54477 | 7.773814953 | 5.79E−08 | 1.33E−05 |
| 2010005H15Rik | gene | MSTRG.55680 | 15.90043987 | 6.14E−08 | 1.41E−05 |
| Gm15843 | gene | MSTRG.3985 | 12.51996544 | 6.20E−08 | 1.42E−05 |
| . | gene | MSTRG.46087 | 20.43163264 | 6.28E−08 | 1.43E−05 |
| Gm11826 | gene | MSTRG.94264 | 32.59890853 | 6.43E−08 | 1.46E−05 |
| Sppl2a | gene | MSTRG.80713 | 10.67661253 | 6.58E−08 | 1.49E−05 |
| . | gene | MSTRG.25168 | 4.283026377 | 6.91E−08 | 1.55E−05 |
| Rpl28-ps1 | gene | MSTRG.7060 | 49.8970738 | 6.93E−08 | 1.55E−05 |
| . | gene | MSTRG.109616 | 6.625821754 | 7.12E−08 | 1.59E−05 |
| Rps24-ps3 | gene | MSTRG.148474 | 45.34636749 | 7.23E−08 | 1.60E−05 |
| Gm13532 | gene | MSTRG.76996 | 5.372084801 | 7.25E−08 | 1.60E−05 |
| Slc15a2 | gene | MSTRG.55726 | 75.02529254 | 7.31E−08 | 1.61E−05 |
| Pramel5 | gene | MSTRG.101943 | 46.49222747 | 7.36E−08 | 1.62E−05 |
| Gm16477 | gene | MSTRG.130017 | 24.49436953 | 7.41E−08 | 1.62E−05 |
| . | gene | MSTRG.46179 | 22.25859996 | 7.53E−08 | 1.64E−05 |
| Gm20900 | gene | MSTRG.41204 | 68.30876646 | 7.74E−08 | 1.68E−05 |
| Gm12458 | gene | MSTRG.96214 | 5.41526742 | 7.75E−08 | 1.68E−05 |
| Gm11448 | gene | MSTRG.82951 | 3.553019323 | 8.19E−08 | 1.76E−05 |
| Gm10268 | gene | MSTRG.66480 | 20.11700033 | 9.63E−08 | 2.05E−05 |
| Gm2710 | gene | MSTRG.9921 | 13.12919299 | 1.08E−07 | 2.29E−05 |
| Rpl21-ps10 | gene | MSTRG.86366 | 11.71585687 | 1.15E−07 | 2.42E−05 |
| Gm13573 | gene | MSTRG.77120 | 6.983247223 | 1.16E−07 | 2.44E−05 |
| . | gene | MSTRG.31139 | 24.2384821 | 1.18E−07 | 2.48E−05 |
| Gm13827 | gene | MSTRG.109533 | 17.14407188 | 1.20E−07 | 2.50E−05 |
| Gm9294 | gene | MSTRG.123897 | 16.08528402 | 1.21E−07 | 2.52E−05 |
| . | gene | MSTRG.124884 | 13.90967009 | 1.22E−07 | 2.52E−05 |
| Eif3s6-ps1 | gene | MSTRG.19665 | 8.590512885 | 1.26E−07 | 2.60E−05 |
| . | gene | MSTRG.101957 | 24.67134735 | 1.30E−07 | 2.67E−05 |
| C230085N15Rik | gene | MSTRG.62188 | 5.152846745 | 1.31E−07 | 2.69E−05 |
| Gm5857 | gene | MSTRG.92913 | 7.077133511 | 1.34E−07 | 2.74E−05 |
| Gpha2 | gene | MSTRG.69890 | 75.84027892 | 1.34E−07 | 2.74E−05 |
| . | gene | MSTRG.72227 | 10.35259269 | 1.46E−07 | 2.96E−05 |
| Gm3139 | gene | MSTRG.108125 | 26.51051523 | 1.47E−07 | 2.97E−05 |
| . | gene | MSTRG.115812 | 9.070238687 | 1.48E−07 | 2.97E−05 |
| . | gene | MSTRG.68058 | 3.098661742 | 1.53E−07 | 3.06E−05 |
| . | gene | MSTRG.70738 | 79.83782706 | 1.54E−07 | 3.07E−05 |
| . | gene | MSTRG.25940 | 16.32953375 | 1.56E−07 | 3.10E−05 |
| . | gene | MSTRG.78555 | 4.576552829 | 1.57E−07 | 3.11E−05 |
| . | gene | MSTRG.93952 | 14.76503968 | 1.58E−07 | 3.11E−05 |
| D10Wsu102e | gene | MSTRG.16287 | 15.33544739 | 1.58E−07 | 3.11E−05 |
| Gm12331 | gene | ENSMUSG00000081932 | 4.91866899 | 1.66E−07 | 3.23E−05 |
| Platr27 | gene | MSTRG.82762 | 18.35993133 | 1.66E−07 | 3.23E−05 |
| . | gene | MSTRG.34903 | 2.852878646 | 1.66E−07 | 3.23E−05 |
| Gm5093 | gene | MSTRG.61801 | 3.368423891 | 1.80E−07 | 3.50E−05 |
| . | gene | MSTRG.82522 | 5.829396709 | 1.83E−07 | 3.54E−05 |
| . | gene | MSTRG.145760 | 15.1533948 | 1.85E−07 | 3.56E−05 |
| Gm27529 | gene | MSTRG.47816 | 102.5432526 | 1.87E−07 | 3.60E−05 |
| . | gene | MSTRG.98499 | 9.297467752 | 1.88E−07 | 3.60E−05 |
| . | gene | MSTRG.34339 | 69.7430883 | 1.90E−07 | 3.62E−05 |
| Gm11539 | gene | MSTRG.25622 | 25.37690834 | 1.91E−07 | 3.63E−05 |
| Gm29257 | gene | MSTRG.7421 | 9.873680591 | 1.92E−07 | 3.65E−05 |
| Gm13370 | gene | MSTRG.74977 | 12.90414999 | 1.95E−07 | 3.70E−05 |
| Timm8a2 | gene | MSTRG.47412 | 57.17355768 | 1.99E−07 | 3.76E−05 |
| Rpsa-ps11 | gene | ENSMUSG00000082978 | 3.523859228 | 2.07E−07 | 3.90E−05 |
| Rpl30-ps9 | gene | MSTRG.149500 | 32.48668922 | 2.12E−07 | 3.97E−05 |
| Gm6341 | gene | MSTRG.127484 | 21.00359204 | 2.16E−07 | 4.03E−05 |
| BC028528 | gene | MSTRG.89731 | 43.65400982 | 2.23E−07 | 4.16E−05 |
| Gm12344 | gene | MSTRG.24240 | 20.5407075 | 2.27E−07 | 4.21E−05 |
| . | gene | MSTRG.142039 | 3.531518469 | 2.39E−07 | 4.43E−05 |
| Gm14138 | gene | MSTRG.80203 | 6.201026658 | 2.45E−07 | 4.53E−05 |
| Gm7808 | gene | MSTRG.138205 | 30.26470439 | 2.53E−07 | 4.64E−05 |
| . | gene | MSTRG.146704 | 16.02661936 | 2.56E−07 | 4.68E−05 |
| . | gene | MSTRG.137431 | 9.593095967 | 2.57E−07 | 4.68E−05 |
| . | gene | MSTRG.96863 | 4.978808499 | 2.58E−07 | 4.69E−05 |
| . | gene | MSTRG.31595 | 3.835229507 | 2.63E−07 | 4.76E−05 |
| . | gene | MSTRG.152381 | 13.74940438 | 2.67E−07 | 4.82E−05 |
| Rpl30-ps8 | gene | MSTRG.32856 | 25.17194559 | 2.72E−07 | 4.89E−05 |
| Gm11954 | gene | MSTRG.19264 | 7.384396773 | 2.79E−07 | 4.99E−05 |
| Gm6083 | gene | MSTRG.104374 | 10.08148056 | 2.82E−07 | 5.04E−05 |
| Gm7429 | gene | MSTRG.149839 | 6.426942475 | 2.84E−07 | 5.06E−05 |
| Rpl21-ps12 | gene | MSTRG.117355 | 4.02425047 | 2.88E−07 | 5.10E−05 |
| Gm14148 | gene | MSTRG.82152 | 31.46495874 | 2.98E−07 | 5.26E−05 |
| AC165294.3 | gene | MSTRG.145590 | 11.57851375 | 3.08E−07 | 5.41E−05 |
| . | gene | MSTRG.150851 | 9.402492326 | 3.09E−07 | 5.42E−05 |
| Gm14251 | gene | MSTRG.82645 | 36.10309573 | 3.12E−07 | 5.46E−05 |
| . | gene | MSTRG.50252 | 10.29926786 | 3.18E−07 | 5.55E−05 |
| Gm6681 | gene | MSTRG.117864 | 11.38875065 | 3.31E−07 | 5.73E−05 |
| Hmgb1-ps2 | gene | MSTRG.150854 | 5.294983122 | 3.42E−07 | 5.91E−05 |
| Sycn | gene | MSTRG.122823 | 34.92663274 | 3.45E−07 | 5.95E−05 |
| mt-Tl2 | gene | MSTRG.145687 | 8.340404653 | 3.51E−07 | 6.02E−05 |
| . | gene | MSTRG.25616 | 24.30577296 | 3.64E−07 | 6.22E−05 |
| . | gene | MSTRG.83509 | 26.74370965 | 3.77E−07 | 6.42E−05 |
| Gldc | gene | MSTRG.71548 | 13.55646942 | 3.84E−07 | 6.51E−05 |
| Asz1 | gene | MSTRG.113260 | 10.55913832 | 3.91E−07 | 6.59E−05 |
| Gm13148 | gene | MSTRG.102863 | 6.496404033 | 4.07E−07 | 6.83E−05 |
| Rps8-ps4 | gene | MSTRG.121571 | 6.446401576 | 4.11E−07 | 6.88E−05 |
| . | gene | MSTRG.146812 | 16.66033635 | 4.41E−07 | 7.33E−05 |
| Gm5580 | gene | MSTRG.118693 | 3.539004055 | 4.42E−07 | 7.33E−05 |
| Vtcn1 | gene | MSTRG.90166 | 9.189384843 | 4.48E−07 | 7.41E−05 |
| Cd63-ps | gene | MSTRG.67275 | 15.72369896 | 4.53E−07 | 7.47E−05 |
| Gm42573 | gene | MSTRG.113467 | 14.28877451 | 4.54E−07 | 7.47E−05 |
| Gm12704 | gene | MSTRG.98331 | 4.913545419 | 4.61E−07 | 7.56E−05 |
| RP23-212H18.2 | gene | MSTRG.124975 | 4.417390178 | 4.73E−07 | 7.74E−05 |
| . | gene | MSTRG.146909 | 3.069535802 | 4.79E−07 | 7.82E−05 |
| Gm13339 | gene | MSTRG.74810 | 42.35330513 | 4.91E−07 | 7.99E−05 |
| . | gene | MSTRG.14565 | 4.423917235 | 5.23E−07 | 8.49E−05 |
| . | gene | MSTRG.48953 | 20.48776831 | 5.32E−07 | 8.61E−05 |
| Zfp839 | gene | MSTRG.33677 | 3.964595657 | 5.42E−07 | 8.75E−05 |
| . | gene | MSTRG.54844 | 4.025296093 | 5.46E−07 | 8.79E−05 |
| Fbxo8 | gene | MSTRG.133418 | 10.01043649 | 5.64E−07 | 9.05E−05 |
| Gm5937 | gene | MSTRG.148423 | 5.034317539 | 5.68E−07 | 9.09E−05 |
| Gm6054 | gene | MSTRG.112257 | 143.1292497 | 5.75E−07 | 9.19E−05 |
| . | gene | MSTRG.2449 | 5.414842803 | 5.94E−07 | 9.46E−05 |
| Xlr4b | gene | MSTRG.148178 | 15.32057226 | 6.06E−07 | 9.60E−05 |
| Gm43471 | gene | MSTRG.87112 | 8.284631377 | 6.07E−07 | 9.60E−05 |
| Gm16165 | gene | MSTRG.138502 | 11.28448862 | 6.17E−07 | 9.75E−05 |
| Gm13268 | gene | MSTRG.74264 | 3.738787697 | 6.36E−07 | 0.000100215 |
| . | gene | MSTRG.28394 | 17.57380854 | 6.48E−07 | 0.000101752 |
| . | gene | MSTRG.53424 | 8.01235608 | 6.78E−07 | 0.000106256 |
| AI662270 | gene | MSTRG.24815 | 17.00406696 | 6.88E−07 | 0.000107167 |
| . | gene | MSTRG.142979 | 4.798654622 | 6.89E−07 | 0.000107167 |
| . | gene | MSTRG.89547 | 33.76259377 | 6.90E−07 | 0.000107167 |
| Gm9104 | gene | MSTRG.61695 | 23.71708949 | 7.02E−07 | 0.000108837 |
| Rps3a3 | gene | MSTRG.40578 | 31.37309219 | 7.06E−07 | 0.000109115 |
| Gm11401 | gene | MSTRG.19473 | 2.742252278 | 7.21E−07 | 0.000110768 |
| Gm12261 | gene | MSTRG.22647 | 14.34235221 | 7.21E−07 | 0.000110768 |
| . | gene | MSTRG.151845 | 13.47674937 | 7.22E−07 | 0.000110768 |
| Morn2 | gene | MSTRG.63805 | 15.99061429 | 7.27E−07 | 0.000111219 |
| Gm44180 | gene | MSTRG.118624 | 6.862593144 | 7.42E−07 | 0.000113225 |
| Gm13578 | gene | MSTRG.77303 | 3.295947319 | 7.44E−07 | 0.000113247 |
| Gm12174 | gene | MSTRG.21847 | 29.26903704 | 7.57E−07 | 0.000114946 |
| . | gene | MSTRG.72111 | 7.489153099 | 7.62E−07 | 0.000115361 |
| . | gene | MSTRG.34222 | 23.28080852 | 7.72E−07 | 0.000116551 |
| Gm13611 | gene | MSTRG.75655 | 70.0899223 | 7.85E−07 | 0.000118317 |
| Gm16288 | gene | MSTRG.104453 | 5.172410834 | 8.29E−07 | 0.00012466 |
| Rps4x-ps | gene | MSTRG.102939 | 5.518865999 | 8.39E−07 | 0.000125702 |
| Gm11449 | gene | MSTRG.82959 | 28.58983429 | 8.67E−07 | 0.000129709 |
| . | gene | MSTRG.17024 | 13.1701118 | 8.75E−07 | 0.000130429 |
| . | gene | MSTRG.152282 | 12.59853985 | 8.84E−07 | 0.000131421 |
| Gm9794 | gene | MSTRG.115913 | 40.4443248 | 8.86E−07 | 0.000131421 |
| Gm21399 | gene | MSTRG.137371 | 22.51966778 | 8.92E−07 | 0.000131681 |
| . | gene | MSTRG.28622 | 37.97296462 | 8.94E−07 | 0.000131681 |
| . | gene | MSTRG.60736 | 3.31390668 | 9.32E−07 | 0.00013694 |
| . | gene | MSTRG.149838 | 3.920104387 | 9.42E−07 | 0.000137774 |
| . | gene | MSTRG.68840 | 11.36717997 | 9.54E−07 | 0.000139159 |
| . | gene | MSTRG.36825 | 0.002313198 | 9.63E−07 | 0.000140041 |
| Anp32-ps | gene | MSTRG.21166 | 6.714375502 | 9.77E−07 | 0.000141712 |
| Reep1 | gene | MSTRG.116225 | 8.923260882 | 1.01E−06 | 0.000145959 |
| Gm5045 | gene | MSTRG.50251 | 7.791935084 | 1.03E−06 | 0.000148599 |
| RP24-269P21.1 | gene | MSTRG.127026 | 6.69284763 | 1.03E−06 | 0.000148599 |
| Rpl30-ps1 | gene | MSTRG.114378 | 18.97615769 | 1.05E−06 | 0.000150724 |
| . | gene | MSTRG.107989 | 4.162142071 | 1.05E−06 | 0.000150724 |
| Gm12618 | gene | MSTRG.22821 | 3.893703497 | 1.05E−06 | 0.000150763 |
| Ppm1k | gene | MSTRG.115593 | 6.113868054 | 1.07E−06 | 0.000152614 |
| Gm5845 | gene | MSTRG.86063 | 3.165091525 | 1.13E−06 | 0.000161485 |
| Gm11222 | gene | MSTRG.97138 | 6.844617786 | 1.14E−06 | 0.000161896 |
| Gm6335 | gene | MSTRG.149739 | 39.01643171 | 1.14E−06 | 0.000161896 |
| Gm12906 | gene | MSTRG.99122 | 5.211808006 | 1.16E−06 | 0.000163996 |
| Gm5160 | gene | MSTRG.65339 | 3.934186349 | 1.19E−06 | 0.000167799 |
| Gm11953 | gene | MSTRG.19256 | 7.78996402 | 1.22E−06 | 0.000171167 |
| . | gene | MSTRG.137433 | 7.082778653 | 1.23E−06 | 0.000172205 |
| Gm11575 | gene | MSTRG.3362 | 8.692442285 | 1.27E−06 | 0.000176467 |
| Rpl7a-ps5 | gene | MSTRG.62530 | 4.345082111 | 1.28E−06 | 0.000178403 |
| AC164084.2 | gene | MSTRG.145582 | 11.12323899 | 1.34E−06 | 0.000185708 |
| Gm5518 | gene | MSTRG.71473 | 3.949262975 | 1.34E−06 | 0.000185708 |
| Rpl18-ps1 | gene | MSTRG.3405 | 11.84709291 | 1.36E−06 | 0.000187263 |
| Rpl7-ps7 | gene | MSTRG.107518 | 17.74479057 | 1.37E−06 | 0.000188897 |
| RP24-427A8.1 | gene | MSTRG.129529 | 4.012392408 | 1.38E−06 | 0.000190013 |
| . | gene | MSTRG.46669 | 3.562481461 | 1.42E−06 | 0.000194567 |
| Rhobtb2 | gene | MSTRG.45168 | 4.598121838 | 1.48E−06 | 0.000201954 |
| Gm9493 | gene | MSTRG.71084 | 29.23981222 | 1.53E−06 | 0.000207611 |
| Tspan8 | gene | MSTRG.18185 | 477.2226838 | 1.56E−06 | 0.000212172 |
| . | gene | MSTRG.1978 | 15.05693106 | 1.59E−06 | 0.000214849 |
| . | gene | MSTRG.54336 | 27.08801344 | 1.61E−06 | 0.000216795 |
| . | gene | MSTRG.28730 | 23.73984922 | 1.64E−06 | 0.000219971 |
| Gm13422 | gene | MSTRG.74848 | 9.603724057 | 1.66E−06 | 0.000222236 |
| Gm9625 | gene | MSTRG.38262 | 23.81072401 | 1.69E−06 | 0.000226239 |
| Rpl7a-ps11 | gene | MSTRG.151710 | 16.8200539 | 1.69E−06 | 0.000226327 |
| Gm8337 | gene | MSTRG.2534 | 5.797116902 | 1.74E−06 | 0.000231652 |
| . | gene | MSTRG.137799 | 7.079945698 | 1.74E−06 | 0.000231652 |
| . | gene | MSTRG.102826 | 9.916781266 | 1.81E−06 | 0.00024033 |
| Ccpg1os | gene | MSTRG.142163 | 16.24850024 | 1.81E−06 | 0.00024033 |
| Rps15a-ps6 | gene | MSTRG.19475 | 21.34357668 | 1.82E−06 | 0.000240428 |
| . | gene | MSTRG.135208 | 10.53914983 | 1.83E−06 | 0.000241309 |
| . | gene | MSTRG.140774 | 26.73486531 | 1.83E−06 | 0.000241373 |
| . | gene | MSTRG.32970 | 5.989693776 | 1.85E−06 | 0.000243279 |
| Gm36964 | gene | MSTRG.368 | 4.058875635 | 1.87E−06 | 0.000245286 |
| Gm11598 | gene | ENSMUSG00000082456 | 2.805662127 | 1.88E−06 | 0.000245638 |
| Gm10073 | gene | MSTRG.136178 | 23.06636219 | 1.89E−06 | 0.000246735 |
| Chga | gene | MSTRG.33095 | 10.99429526 | 1.97E−06 | 0.00025672 |
| Gm3355 | gene | MSTRG.59410 | 3.798551032 | 1.98E−06 | 0.000256961 |
| . | gene | MSTRG.33622 | 5.980706145 | 1.99E−06 | 0.000257827 |
| . | gene | MSTRG.92016 | 13.1257441 | 2.02E−06 | 0.000260397 |
| . | gene | MSTRG.111517 | 4.718294579 | 2.02E−06 | 0.000260397 |
| Gm6166 | gene | MSTRG.141034 | 46.56727865 | 2.07E−06 | 0.000266216 |
| Gm13408 | gene | MSTRG.75922 | 56.77368055 | 2.09E−06 | 0.000267729 |
| Gm15361 | gene | MSTRG.148270 | 5.540222437 | 2.10E−06 | 0.000269105 |
| Rpl30-ps10 | gene | MSTRG.148087 | 23.80653838 | 2.11E−06 | 0.000269105 |
| . | gene | MSTRG.85147 | 6.410258783 | 2.12E−06 | 0.000269105 |
| Dennd5a | gene | MSTRG.128060 | 4.649721693 | 2.12E−06 | 0.000269105 |
| Gm6478 | gene | MSTRG.37982 | 2.941274712 | 2.12E−06 | 0.000269105 |
| Gm13835 | gene | MSTRG.113978 | 12.92736513 | 2.14E−06 | 0.000271271 |
| . | gene | MSTRG.69498 | 4.573594549 | 2.17E−06 | 0.000274039 |
| Gm10240 | gene | MSTRG.50594 | 13.67698682 | 2.20E−06 | 0.000277141 |
| . | gene | MSTRG.124052 | 3.795212105 | 2.22E−06 | 0.000279699 |
| G430049J08Rik | gene | MSTRG.64616 | 12.47199559 | 2.24E−06 | 0.000282105 |
| Gm14300 | gene | MSTRG.84210 | 7.907543867 | 2.26E−06 | 0.000283689 |
| . | gene | MSTRG.18143 | 10.92763334 | 2.31E−06 | 0.000288927 |
| Aldh3b2 | gene | MSTRG.69611 | 7.894837367 | 2.36E−06 | 0.000294758 |
| Snora78 | gene | MSTRG.60004 | 14.07518708 | 2.39E−06 | 0.000297321 |
| Gm5853 | gene | MSTRG.90023 | 4.233824084 | 2.39E−06 | 0.000297475 |
| . | gene | MSTRG.152174 | 12.19147949 | 2.49E−06 | 0.00030882 |
| Gm8399 | gene | MSTRG.38892 | 44.31789867 | 2.61E−06 | 0.000322633 |
| Khdc1a | gene | MSTRG.1101 | 7.908958122 | 2.79E−06 | 0.000343781 |
| Cmbl | gene | MSTRG.48870 | 108.8543601 | 2.83E−06 | 0.000347677 |
| Ccdc170 | gene | MSTRG.11404 | 4.487706013 | 2.87E−06 | 0.000351916 |
| Gm9050 | gene | MSTRG.149394 | 4.439523715 | 2.93E−06 | 0.000358944 |
| . | gene | MSTRG.137430 | 13.77287462 | 2.99E−06 | 0.000365767 |
| Tmem86a | gene | MSTRG.124252 | 10.66134845 | 3.01E−06 | 0.00036602 |
| . | gene | MSTRG.122656 | 4.827919135 | 3.03E−06 | 0.000367446 |
| Gm8865 | gene | MSTRG.44166 | 8.909124662 | 3.07E−06 | 0.000370032 |
| Gm8318 | gene | MSTRG.48341 | 6.589407488 | 3.08E−06 | 0.000370032 |
| . | gene | MSTRG.8872 | 3.908438661 | 3.08E−06 | 0.000370032 |
| . | gene | MSTRG.11583 | 8.520301426 | 3.16E−06 | 0.000378802 |
| Gm10051 | gene | MSTRG.110757 | 48.24691852 | 3.47E−06 | 0.000412475 |
| . | gene | MSTRG.13676 | 5.769496394 | 3.47E−06 | 0.000412475 |
| Eno1 | gene | MSTRG.102303 | 0.078150392 | 3.48E−06 | 0.000412639 |
| Gm43028 | gene | MSTRG.106704 | 7.565619274 | 3.49E−06 | 0.000412639 |
| Gm8226 | gene | MSTRG.142775 | 19.08941796 | 3.52E−06 | 0.000415564 |
| Rpl31-ps4 | gene | MSTRG.57996 | 6.724216059 | 3.53E−06 | 0.000415564 |
| Gm7027 | gene | MSTRG.127498 | 26.1299899 | 3.65E−06 | 0.000429495 |
| Gm5805 | gene | MSTRG.51745 | 28.67777041 | 3.68E−06 | 0.000432205 |
| Rpl21-ps8 | gene | MSTRG.69131 | 34.95492517 | 3.76E−06 | 0.000441094 |
| . | gene | MSTRG.49284 | 5.359216093 | 3.79E−06 | 0.000443122 |
| Cpn1 | gene | MSTRG.72409 | 33.87024708 | 3.86E−06 | 0.000450175 |
| . | gene | MSTRG.73636 | 3.95379373 | 4.03E−06 | 0.000468325 |
| Gm13310 | gene | MSTRG.74160 | 4.001966405 | 4.14E−06 | 0.000480525 |
| . | gene | MSTRG.25156 | 5.505587752 | 4.36E−06 | 0.000505309 |
| . | gene | MSTRG.75901 | 25.95018291 | 4.44E−06 | 0.000513181 |
| BC053393 | gene | MSTRG.21759 | 124.1760129 | 4.47E−06 | 0.000515864 |
| Gm43008 | gene | MSTRG.86420 | 4.981855784 | 4.54E−06 | 0.000521132 |
| Gm3531 | gene | MSTRG.5674 | 33.06404234 | 4.62E−06 | 0.000528626 |
| Gm12254 | gene | MSTRG.22548 | 77.10054991 | 4.62E−06 | 0.000528626 |
| Gm7832 | gene | MSTRG.107940 | 4.699684116 | 4.72E−06 | 0.000539744 |
| Gm15387 | gene | MSTRG.50973 | 2.440043724 | 4.74E−06 | 0.000540198 |
| Gm8623 | gene | MSTRG.133007 | 31.1262599 | 4.92E−06 | 0.000559064 |
| Gm11407 | gene | MSTRG.97462 | 2.94200278 | 4.95E−06 | 0.000561066 |
| . | gene | MSTRG.134534 | 45.92868583 | 4.97E−06 | 0.000562645 |
| Gm10709 | gene | MSTRG.137617 | 18.31302892 | 5.00E−06 | 0.00056425 |
| . | gene | MSTRG.13851 | 9.753229461 | 5.35E−06 | 0.000601873 |
| . | gene | MSTRG.128441 | 4.810132837 | 5.35E−06 | 0.000601873 |
| Gm16378 | gene | MSTRG.140349 | 24.53313739 | 5.39E−06 | 0.000604755 |
| Gm14648 | gene | MSTRG.147204 | 9.180429392 | 5.65E−06 | 0.000632183 |
| Hist1h2ap | gene | MSTRG.35488 | 0.003568328 | 5.68E−06 | 0.000633851 |
| . | gene | MSTRG.122581 | 4.10970017 | 5.84E−06 | 0.000649915 |
| Gm12115 | gene | MSTRG.21100 | 4.609012381 | 5.91E−06 | 0.000656648 |
| Gm7327 | gene | MSTRG.147709 | 6.673732 | 6.30E−06 | 0.000696451 |
| Npr1 | gene | MSTRG.89334 | 17.95944014 | 6.35E−06 | 0.000701099 |
| Gm5873 | gene | ENSMUSG00000093651 | 5.995248662 | 6.40E−06 | 0.000704824 |
| . | gene | MSTRG.48627 | 11.17656258 | 6.43E−06 | 0.000707519 |
| Ttc39c | gene | MSTRG.65256 | 10.42538615 | 6.52E−06 | 0.000715674 |
| Gm13882 | gene | MSTRG.79353 | 7.059110872 | 6.72E−06 | 0.000734676 |
| Ube2l6 | gene | MSTRG.78394 | 43.49877729 | 6.76E−06 | 0.000737987 |
| . | gene | MSTRG.53155 | 3.841763451 | 7.01E−06 | 0.000763988 |
| . | gene | MSTRG.59444 | 9.751893624 | 7.22E−06 | 0.00078496 |
| Gm12712 | gene | MSTRG.23791 | 5.910911271 | 7.24E−06 | 0.000785952 |
| . | gene | MSTRG.63882 | 39.95067079 | 7.29E−06 | 0.000790354 |
| D5Ertd605e | gene | MSTRG.112265 | 26.94770754 | 7.31E−06 | 0.000790598 |
| Gm15198 | gene | MSTRG.13680 | 2.633495603 | 7.34E−06 | 0.000790748 |
| . | gene | MSTRG.136583 | 3.944107297 | 7.44E−06 | 0.000797204 |
| . | gene | MSTRG.14680 | 32.49518041 | 7.50E−06 | 0.000800416 |
| Gm7816 | gene | MSTRG.105263 | 25.84177992 | 7.51E−06 | 0.000800416 |
| Gm37070 | gene | MSTRG.10377 | 2.947115957 | 7.76E−06 | 0.000825992 |
| . | gene | MSTRG.50207 | 8.202740197 | 7.78E−06 | 0.000825992 |
| . | gene | MSTRG.16256 | 5.1673848 | 7.83E−06 | 0.000829984 |
| . | gene | MSTRG.38644 | 66.3043817 | 8.00E−06 | 0.000846533 |
| RP23-207N2.1 | gene | MSTRG.125973 | 17.32102499 | 8.22E−06 | 0.000866344 |
| Rpl9-ps7 | gene | MSTRG.77567 | 51.95189372 | 8.23E−06 | 0.000866344 |
| L2hgdh | gene | MSTRG.31068 | 4.761599757 | 8.23E−06 | 0.000866344 |
| Rpl36a-ps1 | gene | MSTRG.46387 | 22.43566395 | 8.51E−06 | 0.00089437 |
| Slc25a16 | gene | MSTRG.14926 | 13.51407726 | 8.81E−06 | 0.000922968 |
| . | gene | MSTRG.41981 | 33.94205697 | 8.81E−06 | 0.000922968 |
| . | gene | MSTRG.84569 | 4.406089846 | 9.23E−06 | 0.000964378 |
| Gm7180 | gene | MSTRG.126152 | 8.737013432 | 9.51E−06 | 0.000992587 |
| . | gene | MSTRG.63868 | 7.666585189 | 9.56E−06 | 0.000995649 |
| Adap2os | gene | MSTRG.24615 | 9.754428549 | 1.01E−05 | 0.001045718 |
| . | gene | MSTRG.81712 | 40.23863452 | 1.02E−05 | 0.001053684 |
| Xlr4c | gene | MSTRG.148180 | 9.0078151 | 1.02E−05 | 0.001053684 |
| . | gene | MSTRG.38104 | 7.817501269 | 1.06E−05 | 0.001084888 |
| . | gene | MSTRG.48954 | 7.511179972 | 1.09E−05 | 0.001114824 |
| . | gene | MSTRG.132726 | 18.30479169 | 1.10E−05 | 0.0011267 |
| Glipr1 | gene | MSTRG.18019 | 24.86200642 | 1.11E−05 | 0.001135534 |
| Gm6180 | gene | MSTRG.132725 | 8.329537371 | 1.13E−05 | 0.001148029 |
| Ngfrap1 | gene | MSTRG.150458 | 0.003070455 | 1.13E−05 | 0.001153622 |
| . | gene | MSTRG.40137 | 18.51030417 | 1.13E−05 | 0.001153622 |
| . | gene | MSTRG.69617 | 13.93129262 | 1.17E−05 | 0.001190411 |
| . | gene | MSTRG.7549 | 9.759257808 | 1.19E−05 | 0.001202109 |
| Gm3286 | gene | MSTRG.145506 | 11.67827817 | 1.19E−05 | 0.001202109 |
| Gm9385 | gene | MSTRG.144787 | 41.77217153 | 1.19E−05 | 0.001203633 |
| . | gene | MSTRG.60422 | 5.525059862 | 1.21E−05 | 0.00121737 |
| Gm7507 | gene | MSTRG.116889 | 4.410542372 | 1.23E−05 | 0.001235847 |
| . | gene | MSTRG.38098 | 17.76496498 | 1.25E−05 | 0.001245206 |
| . | gene | MSTRG.30413 | 8.945584848 | 1.25E−05 | 0.001245206 |
| Gm37164 | gene | MSTRG.85185 | 10.01200678 | 1.26E−05 | 0.001250091 |
| . | gene | MSTRG.50332 | 12.23023672 | 1.26E−05 | 0.0012531 |
| Gm6467 | gene | MSTRG.54740 | 18.07558107 | 1.26E−05 | 0.0012531 |
| Hmgb1-ps1 | gene | MSTRG.21479 | 8.739110348 | 1.30E−05 | 0.001283728 |
| Pdzk1ip1 | gene | MSTRG.99510 | 59.40726007 | 1.30E−05 | 0.001285962 |
| Gm9238 | gene | MSTRG.34001 | 7.732229886 | 1.30E−05 | 0.001285962 |
| Gm12328 | gene | MSTRG.23898 | 7.171011103 | 1.31E−05 | 0.001288387 |
| Gm26384 | gene | MSTRG.59710 | 21.00868494 | 1.31E−05 | 0.001289547 |
| Acbd3 | gene | MSTRG.10441 | 6.001725449 | 1.35E−05 | 0.001315687 |
| Mmgt1 | gene | MSTRG.147542 | 6.22823561 | 1.35E−05 | 0.001315687 |
| . | gene | MSTRG.137434 | 3.385787981 | 1.38E−05 | 0.001343544 |
| . | gene | MSTRG.108159 | 10.34935341 | 1.39E−05 | 0.001358732 |
| Gm5879 | gene | MSTRG.117139 | 87.67238342 | 1.43E−05 | 0.001392387 |
| . | gene | MSTRG.70528 | 14.82953002 | 1.43E−05 | 0.001393582 |
| . | gene | MSTRG.64593 | 5.181253618 | 1.45E−05 | 0.001406094 |
| Gm13232 | gene | MSTRG.102733 | 19.52481463 | 1.49E−05 | 0.001438362 |
| Nynrin | gene | MSTRG.44289 | 7.763608141 | 1.51E−05 | 0.001457259 |
| . | gene | MSTRG.116670 | 10.99839369 | 1.51E−05 | 0.001457259 |
| Rps15a-ps7 | gene | MSTRG.82214 | 2.953716215 | 1.52E−05 | 0.001457718 |
| Ldha | gene | MSTRG.124313 | 0.00974498 | 1.53E−05 | 0.00146388 |
| Gm6091 | gene | MSTRG.137236 | 17.75536767 | 1.56E−05 | 0.001495854 |
| Bhmt2 | gene | MSTRG.39652 | 21.17965818 | 1.57E−05 | 0.001495854 |
| Gm14681 | gene | MSTRG.147904 | 41.03492692 | 1.58E−05 | 0.001500401 |
| Gyg | gene | MSTRG.85458 | 57.67492234 | 1.58E−05 | 0.001502241 |
| Gm27018 | gene | MSTRG.112773 | 2.310464644 | 1.58E−05 | 0.001502919 |
| . | gene | MSTRG.127177 | 5.800103997 | 1.60E−05 | 0.001517723 |
| Rpap1 | gene | MSTRG.80270 | 10.80228103 | 1.64E−05 | 0.001552181 |
| . | gene | MSTRG.85435 | 4.013938071 | 1.74E−05 | 0.00163867 |
| Gm12169 | gene | MSTRG.21750 | 12.21907141 | 1.75E−05 | 0.001645245 |
| Gm13160 | gene | MSTRG.102814 | 13.17268023 | 1.76E−05 | 0.001653091 |
| Rps11-ps2 | gene | MSTRG.27209 | 4.270472682 | 1.79E−05 | 0.001673538 |
| Gm5564 | gene | MSTRG.111381 | 6.245854224 | 1.79E−05 | 0.001673538 |
| Rps19-ps1 | gene | MSTRG.43934 | 7.211844273 | 1.79E−05 | 0.001673538 |
| . | gene | MSTRG.84381 | 25.46061572 | 1.79E−05 | 0.001673538 |
| Vma21-ps | gene | MSTRG.96332 | 3.294894243 | 1.81E−05 | 0.001682834 |
| Rpl36-ps3 | gene | MSTRG.28228 | 32.47222974 | 1.81E−05 | 0.001684162 |
| . | gene | MSTRG.118783 | 2.82582217 | 1.83E−05 | 0.001696922 |
| Marcksl1 | gene | MSTRG.100709 | 0.123866009 | 1.86E−05 | 0.001722882 |
| . | gene | MSTRG.90165 | 35.40963361 | 1.86E−05 | 0.001722925 |
| Gm11652 | gene | MSTRG.26545 | 4.941340349 | 1.98E−05 | 0.001833636 |
| . | gene | MSTRG.121057 | 20.32096886 | 2.04E−05 | 0.00187925 |
| Gm10343 | gene | MSTRG.53812 | 9.398549491 | 2.04E−05 | 0.001884193 |
| . | gene | MSTRG.22697 | 21.03212868 | 2.06E−05 | 0.00189621 |
| . | gene | MSTRG.31019 | 9.63183809 | 2.08E−05 | 0.001912199 |
| Gm10290 | gene | MSTRG.104332 | 3.8930321 | 2.19E−05 | 0.002001075 |
| . | gene | MSTRG.131120 | 19.7203415 | 2.21E−05 | 0.002020191 |
| Gm8618 | gene | MSTRG.7498 | 3.549081325 | 2.23E−05 | 0.00203664 |
| . | gene | MSTRG.86421 | 4.883615376 | 2.24E−05 | 0.002039789 |
| RP23-473E20.3 | gene | MSTRG.127113 | 7.601234745 | 2.26E−05 | 0.002048849 |
| . | gene | MSTRG.28569 | 10.188736 | 2.29E−05 | 0.002069165 |
| . | gene | MSTRG.146811 | 8.144221453 | 2.40E−05 | 0.002166369 |
| Rps12-ps9 | gene | MSTRG.140401 | 13.41503859 | 2.40E−05 | 0.002166369 |
| . | gene | MSTRG.147208 | 8.877110658 | 2.41E−05 | 0.002172392 |
| Pfkl | gene | MSTRG.15740 | 0.056647946 | 2.42E−05 | 0.00217521 |
| Gm10132 | gene | MSTRG.45553 | 9.356251401 | 2.44E−05 | 0.002188113 |
| Hist1h2ag | gene | MSTRG.35507 | 0.015559514 | 2.46E−05 | 0.002203533 |
| . | gene | MSTRG.59861 | 5.362004782 | 2.50E−05 | 0.002236389 |
| H3f3a-ps2 | gene | MSTRG.58165 | 25.53971119 | 2.54E−05 | 0.002267319 |
| Tmem213 | gene | MSTRG.114371 | 10.86715876 | 2.55E−05 | 0.002267319 |
| Gm5510 | gene | MSTRG.69888 | 4.792631544 | 2.55E−05 | 0.002267319 |
| . | gene | MSTRG.15111 | 8.370107663 | 2.56E−05 | 0.002267319 |
| Hist1h2ao | gene | MSTRG.35484 | 0.003707973 | 2.56E−05 | 0.002267319 |
| Ttc7b | gene | MSTRG.32962 | 27.55428447 | 2.57E−05 | 0.002272878 |
| Gm22614 | gene | MSTRG.89831 | 4.356890562 | 2.61E−05 | 0.002306333 |
| . | gene | MSTRG.108776 | 14.26192917 | 2.61E−05 | 0.002306333 |
| . | gene | MSTRG.92833 | 3.430150384 | 2.64E−05 | 0.002330426 |
| . | gene | MSTRG.119443 | 17.17510526 | 2.67E−05 | 0.0023474 |
| Gm11425 | gene | MSTRG.24788 | 8.660003556 | 2.77E−05 | 0.002425299 |
| Gm4217 | gene | MSTRG.51952 | 4.417318372 | 2.80E−05 | 0.002441018 |
| . | gene | MSTRG.77718 | 16.07487317 | 2.81E−05 | 0.00245085 |
| Hspb1 | gene | MSTRG.111096 | 0.003036519 | 2.86E−05 | 0.002486985 |
| Atp6v0c-ps1 | gene | MSTRG.120968 | 3.442842234 | 2.92E−05 | 0.002524853 |
| Eif5al3-ps | gene | MSTRG.107879 | 3.290074575 | 2.94E−05 | 0.002536462 |
| Gm6170 | gene | MSTRG.7091 | 13.11962007 | 2.97E−05 | 0.002561176 |
| . | gene | MSTRG.70759 | 18.68383205 | 3.00E−05 | 0.002580442 |
| Rps19-ps2 | gene | MSTRG.43910 | 6.086412035 | 3.01E−05 | 0.002581809 |
| . | gene | MSTRG.129168 | 10.92887402 | 3.05E−05 | 0.002611218 |
| Gm5558 | gene | MSTRG.107943 | 6.008259266 | 3.09E−05 | 0.002646027 |
| . | gene | MSTRG.54057 | 17.09284223 | 3.16E−05 | 0.00269862 |
| . | gene | MSTRG.139121 | 108.6398587 | 3.17E−05 | 0.00270902 |
| . | gene | MSTRG.58470 | 3.277009891 | 3.22E−05 | 0.002740273 |
| . | gene | MSTRG.65520 | 6.41908211 | 3.24E−05 | 0.002756418 |
| . | gene | MSTRG.29115 | 5.46926407 | 3.25E−05 | 0.002763746 |
| Gm10689 | gene | MSTRG.131807 | 6.997739113 | 3.27E−05 | 0.002771374 |
| . | gene | MSTRG.139851 | 4.08664458 | 3.31E−05 | 0.002798765 |
| . | gene | MSTRG.110572 | 0.021951672 | 3.31E−05 | 0.002798765 |
| Gm5566 | gene | MSTRG.112432 | 4.299609963 | 3.32E−05 | 0.002800454 |
| Gm14706 | gene | MSTRG.148416 | 2.555795875 | 3.47E−05 | 0.002917854 |
| Tcf23 | gene | MSTRG.104523 | 3.924814746 | 3.51E−05 | 0.002948455 |
| Ypel2 | gene | MSTRG.25075 | 11.11525963 | 3.52E−05 | 0.002949113 |
| Hist1h2ac | gene | MSTRG.35567 | 0.014577314 | 3.59E−05 | 0.003008181 |
| . | gene | MSTRG.28835 | 3.814888414 | 3.62E−05 | 0.003025006 |
| Gm11336 | gene | MSTRG.35578 | 0.023425062 | 3.67E−05 | 0.003067652 |
| . | gene | MSTRG.3084 | 8.914340661 | 3.69E−05 | 0.003076772 |
| . | gene | MSTRG.130619 | 5.932065356 | 3.71E−05 | 0.003083744 |
| . | gene | MSTRG.65726 | 14.9850261 | 3.73E−05 | 0.003097192 |
| Gm11361 | gene | MSTRG.35782 | 14.10380987 | 3.84E−05 | 0.003187311 |
| Gm8121 | gene | MSTRG.106202 | 3.442794873 | 3.87E−05 | 0.003205898 |
| Hist1h2ab | gene | MSTRG.35580 | 0.010797895 | 3.89E−05 | 0.003220321 |
| Gm8444 | gene | MSTRG.51708 | 2.339077562 | 3.93E−05 | 0.003246845 |
| Rpl38-ps2 | gene | MSTRG.120232 | 19.56588793 | 4.06E−05 | 0.003351612 |
| Gm43712 | gene | MSTRG.89308 | 22.60173156 | 4.17E−05 | 0.003433166 |
| Atp5l-ps1 | gene | MSTRG.124924 | 8.939785764 | 4.25E−05 | 0.003492149 |
| Rcor1 | gene | MSTRG.33735 | 5.930248007 | 4.28E−05 | 0.003515162 |
| Csta1 | gene | MSTRG.55669 | 10.10111893 | 4.35E−05 | 0.003568889 |
| Wfdc15a | gene | MSTRG.83138 | 26.83398836 | 4.39E−05 | 0.003597205 |
| Rpl9-ps4 | gene | MSTRG.38456 | 60.39369558 | 4.41E−05 | 0.003610204 |
| Gm9396 | gene | MSTRG.92047 | 9.194492603 | 4.44E−05 | 0.003625917 |
| . | gene | MSTRG.64781 | 7.880101009 | 4.48E−05 | 0.003649372 |
| Gm13680 | gene | MSTRG.78317 | 28.02983858 | 4.54E−05 | 0.003690877 |
| . | gene | MSTRG.29929 | 3.617558447 | 4.56E−05 | 0.003707902 |
| Gm11349 | gene | MSTRG.35690 | 3.473278688 | 4.63E−05 | 0.003756264 |
| Ptgis | gene | MSTRG.83381 | 11.96761794 | 4.65E−05 | 0.003763255 |
| Gm13340 | gene | MSTRG.74809 | 8.946516488 | 4.66E−05 | 0.003763255 |
| Gm11517 | gene | MSTRG.25667 | 12.37451729 | 4.66E−05 | 0.003763255 |
| Gm27219 | gene | MSTRG.139229 | 6.802142962 | 4.68E−05 | 0.00377407 |
| . | gene | MSTRG.67135 | 9.97735574 | 4.74E−05 | 0.003817307 |
| Rbpsuh-rs3 | gene | MSTRG.114915 | 5.433902623 | 4.77E−05 | 0.00383016 |
| . | gene | MSTRG.3192 | 5.363528686 | 4.82E−05 | 0.003871313 |
| Dmc1 | gene | MSTRG.51496 | 7.421151513 | 4.85E−05 | 0.00388275 |
| Gm10335 | gene | MSTRG.12076 | 10.28457528 | 4.85E−05 | 0.00388275 |
| Gm28555 | gene | MSTRG.144032 | 7.931002956 | 4.89E−05 | 0.003905394 |
| . | gene | MSTRG.138042 | 4.401751835 | 4.91E−05 | 0.003922278 |
| Sgpp1 | gene | MSTRG.31423 | 13.95457018 | 4.96E−05 | 0.003951421 |
| Gm14044 | gene | MSTRG.80962 | 5.341244822 | 4.96E−05 | 0.003951435 |
| Gm14539 | gene | MSTRG.146456 | 6.292054623 | 4.98E−05 | 0.003957416 |
| Gm5621 | gene | MSTRG.144031 | 7.878483947 | 5.09E−05 | 0.004039372 |
| Eif1-ps1 | gene | MSTRG.43050 | 8.301777746 | 5.20E−05 | 0.004122378 |
| Eomes | gene | MSTRG.144892 | 28.4149987 | 5.46E−05 | 0.004303288 |
| Bex1 | gene | MSTRG.150456 | 0.01068375 | 5.48E−05 | 0.00431346 |
| . | gene | MSTRG.2352 | 11.36493736 | 5.50E−05 | 0.004327055 |
| . | gene | MSTRG.149498 | 4.70363957 | 5.51E−05 | 0.004331141 |
| . | gene | MSTRG.137591 | 13.11773898 | 5.59E−05 | 0.004386023 |
| Gm5864 | gene | MSTRG.104601 | 8.159001031 | 5.76E−05 | 0.004510478 |
| Gm12191 | gene | MSTRG.21927 | 25.93616586 | 5.80E−05 | 0.004540724 |
| Trmo | gene | MSTRG.96011 | 8.625493154 | 5.89E−05 | 0.004600911 |
| . | gene | MSTRG.99549 | 3.221296361 | 5.99E−05 | 0.004673851 |
| Rps19-ps6 | gene | MSTRG.33693 | 13.59840153 | 6.03E−05 | 0.004698096 |
| Gm5145 | gene | MSTRG.59678 | 3.99729311 | 6.06E−05 | 0.004712894 |
| Rpl31-ps9 | gene | MSTRG.7152 | 37.44701559 | 6.06E−05 | 0.004712894 |
| . | gene | MSTRG.82089 | 5.746457676 | 6.10E−05 | 0.004730512 |
| . | gene | MSTRG.76047 | 7.292251328 | 6.11E−05 | 0.004730691 |
| Rps23-ps1 | gene | MSTRG.86324 | 12.10150112 | 6.18E−05 | 0.004777704 |
| Gm11966 | gene | MSTRG.19438 | 9.933310747 | 6.19E−05 | 0.004782149 |
| Gm7658 | gene | MSTRG.1126 | 5.538575222 | 6.28E−05 | 0.004837197 |
| Gm12663 | gene | MSTRG.19998 | 3.842229404 | 6.28E−05 | 0.004837197 |
| . | gene | MSTRG.59856 | 4.886475734 | 6.32E−05 | 0.004861596 |
| Gm9009 | gene | MSTRG.148981 | 7.098041364 | 6.47E−05 | 0.004955942 |
| . | gene | MSTRG.71172 | 10.18441765 | 6.51E−05 | 0.0049809 |
| . | gene | MSTRG.132515 | 7.378356525 | 6.56E−05 | 0.00500991 |
| Rpl10l | gene | MSTRG.30905 | 28.48466453 | 6.59E−05 | 0.005031396 |
| Gm20430 | gene | MSTRG.44474 | 2.70159402 | 6.65E−05 | 0.005066925 |
| Rhbdl2 | gene | MSTRG.100202 | 14.90244175 | 6.79E−05 | 0.005164306 |
| . | gene | MSTRG.32068 | 3.505879407 | 6.79E−05 | 0.005164348 |
| . | gene | MSTRG.37113 | 21.05557803 | 6.94E−05 | 0.005272227 |
| Gm10247 | gene | MSTRG.53673 | 13.38333196 | 6.99E−05 | 0.005299785 |
| . | gene | MSTRG.9872 | 3.33846345 | 7.00E−05 | 0.005299785 |
| . | gene | MSTRG.67142 | 22.29599794 | 7.05E−05 | 0.005322596 |
| Mgl2 | gene | MSTRG.23571 | 5.053001214 | 7.14E−05 | 0.005387709 |
| Gm13182 | gene | MSTRG.73567 | 5.513924488 | 7.18E−05 | 0.005406991 |
| . | gene | MSTRG.59700 | 4.994155784 | 7.23E−05 | 0.005430927 |
| . | gene | MSTRG.146869 | 3.680658705 | 7.28E−05 | 0.00546557 |
| . | gene | MSTRG.3103 | 26.17183572 | 7.41E−05 | 0.005555634 |
| Rpl10-ps3 | gene | MSTRG.140078 | 7.383974552 | 7.45E−05 | 0.005577293 |
| Nedd4 | gene | MSTRG.142120 | 0.031298323 | 7.56E−05 | 0.0056499 |
| Cgnl1 | gene | MSTRG.141982 | 5.012447068 | 7.63E−05 | 0.005697942 |
| Rpl10-ps1 | gene | MSTRG.79446 | 8.894313686 | 7.78E−05 | 0.005799006 |
| Ly96 | gene | MSTRG.849 | 6.514336558 | 8.19E−05 | 0.006080596 |
| . | gene | MSTRG.85745 | 3.294506065 | 8.19E−05 | 0.006080596 |
| . | gene | MSTRG.47452 | 4.125854927 | 8.20E−05 | 0.006080596 |
| . | gene | MSTRG.85440 | 4.280963908 | 8.24E−05 | 0.006103957 |
| . | gene | MSTRG.63374 | 9.363545474 | 8.25E−05 | 0.006103957 |
| Gm6542 | gene | MSTRG.66439 | 17.35575176 | 8.37E−05 | 0.006174085 |
| Gm29667 | gene | MSTRG.5782 | 7.862084269 | 8.51E−05 | 0.006270059 |
| Hmgb1-ps5 | gene | MSTRG.89678 | 7.650939312 | 8.57E−05 | 0.006286505 |
| . | gene | MSTRG.129948 | 4.067623673 | 8.57E−05 | 0.006286505 |
| Sarm1 | gene | MSTRG.24372 | 6.303537917 | 8.72E−05 | 0.006393287 |
| . | gene | MSTRG.41240 | 13.23406009 | 8.90E−05 | 0.006489397 |
| . | gene | MSTRG.37942 | 15.06425517 | 9.19E−05 | 0.006694535 |
| . | gene | MSTRG.147786 | 15.80537299 | 9.35E−05 | 0.006800694 |
| Gm11703 | gene | MSTRG.27132 | 7.354914137 | 9.48E−05 | 0.006885495 |
| Gm16439 | gene | MSTRG.43315 | 7.079329296 | 9.65E−05 | 0.007003188 |
| . | gene | MSTRG.8106 | 13.52137613 | 9.72E−05 | 0.007037751 |
| . | gene | MSTRG.28636 | 6.997125818 | 9.93E−05 | 0.007176286 |
| Tcea1-ps1 | gene | MSTRG.52373 | 7.48113275 | 9.96E−05 | 0.007192056 |
| . | gene | MSTRG.45642 | 10.14867254 | 0.000100602 | 0.007254888 |
| Gm4754 | gene | MSTRG.105315 | 3.339765921 | 0.000101786 | 0.007331338 |
| Gm14435 | gene | MSTRG.84333 | 5.452698248 | 0.000102114 | 0.007346021 |
| Gm16216 | gene | MSTRG.71134 | 4.487206786 | 0.000103372 | 0.007427473 |
| Rpsa-ps4 | gene | MSTRG.21085 | 7.811496738 | 0.000104324 | 0.007480179 |
| . | gene | MSTRG.35503 | 0.004404143 | 0.000104359 | 0.007480179 |
| Gm7204 | gene | MSTRG.56377 | 55.83491296 | 0.000105181 | 0.00752083 |
| Muc1 | gene | MSTRG.89225 | 6.285526593 | 0.000105983 | 0.007569034 |
| . | gene | MSTRG.79126 | 172.0469179 | 0.000106827 | 0.007620083 |
| . | gene | MSTRG.29702 | 29.24121357 | 0.000107232 | 0.007639765 |
| Gm6822 | gene | MSTRG.3000 | 2.310149737 | 0.000108158 | 0.007677946 |
| . | gene | MSTRG.100914 | 2.57037436 | 0.000109172 | 0.007740604 |
| . | gene | MSTRG.26217 | 9.517828412 | 0.000109803 | 0.007775968 |
| Rpsa-ps9 | gene | MSTRG.75334 | 6.338602448 | 0.000111107 | 0.007858933 |
| Hist1h1b | gene | MSTRG.35480 | 32.64146149 | 0.00011163 | 0.007886481 |
| Gm12734 | gene | MSTRG.24314 | 3.21181238 | 0.000115434 | 0.008145471 |
| . | gene | MSTRG.28635 | 6.262623488 | 0.000116315 | 0.008197848 |
| Gm44484 | gene | MSTRG.35581 | 0.095963146 | 0.000117181 | 0.008249027 |
| Cyb5r3 | gene | MSTRG.51842 | 0.08492338 | 0.000118204 | 0.008311146 |
| Gm17828 | gene | MSTRG.142059 | 8.745318789 | 0.000119268 | 0.008375969 |
| . | gene | MSTRG.140914 | 3.418960911 | 0.000119889 | 0.008409585 |
| . | gene | MSTRG.79405 | 6.879187392 | 0.000120185 | 0.008420348 |
| Gm6304 | gene | MSTRG.65402 | 8.293733868 | 0.00012185 | 0.008526887 |
| . | gene | MSTRG.32003 | 6.577947065 | 0.000122304 | 0.008542797 |
| Gm12643 | gene | MSTRG.98150 | 3.085983754 | 0.000122366 | 0.008542797 |
| Gm42992 | gene | MSTRG.110839 | 4.433261452 | 0.000126388 | 0.00881314 |
| Pnliprp2 | gene | MSTRG.73351 | 27.63546897 | 0.000127143 | 0.008855296 |
| Alox12 | gene | MSTRG.23604 | 9.164995102 | 0.000127417 | 0.008863979 |
| Dclre1b | gene | MSTRG.90383 | 6.451005658 | 0.000128102 | 0.008901144 |
| Glns-ps1 | gene | MSTRG.20170 | 3.987640883 | 0.000130099 | 0.009029255 |
| Foxh1 | gene | MSTRG.51229 | 12.30261271 | 0.000130965 | 0.009070352 |
| . | gene | MSTRG.80719 | 32.15338043 | 0.000131061 | 0.009070352 |
| Gm9727 | gene | MSTRG.108892 | 6.195991786 | 0.000131199 | 0.009070352 |
| . | gene | MSTRG.17922 | 7.708956614 | 0.000131305 | 0.009070352 |
| . | gene | MSTRG.28568 | 3.160300781 | 0.000132083 | 0.009102799 |
| Gm10443 | gene | MSTRG.116987 | 21.25414162 | 0.000132711 | 0.009130466 |
| Gm8292 | gene | MSTRG.3019 | 4.07292904 | 0.000132794 | 0.009130466 |
| . | gene | MSTRG.111516 | 6.356954097 | 0.000133363 | 0.009158957 |
| Wbp5 | gene | MSTRG.150466 | 0.002396671 | 0.000137787 | 0.009451788 |
| Bsg | gene | MSTRG.15886 | 0.027726177 | 0.000138586 | 0.009495569 |
| . | gene | MSTRG.146783 | 9.445727621 | 0.000139917 | 0.009575616 |
| . | gene | MSTRG.136833 | 3.004413648 | 0.000141332 | 0.00966127 |
| Rps12-ps3 | gene | MSTRG.73399 | 12.54449809 | 0.00014221 | 0.009707203 |
| Hist1h4h | gene | MSTRG.35520 | 10.77936854 | 0.000142332 | 0.009707203 |
| . | gene | MSTRG.52096 | 2.662830984 | 0.000145047 | 0.009869569 |
| Mras | gene | MSTRG.143642 | 9.793297724 | 0.000145735 | 0.009904972 |
| . | gene | MSTRG.120824 | 6.281789313 | 0.000147323 | 0.010001397 |
| Gm16089 | gene | MSTRG.111295 | 8.132902568 | 0.000150239 | 0.010187594 |
| Rps27a-ps2 | gene | MSTRG.142732 | 41.02845215 | 0.000153531 | 0.010398909 |
| . | gene | MSTRG.84453 | 8.921952214 | 0.000155092 | 0.010492596 |
| . | gene | MSTRG.72245 | 3.919418603 | 0.000158718 | 0.010701143 |
| Gm6204 | gene | MSTRG.87125 | 18.53706721 | 0.000160183 | 0.010787623 |
| . | gene | MSTRG.44535 | 25.50223772 | 0.000165391 | 0.011125621 |
| Oaz1-ps | gene | MSTRG.59506 | 4.870830293 | 0.00016624 | 0.011170037 |
| Gm9703 | gene | MSTRG.60231 | 2.971201248 | 0.000166754 | 0.011191874 |
| Rps6-ps1 | gene | MSTRG.51129 | 7.418056451 | 0.00016904 | 0.011319522 |
| Plac9a | gene | MSTRG.42288 | 6.566094883 | 0.000169684 | 0.011349777 |
| . | gene | MSTRG.44065 | 3.70224273 | 0.000174438 | 0.011654576 |
| . | gene | MSTRG.26153 | 10.64341137 | 0.00017586 | 0.01173629 |
| . | gene | MSTRG.85025 | 2.8063171 | 0.000176143 | 0.011741942 |
| . | gene | MSTRG.15052 | 3.007927266 | 0.000181028 | 0.011995482 |
| Gm5560 | gene | MSTRG.108248 | 72.35543311 | 0.000181165 | 0.011995482 |
| Gm5809 | gene | MSTRG.54804 | 18.1593854 | 0.000182023 | 0.012025359 |
| . | gene | MSTRG.64592 | 7.687837263 | 0.000183196 | 0.012089309 |
| Gm8849 | gene | MSTRG.52591 | 3.006462665 | 0.000184677 | 0.012173398 |
| . | gene | MSTRG.18999 | 9.143443515 | 0.00018933 | 0.012410878 |
| Hist1h2ad | gene | MSTRG.35561 | 0.021675501 | 0.000191314 | 0.012527058 |
| . | gene | MSTRG.101017 | 5.878251877 | 0.000191745 | 0.012541387 |
| . | gene | MSTRG.25160 | 15.54323803 | 0.000192081 | 0.012549407 |
| . | gene | MSTRG.39581 | 19.80057792 | 0.000199271 | 0.012978264 |
| Gm12416 | gene | MSTRG.97753 | 5.821704601 | 0.000199304 | 0.012978264 |
| Mrto4-ps2 | gene | MSTRG.28620 | 11.50478685 | 0.000199647 | 0.012986309 |
| Gm4332 | gene | MSTRG.91384 | 55.12864471 | 0.0002022 | 0.013123428 |
| Rpl17-ps9 | gene | MSTRG.123349 | 17.2078515 | 0.000206472 | 0.013371305 |
| Gm8172 | gene | MSTRG.148000 | 5.814534709 | 0.00020867 | 0.013470147 |
| . | gene | MSTRG.30552 | 2.875086578 | 0.000208682 | 0.013470147 |
| . | gene | MSTRG.44413 | 8.160893694 | 0.000209725 | 0.013522658 |
| Gm3362 | gene | MSTRG.49331 | 7.263335724 | 0.000210864 | 0.013581299 |
| . | gene | MSTRG.145704 | 11.39366719 | 0.00021179 | 0.01362605 |
| . | gene | MSTRG.37542 | 6.789654082 | 0.000214098 | 0.013744603 |
| . | gene | MSTRG.33341 | 6.502460088 | 0.000215241 | 0.013802085 |
| Myd88 | gene | MSTRG.144974 | 14.54437682 | 0.00021546 | 0.013802085 |
| . | gene | MSTRG.72303 | 4.233304818 | 0.000216455 | 0.013850744 |
| . | gene | MSTRG.52881 | 11.78759327 | 0.000218604 | 0.013973118 |
| Hmgb1-ps6 | gene | MSTRG.55315 | 3.871366334 | 0.000219313 | 0.013982322 |
| Retsat | gene | MSTRG.116301 | 3.454032093 | 0.000219466 | 0.013982322 |
| Spsb4 | gene | MSTRG.143459 | 20.49971103 | 0.000224523 | 0.014274278 |
| . | gene | MSTRG.42622 | 6.466781611 | 0.000225626 | 0.01432894 |
| Rpl10-ps6 | gene | MSTRG.71904 | 8.303995067 | 0.00022707 | 0.014405159 |
| . | gene | MSTRG.111871 | 16.27406319 | 0.00022948 | 0.014526849 |
| Gm13493 | gene | MSTRG.76734 | 6.990297197 | 0.000230505 | 0.014560535 |
| Rpl34-ps1 | gene | MSTRG.116153 | 11.45775084 | 0.000236157 | 0.014869814 |
| Gm6900 | gene | MSTRG.121360 | 7.281007852 | 0.000239245 | 0.015048219 |
| . | gene | MSTRG.104850 | 4.143886237 | 0.000240282 | 0.015081322 |
| Epha4 | gene | MSTRG.4321 | 4.353776708 | 0.000242548 | 0.015207375 |
| 2810001G20Rik | gene | MSTRG.23128 | 16.13539722 | 0.000242919 | 0.01521453 |
| . | gene | MSTRG.26993 | 0.116678299 | 0.000244529 | 0.015286493 |
| . | gene | MSTRG.72037 | 12.15520247 | 0.000244586 | 0.015286493 |
| . | gene | MSTRG.104948 | 62.30446843 | 0.000251063 | 0.015674722 |
| . | gene | MSTRG.35278 | 4.046191191 | 0.000252808 | 0.015767003 |
| Gm14414 | gene | MSTRG.84389 | 8.834271789 | 0.000254255 | 0.01582384 |
| Gm10913 | gene | MSTRG.55682 | 8.340740907 | 0.000255195 | 0.015865597 |
| Gm15452 | gene | MSTRG.366 | 24.85960007 | 0.000257386 | 0.015984971 |
| Rpl31-ps14 | gene | MSTRG.3745 | 20.67730452 | 0.000258781 | 0.016054779 |
| Chdh | gene | MSTRG.42549 | 5.463483597 | 0.000261081 | 0.01618042 |
| . | gene | MSTRG.137266 | 17.73280544 | 0.000261906 | 0.016214539 |
| Gm27684 | gene | MSTRG.128363 | 3.716927556 | 0.000266792 | 0.016499767 |
| Gm33051 | gene | MSTRG.85822 | 6.390255129 | 0.000275239 | 0.016986606 |
| Rpl23a-ps3 | gene | MSTRG.42825 | 7.400013699 | 0.000276353 | 0.017019772 |
| Gm5422 | gene | MSTRG.13004 | 3.518227906 | 0.000280978 | 0.017231662 |
| Med18 | gene | MSTRG.100912 | 31.61944078 | 0.000281252 | 0.017231662 |
| Gm6266 | gene | MSTRG.120585 | 2.438950134 | 0.000284645 | 0.017385426 |
| . | gene | MSTRG.150017 | 14.20734012 | 0.000285263 | 0.017405193 |
| . | gene | MSTRG.32085 | 10.43106958 | 0.000290301 | 0.017679153 |
| . | gene | MSTRG.43801 | 4.570705762 | 0.00029092 | 0.017695491 |
| Gm7331 | gene | MSTRG.151543 | 4.690821275 | 0.000296053 | 0.017933844 |
| Rpl27-ps1 | gene | MSTRG.9774 | 12.22984329 | 0.000296703 | 0.017954781 |
| . | gene | MSTRG.24936 | 5.032635624 | 0.000302944 | 0.018294955 |
| Parvb | gene | MSTRG.51923 | 7.914790079 | 0.000303817 | 0.018328953 |
| Kdm3b | gene | MSTRG.66251 | 9.025127958 | 0.00030555 | 0.018414664 |
| . | gene | MSTRG.68019 | 2.70274614 | 0.000315712 | 0.018988395 |
| Tdpx-ps1 | gene | MSTRG.5764 | 8.856350827 | 0.000320339 | 0.019207955 |
| Pdia3 | gene | MSTRG.80388 | 0.055625946 | 0.000327273 | 0.019564191 |
| . | gene | MSTRG.53366 | 4.925597171 | 0.000331784 | 0.019813751 |
| Rps3a2 | gene | MSTRG.45997 | 13.87240314 | 0.000334745 | 0.019970404 |
| . | gene | MSTRG.54138 | 6.723384987 | 0.000336691 | 0.020066236 |
| Gm10177 | gene | MSTRG.139258 | 4.954871618 | 0.000337403 | 0.020088416 |
| Gm44122 | gene | MSTRG.118300 | 11.02528821 | 0.000338192 | 0.020115119 |
| Gm15427 | gene | MSTRG.5548 | 2.936597096 | 0.00034734 | 0.020617671 |
| Trmt112-ps2 | gene | MSTRG.102209 | 5.664847403 | 0.000352942 | 0.020908162 |
| . | gene | MSTRG.51915 | 2.657508619 | 0.00035704 | 0.021129771 |
| Rnf38 | gene | MSTRG.95830 | 4.063629696 | 0.000361025 | 0.021301553 |
| . | gene | MSTRG.108890 | 11.39283267 | 0.00037592 | 0.022048277 |
| . | gene | MSTRG.138909 | 3.884970335 | 0.000388868 | 0.022694988 |
| . | gene | MSTRG.68102 | 3.292868868 | 0.000389809 | 0.022712232 |
| . | gene | MSTRG.72656 | 15.63811163 | 0.000392918 | 0.022841073 |
| . | gene | MSTRG.44976 | 5.183280315 | 0.000395108 | 0.022945802 |
| Tigar | gene | MSTRG.119437 | 29.6120272 | 0.000395774 | 0.022961868 |
| Slc28a3 | gene | MSTRG.37753 | 4.675651182 | 0.000397809 | 0.02305729 |
| . | gene | MSTRG.55279 | 2.673190358 | 0.000398655 | 0.023080472 |
| Pex11b | gene | MSTRG.89860 | 9.51928392 | 0.00039899 | 0.023080472 |
| . | gene | MSTRG.43060 | 13.28940145 | 0.000402467 | 0.023236093 |
| Gm13007 | gene | MSTRG.101280 | 3.27236978 | 0.000405876 | 0.023409993 |
| . | gene | MSTRG.36487 | 3.46301848 | 0.000407887 | 0.023503022 |
| . | gene | MSTRG.24456 | 10.92904021 | 0.000412584 | 0.023727382 |
| . | gene | MSTRG.45360 | 3.285683894 | 0.000422973 | 0.024193913 |
| Atp5l-ps2 | gene | MSTRG.25027 | 2.50482589 | 0.000423668 | 0.024199907 |
| . | gene | MSTRG.91281 | 6.746435894 | 0.00042806 | 0.024421243 |
| . | gene | MSTRG.40563 | 4.292886465 | 0.00042837 | 0.024421243 |
| . | gene | MSTRG.23971 | 5.343684251 | 0.000429152 | 0.024442194 |
| Gm12715 | gene | MSTRG.98837 | 6.694139063 | 0.000430275 | 0.024482546 |
| . | gene | MSTRG.115132 | 6.939232094 | 0.000430772 | 0.024487281 |
| Stmn3 | gene | MSTRG.84425 | 100.8114493 | 0.000431456 | 0.024502558 |
| . | gene | MSTRG.7313 | 18.7102902 | 0.000438285 | 0.024866447 |
| Hs3st3b1 | gene | MSTRG.23087 | 3.539858422 | 0.000438974 | 0.02487669 |
| Gm8974 | gene | MSTRG.72631 | 2.265791651 | 0.000439308 | 0.02487669 |
| Rps15a-ps4 | gene | MSTRG.100853 | 2.039545413 | 0.000448085 | 0.025325166 |
| . | gene | MSTRG.20231 | 17.49357502 | 0.000450702 | 0.025448747 |
| . | gene | MSTRG.32080 | 446.0573174 | 0.000452469 | 0.0255241 |
| . | gene | MSTRG.40076 | 8.146274132 | 0.000454937 | 0.025614397 |
| . | gene | MSTRG.52026 | 3.989799549 | 0.000459699 | 0.025857829 |
| Plac9b | gene | MSTRG.42328 | 17.56558344 | 0.000461389 | 0.025928236 |
| . | gene | MSTRG.149830 | 5.43909597 | 0.000464513 | 0.026054216 |
| Gm6450 | gene | MSTRG.108083 | 6.254255929 | 0.000477911 | 0.026704216 |
| . | gene | MSTRG.101881 | 3.292590494 | 0.000480276 | 0.026811029 |
| Tspan1 | gene | MSTRG.99600 | 37.14631261 | 0.000481178 | 0.026836015 |
| . | gene | MSTRG.4393 | 6.010682214 | 0.000489465 | 0.02722108 |
| . | gene | MSTRG.74270 | 3.41931248 | 0.000505059 | 0.028061887 |
| Gm14438 | gene | MSTRG.84089 | 13.21842312 | 0.000508638 | 0.028207665 |
| . | gene | MSTRG.62214 | 9.954404702 | 0.000511563 | 0.028316739 |
| Klhl15 | gene | MSTRG.148941 | 6.593561089 | 0.000523076 | 0.028845831 |
| . | gene | MSTRG.63435 | 4.975438429 | 0.000528511 | 0.02911841 |
| Tubb4b-ps2 | gene | MSTRG.1290 | 6.615415405 | 0.000535691 | 0.029363198 |
| . | gene | MSTRG.88947 | 6.237491191 | 0.00053901 | 0.029476884 |
| . | gene | MSTRG.64909 | 6.34257408 | 0.000540112 | 0.029509799 |
| Rps13-ps5 | gene | MSTRG.22858 | 8.519964125 | 0.000543914 | 0.029662656 |
| Fahd1 | gene | MSTRG.60019 | 7.850803768 | 0.000549065 | 0.029915941 |
| . | gene | MSTRG.14606 | 3.47194251 | 0.000558288 | 0.030385624 |
| Rpl28-ps3 | gene | MSTRG.100385 | 6.450928603 | 0.000559905 | 0.03042237 |
| Rpl7 | gene | MSTRG.806 | 5.558844282 | 0.000562227 | 0.030510871 |
| Gm4575 | gene | MSTRG.117467 | 6.652558493 | 0.000562567 | 0.030510871 |
| Gm5239 | gene | MSTRG.66311 | 3.241632628 | 0.000569246 | 0.030816481 |
| Gm10093 | gene | MSTRG.63639 | 9.380325612 | 0.000574666 | 0.031053005 |
| Rps13-ps4 | gene | MSTRG.137051 | 8.254930861 | 0.000578163 | 0.031213395 |
| Gm44357 | gene | MSTRG.35471 | 0.026659763 | 0.000603975 | 0.032458576 |
| Gm16409 | gene | MSTRG.45348 | 5.126077219 | 0.000612255 | 0.032813967 |
| Gm4613 | gene | MSTRG.122511 | 70.64793338 | 0.000614443 | 0.032901379 |
| Gm7536 | gene | MSTRG.85705 | 7.30983431 | 0.000648967 | 0.034406879 |
| . | gene | MSTRG.144745 | 29.15471839 | 0.000669782 | 0.035320253 |
| Gm20305 | gene | MSTRG.10473 | 3.53644534 | 0.000698749 | 0.036618902 |
| Tbx20 | gene | MSTRG.138601 | 5.985291792 | 0.000861701 | 0.043430737 |
| Ptpn12 | gene | MSTRG.103778 | 10.56566016 | 0.000862361 | 0.043430737 |
| Bex4 | gene | MSTRG.150461 | 0.002701597 | 0.000868148 | 0.043600818 |
| . | gene | MSTRG.115386 | 10.9632341 | 0.000944638 | 0.045407621 |
| Rpl18a-ps1 | gene | MSTRG.30435 | 2.609511325 | 0.001003829 | 0.045407621 |
| . | gene | MSTRG.88994 | 3.788638666 | 0.001006887 | 0.045407621 |
| . | gene | MSTRG.133600 | 0.076636466 | 0.001041234 | 0.045407621 |
| . | gene | MSTRG.116213 | 7.5335146 | 0.001046169 | 0.045407621 |
| . | gene | MSTRG.37968 | 8.745440791 | 0.001211765 | 0.045407621 |
| Rpl39-ps | gene | MSTRG.53188 | 14.14656483 | 0.001424018 | 0.045407621 |
| Gm5148 | gene | MSTRG.86323 | 7.304632269 | 0.001572076 | 0.045407621 |
| . | gene | MSTRG.89541 | 3.1605165 | 0.001585789 | 0.045407621 |
| Lgals4 | gene | MSTRG.122834 | 66.11772052 | 0.001706548 | 0.045407621 |
| . | gene | MSTRG.38643 | 113.8967697 | 0.002080067 | 0.045407621 |
| Map1lc3b | gene | MSTRG.140232 | 9.58295859 | 0.002144922 | 0.045407621 |
| Gm15698 | gene | MSTRG.25263 | 39.17955614 | 0.002222007 | 0.045407621 |

TABLE 7
List of DEGs Between EPS-blastoids and Morulae

| GeneName | Feature | GeneId | FC | pval | qval |
|---|---|---|---|---|---|
| Calcoco2 | gene | MSTRG.25615 | 1411.692516 | 0 | 0 |
| Gm15981 | gene | MSTRG.107116 | 10.60330116 | 3.11E−15 | 9.18E−11 |
| . | gene | MSTRG.143119 | 147.7272067 | 4.66E−15 | 9.18E−11 |
| Pramel4 | gene | MSTRG.101970 | 166.4512166 | 3.18E−14 | 4.69E−10 |
| Gm16166 | gene | MSTRG.18583 | 5.866517892 | 5.42E−14 | 6.40E−10 |
| Fbp2 | gene | MSTRG.37912 | 642.0575405 | 6.64E−14 | 6.54E−10 |
| Gm13740 | gene | MSTRG.78564 | 31.15041552 | 5.76E−13 | 4.86E−09 |
| AA467197 | gene | MSTRG.80472 | 244.3011412 | 7.84E−13 | 5.79E−09 |
| . | gene | MSTRG.17028 | 5.271174142 | 1.21E−12 | 7.96E−09 |
| Gm13039 | gene | MSTRG.101870 | 12.44922929 | 1.82E−12 | 1.04E−08 |
| Rpl5-ps1 | gene | MSTRG.694 | 126.238537 | 1.94E−12 | 1.04E−08 |
| Rps12-ps10 | gene | MSTRG.80441 | 14.04028913 | 3.47E−12 | 1.59E−08 |
| RP23-181P23.2 | gene | MSTRG.124667 | 4.84719641 | 3.51E−12 | 1.59E−08 |
| Rpl39l | gene | MSTRG.53755 | 274.2806684 | 4.56E−12 | 1.92E−08 |
| Gm9057 | gene | MSTRG.103906 | 22.55578786 | 5.81E−12 | 2.29E−08 |
| . | gene | MSTRG.120965 | 6.093912636 | 1.10E−11 | 4.06E−08 |
| . | gene | MSTRG.21764 | 436.8033656 | 1.62E−11 | 5.30E−08 |
| . | gene | MSTRG.3931 | 20.70063296 | 1.85E−11 | 5.37E−08 |
| Clic3 | gene | MSTRG.75028 | 44.43778238 | 1.90E−11 | 5.37E−08 |
| Gm8250 | gene | MSTRG.70797 | 4.924399171 | 1.94E−11 | 5.37E−08 |
| Crxos | gene | MSTRG.121773 | 341.2191274 | 2.04E−11 | 5.37E−08 |
| Trim43c | gene | MSTRG.143079 | 52.42820835 | 2.09E−11 | 5.37E−08 |
| Dppa3-ps | gene | MSTRG.146914 | 20.20210097 | 2.31E−11 | 5.69E−08 |
| Fam107b | gene | MSTRG.73600 | 14.82419282 | 2.93E−11 | 6.66E−08 |
| Gm16471 | gene | MSTRG.149327 | 6.377290781 | 3.39E−11 | 7.41E−08 |
| . | gene | MSTRG.18159 | 59.0671765 | 3.67E−11 | 7.41E−08 |
| Gm17786 | gene | MSTRG.140228 | 17.18183966 | 3.94E−11 | 7.41E−08 |
| . | gene | MSTRG.37937 | 26.60801659 | 3.94E−11 | 7.41E−08 |
| Gm8482 | gene | MSTRG.32977 | 22.6516874 | 3.97E−11 | 7.41E−08 |
| Gm24276 | gene | MSTRG.369 | 14.24849995 | 4.12E−11 | 7.41E−08 |
| Rpl21-ps14 | gene | ENSMUSG00000062152 | 6.581647496 | 4.14E−11 | 7.41E−08 |
| Gm13413 | gene | MSTRG.75676 | 15.85047361 | 4.62E−11 | 8.03E−08 |
| Gm11955 | gene | MSTRG.19277 | 12.83058832 | 5.20E−11 | 8.77E−08 |
| Gm32536 | gene | MSTRG.119301 | 9.721144665 | 5.41E−11 | 8.88E−08 |
| Rpl5-ps2 | gene | MSTRG.82501 | 23.17260448 | 5.90E−11 | 9.42E−08 |
| Gm9083 | gene | MSTRG.146435 | 14.83694674 | 6.43E−11 | 1.00E−07 |
| Gm8425 | gene | MSTRG.55729 | 3.859639548 | 6.80E−11 | 1.03E−07 |
| Gm6425 | gene | MSTRG.70153 | 42.8184629 | 7.34E−11 | 1.08E−07 |
| Apoa1 | gene | MSTRG.139873 | 88.16386749 | 7.81E−11 | 1.11E−07 |
| . | gene | MSTRG.145763 | 3.442317421 | 7.90E−11 | 1.11E−07 |
| . | gene | MSTRG.130919 | 10.82223729 | 8.09E−11 | 1.11E−07 |
| Pramel7 | gene | MSTRG.78549 | 81.93891785 | 8.27E−11 | 1.11E−07 |
| Gm6375 | gene | MSTRG.119819 | 32.03721076 | 9.08E−11 | 1.19E−07 |
| Gm4222 | gene | MSTRG.78653 | 108.3603286 | 9.30E−11 | 1.19E−07 |
| RP23-363E22.1 | gene | MSTRG.126762 | 9.018696869 | 1.18E−10 | 1.47E−07 |
| Gm12447 | gene | MSTRG.96005 | 14.79812918 | 1.20E−10 | 1.47E−07 |
| . | gene | MSTRG.64779 | 9.160686399 | 1.28E−10 | 1.54E−07 |
| Cyp2s1 | gene | MSTRG.122506 | 29.76715914 | 1.34E−10 | 1.59E−07 |
| RP23-412J12.1 | gene | MSTRG.129333 | 3.796346221 | 1.46E−10 | 1.69E−07 |
| Ywhaq-ps2 | gene | MSTRG.109517 | 9.331129762 | 1.65E−10 | 1.87E−07 |
| mt-Th | gene | MSTRG.145686 | 13.11214778 | 1.69E−10 | 1.88E−07 |
| Pramel6 | gene | MSTRG.78552 | 78.80313426 | 1.76E−10 | 1.93E−07 |
| Gm13653 | gene | MSTRG.77943 | 14.48474865 | 2.10E−10 | 2.26E−07 |
| Gm11979 | gene | MSTRG.19533 | 4.519779232 | 2.27E−10 | 2.36E−07 |
| . | gene | MSTRG.94783 | 24.37740756 | 2.28E−10 | 2.36E−07 |
| . | gene | MSTRG.138098 | 31.06088428 | 2.66E−10 | 2.71E−07 |
| . | gene | MSTRG.120793 | 7.740931491 | 2.78E−10 | 2.79E−07 |
| Gm15267 | gene | MSTRG.151082 | 17.68181941 | 2.89E−10 | 2.84E−07 |
| Gm28530 | gene | MSTRG.143521 | 38.90915049 | 3.06E−10 | 2.93E−07 |
| Gm6285 | gene | MSTRG.149492 | 11.59123954 | 3.08E−10 | 2.93E−07 |
| Rpl7a-ps3 | gene | MSTRG.49185 | 11.15301393 | 3.24E−10 | 3.04E−07 |
| Gm44078 | gene | MSTRG.119573 | 5.986091119 | 3.37E−10 | 3.11E−07 |
| . | gene | MSTRG.135989 | 50.90039662 | 3.92E−10 | 3.56E−07 |
| . | gene | MSTRG.69293 | 169.2277649 | 4.58E−10 | 4.10E−07 |
| . | gene | MSTRG.3933 | 111.8313999 | 4.79E−10 | 4.22E−07 |
| Gm6265 | gene | MSTRG.30496 | 55.76095259 | 5.27E−10 | 4.58E−07 |
| Rpl35a-ps7 | gene | MSTRG.114963 | 7.920204075 | 5.39E−10 | 4.61E−07 |
| Rpsa-ps5 | gene | MSTRG.20793 | 15.45109581 | 5.51E−10 | 4.62E−07 |
| Gm14541 | gene | MSTRG.146884 | 14.94008649 | 5.55E−10 | 4.62E−07 |
| Gm12816 | gene | MSTRG.99438 | 33.56278245 | 6.07E−10 | 4.96E−07 |
| Gm7792 | gene | MSTRG.108148 | 90.84364835 | 6.13E−10 | 4.96E−07 |
| Gm15616 | gene | MSTRG.106923 | 43.37479483 | 6.61E−10 | 5.20E−07 |
| Fam151a | gene | MSTRG.99011 | 82.00160661 | 6.66E−10 | 5.20E−07 |
| Gm11971 | gene | MSTRG.19537 | 3.852641269 | 6.69E−10 | 5.20E−07 |
| Gm12891 | gene | MSTRG.100136 | 19.47512136 | 6.85E−10 | 5.26E−07 |
| Gm7589 | gene | MSTRG.141199 | 29.29165227 | 7.01E−10 | 5.31E−07 |
| Gm6051 | gene | MSTRG.107265 | 376.8267244 | 7.14E−10 | 5.34E−07 |
| . | gene | MSTRG.95106 | 49.0017705 | 7.55E−10 | 5.51E−07 |
| RP24-490A22.9 | gene | MSTRG.121830 | 86.50571767 | 8.12E−10 | 5.85E−07 |
| Gm7867 | gene | MSTRG.5443 | 34.26990889 | 8.97E−10 | 6.37E−07 |
| Gm13675 | gene | MSTRG.78278 | 7.638556686 | 9.06E−10 | 6.37E−07 |
| . | gene | MSTRG.79122 | 155.155452 | 9.39E−10 | 6.52E−07 |
| RP23-375O10.1 | gene | MSTRG.124441 | 19.86615409 | 9.61E−10 | 6.60E−07 |
| Mir684-2 | gene | MSTRG.94131 | 6.448624304 | 1.05E−09 | 7.05E−07 |
| Rpl7a-ps10 | gene | MSTRG.143475 | 9.728285801 | 1.05E−09 | 7.05E−07 |
| Gm5218 | gene | MSTRG.51633 | 8.648342802 | 1.08E−09 | 7.15E−07 |
| . | gene | MSTRG.145569 | 9.884276478 | 1.09E−09 | 7.15E−07 |
| Rpl21-ps6 | gene | MSTRG.62345 | 7.384669489 | 1.11E−09 | 7.22E−07 |
| . | gene | MSTRG.93412 | 15.70039338 | 1.13E−09 | 7.22E−07 |
| Gm5981 | gene | MSTRG.91562 | 6.356673852 | 1.19E−09 | 7.48E−07 |
| Gm5529 | gene | MSTRG.4425 | 81.41194686 | 1.21E−09 | 7.52E−07 |
| . | gene | MSTRG.22814 | 254.6099724 | 1.25E−09 | 7.68E−07 |
| Gm15483 | gene | MSTRG.129506 | 43.71464437 | 1.27E−09 | 7.72E−07 |
| Gm5619 | gene | MSTRG.142854 | 17.11934296 | 1.32E−09 | 7.98E−07 |
| Gm6274 | gene | MSTRG.146857 | 52.70030827 | 1.36E−09 | 8.11E−07 |
| Gm5943 | gene | MSTRG.149840 | 14.64506788 | 1.43E−09 | 8.46E−07 |
| Gm7866 | gene | MSTRG.141993 | 43.82172109 | 1.48E−09 | 8.66E−07 |
| Gm13921 | gene | MSTRG.79226 | 21.61130662 | 1.54E−09 | 8.90E−07 |
| Gm42845 | gene | MSTRG.107736 | 19.46000148 | 1.82E−09 | 1.03E−06 |
| Gm37599 | gene | MSTRG.8036 | 7.728541222 | 1.85E−09 | 1.03E−06 |
| . | gene | MSTRG.71972 | 18.15250018 | 1.85E−09 | 1.03E−06 |
| Gm15013 | gene | MSTRG.150582 | 5.759510012 | 1.87E−09 | 1.03E−06 |
| AC125178.1 | gene | MSTRG.145588 | 25.19411142 | 1.96E−09 | 1.07E−06 |
| Slc30a4 | gene | MSTRG.80485 | 4.214866018 | 2.17E−09 | 1.17E−06 |
| Gm8282 | gene | MSTRG.142978 | 8.866420019 | 2.24E−09 | 1.20E−06 |
| Bhmt | gene | MSTRG.39629 | 627.3137472 | 2.28E−09 | 1.21E−06 |
| AC123873.2 | gene | MSTRG.145597 | 24.17372875 | 2.42E−09 | 1.28E−06 |
| Gm5842 | gene | MSTRG.85612 | 8.844373614 | 2.51E−09 | 1.30E−06 |
| Gm12624 | gene | MSTRG.22808 | 96.67762949 | 2.62E−09 | 1.35E−06 |
| Gm5829 | gene | MSTRG.3932 | 12.35757236 | 2.64E−09 | 1.35E−06 |
| Gm38358 | gene | MSTRG.10668 | 3.791828901 | 2.82E−09 | 1.42E−06 |
| Rpsa-ps1 | gene | MSTRG.9329 | 17.22532595 | 2.87E−09 | 1.44E−06 |
| Gm10045 | gene | MSTRG.60459 | 18.37760371 | 2.92E−09 | 1.45E−06 |
| Gm12568 | gene | MSTRG.21720 | 6.882912319 | 3.01E−09 | 1.48E−06 |
| Rps25-ps1 | gene | MSTRG.120496 | 23.335267 | 3.12E−09 | 1.51E−06 |
| Gm7384 | gene | MSTRG.120253 | 9.636234472 | 3.12E−09 | 1.51E−06 |
| Rpl31-ps10 | gene | MSTRG.101416 | 13.37177443 | 3.30E−09 | 1.57E−06 |
| Gm7899 | gene | ENSMUSG00000103823 | 15.37264633 | 3.75E−09 | 1.77E−06 |
| . | gene | MSTRG.9539 | 43.89576775 | 3.90E−09 | 1.81E−06 |
| Gm16367 | gene | MSTRG.145619 | 25.99951195 | 3.93E−09 | 1.81E−06 |
| Gm12013 | gene | MSTRG.20012 | 48.67097349 | 3.94E−09 | 1.81E−06 |
| Rps6-ps2 | gene | MSTRG.135185 | 7.807796851 | 4.03E−09 | 1.83E−06 |
| Rnf34 | gene | MSTRG.110151 | 8.015227991 | 4.07E−09 | 1.83E−06 |
| Bin2 | gene | MSTRG.52987 | 36.40073939 | 4.31E−09 | 1.93E−06 |
| . | gene | MSTRG.68375 | 8.438355532 | 4.39E−09 | 1.95E−06 |
| Gm6794 | gene | MSTRG.115904 | 27.76654328 | 4.43E−09 | 1.95E−06 |
| Rps8-ps1 | gene | MSTRG.14347 | 27.96048404 | 4.51E−09 | 1.97E−06 |
| 1700029P11Rik | gene | MSTRG.51747 | 61.51014865 | 4.67E−09 | 2.03E−06 |
| Foxm1 | gene | MSTRG.119521 | 4.369370331 | 4.96E−09 | 2.13E−06 |
| Gm3183 | gene | MSTRG.108128 | 26.20847273 | 4.97E−09 | 2.13E−06 |
| Gm6223 | gene | MSTRG.143117 | 17.78493033 | 5.00E−09 | 2.13E−06 |
| Gm7299 | gene | MSTRG.9714 | 19.21486957 | 5.04E−09 | 2.13E−06 |
| . | gene | MSTRG.8055 | 4.498433059 | 5.31E−09 | 2.22E−06 |
| E530001F21Rik | gene | MSTRG.149499 | 8.289349975 | 5.65E−09 | 2.35E−06 |
| Gm13215 | gene | MSTRG.100850 | 12.40246845 | 5.78E−09 | 2.39E−06 |
| Gm11478 | gene | MSTRG.25041 | 30.24897067 | 5.83E−09 | 2.39E−06 |
| Slc43a3 | gene | MSTRG.78418 | 38.82877969 | 5.98E−09 | 2.43E−06 |
| Gm12627 | gene | MSTRG.22799 | 65.63738705 | 6.08E−09 | 2.46E−06 |
| Gm12619 | gene | MSTRG.22807 | 59.54391778 | 6.77E−09 | 2.72E−06 |
| Rps12-ps24 | gene | MSTRG.132424 | 16.40211244 | 7.23E−09 | 2.87E−06 |
| . | gene | MSTRG.53937 | 6.012754444 | 7.24E−09 | 2.87E−06 |
| Gm11824 | gene | MSTRG.94282 | 15.12183599 | 7.34E−09 | 2.89E−06 |
| Hmgb1-ps4 | gene | MSTRG.69931 | 10.37451922 | 7.65E−09 | 2.99E−06 |
| . | gene | MSTRG.28990 | 22.51966592 | 7.72E−09 | 3.00E−06 |
| Gm8652 | gene | MSTRG.112776 | 7.316374444 | 7.88E−09 | 3.02E−06 |
| Gm14056 | gene | MSTRG.81365 | 3.515530139 | 7.88E−09 | 3.02E−06 |
| Gm12094 | gene | MSTRG.20920 | 8.080286144 | 8.13E−09 | 3.08E−06 |
| Gm12171 | gene | MSTRG.21746 | 10.20576029 | 8.20E−09 | 3.08E−06 |
| Rpl27a-ps1 | gene | ENSMUSG00000061488 | 13.241642 | 8.51E−09 | 3.18E−06 |
| Gm3160 | gene | MSTRG.131153 | 2.857994751 | 8.62E−09 | 3.20E−06 |
| Gm16427 | gene | MSTRG.108157 | 133.6602608 | 8.80E−09 | 3.25E−06 |
| Gm5883 | gene | MSTRG.119164 | 12.92558535 | 9.17E−09 | 3.35E−06 |
| Gm5851 | gene | MSTRG.89409 | 8.585927544 | 9.18E−09 | 3.35E−06 |
| . | gene | MSTRG.41983 | 11.44865834 | 9.26E−09 | 3.36E−06 |
| Gm5803 | gene | MSTRG.48551 | 14.66675556 | 9.45E−09 | 3.41E−06 |
| Gm12034 | gene | MSTRG.20214 | 26.15463555 | 1.00E−08 | 3.54E−06 |
| Gulo | gene | MSTRG.44961 | 44.73599236 | 1.00E−08 | 3.54E−06 |
| Rps4l-ps | gene | MSTRG.128348 | 9.521225645 | 1.01E−08 | 3.54E−06 |
| Gm13545 | gene | MSTRG.77024 | 4.091343779 | 1.01E−08 | 3.54E−06 |
| Gm14279 | gene | MSTRG.82726 | 33.42541309 | 1.06E−08 | 3.72E−06 |
| Gm42814 | gene | MSTRG.89113 | 3.515755864 | 1.08E−08 | 3.75E−06 |
| . | gene | MSTRG.56455 | 45.93854896 | 1.11E−08 | 3.82E−06 |
| . | gene | MSTRG.82927 | 4.551070847 | 1.12E−08 | 3.84E−06 |
| Gm8062 | gene | MSTRG.38523 | 6.366094152 | 1.15E−08 | 3.93E−06 |
| Gm3106 | gene | MSTRG.108114 | 22.12433121 | 1.17E−08 | 3.98E−06 |
| . | gene | MSTRG.76410 | 29.68212056 | 1.19E−08 | 4.00E−06 |
| Gm14383 | gene | MSTRG.80230 | 33.46685834 | 1.19E−08 | 4.00E−06 |
| . | gene | MSTRG.145635 | 18.26501038 | 1.20E−08 | 4.00E−06 |
| . | gene | MSTRG.29251 | 15.25836502 | 1.23E−08 | 4.08E−06 |
| Gm5850 | gene | MSTRG.89413 | 8.430614086 | 1.35E−08 | 4.47E−06 |
| Gm10224 | gene | MSTRG.119141 | 31.84774522 | 1.38E−08 | 4.52E−06 |
| Gm13226 | gene | MSTRG.102719 | 39.5510744 | 1.41E−08 | 4.61E−06 |
| . | gene | MSTRG.145637 | 17.37468008 | 1.47E−08 | 4.76E−06 |
| Gm8624 | gene | MSTRG.27312 | 6.975324624 | 1.48E−08 | 4.77E−06 |
| Gm23374 | gene | MSTRG.57114 | 120.5661687 | 1.49E−08 | 4.77E−06 |
| Gm6133 | gene | MSTRG.68887 | 6.237221571 | 1.53E−08 | 4.88E−06 |
| Rpl31-ps16 | gene | MSTRG.64064 | 22.62131815 | 1.54E−08 | 4.88E−06 |
| Gm28911 | gene | MSTRG.6813 | 142.2721936 | 1.56E−08 | 4.89E−06 |
| Rpl31-ps13 | gene | MSTRG.40063 | 33.90652677 | 1.56E−08 | 4.89E−06 |
| Slc5a11 | gene | MSTRG.128912 | 87.17259371 | 1.57E−08 | 4.89E−06 |
| Gm17511 | gene | MSTRG.129193 | 5.291499115 | 1.59E−08 | 4.92E−06 |
| Rps12-ps5 | gene | MSTRG.122988 | 7.455403012 | 1.59E−08 | 4.92E−06 |
| Hmgb1-ps9 | gene | MSTRG.40105 | 11.92551043 | 1.62E−08 | 4.97E−06 |
| Gm16513 | gene | MSTRG.108177 | 21.32902392 | 1.64E−08 | 5.02E−06 |
| Gm8696 | gene | MSTRG.61106 | 8.088619265 | 1.69E−08 | 5.15E−06 |
| Gm15464 | gene | MSTRG.3307 | 16.11013968 | 1.71E−08 | 5.17E−06 |
| Tnfrsf9 | gene | MSTRG.102353 | 14.99325282 | 1.73E−08 | 5.22E−06 |
| Ccdc42 | gene | MSTRG.23369 | 77.00676956 | 1.79E−08 | 5.37E−06 |
| . | gene | MSTRG.145761 | 5.098291638 | 1.88E−08 | 5.62E−06 |
| Gm9701 | gene | MSTRG.87708 | 12.03855548 | 1.90E−08 | 5.63E−06 |
| . | gene | MSTRG.43662 | 65.91104294 | 1.91E−08 | 5.63E−06 |
| BC048679 | gene | MSTRG.126200 | 230.3401132 | 1.94E−08 | 5.70E−06 |
| Tab1 | gene | MSTRG.51548 | 5.76333006 | 2.00E−08 | 5.84E−06 |
| Rpl19-ps1 | gene | MSTRG.4873 | 52.82626134 | 2.05E−08 | 5.97E−06 |
| Gm10237 | gene | MSTRG.55654 | 13.76838525 | 2.07E−08 | 5.99E−06 |
| Gm12727 | gene | MSTRG.98964 | 44.28340033 | 2.10E−08 | 6.04E−06 |
| RP24-144C5.3 | gene | MSTRG.129310 | 25.92869444 | 2.24E−08 | 6.40E−06 |
| . | gene | MSTRG.98736 | 78.35758461 | 2.27E−08 | 6.43E−06 |
| Actg-ps1 | gene | MSTRG.132929 | 97.89772726 | 2.28E−08 | 6.45E−06 |
| Gm5445 | gene | MSTRG.34753 | 5.266884358 | 2.36E−08 | 6.64E−06 |
| Rpl10-ps2 | gene | MSTRG.136240 | 13.02117005 | 2.40E−08 | 6.73E−06 |
| Cd81 | gene | MSTRG.130268 | 0.002077654 | 2.49E−08 | 6.90E−06 |
| . | gene | MSTRG.55443 | 9.231770697 | 2.50E−08 | 6.90E−06 |
| Rpl17-ps4 | gene | MSTRG.96939 | 21.03405864 | 2.51E−08 | 6.90E−06 |
| Trim38 | gene | MSTRG.35535 | 25.8397546 | 2.51E−08 | 6.90E−06 |
| Gm12337 | gene | MSTRG.24012 | 25.11963999 | 2.63E−08 | 7.20E−06 |
| . | gene | MSTRG.47212 | 10.48154537 | 2.68E−08 | 7.29E−06 |
| Timd2 | gene | MSTRG.21843 | 316.9369166 | 2.70E−08 | 7.31E−06 |
| . | gene | MSTRG.137500 | 4.077576158 | 2.78E−08 | 7.47E−06 |
| Gm6652 | gene | MSTRG.8646 | 89.04499214 | 2.78E−08 | 7.47E−06 |
| . | gene | MSTRG.28565 | 19.89300469 | 2.81E−08 | 7.51E−06 |
| RP24-471H15.4 | gene | MSTRG.123043 | 23.5004527 | 2.86E−08 | 7.61E−06 |
| Gm14870 | gene | MSTRG.149669 | 3.146426809 | 2.97E−08 | 7.84E−06 |
| Gm11675 | gene | MSTRG.26894 | 7.151188684 | 2.97E−08 | 7.84E−06 |
| Gm3076 | gene | MSTRG.93428 | 11.43884432 | 3.01E−08 | 7.89E−06 |
| Gm14036 | gene | MSTRG.81330 | 37.41937884 | 3.05E−08 | 7.97E−06 |
| . | gene | MSTRG.138135 | 85.2439273 | 3.11E−08 | 8.06E−06 |
| Gm10163 | gene | MSTRG.143042 | 16.26947101 | 3.12E−08 | 8.06E−06 |
| Gm6286 | gene | MSTRG.9536 | 15.18564043 | 3.13E−08 | 8.06E−06 |
| . | gene | MSTRG.28701 | 7.232213192 | 3.28E−08 | 8.40E−06 |
| Gm10250 | gene | MSTRG.47660 | 21.10034626 | 3.54E−08 | 9.02E−06 |
| Rplp0-ps1 | gene | MSTRG.89456 | 5.127509881 | 3.69E−08 | 9.34E−06 |
| Gm5527 | gene | MSTRG.2807 | 10.52281879 | 3.76E−08 | 9.49E−06 |
| Rps19-ps4 | gene | MSTRG.66809 | 46.34237045 | 3.80E−08 | 9.54E−06 |
| Slc35g1 | gene | MSTRG.72023 | 19.94393814 | 3.81E−08 | 9.54E−06 |
| Rpl31-ps12 | gene | MSTRG.54288 | 35.75808386 | 3.84E−08 | 9.58E−06 |
| Alppl2 | gene | MSTRG.4936 | 62.71471227 | 4.08E−08 | 1.01E−05 |
| . | gene | MSTRG.84211 | 6.818934061 | 4.11E−08 | 1.02E−05 |
| . | gene | MSTRG.17163 | 203.0090238 | 4.38E−08 | 1.08E−05 |
| Ube2nl | gene | MSTRG.124885 | 5.038327057 | 4.45E−08 | 1.09E−05 |
| Gm14633 | gene | MSTRG.147532 | 69.11395493 | 4.47E−08 | 1.09E−05 |
| Hmgb1-ps3 | gene | MSTRG.23083 | 11.34839443 | 4.47E−08 | 1.09E−05 |
| . | gene | MSTRG.35050 | 40.244115 | 4.58E−08 | 1.11E−05 |
| Sycp3 | gene | MSTRG.16696 | 38.55433729 | 4.69E−08 | 1.13E−05 |
| Gm12231 | gene | MSTRG.22337 | 62.65548657 | 4.78E−08 | 1.15E−05 |
| Gm8213 | gene | MSTRG.118666 | 12.30297882 | 4.82E−08 | 1.15E−05 |
| . | gene | MSTRG.25510 | 10.4750226 | 4.93E−08 | 1.18E−05 |
| Gm14173 | gene | MSTRG.82634 | 30.48427713 | 4.97E−08 | 1.18E−05 |
| . | gene | MSTRG.121732 | 3.388212175 | 5.06E−08 | 1.19E−05 |
| Gm10288 | gene | MSTRG.93086 | 64.22385525 | 5.19E−08 | 1.22E−05 |
| Gm43097 | gene | MSTRG.90884 | 5.952401444 | 5.33E−08 | 1.25E−05 |
| . | gene | MSTRG.70232 | 22.5671172 | 5.52E−08 | 1.29E−05 |
| Gm3851 | gene | MSTRG.6654 | 14.78564041 | 5.63E−08 | 1.30E−05 |
| Gm9575 | gene | MSTRG.56527 | 6.803160787 | 5.63E−08 | 1.30E−05 |
| . | gene | MSTRG.54477 | 7.773814953 | 5.79E−08 | 1.33E−05 |
| 2010005H15Rik | gene | MSTRG.55680 | 15.90043987 | 6.14E−08 | 1.41E−05 |
| Gm15843 | gene | MSTRG.3985 | 12.51996544 | 6.20E−08 | 1.42E−05 |
| . | gene | MSTRG.46087 | 20.43163264 | 6.28E−08 | 1.43E−05 |
| Gm11826 | gene | MSTRG.94264 | 32.59890853 | 6.43E−08 | 1.46E−05 |
| Sppl2a | gene | MSTRG.80713 | 10.67661253 | 6.58E−08 | 1.49E−05 |
| . | gene | MSTRG.25168 | 4.283026377 | 6.91E−08 | 1.55E−05 |
| Rpl28-ps1 | gene | MSTRG.7060 | 49.8970738 | 6.93E−08 | 1.55E−05 |
| . | gene | MSTRG.109616 | 6.625821754 | 7.12E−08 | 1.59E−05 |
| Rps24-ps3 | gene | MSTRG.148474 | 45.34636749 | 7.23E−08 | 1.60E−05 |
| Gm13532 | gene | MSTRG.76996 | 5.372084801 | 7.25E−08 | 1.60E−05 |
| Slc15a2 | gene | MSTRG.55726 | 75.02529254 | 7.31E−08 | 1.61E−05 |
| Pramel5 | gene | MSTRG.101943 | 46.49222747 | 7.36E−08 | 1.62E−05 |
| Gm16477 | gene | MSTRG.130017 | 24.49436953 | 7.41E−08 | 1.62E−05 |
| . | gene | MSTRG.46179 | 22.25859996 | 7.53E−08 | 1.64E−05 |
| Gm20900 | gene | MSTRG.41204 | 68.30876646 | 7.74E−08 | 1.68E−05 |
| Gm12458 | gene | MSTRG.96214 | 5.41526742 | 7.75E−08 | 1.68E−05 |
| Gm11448 | gene | MSTRG.82951 | 3.553019323 | 8.19E−08 | 1.76E−05 |
| Gm10268 | gene | MSTRG.66480 | 20.11700033 | 9.63E−08 | 2.05E−05 |
| Gm2710 | gene | MSTRG.9921 | 13.12919299 | 1.08E−07 | 2.29E−05 |
| Rpl21-ps10 | gene | MSTRG.86366 | 11.71585687 | 1.15E−07 | 2.42E−05 |
| Gm13573 | gene | MSTRG.77120 | 6.983247223 | 1.16E−07 | 2.44E−05 |
| . | gene | MSTRG.31139 | 24.2384821 | 1.18E−07 | 2.48E−05 |
| Gm13827 | gene | MSTRG.109533 | 17.14407188 | 1.20E−07 | 2.50E−05 |
| Gm9294 | gene | MSTRG.123897 | 16.08528402 | 1.21E−07 | 2.52E−05 |
| . | gene | MSTRG.124884 | 13.90967009 | 1.22E−07 | 2.52E−05 |
| Eif3s6-ps1 | gene | MSTRG.19665 | 8.590512885 | 1.26E−07 | 2.60E−05 |
| . | gene | MSTRG.101957 | 24.67134735 | 1.30E−07 | 2.67E−05 |
| C230085N15Rik | gene | MSTRG.62188 | 5.152846745 | 1.31E−07 | 2.69E−05 |
| Gm5857 | gene | MSTRG.92913 | 7.077133511 | 1.34E−07 | 2.74E−05 |
| Gpha2 | gene | MSTRG.69890 | 75.84027892 | 1.34E−07 | 2.74E−05 |
| . | gene | MSTRG.72227 | 10.35259269 | 1.46E−07 | 2.96E−05 |
| Gm3139 | gene | MSTRG.108125 | 26.51051523 | 1.47E−07 | 2.97E−05 |
| . | gene | MSTRG.115812 | 9.070238687 | 1.48E−07 | 2.97E−05 |
| . | gene | MSTRG.68058 | 3.098661742 | 1.53E−07 | 3.06E−05 |
| . | gene | MSTRG.70738 | 79.83782706 | 1.54E−07 | 3.07E−05 |
| . | gene | MSTRG.25940 | 16.32953375 | 1.56E−07 | 3.10E−05 |
| . | gene | MSTRG.78555 | 4.576552829 | 1.57E−07 | 3.11E−05 |
| . | gene | MSTRG.93952 | 14.76503968 | 1.58E−07 | 3.11E−05 |
| D10Wsu102e | gene | MSTRG.16287 | 15.33544739 | 1.58E−07 | 3.11E−05 |
| Gm12331 | gene | ENSMUSG00000081932 | 4.91866899 | 1.66E−07 | 3.23E−05 |
| Platr27 | gene | MSTRG.82762 | 18.35993133 | 1.66E−07 | 3.23E−05 |
| . | gene | MSTRG.34903 | 2.852878646 | 1.66E−07 | 3.23E−05 |
| Gm5093 | gene | MSTRG.61801 | 3.368423891 | 1.80E−07 | 3.50E−05 |
| . | gene | MSTRG.82522 | 5.829396709 | 1.83E−07 | 3.54E−05 |
| . | gene | MSTRG.145760 | 15.1533948 | 1.85E−07 | 3.56E−05 |
| Gm27529 | gene | MSTRG.47816 | 102.5432526 | 1.87E−07 | 3.60E−05 |
| . | gene | MSTRG.98499 | 9.297467752 | 1.88E−07 | 3.60E−05 |
| . | gene | MSTRG.34339 | 69.7430883 | 1.90E−07 | 3.62E−05 |
| Gm11539 | gene | MSTRG.25622 | 25.37690834 | 1.91E−07 | 3.63E−05 |
| Gm29257 | gene | MSTRG.7421 | 9.873680591 | 1.92E−07 | 3.65E−05 |
| Gm13370 | gene | MSTRG.74977 | 12.90414999 | 1.95E−07 | 3.70E−05 |
| Timm8a2 | gene | MSTRG.47412 | 57.17355768 | 1.99E−07 | 3.76E−05 |
| Rpsa-ps11 | gene | ENSMUSG00000082978 | 3.523859228 | 2.07E−07 | 3.90E−05 |
| Rpl30-ps9 | gene | MSTRG.149500 | 32.48668922 | 2.12E−07 | 3.97E−05 |
| Gm6341 | gene | MSTRG.127484 | 21.00359204 | 2.16E−07 | 4.03E−05 |
| BC028528 | gene | MSTRG.89731 | 43.65400982 | 2.23E−07 | 4.16E−05 |
| Gm12344 | gene | MSTRG.24240 | 20.5407075 | 2.27E−07 | 4.21E−05 |
| . | gene | MSTRG.142039 | 3.531518469 | 2.39E−07 | 4.43E−05 |
| Gm14138 | gene | MSTRG.80203 | 6.201026658 | 2.45E−07 | 4.53E−05 |
| Gm7808 | gene | MSTRG.138205 | 30.26470439 | 2.53E−07 | 4.64E−05 |
| . | gene | MSTRG.146704 | 16.02661936 | 2.56E−07 | 4.68E−05 |
| . | gene | MSTRG.137431 | 9.593095967 | 2.57E−07 | 4.68E−05 |
| . | gene | MSTRG.96863 | 4.978808499 | 2.58E−07 | 4.69E−05 |
| . | gene | MSTRG.31595 | 3.835229507 | 2.63E−07 | 4.76E−05 |
| . | gene | MSTRG.152381 | 13.74940438 | 2.67E−07 | 4.82E−05 |
| Rpl30-ps8 | gene | MSTRG.32856 | 25.17194559 | 2.72E−07 | 4.89E−05 |
| Gm11954 | gene | MSTRG.19264 | 7.384396773 | 2.79E−07 | 4.99E−05 |
| Gm6083 | gene | MSTRG.104374 | 10.08148056 | 2.82E−07 | 5.04E−05 |
| Gm7429 | gene | MSTRG.149839 | 6.426942475 | 2.84E−07 | 5.06E−05 |
| Rpl21-ps12 | gene | MSTRG.117355 | 4.02425047 | 2.88E−07 | 5.10E−05 |
| Gm14148 | gene | MSTRG.82152 | 31.46495874 | 2.98E−07 | 5.26E−05 |
| AC165294.3 | gene | MSTRG.145590 | 11.57851375 | 3.08E−07 | 5.41E−05 |
| . | gene | MSTRG.150851 | 9.402492326 | 3.09E−07 | 5.42E−05 |
| Gm14251 | gene | MSTRG.82645 | 36.10309573 | 3.12E−07 | 5.46E−05 |
| . | gene | MSTRG.50252 | 10.29926786 | 3.18E−07 | 5.55E−05 |
| Gm6681 | gene | MSTRG.117864 | 11.38875065 | 3.31E−07 | 5.73E−05 |
| Hmgb1-ps2 | gene | MSTRG.150854 | 5.294983122 | 3.42E−07 | 5.91E−05 |
| Sycn | gene | MSTRG.122823 | 34.92663274 | 3.45E−07 | 5.95E−05 |
| mt-Tl2 | gene | MSTRG.145687 | 8.340404653 | 3.51E−07 | 6.02E−05 |
| . | gene | MSTRG.25616 | 24.30577296 | 3.64E−07 | 6.22E−05 |
| . | gene | MSTRG.83509 | 26.74370965 | 3.77E−07 | 6.42E−05 |
| Gldc | gene | MSTRG.71548 | 13.55646942 | 3.84E−07 | 6.51E−05 |
| Asz1 | gene | MSTRG.113260 | 10.55913832 | 3.91E−07 | 6.59E−05 |
| Gm13148 | gene | MSTRG.102863 | 6.496404033 | 4.07E−07 | 6.83E−05 |
| Rps8-ps4 | gene | MSTRG.121571 | 6.446401576 | 4.11E−07 | 6.88E−05 |
| . | gene | MSTRG.146812 | 16.66033635 | 4.41E−07 | 7.33E−05 |
| Gm5580 | gene | MSTRG.118693 | 3.539004055 | 4.42E−07 | 7.33E−05 |
| Vtcn1 | gene | MSTRG.90166 | 9.189384843 | 4.48E−07 | 7.41E−05 |
| Cd63-ps | gene | MSTRG.67275 | 15.72369896 | 4.53E−07 | 7.47E−05 |
| Gm42573 | gene | MSTRG.113467 | 14.28877451 | 4.54E−07 | 7.47E−05 |
| Gm12704 | gene | MSTRG.98331 | 4.913545419 | 4.61E−07 | 7.56E−05 |
| RP23-212H18.2 | gene | MSTRG.124975 | 4.417390178 | 4.73E−07 | 7.74E−05 |
| . | gene | MSTRG.146909 | 3.069535802 | 4.79E−07 | 7.82E−05 |
| Gm13339 | gene | MSTRG.74810 | 42.35330513 | 4.91E−07 | 7.99E−05 |
| . | gene | MSTRG.14565 | 4.423917235 | 5.23E−07 | 8.49E−05 |
| . | gene | MSTRG.48953 | 20.48776831 | 5.32E−07 | 8.61E−05 |
| Zfp839 | gene | MSTRG.33677 | 3.964595657 | 5.42E−07 | 8.75E−05 |
| . | gene | MSTRG.54844 | 4.025296093 | 5.46E−07 | 8.79E−05 |
| Fbxo8 | gene | MSTRG.133418 | 10.01043649 | 5.64E−07 | 9.05E−05 |
| Gm5937 | gene | MSTRG.148423 | 5.034317539 | 5.68E−07 | 9.09E−05 |
| Gm6054 | gene | MSTRG.112257 | 143.1292497 | 5.75E−07 | 9.19E−05 |
| . | gene | MSTRG.2449 | 5.414842803 | 5.94E−07 | 9.46E−05 |
| Xlr4b | gene | MSTRG.148178 | 15.32057226 | 6.06E−07 | 9.60E−05 |
| Gm43471 | gene | MSTRG.87112 | 8.284631377 | 6.07E−07 | 9.60E−05 |
| Gm16165 | gene | MSTRG.138502 | 11.28448862 | 6.17E−07 | 9.75E−05 |
| Gm13268 | gene | MSTRG.74264 | 3.738787697 | 6.36E−07 | 0.000100215 |
| . | gene | MSTRG.28394 | 17.57380854 | 6.48E−07 | 0.000101752 |
| . | gene | MSTRG.53424 | 8.01235608 | 6.78E−07 | 0.000106256 |
| AI662270 | gene | MSTRG.24815 | 17.00406696 | 6.88E−07 | 0.000107167 |
| . | gene | MSTRG.142979 | 4.798654622 | 6.89E−07 | 0.000107167 |
| . | gene | MSTRG.89547 | 33.76259377 | 6.90E−07 | 0.000107167 |
| Gm9104 | gene | MSTRG.61695 | 23.71708949 | 7.02E−07 | 0.000108837 |
| Rps3a3 | gene | MSTRG.40578 | 31.37309219 | 7.06E−07 | 0.000109115 |
| Gm11401 | gene | MSTRG.19473 | 2.742252278 | 7.21E−07 | 0.000110768 |
| Gm12261 | gene | MSTRG.22647 | 14.34235221 | 7.21E−07 | 0.000110768 |
| . | gene | MSTRG.151845 | 13.47674937 | 7.22E−07 | 0.000110768 |
| Morn2 | gene | MSTRG.63805 | 15.99061429 | 7.27E−07 | 0.000111219 |
| Gm44180 | gene | MSTRG.118624 | 6.862593144 | 7.42E−07 | 0.000113225 |
| Gm13578 | gene | MSTRG.77303 | 3.295947319 | 7.44E−07 | 0.000113247 |
| Gm12174 | gene | MSTRG.21847 | 29.26903704 | 7.57E−07 | 0.000114946 |
| . | gene | MSTRG.72111 | 7.489153099 | 7.62E−07 | 0.000115361 |
| . | gene | MSTRG.34222 | 23.28080852 | 7.72E−07 | 0.000116551 |
| Gm13611 | gene | MSTRG.75655 | 70.0899223 | 7.85E−07 | 0.000118317 |
| Gm16288 | gene | MSTRG.104453 | 5.172410834 | 8.29E−07 | 0.00012466 |
| Rps4x-ps | gene | MSTRG.102939 | 5.518865999 | 8.39E−07 | 0.000125702 |
| Gm11449 | gene | MSTRG.82959 | 28.58983429 | 8.67E−07 | 0.000129709 |
| . | gene | MSTRG.17024 | 13.1701118 | 8.75E−07 | 0.000130429 |
| . | gene | MSTRG.152282 | 12.59853985 | 8.84E−07 | 0.000131421 |
| Gm9794 | gene | MSTRG.115913 | 40.4443248 | 8.86E−07 | 0.000131421 |
| Gm21399 | gene | MSTRG.137371 | 22.51966778 | 8.92E−07 | 0.000131681 |
| . | gene | MSTRG.28622 | 37.97296462 | 8.94E−07 | 0.000131681 |
| . | gene | MSTRG.60736 | 3.31390668 | 9.32E−07 | 0.00013694 |
| . | gene | MSTRG.149838 | 3.920104387 | 9.42E−07 | 0.000137774 |
| . | gene | MSTRG.68840 | 11.36717997 | 9.54E−07 | 0.000139159 |
| . | gene | MSTRG.36825 | 0.002313198 | 9.63E−07 | 0.000140041 |
| Anp32-ps | gene | MSTRG.21166 | 6.714375502 | 9.77E−07 | 0.000141712 |
| Reep1 | gene | MSTRG.116225 | 8.923260882 | 1.01E−06 | 0.000145959 |
| Gm5045 | gene | MSTRG.50251 | 7.791935084 | 1.03E−06 | 0.000148599 |
| RP24-269P21.1 | gene | MSTRG.127026 | 6.69284763 | 1.03E−06 | 0.000148599 |
| Rpl30-ps1 | gene | MSTRG.114378 | 18.97615769 | 1.05E−06 | 0.000150724 |
| . | gene | MSTRG.107989 | 4.162142071 | 1.05E−06 | 0.000150724 |
| Gm12618 | gene | MSTRG.22821 | 3.893703497 | 1.05E−06 | 0.000150763 |
| Ppm1k | gene | MSTRG.115593 | 6.113868054 | 1.07E−06 | 0.000152614 |
| Gm5845 | gene | MSTRG.86063 | 3.165091525 | 1.13E−06 | 0.000161485 |
| Gm11222 | gene | MSTRG.97138 | 6.844617786 | 1.14E−06 | 0.000161896 |
| Gm6335 | gene | MSTRG.149739 | 39.01643171 | 1.14E−06 | 0.000161896 |
| Gm12906 | gene | MSTRG.99122 | 5.211808006 | 1.16E−06 | 0.000163996 |
| Gm5160 | gene | MSTRG.65339 | 3.934186349 | 1.19E−06 | 0.000167799 |
| Gm11953 | gene | MSTRG.19256 | 7.78996402 | 1.22E−06 | 0.000171167 |
| . | gene | MSTRG.137433 | 7.082778653 | 1.23E−06 | 0.000172205 |
| Gm11575 | gene | MSTRG.3362 | 8.692442285 | 1.27E−06 | 0.000176467 |
| Rpl7a-ps5 | gene | MSTRG.62530 | 4.345082111 | 1.28E−06 | 0.000178403 |
| AC164084.2 | gene | MSTRG.145582 | 11.12323899 | 1.34E−06 | 0.000185708 |
| Gm5518 | gene | MSTRG.71473 | 3.949262975 | 1.34E−06 | 0.000185708 |
| Rpl18-ps1 | gene | MSTRG.3405 | 11.84709291 | 1.36E−06 | 0.000187263 |
| Rpl7-ps7 | gene | MSTRG.107518 | 17.74479057 | 1.37E−06 | 0.000188897 |
| RP24-427A8.1 | gene | MSTRG.129529 | 4.012392408 | 1.38E−06 | 0.000190013 |
| . | gene | MSTRG.46669 | 3.562481461 | 1.42E−06 | 0.000194567 |
| Rhobtb2 | gene | MSTRG.45168 | 4.598121838 | 1.48E−06 | 0.000201954 |
| Gm9493 | gene | MSTRG.71084 | 29.23981222 | 1.53E−06 | 0.000207611 |
| Tspan8 | gene | MSTRG.18185 | 477.2226838 | 1.56E−06 | 0.000212172 |
| . | gene | MSTRG.1978 | 15.05693106 | 1.59E−06 | 0.000214849 |
| . | gene | MSTRG.54336 | 27.08801344 | 1.61E−06 | 0.000216795 |
| . | gene | MSTRG.28730 | 23.73984922 | 1.64E−06 | 0.000219971 |
| Gm13422 | gene | MSTRG.74848 | 9.603724057 | 1.66E−06 | 0.000222236 |
| Gm9625 | gene | MSTRG.38262 | 23.81072401 | 1.69E−06 | 0.000226239 |
| Rpl7a-ps11 | gene | MSTRG.151710 | 16.8200539 | 1.69E−06 | 0.000226327 |
| Gm8337 | gene | MSTRG.2534 | 5.797116902 | 1.74E−06 | 0.000231652 |
| . | gene | MSTRG.137799 | 7.079945698 | 1.74E−06 | 0.000231652 |
| . | gene | MSTRG.102826 | 9.916781266 | 1.81E−06 | 0.00024033 |
| Ccpg1os | gene | MSTRG.142163 | 16.24850024 | 1.81E−06 | 0.00024033 |
| Rps15a-ps6 | gene | MSTRG.19475 | 21.34357668 | 1.82E−06 | 0.000240428 |
| . | gene | MSTRG.135208 | 10.53914983 | 1.83E−06 | 0.000241309 |
| . | gene | MSTRG.140774 | 26.73486531 | 1.83E−06 | 0.000241373 |
| . | gene | MSTRG.32970 | 5.989693776 | 1.85E−06 | 0.000243279 |
| Gm36964 | gene | MSTRG.368 | 4.058875635 | 1.87E−06 | 0.000245286 |
| Gm11598 | gene | ENSMUSG00000082456 | 2.805662127 | 1.88E−06 | 0.000245638 |
| Gm10073 | gene | MSTRG.136178 | 23.06636219 | 1.89E−06 | 0.000246735 |
| Chga | gene | MSTRG.33095 | 10.99429526 | 1.97E−06 | 0.00025672 |
| Gm3355 | gene | MSTRG.59410 | 3.798551032 | 1.98E−06 | 0.000256961 |
| . | gene | MSTRG.33622 | 5.980706145 | 1.99E−06 | 0.000257827 |
| . | gene | MSTRG.92016 | 13.1257441 | 2.02E−06 | 0.000260397 |
| . | gene | MSTRG.111517 | 4.718294579 | 2.02E−06 | 0.000260397 |
| Gm6166 | gene | MSTRG.141034 | 46.56727865 | 2.07E−06 | 0.000266216 |
| Gm13408 | gene | MSTRG.75922 | 56.77368055 | 2.09E−06 | 0.000267729 |
| Gm15361 | gene | MSTRG.148270 | 5.540222437 | 2.10E−06 | 0.000269105 |
| Rpl30-ps10 | gene | MSTRG.148087 | 23.80653838 | 2.11E−06 | 0.000269105 |
| . | gene | MSTRG.85147 | 6.410258783 | 2.12E−06 | 0.000269105 |
| Dennd5a | gene | MSTRG.128060 | 4.649721693 | 2.12E−06 | 0.000269105 |
| Gm6478 | gene | MSTRG.37982 | 2.941274712 | 2.12E−06 | 0.000269105 |
| Gm13835 | gene | MSTRG.113978 | 12.92736513 | 2.14E−06 | 0.000271271 |
| . | gene | MSTRG.69498 | 4.573594549 | 2.17E−06 | 0.000274039 |
| Gm10240 | gene | MSTRG.50594 | 13.67698682 | 2.20E−06 | 0.000277141 |
| . | gene | MSTRG.124052 | 3.795212105 | 2.22E−06 | 0.000279699 |
| G430049J08Rik | gene | MSTRG.64616 | 12.47199559 | 2.24E−06 | 0.000282105 |
| Gm14300 | gene | MSTRG.84210 | 7.907543867 | 2.26E−06 | 0.000283689 |
| . | gene | MSTRG.18143 | 10.92763334 | 2.31E−06 | 0.000288927 |
| Aldh3b2 | gene | MSTRG.69611 | 7.894837367 | 2.36E−06 | 0.000294758 |
| Snora78 | gene | MSTRG.60004 | 14.07518708 | 2.39E−06 | 0.000297321 |
| Gm5853 | gene | MSTRG.90023 | 4.233824084 | 2.39E−06 | 0.000297475 |
| . | gene | MSTRG.152174 | 12.19147949 | 2.49E−06 | 0.00030882 |
| Gm8399 | gene | MSTRG.38892 | 44.31789867 | 2.61E−06 | 0.000322633 |
| Khdc1a | gene | MSTRG.1101 | 7.908958122 | 2.79E−06 | 0.000343781 |
| Cmbl | gene | MSTRG.48870 | 108.8543601 | 2.83E−06 | 0.000347677 |
| Ccdc170 | gene | MSTRG.11404 | 4.487706013 | 2.87E−06 | 0.000351916 |
| Gm9050 | gene | MSTRG.149394 | 4.439523715 | 2.93E−06 | 0.000358944 |
| . | gene | MSTRG.137430 | 13.77287462 | 2.99E−06 | 0.000365767 |
| Tmem86a | gene | MSTRG.124252 | 10.66134845 | 3.01E−06 | 0.00036602 |
| . | gene | MSTRG.122656 | 4.827919135 | 3.03E−06 | 0.000367446 |
| Gm8865 | gene | MSTRG.44166 | 8.909124662 | 3.07E−06 | 0.000370032 |
| Gm8318 | gene | MSTRG.48341 | 6.589407488 | 3.08E−06 | 0.000370032 |
| . | gene | MSTRG.8872 | 3.908438661 | 3.08E−06 | 0.000370032 |
| . | gene | MSTRG.11583 | 8.520301426 | 3.16E−06 | 0.000378802 |
| Gm10051 | gene | MSTRG.110757 | 48.24691852 | 3.47E−06 | 0.000412475 |
| . | gene | MSTRG.13676 | 5.769496394 | 3.47E−06 | 0.000412475 |
| Eno1 | gene | MSTRG.102303 | 0.078150392 | 3.48E−06 | 0.000412639 |
| Gm43028 | gene | MSTRG.106704 | 7.565619274 | 3.49E−06 | 0.000412639 |
| Gm8226 | gene | MSTRG.142775 | 19.08941796 | 3.52E−06 | 0.000415564 |
| Rpl31-ps4 | gene | MSTRG.57996 | 6.724216059 | 3.53E−06 | 0.000415564 |
| Gm7027 | gene | MSTRG.127498 | 26.1299899 | 3.65E−06 | 0.000429495 |
| Gm5805 | gene | MSTRG.51745 | 28.67777041 | 3.68E−06 | 0.000432205 |
| Rpl21-ps8 | gene | MSTRG.69131 | 34.95492517 | 3.76E−06 | 0.000441094 |
| . | gene | MSTRG.49284 | 5.359216093 | 3.79E−06 | 0.000443122 |
| Cpn1 | gene | MSTRG.72409 | 33.87024708 | 3.86E−06 | 0.000450175 |
| . | gene | MSTRG.73636 | 3.95379373 | 4.03E−06 | 0.000468325 |
| Gm13310 | gene | MSTRG.74160 | 4.001966405 | 4.14E−06 | 0.000480525 |
| . | gene | MSTRG.25156 | 5.505587752 | 4.36E−06 | 0.000505309 |
| . | gene | MSTRG.75901 | 25.95018291 | 4.44E−06 | 0.000513181 |
| BC053393 | gene | MSTRG.21759 | 124.1760129 | 4.47E−06 | 0.000515864 |
| Gm43008 | gene | MSTRG.86420 | 4.981855784 | 4.54E−06 | 0.000521132 |
| Gm3531 | gene | MSTRG.5674 | 33.06404234 | 4.62E−06 | 0.000528626 |
| Gm12254 | gene | MSTRG.22548 | 77.10054991 | 4.62E−06 | 0.000528626 |
| Gm7832 | gene | MSTRG.107940 | 4.699684116 | 4.72E−06 | 0.000539744 |
| Gm15387 | gene | MSTRG.50973 | 2.440043724 | 4.74E−06 | 0.000540198 |
| Gm8623 | gene | MSTRG.133007 | 31.1262599 | 4.92E−06 | 0.000559064 |
| Gm11407 | gene | MSTRG.97462 | 2.94200278 | 4.95E−06 | 0.000561066 |
| . | gene | MSTRG.134534 | 45.92868583 | 4.97E−06 | 0.000562645 |
| Gm10709 | gene | MSTRG.137617 | 18.31302892 | 5.00E−06 | 0.00056425 |
| . | gene | MSTRG.13851 | 9.753229461 | 5.35E−06 | 0.000601873 |
| . | gene | MSTRG.128441 | 4.810132837 | 5.35E−06 | 0.000601873 |
| Gm16378 | gene | MSTRG.140349 | 24.53313739 | 5.39E−06 | 0.000604755 |
| Gm14648 | gene | MSTRG.147204 | 9.180429392 | 5.65E−06 | 0.000632183 |
| Hist1h2ap | gene | MSTRG.35488 | 0.003568328 | 5.68E−06 | 0.000633851 |
| . | gene | MSTRG.122581 | 4.10970017 | 5.84E−06 | 0.000649915 |
| Gm12115 | gene | MSTRG.21100 | 4.609012381 | 5.91E−06 | 0.000656648 |
| Gm7327 | gene | MSTRG.147709 | 6.673732 | 6.30E−06 | 0.000696451 |
| Npr1 | gene | MSTRG.89334 | 17.95944014 | 6.35E−06 | 0.000701099 |
| Gm5873 | gene | ENSMUSG00000093651 | 5.995248662 | 6.40E−06 | 0.000704824 |
| . | gene | MSTRG.48627 | 11.17656258 | 6.43E−06 | 0.000707519 |
| Ttc39c | gene | MSTRG.65256 | 10.42538615 | 6.52E−06 | 0.000715674 |
| Gm13882 | gene | MSTRG.79353 | 7.059110872 | 6.72E−06 | 0.000734676 |
| Ube2l6 | gene | MSTRG.78394 | 43.49877729 | 6.76E−06 | 0.000737987 |
| . | gene | MSTRG.53155 | 3.841763451 | 7.01E−06 | 0.000763988 |
| . | gene | MSTRG.59444 | 9.751893624 | 7.22E−06 | 0.00078496 |
| Gm12712 | gene | MSTRG.23791 | 5.910911271 | 7.24E−06 | 0.000785952 |
| . | gene | MSTRG.63882 | 39.95067079 | 7.29E−06 | 0.000790354 |
| D5Ertd605e | gene | MSTRG.112265 | 26.94770754 | 7.31E−06 | 0.000790598 |
| Gm15198 | gene | MSTRG.13680 | 2.633495603 | 7.34E−06 | 0.000790748 |
| . | gene | MSTRG.136583 | 3.944107297 | 7.44E−06 | 0.000797204 |
| . | gene | MSTRG.14680 | 32.49518041 | 7.50E−06 | 0.000800416 |
| Gm7816 | gene | MSTRG.105263 | 25.84177992 | 7.51E−06 | 0.000800416 |
| Gm37070 | gene | MSTRG.10377 | 2.947115957 | 7.76E−06 | 0.000825992 |
| . | gene | MSTRG.50207 | 8.202740197 | 7.78E−06 | 0.000825992 |
| . | gene | MSTRG.16256 | 5.1673848 | 7.83E−06 | 0.000829984 |
| . | gene | MSTRG.38644 | 66.3043817 | 8.00E−06 | 0.000846533 |
| RP23-207N2.1 | gene | MSTRG.125973 | 17.32102499 | 8.22E−06 | 0.000866344 |
| Rpl9-ps7 | gene | MSTRG.77567 | 51.95189372 | 8.23E−06 | 0.000866344 |
| L2hgdh | gene | MSTRG.31068 | 4.761599757 | 8.23E−06 | 0.000866344 |
| Rpl36a-ps1 | gene | MSTRG.46387 | 22.43566395 | 8.51E−06 | 0.00089437 |
| Slc25a16 | gene | MSTRG.14926 | 13.51407726 | 8.81E−06 | 0.000922968 |
| . | gene | MSTRG.41981 | 33.94205697 | 8.81E−06 | 0.000922968 |
| . | gene | MSTRG.84569 | 4.406089846 | 9.23E−06 | 0.000964378 |
| Gm7180 | gene | MSTRG.126152 | 8.737013432 | 9.51E−06 | 0.000992587 |
| . | gene | MSTRG.63868 | 7.666585189 | 9.56E−06 | 0.000995649 |
| Adap2os | gene | MSTRG.24615 | 9.754428549 | 1.01E−05 | 0.001045718 |
| . | gene | MSTRG.81712 | 40.23863452 | 1.02E−05 | 0.001053684 |
| Xlr4c | gene | MSTRG.148180 | 9.0078151 | 1.02E−05 | 0.001053684 |
| . | gene | MSTRG.38104 | 7.817501269 | 1.06E−05 | 0.001084888 |
| . | gene | MSTRG.48954 | 7.511179972 | 1.09E−05 | 0.001114824 |
| . | gene | MSTRG.132726 | 18.30479169 | 1.10E−05 | 0.0011267 |
| Glipr1 | gene | MSTRG.18019 | 24.86200642 | 1.11E−05 | 0.001135534 |
| Gm6180 | gene | MSTRG.132725 | 8.329537371 | 1.13E−05 | 0.001148029 |
| Ngfrap1 | gene | MSTRG.150458 | 0.003070455 | 1.13E−05 | 0.001153622 |
| . | gene | MSTRG.40137 | 18.51030417 | 1.13E−05 | 0.001153622 |
| . | gene | MSTRG.69617 | 13.93129262 | 1.17E−05 | 0.001190411 |
| . | gene | MSTRG.7549 | 9.759257808 | 1.19E−05 | 0.001202109 |
| Gm3286 | gene | MSTRG.145506 | 11.67827817 | 1.19E−05 | 0.001202109 |
| Gm9385 | gene | MSTRG.144787 | 41.77217153 | 1.19E−05 | 0.001203633 |
| . | gene | MSTRG.60422 | 5.525059862 | 1.21E−05 | 0.00121737 |
| Gm7507 | gene | MSTRG.116889 | 4.410542372 | 1.23E−05 | 0.001235847 |
| . | gene | MSTRG.38098 | 17.76496498 | 1.25E−05 | 0.001245206 |
| . | gene | MSTRG.30413 | 8.945584848 | 1.25E−05 | 0.001245206 |
| Gm37164 | gene | MSTRG.85185 | 10.01200678 | 1.26E−05 | 0.001250091 |
| . | gene | MSTRG.50332 | 12.23023672 | 1.26E−05 | 0.0012531 |
| Gm6467 | gene | MSTRG.54740 | 18.07558107 | 1.26E−05 | 0.0012531 |
| Hmgb1-ps1 | gene | MSTRG.21479 | 8.739110348 | 1.30E−05 | 0.001283728 |
| Pdzk1ip1 | gene | MSTRG.99510 | 59.40726007 | 1.30E−05 | 0.001285962 |
| Gm9238 | gene | MSTRG.34001 | 7.732229886 | 1.30E−05 | 0.001285962 |
| Gm12328 | gene | MSTRG.23898 | 7.171011103 | 1.31E−05 | 0.001288387 |
| Gm26384 | gene | MSTRG.59710 | 21.00868494 | 1.31E−05 | 0.001289547 |
| Acbd3 | gene | MSTRG.10441 | 6.001725449 | 1.35E−05 | 0.001315687 |
| Mmgt1 | gene | MSTRG.147542 | 6.22823561 | 1.35E−05 | 0.001315687 |
| . | gene | MSTRG.137434 | 3.385787981 | 1.38E−05 | 0.001343544 |
| . | gene | MSTRG.108159 | 10.34935341 | 1.39E−05 | 0.001358732 |
| Gm5879 | gene | MSTRG.117139 | 87.67238342 | 1.43E−05 | 0.001392387 |
| . | gene | MSTRG.70528 | 14.82953002 | 1.43E−05 | 0.001393582 |
| . | gene | MSTRG.64593 | 5.181253618 | 1.45E−05 | 0.001406094 |
| Gm13232 | gene | MSTRG.102733 | 19.52481463 | 1.49E−05 | 0.001438362 |
| Nynrin | gene | MSTRG.44289 | 7.763608141 | 1.51E−05 | 0.001457259 |
| . | gene | MSTRG.116670 | 10.99839369 | 1.51E−05 | 0.001457259 |
| Rps15a-ps7 | gene | MSTRG.82214 | 2.953716215 | 1.52E−05 | 0.001457718 |
| Ldha | gene | MSTRG.124313 | 0.00974498 | 1.53E−05 | 0.00146388 |
| Gm6091 | gene | MSTRG.137236 | 17.75536767 | 1.56E−05 | 0.001495854 |
| Bhmt2 | gene | MSTRG.39652 | 21.17965818 | 1.57E−05 | 0.001495854 |
| Gm14681 | gene | MSTRG.147904 | 41.03492692 | 1.58E−05 | 0.001500401 |
| Gyg | gene | MSTRG.85458 | 57.67492234 | 1.58E−05 | 0.001502241 |
| Gm27018 | gene | MSTRG.112773 | 2.310464644 | 1.58E−05 | 0.001502919 |
| . | gene | MSTRG.127177 | 5.800103997 | 1.60E−05 | 0.001517723 |
| Rpap1 | gene | MSTRG.80270 | 10.80228103 | 1.64E−05 | 0.001552181 |
| . | gene | MSTRG.85435 | 4.013938071 | 1.74E−05 | 0.00163867 |
| Gm12169 | gene | MSTRG.21750 | 12.21907141 | 1.75E−05 | 0.001645245 |
| Gm13160 | gene | MSTRG.102814 | 13.17268023 | 1.76E−05 | 0.001653091 |
| Rps11-ps2 | gene | MSTRG.27209 | 4.270472682 | 1.79E−05 | 0.001673538 |
| Gm5564 | gene | MSTRG.111381 | 6.245854224 | 1.79E−05 | 0.001673538 |
| Rps19-ps1 | gene | MSTRG.43934 | 7.211844273 | 1.79E−05 | 0.001673538 |
| . | gene | MSTRG.84381 | 25.46061572 | 1.79E−05 | 0.001673538 |
| Vma21-ps | gene | MSTRG.96332 | 3.294894243 | 1.81E−05 | 0.001682834 |
| Rpl36-ps3 | gene | MSTRG.28228 | 32.47222974 | 1.81E−05 | 0.001684162 |
| . | gene | MSTRG.118783 | 2.82582217 | 1.83E−05 | 0.001696922 |
| Marcksl1 | gene | MSTRG.100709 | 0.123866009 | 1.86E−05 | 0.001722882 |
| . | gene | MSTRG.90165 | 35.40963361 | 1.86E−05 | 0.001722925 |
| Gm11652 | gene | MSTRG.26545 | 4.941340349 | 1.98E−05 | 0.001833636 |
| . | gene | MSTRG.121057 | 20.32096886 | 2.04E−05 | 0.00187925 |
| Gm10343 | gene | MSTRG.53812 | 9.398549491 | 2.04E−05 | 0.001884193 |
| . | gene | MSTRG.22697 | 21.03212868 | 2.06E−05 | 0.00189621 |
| . | gene | MSTRG.31019 | 9.63183809 | 2.08E−05 | 0.001912199 |
| Gm10290 | gene | MSTRG.104332 | 3.8930321 | 2.19E−05 | 0.002001075 |
| . | gene | MSTRG.131120 | 19.7203415 | 2.21E−05 | 0.002020191 |
| Gm8618 | gene | MSTRG.7498 | 3.549081325 | 2.23E−05 | 0.00203664 |
| . | gene | MSTRG.86421 | 4.883615376 | 2.24E−05 | 0.002039789 |
| RP23-473E20.3 | gene | MSTRG.127113 | 7.601234745 | 2.26E−05 | 0.002048849 |
| . | gene | MSTRG.28569 | 10.188736 | 2.29E−05 | 0.002069165 |
| . | gene | MSTRG.146811 | 8.144221453 | 2.40E−05 | 0.002166369 |
| Rps12-ps9 | gene | MSTRG.140401 | 13.41503859 | 2.40E−05 | 0.002166369 |
| . | gene | MSTRG.147208 | 8.877110658 | 2.41E−05 | 0.002172392 |
| Pfkl | gene | MSTRG.15740 | 0.056647946 | 2.42E−05 | 0.00217521 |
| Gm10132 | gene | MSTRG.45553 | 9.356251401 | 2.44E−05 | 0.002188113 |
| Hist1h2ag | gene | MSTRG.35507 | 0.015559514 | 2.46E−05 | 0.002203533 |
| . | gene | MSTRG.59861 | 5.362004782 | 2.50E−05 | 0.002236389 |
| H3f3a-ps2 | gene | MSTRG.58165 | 25.53971119 | 2.54E−05 | 0.002267319 |
| Tmem213 | gene | MSTRG.114371 | 10.86715876 | 2.55E−05 | 0.002267319 |
| Gm5510 | gene | MSTRG.69888 | 4.792631544 | 2.55E−05 | 0.002267319 |
| . | gene | MSTRG.15111 | 8.370107663 | 2.56E−05 | 0.002267319 |
| Hist1h2ao | gene | MSTRG.35484 | 0.003707973 | 2.56E−05 | 0.002267319 |
| Ttc7b | gene | MSTRG.32962 | 27.55428447 | 2.57E−05 | 0.002272878 |
| Gm22614 | gene | MSTRG.89831 | 4.356890562 | 2.61E−05 | 0.002306333 |
| . | gene | MSTRG.108776 | 14.26192917 | 2.61E−05 | 0.002306333 |
| . | gene | MSTRG.92833 | 3.430150384 | 2.64E−05 | 0.002330426 |
| . | gene | MSTRG.119443 | 17.17510526 | 2.67E−05 | 0.0023474 |
| Gm11425 | gene | MSTRG.24788 | 8.660003556 | 2.77E−05 | 0.002425299 |
| Gm4217 | gene | MSTRG.51952 | 4.417318372 | 2.80E−05 | 0.002441018 |
| . | gene | MSTRG.77718 | 16.07487317 | 2.81E−05 | 0.00245085 |
| Hspb1 | gene | MSTRG.111096 | 0.003036519 | 2.86E−05 | 0.002486985 |
| Atp6v0c-ps1 | gene | MSTRG.120968 | 3.442842234 | 2.92E−05 | 0.002524853 |
| Eif5al3-ps | gene | MSTRG.107879 | 3.290074575 | 2.94E−05 | 0.002536462 |
| Gm6170 | gene | MSTRG.7091 | 13.11962007 | 2.97E−05 | 0.002561176 |
| . | gene | MSTRG.70759 | 18.68383205 | 3.00E−05 | 0.002580442 |
| Rps19-ps2 | gene | MSTRG.43910 | 6.086412035 | 3.01E−05 | 0.002581809 |
| . | gene | MSTRG.129168 | 10.92887402 | 3.05E−05 | 0.002611218 |
| Gm5558 | gene | MSTRG.107943 | 6.008259266 | 3.09E−05 | 0.002646027 |
| . | gene | MSTRG.54057 | 17.09284223 | 3.16E−05 | 0.00269862 |
| . | gene | MSTRG.139121 | 108.6398587 | 3.17E−05 | 0.00270902 |
| . | gene | MSTRG.58470 | 3.277009891 | 3.22E−05 | 0.002740273 |
| . | gene | MSTRG.65520 | 6.41908211 | 3.24E−05 | 0.002756418 |
| . | gene | MSTRG.29115 | 5.46926407 | 3.25E−05 | 0.002763746 |
| Gm10689 | gene | MSTRG.131807 | 6.997739113 | 3.27E−05 | 0.002771374 |
| . | gene | MSTRG.139851 | 4.08664458 | 3.31E−05 | 0.002798765 |
| . | gene | MSTRG.110572 | 0.021951672 | 3.31E−05 | 0.002798765 |
| Gm5566 | gene | MSTRG.112432 | 4.299609963 | 3.32E−05 | 0.002800454 |
| Gm14706 | gene | MSTRG.148416 | 2.555795875 | 3.47E−05 | 0.002917854 |
| Tcf23 | gene | MSTRG.104523 | 3.924814746 | 3.51E−05 | 0.002948455 |
| Ypel2 | gene | MSTRG.25075 | 11.11525963 | 3.52E−05 | 0.002949113 |
| Hist1h2ac | gene | MSTRG.35567 | 0.014577314 | 3.59E−05 | 0.003008181 |
| . | gene | MSTRG.28835 | 3.814888414 | 3.62E−05 | 0.003025006 |
| Gm11336 | gene | MSTRG.35578 | 0.023425062 | 3.67E−05 | 0.003067652 |
| . | gene | MSTRG.3084 | 8.914340661 | 3.69E−05 | 0.003076772 |
| . | gene | MSTRG.130619 | 5.932065356 | 3.71E−05 | 0.003083744 |
| . | gene | MSTRG.65726 | 14.9850261 | 3.73E−05 | 0.003097192 |
| Gm11361 | gene | MSTRG.35782 | 14.10380987 | 3.84E−05 | 0.003187311 |
| Gm8121 | gene | MSTRG.106202 | 3.442794873 | 3.87E−05 | 0.003205898 |
| Hist1h2ab | gene | MSTRG.35580 | 0.010797895 | 3.89E−05 | 0.003220321 |
| Gm8444 | gene | MSTRG.51708 | 2.339077562 | 3.93E−05 | 0.003246845 |
| Rpl38-ps2 | gene | MSTRG.120232 | 19.56588793 | 4.06E−05 | 0.003351612 |
| Gm43712 | gene | MSTRG.89308 | 22.60173156 | 4.17E−05 | 0.003433166 |
| Atp5l-ps1 | gene | MSTRG.124924 | 8.939785764 | 4.25E−05 | 0.003492149 |
| Rcor1 | gene | MSTRG.33735 | 5.930248007 | 4.28E−05 | 0.003515162 |
| Csta1 | gene | MSTRG.55669 | 10.10111893 | 4.35E−05 | 0.003568889 |
| Wfdc15a | gene | MSTRG.83138 | 26.83398836 | 4.39E−05 | 0.003597205 |
| Rpl9-ps4 | gene | MSTRG.38456 | 60.39369558 | 4.41E−05 | 0.003610204 |
| Gm9396 | gene | MSTRG.92047 | 9.194492603 | 4.44E−05 | 0.003625917 |
| . | gene | MSTRG.64781 | 7.880101009 | 4.48E−05 | 0.003649372 |
| Gm13680 | gene | MSTRG.78317 | 28.02983858 | 4.54E−05 | 0.003690877 |
| . | gene | MSTRG.29929 | 3.617558447 | 4.56E−05 | 0.003707902 |
| Gm11349 | gene | MSTRG.35690 | 3.473278688 | 4.63E−05 | 0.003756264 |
| Ptgis | gene | MSTRG.83381 | 11.96761794 | 4.65E−05 | 0.003763255 |
| Gm13340 | gene | MSTRG.74809 | 8.946516488 | 4.66E−05 | 0.003763255 |
| Gm11517 | gene | MSTRG.25667 | 12.37451729 | 4.66E−05 | 0.003763255 |
| Gm27219 | gene | MSTRG.139229 | 6.802142962 | 4.68E−05 | 0.00377407 |
| . | gene | MSTRG.67135 | 9.97735574 | 4.74E−05 | 0.003817307 |
| Rbpsuh-rs3 | gene | MSTRG.114915 | 5.433902623 | 4.77E−05 | 0.00383016 |
| . | gene | MSTRG.3192 | 5.363528686 | 4.82E−05 | 0.003871313 |
| Dmc1 | gene | MSTRG.51496 | 7.421151513 | 4.85E−05 | 0.00388275 |
| Gm10335 | gene | MSTRG.12076 | 10.28457528 | 4.85E−05 | 0.00388275 |
| Gm28555 | gene | MSTRG.144032 | 7.931002956 | 4.89E−05 | 0.003905394 |
| . | gene | MSTRG.138042 | 4.401751835 | 4.91E−05 | 0.003922278 |
| Sgpp1 | gene | MSTRG.31423 | 13.95457018 | 4.96E−05 | 0.003951421 |
| Gm14044 | gene | MSTRG.80962 | 5.341244822 | 4.96E−05 | 0.003951435 |
| Gm14539 | gene | MSTRG.146456 | 6.292054623 | 4.98E−05 | 0.003957416 |
| Gm5621 | gene | MSTRG.144031 | 7.878483947 | 5.09E−05 | 0.004039372 |
| Eif1-ps1 | gene | MSTRG.43050 | 8.301777746 | 5.20E−05 | 0.004122378 |
| Eomes | gene | MSTRG.144892 | 28.4149987 | 5.46E−05 | 0.004303288 |
| Bex1 | gene | MSTRG.150456 | 0.01068375 | 5.48E−05 | 0.00431346 |
| . | gene | MSTRG.2352 | 11.36493736 | 5.50E−05 | 0.004327055 |
| . | gene | MSTRG.149498 | 4.70363957 | 5.51E−05 | 0.004331141 |
| . | gene | MSTRG.137591 | 13.11773898 | 5.59E−05 | 0.004386023 |
| Gm5864 | gene | MSTRG.104601 | 8.159001031 | 5.76E−05 | 0.004510478 |
| Gm12191 | gene | MSTRG.21927 | 25.93616586 | 5.80E−05 | 0.004540724 |
| Trmo | gene | MSTRG.96011 | 8.625493154 | 5.89E−05 | 0.004600911 |
| . | gene | MSTRG.99549 | 3.221296361 | 5.99E−05 | 0.004673851 |
| Rps19-ps6 | gene | MSTRG.33693 | 13.59840153 | 6.03E−05 | 0.004698096 |
| Gm5145 | gene | MSTRG.59678 | 3.99729311 | 6.06E−05 | 0.004712894 |
| Rpl31-ps9 | gene | MSTRG.7152 | 37.44701559 | 6.06E−05 | 0.004712894 |
| . | gene | MSTRG.82089 | 5.746457676 | 6.10E−05 | 0.004730512 |
| . | gene | MSTRG.76047 | 7.292251328 | 6.11E−05 | 0.004730691 |
| Rps23-ps1 | gene | MSTRG.86324 | 12.10150112 | 6.18E−05 | 0.004777704 |
| Gm11966 | gene | MSTRG.19438 | 9.933310747 | 6.19E−05 | 0.004782149 |
| Gm7658 | gene | MSTRG.1126 | 5.538575222 | 6.28E−05 | 0.004837197 |
| Gm12663 | gene | MSTRG.19998 | 3.842229404 | 6.28E−05 | 0.004837197 |
| . | gene | MSTRG.59856 | 4.886475734 | 6.32E−05 | 0.004861596 |
| Gm9009 | gene | MSTRG.148981 | 7.098041364 | 6.47E−05 | 0.004955942 |
| . | gene | MSTRG.71172 | 10.18441765 | 6.51E−05 | 0.0049809 |
| . | gene | MSTRG.132515 | 7.378356525 | 6.56E−05 | 0.00500991 |
| Rpl10l | gene | MSTRG.30905 | 28.48466453 | 6.59E−05 | 0.005031396 |
| Gm20430 | gene | MSTRG.44474 | 2.70159402 | 6.65E−05 | 0.005066925 |
| Rhbdl2 | gene | MSTRG.100202 | 14.90244175 | 6.79E−05 | 0.005164306 |
| . | gene | MSTRG.32068 | 3.505879407 | 6.79E−05 | 0.005164348 |
| . | gene | MSTRG.37113 | 21.05557803 | 6.94E−05 | 0.005272227 |
| Gm10247 | gene | MSTRG.53673 | 13.38333196 | 6.99E−05 | 0.005299785 |
| . | gene | MSTRG.9872 | 3.33846345 | 7.00E−05 | 0.005299785 |
| . | gene | MSTRG.67142 | 22.29599794 | 7.05E−05 | 0.005322596 |
| Mgl2 | gene | MSTRG.23571 | 5.053001214 | 7.14E−05 | 0.005387709 |
| Gm13182 | gene | MSTRG.73567 | 5.513924488 | 7.18E−05 | 0.005406991 |
| . | gene | MSTRG.59700 | 4.994155784 | 7.23E−05 | 0.005430927 |
| . | gene | MSTRG.146869 | 3.680658705 | 7.28E−05 | 0.00546557 |
| . | gene | MSTRG.3103 | 26.17183572 | 7.41E−05 | 0.005555634 |
| Rpl10-ps3 | gene | MSTRG.140078 | 7.383974552 | 7.45E−05 | 0.005577293 |
| Nedd4 | gene | MSTRG.142120 | 0.031298323 | 7.56E−05 | 0.0056499 |
| Cgnl1 | gene | MSTRG.141982 | 5.012447068 | 7.63E−05 | 0.005697942 |
| Rpl10-ps1 | gene | MSTRG.79446 | 8.894313686 | 7.78E−05 | 0.005799006 |
| Ly96 | gene | MSTRG.849 | 6.514336558 | 8.19E−05 | 0.006080596 |
| . | gene | MSTRG.85745 | 3.294506065 | 8.19E−05 | 0.006080596 |
| . | gene | MSTRG.47452 | 4.125854927 | 8.20E−05 | 0.006080596 |
| . | gene | MSTRG.85440 | 4.280963908 | 8.24E−05 | 0.006103957 |
| . | gene | MSTRG.63374 | 9.363545474 | 8.25E−05 | 0.006103957 |
| Gm6542 | gene | MSTRG.66439 | 17.35575176 | 8.37E−05 | 0.006174085 |
| Gm29667 | gene | MSTRG.5782 | 7.862084269 | 8.51E−05 | 0.006270059 |
| Hmgb1-ps5 | gene | MSTRG.89678 | 7.650939312 | 8.57E−05 | 0.006286505 |
| . | gene | MSTRG.129948 | 4.067623673 | 8.57E−05 | 0.006286505 |
| Sarm1 | gene | MSTRG.24372 | 6.303537917 | 8.72E−05 | 0.006393287 |
| . | gene | MSTRG.41240 | 13.23406009 | 8.90E−05 | 0.006489397 |
| . | gene | MSTRG.37942 | 15.06425517 | 9.19E−05 | 0.006694535 |
| . | gene | MSTRG.147786 | 15.80537299 | 9.35E−05 | 0.006800694 |
| Gm11703 | gene | MSTRG.27132 | 7.354914137 | 9.48E−05 | 0.006885495 |
| Gm16439 | gene | MSTRG.43315 | 7.079329296 | 9.65E−05 | 0.007003188 |
| . | gene | MSTRG.8106 | 13.52137613 | 9.72E−05 | 0.007037751 |
| . | gene | MSTRG.28636 | 6.997125818 | 9.93E−05 | 0.007176286 |
| Tcea1-ps1 | gene | MSTRG.52373 | 7.48113275 | 9.96E−05 | 0.007192056 |
| . | gene | MSTRG.45642 | 10.14867254 | 0.000100602 | 0.007254888 |
| Gm4754 | gene | MSTRG.105315 | 3.339765921 | 0.000101786 | 0.007331338 |
| Gm14435 | gene | MSTRG.84333 | 5.452698248 | 0.000102114 | 0.007346021 |
| Gm16216 | gene | MSTRG.71134 | 4.487206786 | 0.000103372 | 0.007427473 |
| Rpsa-ps4 | gene | MSTRG.21085 | 7.811496738 | 0.000104324 | 0.007480179 |
| . | gene | MSTRG.35503 | 0.004404143 | 0.000104359 | 0.007480179 |
| Gm7204 | gene | MSTRG.56377 | 55.83491296 | 0.000105181 | 0.00752083 |
| Muc1 | gene | MSTRG.89225 | 6.285526593 | 0.000105983 | 0.007569034 |
| . | gene | MSTRG.79126 | 172.0469179 | 0.000106827 | 0.007620083 |
| . | gene | MSTRG.29702 | 29.24121357 | 0.000107232 | 0.007639765 |
| Gm6822 | gene | MSTRG.3000 | 2.310149737 | 0.000108158 | 0.007677946 |
| . | gene | MSTRG.100914 | 2.57037436 | 0.000109172 | 0.007740604 |
| . | gene | MSTRG.26217 | 9.517828412 | 0.000109803 | 0.007775968 |
| Rpsa-ps9 | gene | MSTRG.75334 | 6.338602448 | 0.000111107 | 0.007858933 |
| Hist1h1b | gene | MSTRG.35480 | 32.64146149 | 0.00011163 | 0.007886481 |
| Gm12734 | gene | MSTRG.24314 | 3.21181238 | 0.000115434 | 0.008145471 |
| . | gene | MSTRG.28635 | 6.262623488 | 0.000116315 | 0.008197848 |
| Gm44484 | gene | MSTRG.35581 | 0.095963146 | 0.000117181 | 0.008249027 |
| Cyb5r3 | gene | MSTRG.51842 | 0.08492338 | 0.000118204 | 0.008311146 |
| Gm17828 | gene | MSTRG.142059 | 8.745318789 | 0.000119268 | 0.008375969 |
| . | gene | MSTRG.140914 | 3.418960911 | 0.000119889 | 0.008409585 |
| . | gene | MSTRG.79405 | 6.879187392 | 0.000120185 | 0.008420348 |
| Gm6304 | gene | MSTRG.65402 | 8.293733868 | 0.00012185 | 0.008526887 |
| . | gene | MSTRG.32003 | 6.577947065 | 0.000122304 | 0.008542797 |
| Gm12643 | gene | MSTRG.98150 | 3.085983754 | 0.000122366 | 0.008542797 |
| Gm42992 | gene | MSTRG.110839 | 4.433261452 | 0.000126388 | 0.00881314 |
| Pnliprp2 | gene | MSTRG.73351 | 27.63546897 | 0.000127143 | 0.008855296 |
| Alox12 | gene | MSTRG.23604 | 9.164995102 | 0.000127417 | 0.008863979 |
| Dclre1b | gene | MSTRG.90383 | 6.451005658 | 0.000128102 | 0.008901144 |
| Glns-ps1 | gene | MSTRG.20170 | 3.987640883 | 0.000130099 | 0.009029255 |
| Foxh1 | gene | MSTRG.51229 | 12.30261271 | 0.000130965 | 0.009070352 |
| . | gene | MSTRG.80719 | 32.15338043 | 0.000131061 | 0.009070352 |
| Gm9727 | gene | MSTRG.108892 | 6.195991786 | 0.000131199 | 0.009070352 |
| . | gene | MSTRG.17922 | 7.708956614 | 0.000131305 | 0.009070352 |
| . | gene | MSTRG.28568 | 3.160300781 | 0.000132083 | 0.009102799 |
| Gm10443 | gene | MSTRG.116987 | 21.25414162 | 0.000132711 | 0.009130466 |
| Gm8292 | gene | MSTRG.3019 | 4.07292904 | 0.000132794 | 0.009130466 |
| . | gene | MSTRG.111516 | 6.356954097 | 0.000133363 | 0.009158957 |
| Wbp5 | gene | MSTRG.150466 | 0.002396671 | 0.000137787 | 0.009451788 |
| Bsg | gene | MSTRG.15886 | 0.027726177 | 0.000138586 | 0.009495569 |
| . | gene | MSTRG.146783 | 9.445727621 | 0.000139917 | 0.009575616 |
| . | gene | MSTRG.136833 | 3.004413648 | 0.000141332 | 0.00966127 |
| Rps12-ps3 | gene | MSTRG.73399 | 12.54449809 | 0.00014221 | 0.009707203 |
| Hist1h4h | gene | MSTRG.35520 | 10.77936854 | 0.000142332 | 0.009707203 |
| . | gene | MSTRG.52096 | 2.662830984 | 0.000145047 | 0.009869569 |
| Mras | gene | MSTRG.143642 | 9.793297724 | 0.000145735 | 0.009904972 |
| . | gene | MSTRG.120824 | 6.281789313 | 0.000147323 | 0.010001397 |
| Gm16089 | gene | MSTRG.111295 | 8.132902568 | 0.000150239 | 0.010187594 |
| Rps27a-ps2 | gene | MSTRG.142732 | 41.02845215 | 0.000153531 | 0.010398909 |
| . | gene | MSTRG.84453 | 8.921952214 | 0.000155092 | 0.010492596 |
| . | gene | MSTRG.72245 | 3.919418603 | 0.000158718 | 0.010701143 |
| Gm6204 | gene | MSTRG.87125 | 18.53706721 | 0.000160183 | 0.010787623 |
| . | gene | MSTRG.44535 | 25.50223772 | 0.000165391 | 0.011125621 |
| Oaz1-ps | gene | MSTRG.59506 | 4.870830293 | 0.00016624 | 0.011170037 |
| Gm9703 | gene | MSTRG.60231 | 2.971201248 | 0.000166754 | 0.011191874 |
| Rps6-ps1 | gene | MSTRG.51129 | 7.418056451 | 0.00016904 | 0.011319522 |
| Plac9a | gene | MSTRG.42288 | 6.566094883 | 0.000169684 | 0.011349777 |
| . | gene | MSTRG.44065 | 3.70224273 | 0.000174438 | 0.011654576 |
| . | gene | MSTRG.26153 | 10.64341137 | 0.00017586 | 0.01173629 |
| . | gene | MSTRG.85025 | 2.8063171 | 0.000176143 | 0.011741942 |
| . | gene | MSTRG.15052 | 3.007927266 | 0.000181028 | 0.011995482 |
| Gm5560 | gene | MSTRG.108248 | 72.35543311 | 0.000181165 | 0.011995482 |
| Gm5809 | gene | MSTRG.54804 | 18.1593854 | 0.000182023 | 0.012025359 |
| . | gene | MSTRG.64592 | 7.687837263 | 0.000183196 | 0.012089309 |
| Gm8849 | gene | MSTRG.52591 | 3.006462665 | 0.000184677 | 0.012173398 |
| . | gene | MSTRG.18999 | 9.143443515 | 0.00018933 | 0.012410878 |
| Hist1h2ad | gene | MSTRG.35561 | 0.021675501 | 0.000191314 | 0.012527058 |
| . | gene | MSTRG.101017 | 5.878251877 | 0.000191745 | 0.012541387 |
| . | gene | MSTRG.25160 | 15.54323803 | 0.000192081 | 0.012549407 |
| . | gene | MSTRG.39581 | 19.80057792 | 0.000199271 | 0.012978264 |
| Gm12416 | gene | MSTRG.97753 | 5.821704601 | 0.000199304 | 0.012978264 |
| Mrto4-ps2 | gene | MSTRG.28620 | 11.50478685 | 0.000199647 | 0.012986309 |
| Gm4332 | gene | MSTRG.91384 | 55.12864471 | 0.0002022 | 0.013123428 |
| Rpl17-ps9 | gene | MSTRG.123349 | 17.2078515 | 0.000206472 | 0.013371305 |
| Gm8172 | gene | MSTRG.148000 | 5.814534709 | 0.00020867 | 0.013470147 |
| . | gene | MSTRG.30552 | 2.875086578 | 0.000208682 | 0.013470147 |
| . | gene | MSTRG.44413 | 8.160893694 | 0.000209725 | 0.013522658 |
| Gm3362 | gene | MSTRG.49331 | 7.263335724 | 0.000210864 | 0.013581299 |
| . | gene | MSTRG.145704 | 11.39366719 | 0.00021179 | 0.01362605 |
| . | gene | MSTRG.37542 | 6.789654082 | 0.000214098 | 0.013744603 |
| . | gene | MSTRG.33341 | 6.502460088 | 0.000215241 | 0.013802085 |
| Myd88 | gene | MSTRG.144974 | 14.54437682 | 0.00021546 | 0.013802085 |
| . | gene | MSTRG.72303 | 4.233304818 | 0.000216455 | 0.013850744 |
| . | gene | MSTRG.52881 | 11.78759327 | 0.000218604 | 0.013973118 |
| Hmgb1-ps6 | gene | MSTRG.55315 | 3.871366334 | 0.000219313 | 0.013982322 |
| Retsat | gene | MSTRG.116301 | 3.454032093 | 0.000219466 | 0.013982322 |
| Spsb4 | gene | MSTRG.143459 | 20.49971103 | 0.000224523 | 0.014274278 |
| . | gene | MSTRG.42622 | 6.466781611 | 0.000225626 | 0.01432894 |
| Rpl10-ps6 | gene | MSTRG.71904 | 8.303995067 | 0.00022707 | 0.014405159 |
| . | gene | MSTRG.111871 | 16.27406319 | 0.00022948 | 0.014526849 |
| Gm13493 | gene | MSTRG.76734 | 6.990297197 | 0.000230505 | 0.014560535 |
| Rpl34-ps1 | gene | MSTRG.116153 | 11.45775084 | 0.000236157 | 0.014869814 |
| Gm6900 | gene | MSTRG.121360 | 7.281007852 | 0.000239245 | 0.015048219 |
| . | gene | MSTRG.104850 | 4.143886237 | 0.000240282 | 0.015081322 |
| Epha4 | gene | MSTRG.4321 | 4.353776708 | 0.000242548 | 0.015207375 |
| 2810001G20Rik | gene | MSTRG.23128 | 16.13539722 | 0.000242919 | 0.01521453 |
| . | gene | MSTRG.26993 | 0.116678299 | 0.000244529 | 0.015286493 |
| . | gene | MSTRG.72037 | 12.15520247 | 0.000244586 | 0.015286493 |
| . | gene | MSTRG.104948 | 62.30446843 | 0.000251063 | 0.015674722 |
| . | gene | MSTRG.35278 | 4.046191191 | 0.000252808 | 0.015767003 |
| Gm14414 | gene | MSTRG.84389 | 8.834271789 | 0.000254255 | 0.01582384 |
| Gm10913 | gene | MSTRG.55682 | 8.340740907 | 0.000255195 | 0.015865597 |
| Gm15452 | gene | MSTRG.366 | 24.85960007 | 0.000257386 | 0.015984971 |
| Rpl31-ps14 | gene | MSTRG.3745 | 20.67730452 | 0.000258781 | 0.016054779 |
| Chdh | gene | MSTRG.42549 | 5.463483597 | 0.000261081 | 0.01618042 |
| . | gene | MSTRG.137266 | 17.73280544 | 0.000261906 | 0.016214539 |
| Gm27684 | gene | MSTRG.128363 | 3.716927556 | 0.000266792 | 0.016499767 |
| Gm33051 | gene | MSTRG.85822 | 6.390255129 | 0.000275239 | 0.016986606 |
| Rpl23a-ps3 | gene | MSTRG.42825 | 7.400013699 | 0.000276353 | 0.017019772 |
| Gm5422 | gene | MSTRG.13004 | 3.518227906 | 0.000280978 | 0.017231662 |
| Med18 | gene | MSTRG.100912 | 31.61944078 | 0.000281252 | 0.017231662 |
| Gm6266 | gene | MSTRG.120585 | 2.438950134 | 0.000284645 | 0.017385426 |
| . | gene | MSTRG.150017 | 14.20734012 | 0.000285263 | 0.017405193 |
| . | gene | MSTRG.32085 | 10.43106958 | 0.000290301 | 0.017679153 |
| . | gene | MSTRG.43801 | 4.570705762 | 0.00029092 | 0.017695491 |
| Gm7331 | gene | MSTRG.151543 | 4.690821275 | 0.000296053 | 0.017933844 |
| Rpl27-ps1 | gene | MSTRG.9774 | 12.22984329 | 0.000296703 | 0.017954781 |
| . | gene | MSTRG.24936 | 5.032635624 | 0.000302944 | 0.018294955 |
| Parvb | gene | MSTRG.51923 | 7.914790079 | 0.000303817 | 0.018328953 |
| Kdm3b | gene | MSTRG.66251 | 9.025127958 | 0.00030555 | 0.018414664 |
| . | gene | MSTRG.68019 | 2.70274614 | 0.000315712 | 0.018988395 |
| Tdpx-ps1 | gene | MSTRG.5764 | 8.856350827 | 0.000320339 | 0.019207955 |
| Pdia3 | gene | MSTRG.80388 | 0.055625946 | 0.000327273 | 0.019564191 |
| . | gene | MSTRG.53366 | 4.925597171 | 0.000331784 | 0.019813751 |
| Rps3a2 | gene | MSTRG.45997 | 13.87240314 | 0.000334745 | 0.019970404 |
| . | gene | MSTRG.54138 | 6.723384987 | 0.000336691 | 0.020066236 |
| Gm10177 | gene | MSTRG.139258 | 4.954871618 | 0.000337403 | 0.020088416 |
| Gm44122 | gene | MSTRG.118300 | 11.02528821 | 0.000338192 | 0.020115119 |
| Gm15427 | gene | MSTRG.5548 | 2.936597096 | 0.00034734 | 0.020617671 |
| Trmt112-ps2 | gene | MSTRG.102209 | 5.664847403 | 0.000352942 | 0.020908162 |
| . | gene | MSTRG.51915 | 2.657508619 | 0.00035704 | 0.021129771 |
| Rnf38 | gene | MSTRG.95830 | 4.063629696 | 0.000361025 | 0.021301553 |
| . | gene | MSTRG.108890 | 11.39283267 | 0.00037592 | 0.022048277 |
| . | gene | MSTRG.138909 | 3.884970335 | 0.000388868 | 0.022694988 |
| . | gene | MSTRG.68102 | 3.292868868 | 0.000389809 | 0.022712232 |
| . | gene | MSTRG.72656 | 15.63811163 | 0.000392918 | 0.022841073 |
| . | gene | MSTRG.44976 | 5.183280315 | 0.000395108 | 0.022945802 |
| Tigar | gene | MSTRG.119437 | 29.6120272 | 0.000395774 | 0.022961868 |
| Slc28a3 | gene | MSTRG.37753 | 4.675651182 | 0.000397809 | 0.02305729 |
| . | gene | MSTRG.55279 | 2.673190358 | 0.000398655 | 0.023080472 |
| Pex11b | gene | MSTRG.89860 | 9.51928392 | 0.00039899 | 0.023080472 |
| . | gene | MSTRG.43060 | 13.28940145 | 0.000402467 | 0.023236093 |
| Gm13007 | gene | MSTRG.101280 | 3.27236978 | 0.000405876 | 0.023409993 |
| . | gene | MSTRG.36487 | 3.46301848 | 0.000407887 | 0.023503022 |
| . | gene | MSTRG.24456 | 10.92904021 | 0.000412584 | 0.023727382 |
| . | gene | MSTRG.45360 | 3.285683894 | 0.000422973 | 0.024193913 |
| Atp5l-ps2 | gene | MSTRG.25027 | 2.50482589 | 0.000423668 | 0.024199907 |
| . | gene | MSTRG.91281 | 6.746435894 | 0.00042806 | 0.024421243 |
| . | gene | MSTRG.40563 | 4.292886465 | 0.00042837 | 0.024421243 |
| . | gene | MSTRG.23971 | 5.343684251 | 0.000429152 | 0.024442194 |
| Gm12715 | gene | MSTRG.98837 | 6.694139063 | 0.000430275 | 0.024482546 |
| . | gene | MSTRG.115132 | 6.939232094 | 0.000430772 | 0.024487281 |
| Stmn3 | gene | MSTRG.84425 | 100.8114493 | 0.000431456 | 0.024502558 |
| . | gene | MSTRG.7313 | 18.7102902 | 0.000438285 | 0.024866447 |
| Hs3st3b1 | gene | MSTRG.23087 | 3.539858422 | 0.000438974 | 0.02487669 |
| Gm8974 | gene | MSTRG.72631 | 2.265791651 | 0.000439308 | 0.02487669 |
| Rps15a-ps4 | gene | MSTRG.100853 | 2.039545413 | 0.000448085 | 0.025325166 |
| . | gene | MSTRG.20231 | 17.49357502 | 0.000450702 | 0.025448747 |
| . | gene | MSTRG.32080 | 446.0573174 | 0.000452469 | 0.0255241 |
| . | gene | MSTRG.40076 | 8.146274132 | 0.000454937 | 0.025614397 |
| . | gene | MSTRG.52026 | 3.989799549 | 0.000459699 | 0.025857829 |
| Plac9b | gene | MSTRG.42328 | 17.56558344 | 0.000461389 | 0.025928236 |
| . | gene | MSTRG.149830 | 5.43909597 | 0.000464513 | 0.026054216 |
| Gm6450 | gene | MSTRG.108083 | 6.254255929 | 0.000477911 | 0.026704216 |
| . | gene | MSTRG.101881 | 3.292590494 | 0.000480276 | 0.026811029 |
| Tspan1 | gene | MSTRG.99600 | 37.14631261 | 0.000481178 | 0.026836015 |
| . | gene | MSTRG.4393 | 6.010682214 | 0.000489465 | 0.02722108 |
| . | gene | MSTRG.74270 | 3.41931248 | 0.000505059 | 0.028061887 |
| Gm14438 | gene | MSTRG.84089 | 13.21842312 | 0.000508638 | 0.028207665 |
| . | gene | MSTRG.62214 | 9.954404702 | 0.000511563 | 0.028316739 |
| Klhl15 | gene | MSTRG.148941 | 6.593561089 | 0.000523076 | 0.028845831 |
| . | gene | MSTRG.63435 | 4.975438429 | 0.000528511 | 0.02911841 |
| Tubb4b-ps2 | gene | MSTRG.1290 | 6.615415405 | 0.000535691 | 0.029363198 |
| . | gene | MSTRG.88947 | 6.237491191 | 0.00053901 | 0.029476884 |
| . | gene | MSTRG.64909 | 6.34257408 | 0.000540112 | 0.029509799 |
| Rps13-ps5 | gene | MSTRG.22858 | 8.519964125 | 0.000543914 | 0.029662656 |
| Fahd1 | gene | MSTRG.60019 | 7.850803768 | 0.000549065 | 0.029915941 |
| . | gene | MSTRG.14606 | 3.47194251 | 0.000558288 | 0.030385624 |
| Rpl28-ps3 | gene | MSTRG.100385 | 6.450928603 | 0.000559905 | 0.03042237 |
| Rpl7 | gene | MSTRG.806 | 5.558844282 | 0.000562227 | 0.030510871 |
| Gm4575 | gene | MSTRG.117467 | 6.652558493 | 0.000562567 | 0.030510871 |
| Gm5239 | gene | MSTRG.66311 | 3.241632628 | 0.000569246 | 0.030816481 |
| Gm10093 | gene | MSTRG.63639 | 9.380325612 | 0.000574666 | 0.031053005 |
| Rps13-ps4 | gene | MSTRG.137051 | 8.254930861 | 0.000578163 | 0.031213395 |
| Gm44357 | gene | MSTRG.35471 | 0.026659763 | 0.000603975 | 0.032458576 |
| Gm16409 | gene | MSTRG.45348 | 5.126077219 | 0.000612255 | 0.032813967 |
| Gm4613 | gene | MSTRG.122511 | 70.64793338 | 0.000614443 | 0.032901379 |
| Gm7536 | gene | MSTRG.85705 | 7.30983431 | 0.000648967 | 0.034406879 |
| . | gene | MSTRG.144745 | 29.15471839 | 0.000669782 | 0.035320253 |
| Gm20305 | gene | MSTRG.10473 | 3.53644534 | 0.000698749 | 0.036618902 |
| Tbx20 | gene | MSTRG.138601 | 5.985291792 | 0.000861701 | 0.043430737 |
| Ptpn12 | gene | MSTRG.103778 | 10.56566016 | 0.000862361 | 0.043430737 |
| Bex4 | gene | MSTRG.150461 | 0.002701597 | 0.000868148 | 0.043600818 |
| . | gene | MSTRG.115386 | 10.9632341 | 0.000944638 | 0.045407621 |
| Rpl18a-ps1 | gene | MSTRG.30435 | 2.609511325 | 0.001003829 | 0.045407621 |
| . | gene | MSTRG.88994 | 3.788638666 | 0.001006887 | 0.045407621 |
| . | gene | MSTRG.133600 | 0.076636466 | 0.001041234 | 0.045407621 |
| . | gene | MSTRG.116213 | 7.5335146 | 0.001046169 | 0.045407621 |
| . | gene | MSTRG.37968 | 8.745440791 | 0.001211765 | 0.045407621 |
| Rpl39-ps | gene | MSTRG.53188 | 14.14656483 | 0.001424018 | 0.045407621 |
| Gm5148 | gene | MSTRG.86323 | 7.304632269 | 0.001572076 | 0.045407621 |
| . | gene | MSTRG.89541 | 3.1605165 | 0.001585789 | 0.045407621 |
| Lgals4 | gene | MSTRG.122834 | 66.11772052 | 0.001706548 | 0.045407621 |
| . | gene | MSTRG.38643 | 113.8967697 | 0.002080067 | 0.045407621 |
| Map1lc3b | gene | MSTRG.140232 | 9.58295859 | 0.002144922 | 0.045407621 |
| Gm15698 | gene | MSTRG.25263 | 39.17955614 | 0.002222007 | 0.045407621 |

928 DEGs (including novel genes from a de novo transcriptome assembly; 1.8% of all genes) were identified between EPS-blastoids and blastocysts (FIG. 4C and FIG. 4D). In contrast, 4707 DEGs (8.9% of all genes) were found between EPS-blastoids and morulae (FIG. 4E). DEGs between EPS-blastoids and blastocysts were enriched in pathways related to metabolism (FIG. 4F). Thus, at the bulk RNA-Seq level, EPS-blastoids more resembled blastocysts than morulae. To gain insights into the similarities and the differences within each lineage between EPS-blastoids and blastocysts, single-cell RNA-Seq was performed, Profiles of the transcriptomes of over 2700 single cells collected from EPS-blastoids and blastocysts. Integrated analysis using SEURAT revealed that the cells from EPS-blastoids and blastocysts largely overlapped with each other (FIG. 4G). Clustering analysis divided all cells into 7 clusters, with 4 of them shared by both EPS-blastoids and blastocysts (FIG. 4H and FIG. 4I). Expression of a panel of 15 lineage markers was examined and the lineages associated with each cluster was determined (FIG. 4J). Based on the expression pattern of marker genes one cluster was identified as ICM/EPI, one cluster was identified as TE, and two clusters identified as PE. The remaining three clusters, which came mostly from EPS-blastoids, showed mixed expression of both ICM/EPI and TE markers and represent intermediate and/or uncommitted cell types (FIG. 4H). In addition, unsupervised clustering analysis was performed and the clustering of analogous lineages from both samples was confirmed (FIG. 4K). Overall these results confirm that EPS-blastoids contain all three blastocyst cell lineages.

To uncover differences within each lineage between EPS-blastoids and blastocysts, functional annotation of DEGs was performed. 53 DEGs were identified for the ICM/EPI lineage between the two samples (FIG. 4L, Table 8A, and Table 8B), which were enriched with functional terms related to stem cell maintenance, reproduction, and DNA methylation (FIG. 4M, Table 8A, and Table 8B). Two pluripotency transcription factors Sox2 and Klf2 were expressed at lower levels (by ˜28% and ˜43% respectively), and Tet1 and Dnmt3L, two DNA methylation related enzyme genes, showed ˜18% decreased level in EPS-blastoids (FIG. 4L).

TABLE 8A
Summary of Gene Analysis of DEGs for Each Lineage
Between Blastocysts and EPS-blastoids

| Gene_symbol | log2FoldChange | p_val | p_val_adj |
|---|---|---|---|
| Lrp2 | −0.264706756 | 8.34E−21 | 1.67E−17 |
| Dppa5a | 0.796543149 | 2.69E−19 | 5.37E−16 |
| Tdh | 0.71292717 | 1.11E−18 | 2.21E−15 |
| Etv5 | 0.386864001 | 5.40E−17 | 1.08E−13 |
| Klf2 | 0.795546084 | 1.60E−16 | 3.20E−13 |
| H2-K1 | −0.287170757 | 2.22E−16 | 4.44E−13 |
| Mybl2 | 0.433505009 | 6.66E−16 | 1.33E−12 |
| Reep5 | −0.339349854 | 2.21E−14 | 4.42E−11 |
| Mt1 | 0.615371859 | 1.43E−13 | 2.87E−10 |
| Morc1 | 0.349663867 | 3.60E−13 | 7.19E−10 |
| 1700097N02Rik | 0.43737908 | 4.98E−13 | 9.97E−10 |
| Hmga2 | −0.277549493 | 7.18E−13 | 1.44E−09 |
| Sox2 | 0.459263004 | 2.20E−12 | 4.40E−09 |
| Psrc1 | −0.25813391 | 3.06E−12 | 6.11E−09 |
| Rif1 | 0.435314588 | 5.06E−12 | 1.01E−08 |
| Slc7a3 | 0.276624256 | 8.71E−12 | 1.74E−08 |
| Col4a1 | −0.455427579 | 1.62E−11 | 3.24E−08 |
| Mylpf | 1.048601884 | 2.99E−11 | 5.99E−08 |
| Usp9x | 0.393572501 | 3.43E−11 | 6.86E−08 |
| Ifitm2 | 0.396157599 | 5.94E−11 | 1.19E−07 |
| Asns | 0.43681023 | 7.46E−11 | 1.49E−07 |
| Sms | 0.389856118 | 9.45E−11 | 1.89E−07 |
| Dhx16 | 0.303827488 | 1.13E−10 | 2.25E−07 |
| Clic1 | −0.270216407 | 1.36E−10 | 2.73E−07 |
| Ubxn2a | 0.285240507 | 1.63E−10 | 3.26E−07 |
| Gdf3 | 0.260174702 | 2.14E−10 | 4.27E−07 |
| Alpl | 0.283490518 | 2.85E−10 | 5.71E−07 |
| Ooep | 0.390568075 | 1.25E−09 | 2.49E−06 |
| Jarid2 | 0.334317789 | 1.98E−09 | 3.96E−06 |
| Zfp981 | 0.264873906 | 2.61E−09 | 5.23E−06 |
| Rbmxl2 | 0.310391066 | 3.11E−09 | 6.23E−06 |
| Gsta4 | 0.315163134 | 3.26E−09 | 6.51E−06 |
| Mtf2 | 0.345511685 | 5.14E−09 | 1.03E−05 |
| Tdgf1 | 0.601276977 | 1.09E−08 | 2.17E−05 |
| Utf1 | 0.337256109 | 1.33E−08 | 2.66E−05 |
| Mt2 | 0.425082373 | 2.47E−08 | 4.93E−05 |
| Serpinh1 | −0.432149559 | 4.14E−08 | 8.27E−05 |
| Sgk1 | 0.348470719 | 4.21E−08 | 8.42E−05 |
| Slc38a2 | 0.332599444 | 4.27E−08 | 8.54E−05 |
| Cd63 | −0.29806564 | 5.49E−08 | 0.000109723 |
| Zfp42 | 0.501806766 | 6.53E−08 | 0.000130565 |
| Bcat1 | 0.297005535 | 7.15E−08 | 0.000143006 |
| Ifitm1 | 0.7233972 | 8.63E−08 | 0.000172625 |
| Dnmt3l | 0.283814026 | 1.41E−07 | 0.000281049 |
| Upp1 | 0.31017406 | 1.69E−07 | 0.000338764 |
| Gpx4 | 0.641197187 | 1.99E−07 | 0.000397394 |
| Mkrn1 | 0.282072716 | 2.56E−07 | 0.000512395 |
| Atrx | 0.366861672 | 2.64E−07 | 0.000528855 |
| Tdrd12 | 0.26455784 | 2.88E−07 | 0.000576988 |
| Col4a2 | −0.287173951 | 3.08E−07 | 0.000616454 |
| Tmsb4x | 0.432706722 | 3.14E−07 | 0.000628837 |
| Hspb1 | 0.355525756 | 3.43E−07 | 0.000685574 |
| Grsf1 | 0.313003144 | 1.58E−06 | 0.003168643 |
| Igfbp2 | 0.332610563 | 2.24E−06 | 0.004474681 |
| Pdk1 | 0.275323012 | 3.28E−06 | 0.006557031 |
| Hsp90aa1 | 0.251227883 | 3.77E−06 | 0.007533056 |
| Tet1 | 0.282003234 | 6.44E−06 | 0.012875491 |
| Ctsd | −0.298127811 | 1.26E−05 | 0.02514149 |
| Sparc | −0.305241713 | 4.48E−05 | 0.089551981 |
| Tex19.1 | 0.289716084 | 5.72E−05 | 0.114393359 |
| Ifitm3 | 0.38379351 | 0.000129285 | 0.258570159 |
| Xist | −0.466075052 | 0.000129299 | 0.258598933 |
| Gabarapl2 | 0.407097448 | 0.000140647 | 0.281293428 |
| Pdgfa | 0.269207519 | 0.000160336 | 0.320672906 |
| Spp1 | 0.269143873 | 0.001709099 | 1 |
| Klhl13 | 0.31356427 | 0.002463394 | 1 |
| Dppa3 | 0.356770283 | 0.002652934 | 1 |
| Ube2c | 0.258391232 | 0.004240171 | 1 |
| Trh | 0.973137483 | 0.00513798 | 1 |
| Rhox5 | 0.716922201 | 0.008976247 | 1 |
| Mycn | 0.266054104 | 0.011106397 | 1 |
| Gm26917 | 0.431217793 | 0.071202548 | 1 |
| Trim43a | −0.255576142 | 0.107214547 | 1 |
| Pramel4 | −0.272449464 | 0.172915916 | 1 |
| Bhmt | −0.479343816 | 0.399387909 | 1 |
| Nodal | 0.2810304 | 0.577109368 | 1 |
| Calcoco2 | 0.451545661 | 0.668712064 | 1 |
| Olfr1459 | −0.282724165 | 0.912417574 | 1 |
| Phlda2 | 0.335533412 | 0.916163096 | 1 |
| Stmn2 | 0.274634548 | 0.942034758 | 1 |

TABLE 8B
Summary of GO Term Analysis of DEGs for Each Lineage Between Blastocysts and EPS-blastoids

| | | | | | q-value |
|---|---|---|---|---|---|
| | | | q-value | q-value | FDR |
| GO_BP_ID | Name | p-value | Bonferroni | FDR B&H | B&Y |
| GO:0019827 | stem cell population maintenance | 1.21E−05 | 2.26E−02 | 9.58E−03 | 7.77E−02 |
| GO:0098727 | maintenance of cell number | 1.33E−05 | 2.49E−02 | 9.58E−03 | 7.77E−02 |
| GO:0022414 | reproductive process | 2.02E−05 | 3.77E−02 | 9.58E−03 | 7.77E−02 |
| GO:0000003 | reproduction | 2.05E−05 | 3.83E−02 | 9.58E−03 | 7.77E−02 |
| GO:0001701 | in utero embryonic development | 2.86E−05 | 5.34E−02 | 1.06E−02 | 8.60E−02 |
| GO:0044703 | multi-organism reproductive process | 4.06E−05 | 7.58E−02 | 1.06E−02 | 8.60E−02 |
| GO:0006305 | DNA alkylation | 4.55E−05 | 8.48E−02 | 1.06E−02 | 8.60E−02 |
| GO:0006306 | DNA methylation | 4.55E−05 | 8.48E−02 | 1.06E−02 | 8.60E−02 |
| GO:0071514 | genetic imprinting | 8.44E−05 | 1.57E−01 | 1.62E−02 | 1.32E−01 |
| GO:0009790 | embryo development | 9.45E−05 | 1.76E−01 | 1.62E−02 | 1.32E−01 |
| GO:0044728 | DNA methylation or demethylation | 9.57E−05 | 1.79E−01 | 1.62E−02 | 1.32E−01 |
| GO:2000653 | regulation of genetic imprinting | 1.28E−04 | 2.38E−01 | 1.98E−02 | 1.61E−01 |
| GO:0007492 | endoderm development | 1.44E−04 | 2.69E−01 | 1.99E−02 | 1.61E−01 |
| GO:0043414 | macromolecule methylation | 1.49E−04 | 2.78E−01 | 1.99E−02 | 1.61E−01 |
| GO:0031062 | positive regulation of histone methylation | 1.76E−04 | 3.29E−01 | 2.08E−02 | 1.69E−01 |
| GO:2001032 | regulation of double-strand break repair | 1.78E−04 | 3.32E−01 | 2.08E−02 | 1.69E−01 |
| | via nonhomologous end joining | | | | |
| GO:0006304 | DNA modification | 2.25E−04 | 4.19E−01 | 2.46E−02 | 1.99E−01 |
| GO:0038063 | collagen-activated tyrosine kinase | 2.37E−04 | 4.42E−01 | 2.46E−02 | 1.99E−01 |
| | receptor signaling pathway | | | | |
| GO:0007369 | gastrulation | 2.53E−04 | 4.73E−01 | 2.49E−02 | 2.02E−01 |
| GO:0035987 | endodermal cell differentiation | 3.17E−04 | 5.91E−01 | 2.96E−02 | 2.40E−01 |
| GO:0038065 | collagen-activated signaling pathway | 3.80E−04 | 7.08E−01 | 3.34E−02 | 2.71E−01 |
| GO:2001251 | negative regulation of chromosome | 3.94E−04 | 7.36E−01 | 3.34E−02 | 2.71E−01 |
| | organization | | | | |
| GO:0032963 | collagen metabolic process | 4.62E−04 | 8.62E−01 | 3.61E−02 | 2.93E−01 |
| GO:0007049 | cell cycle | 4.71E−04 | 8.80E−01 | 3.61E−02 | 2.93E−01 |
| GO:0045071 | negative regulation of viral genome | 4.86E−04 | 9.07E−01 | 3.61E−02 | 2.93E−01 |
| | replication | | | | |
| GO:0001649 | osteoblast differentiation | 5.03E−04 | 9.39E−01 | 3.61E−02 | 2.93E−01 |
| GO:0032259 | methylation | 5.55E−04 | 1.00E+00 | 3.84E−02 | 3.11E−01 |
| GO:0009066 | aspartate family amino acid metabolic | 6.05E−04 | 1.00E+00 | 3.89E−02 | 3.16E−01 |
| | process | | | | |
| GO:0001706 | endoderm formation | 6.05E−04 | 1.00E+00 | 3.89E−02 | 3.16E−01 |
| GO:0001763 | morphogenesis of a branching structure | 6.63E−04 | 1.00E+00 | 4.12E−02 | 3.34E−01 |
| GO:0019953 | sexual reproduction | 8.40E−04 | 1.00E+00 | 4.98E−02 | 4.04E−01 |
| GO:0031060 | regulation of histone methylation | 8.54E−04 | 1.00E+00 | 4.98E−02 | 4.04E−01 |
| GO:0043009 | chordate embryonic development | 8.99E−04 | 1.00E+00 | 4.98E−02 | 4.04E−01 |
| GO:0007276 | gamete generation | 9.07E−04 | 1.00E+00 | 4.98E−02 | 4.04E−01 |
| GO:0005587 | collagen type IV trimer | 1.22E−04 | 2.84E−02 | 1.32E−02 | 7.95E−02 |
| GO:0098645 | collagen network | 1.70E−04 | 3.96E−02 | 1.32E−02 | 7.95E−02 |
| GO:0098642 | network-forming collagen trimer | 1.70E−04 | 3.96E−02 | 1.32E−02 | 7.95E−02 |
| GO:0098651 | basement membrane collagen trimer | 2.26E−04 | 5.27E−02 | 1.32E−02 | 7.95E−02 |
| GO:0005903 | brush border | 5.23E−04 | 1.22E−01 | 2.44E−02 | 1.47E−01 |
| GO:0035098 | ESC/E(Z) complex | 9.56E−04 | 2.23E−01 | 3.71E−02 | 2.24E−01 |
| GO:0045177 | apical part of cell | 1.42E−03 | 3.30E−01 | 4.18E−02 | 2.52E−01 |
| GO:0098862 | cluster of actin-based cell projections | 1.63E−03 | 3.79E−01 | 4.18E−02 | 2.52E−01 |
| GO:0098644 | complex of collagen trimers | 1.99E−03 | 4.64E−01 | 4.18E−02 | 2.52E−01 |
| GO:0000792 | heterochromatin | 2.03E−03 | 4.73E−01 | 4.18E−02 | 2.52E−01 |
| GO:0005615 | extracellular space | 2.51E−03 | 5.85E−01 | 4.18E−02 | 2.52E−01 |
| GO:0098839 | postsynaptic density membrane | 2.89E−03 | 6.74E−01 | 4.18E−02 | 2.52E−01 |
| GO:1990707 | nuclear subtelomeric heterochromatin | 2.89E−03 | 6.74E−01 | 4.18E−02 | 2.52E−01 |
| GO:1990421 | subtelomeric heterochromatin | 2.89E−03 | 6.74E−01 | 4.18E−02 | 2.52E−01 |
| GO:0099634 | postsynaptic specialization membrane | 2.89E−03 | 6.74E−01 | 4.18E−02 | 2.52E−01 |
| GO:0098857 | membrane microdomain | 3.05E−03 | 7.11E−01 | 4.18E−02 | 2.52E−01 |

For the PE lineage, 67 DEGs were identified between EPS-blastoids and blastocysts, which were mostly enriched in terms related to vesicle transport and endocytosis (FIG. 4N, FIG. 4O, Table 9A, and Table 9B).

TABLE 9A

| Gene_symbol | log2FoldChange | p_val | p_val_adj |
|---|---|---|---|
| Gng2 | −0.253120263 | 3.97E−34 | 7.95E−31 |
| Pou5f1 | −0.442208454 | 2.21E−17 | 4.42E−14 |
| Ctsh | 0.927355914 | 7.90E−16 | 1.58E−12 |
| Lgmn | 0.741507148 | 1.38E−13 | 2.76E−10 |
| Spink1 | 1.247513242 | 6.51E−13 | 1.30E−09 |
| Cldn6 | 0.51806942 | 5.06E−12 | 1.01E−08 |
| Morf4l2 | 0.740828703 | 1.32E−11 | 2.64E−08 |
| Amn | 0.831947727 | 1.55E−11 | 3.10E−08 |
| Glipr2 | −0.310090445 | 1.76E−11 | 3.51E−08 |
| Cited1 | 0.803811126 | 3.07E−11 | 6.13E−08 |
| Gpc3 | 0.70191946 | 5.09E−11 | 1.02E−07 |
| Aldh7a1 | 0.538249896 | 7.46E−10 | 1.49E−06 |
| Dnmt3l | −0.259986382 | 9.85E−10 | 1.97E−06 |
| Slc16a1 | 0.512569884 | 1.57E−09 | 3.14E−06 |
| Ctsd | 0.501631779 | 3.15E−09 | 6.31E−06 |
| Neu1 | 0.371669416 | 3.27E−09 | 6.55E−06 |
| Apoe | 0.617838596 | 4.45E−09 | 8.89E−06 |
| Ctsl | 0.577310608 | 5.56E−09 | 1.11E−05 |
| Grn | 0.423541489 | 6.11E−09 | 1.22E−05 |
| Slc9a3r1 | 0.483068858 | 3.51E−08 | 7.02E−05 |
| Tmprss2 | 0.324644259 | 4.67E−08 | 9.34E−05 |
| Krt18 | 0.597508533 | 5.76E−08 | 0.000115171 |
| Ctsb | 0.579212246 | 6.12E−08 | 0.000122405 |
| Mfsd1 | 0.371932469 | 6.43E−08 | 0.00012853 |
| Vamp8 | 0.421599087 | 8.99E−08 | 0.00017981 |
| Ctsz | 0.494701392 | 1.70E−07 | 0.000339611 |
| Epb41l3 | 0.346761061 | 1.78E−07 | 0.000356138 |
| Cd63 | 0.48617749 | 1.89E−07 | 0.000377459 |
| S100a1 | 0.776954635 | 2.53E−07 | 0.00050665 |
| Clic6 | 0.42123544 | 2.69E−07 | 0.000538012 |
| Cd81 | 0.502094167 | 4.05E−07 | 0.000810575 |
| Slc2a3 | 0.430114774 | 4.11E−07 | 0.000822488 |
| Emb | 0.452796146 | 5.10E−07 | 0.001019982 |
| Ctsa | 0.324983656 | 5.36E−07 | 0.00107111 |
| Rab11a | 0.386443098 | 6.17E−07 | 0.00123483 |
| Npl | 0.616435409 | 6.34E−07 | 0.001267763 |
| Dab2 | 0.422636416 | 8.16E−07 | 0.00163269 |
| Lamp1 | 0.380407457 | 9.02E−07 | 0.001804428 |
| Ttr | 1.399555399 | 9.86E−07 | 0.001971743 |
| Atp6v0b | 0.322265381 | 1.04E−06 | 0.002077182 |
| Rhou | 0.332461686 | 1.29E−06 | 0.002579255 |
| Cyba | 0.985287408 | 2.83E−06 | 0.005669492 |
| Fbln1 | 0.403756459 | 2.89E−06 | 0.005787216 |
| 2210011C24Rik | 0.340274902 | 2.89E−06 | 0.00578951 |
| Bag2 | 0.26539222 | 2.90E−06 | 0.005798587 |
| Apoa1 | 0.328753827 | 3.08E−06 | 0.006166527 |
| Morc4 | 0.294481261 | 3.24E−06 | 0.006482943 |
| Cotl1 | 0.389352729 | 3.62E−06 | 0.007246171 |
| Stx3 | 0.285336128 | 4.00E−06 | 0.007992475 |
| Gng12 | 0.30198474 | 5.01E−06 | 0.010019076 |
| Abracl | 0.290321835 | 5.66E−06 | 0.011321702 |
| Tnfrsf12a | 0.438920197 | 7.20E−06 | 0.014398456 |
| Krt8 | 0.44266351 | 7.23E−06 | 0.014452389 |
| Gpx3 | 0.534203154 | 7.30E−06 | 0.014599014 |
| Agpat3 | 0.272594807 | 7.78E−06 | 0.015565418 |
| Mkrn1 | −0.347818786 | 7.90E−06 | 0.015806329 |
| Ctsc | 0.495079318 | 9.34E−06 | 0.018679341 |
| Ubqln2 | 0.305792276 | 1.15E−05 | 0.022952221 |
| Aprt | 0.351904883 | 1.33E−05 | 0.026596243 |
| Klhl2 | 0.320526805 | 1.42E−05 | 0.028406712 |
| Car4 | 0.673822863 | 1.46E−05 | 0.029169138 |
| Folr1 | 0.405891835 | 1.53E−05 | 0.030602133 |
| Tcn2 | 0.267506613 | 1.71E−05 | 0.034164424 |
| Bex4 | 0.436734313 | 1.80E−05 | 0.036003103 |
| Map1lc3b | 0.391947905 | 1.86E−05 | 0.037219613 |
| Gaa | 0.290141254 | 2.00E−05 | 0.040060643 |
| Rab4a | 0.327341871 | 2.32E−05 | 0.046311767 |
| Fth1 | 0.391880236 | 2.82E−05 | 0.056336469 |
| Atp6v0d1 | 0.329331156 | 2.94E−05 | 0.05871379 |
| Cldn7 | 0.615942809 | 3.02E−05 | 0.060312776 |
| Cltc | 0.251453819 | 3.31E−05 | 0.066298499 |
| Degs1 | 0.257510914 | 3.69E−05 | 0.073862936 |
| Clcn5 | 0.269774527 | 3.88E−05 | 0.07768341 |
| Bex2 | 0.749025685 | 4.04E−05 | 0.08070004 |
| Ckb | −0.259765488 | 4.70E−05 | 0.094053067 |
| Itm2b | 0.304765885 | 5.25E−05 | 0.105044626 |
| Gm26870 | −0.291965703 | 5.37E−05 | 0.107374389 |
| Cst3 | 0.373003009 | 5.96E−05 | 0.119210239 |
| 2-Mar | 0.312967699 | 6.41E−05 | 0.128180632 |
| Rhox5 | 0.418060247 | 6.45E−05 | 0.129052813 |
| Xist | −1.015399835 | 7.23E−05 | 0.144593238 |
| 1810058I24Rik | 0.331651914 | 7.39E−05 | 0.147834104 |
| Cubn | 0.413701899 | 8.78E−05 | 0.175689563 |
| Myo6 | 0.262747249 | 0.000104999 | 0.209997569 |
| Bnip3 | 0.353978856 | 0.000110253 | 0.220505602 |
| Gm26917 | −0.421639152 | 0.000114318 | 0.228635654 |
| Slc39a4 | 0.251064076 | 0.000122892 | 0.245784105 |
| Sparc | 0.41997074 | 0.000125629 | 0.251258989 |
| Bex1 | 0.528869498 | 0.000131615 | 0.263229045 |
| Cystm1 | 0.356153262 | 0.000137355 | 0.274709759 |
| Tpi1 | 0.291239717 | 0.000151674 | 0.303347264 |
| Cstb | 0.330124344 | 0.000171042 | 0.342083969 |
| Retreg1 | 0.302636618 | 0.000176017 | 0.3520344 |
| Cndp2 | 0.272601912 | 0.000194617 | 0.389233894 |
| Pcbd1 | 0.278525086 | 0.000257721 | 0.515441951 |
| Kif21a | 0.302553255 | 0.000263483 | 0.526965069 |
| S100g | 1.148031084 | 0.000266336 | 0.532672535 |
| Apom | 0.883507078 | 0.000271449 | 0.542898688 |
| Nxf7 | 0.279282533 | 0.000273324 | 0.546648377 |
| Snx3 | 0.31495066 | 0.000282216 | 0.564432388 |
| Amot | 0.393777074 | 0.000419151 | 0.838302741 |
| Tdh | 0.298128542 | 0.000430775 | 0.861550978 |
| Dpp4 | 0.262180914 | 0.000494137 | 0.988273297 |
| P4ha1 | 0.273290951 | 0.000539396 | 1 |
| Rhoc | 0.32087416 | 0.000600731 | 1 |
| Prss12 | 0.312627818 | 0.000604128 | 1 |
| Clic1 | 0.261693299 | 0.000689872 | 1 |
| Slc2a1 | 0.308069337 | 0.000721383 | 1 |
| Col4a1 | 0.452986762 | 0.000809011 | 1 |
| Dbi | 0.272846489 | 0.000816918 | 1 |
| Tmem37 | 0.418106441 | 0.000828786 | 1 |
| Jpt1 | 0.256370929 | 0.000833947 | 1 |
| Atp6v0e | 0.282032735 | 0.000857064 | 1 |
| Kdelr3 | 0.260938349 | 0.000980061 | 1 |
| Abcg2 | 0.320106428 | 0.001399126 | 1 |
| Col4a2 | 0.443837531 | 0.001407345 | 1 |
| H2-D1 | 0.298854814 | 0.001476607 | 1 |
| Glul | 0.265984204 | 0.001750367 | 1 |
| Tmsb10 | −0.265006952 | 0.002045984 | 1 |
| Jade1 | 0.325682312 | 0.00221684 | 1 |
| Peg3 | 0.330020158 | 0.003433876 | 1 |
| Lxn | 0.272907084 | 0.003449343 | 1 |
| Asns | −0.301842863 | 0.00347739 | 1 |
| Slc7a7 | 0.381317788 | 0.003701381 | 1 |
| Podxl | 0.275776482 | 0.006222438 | 1 |
| Nid2 | 0.28449161 | 0.006393527 | 1 |
| Rdx | 0.285571382 | 0.009829248 | 1 |
| Pkdcc | 0.327688872 | 0.011101177 | 1 |
| Meg3 | 0.438964195 | 0.013018184 | 1 |
| Trap1a | 0.291193553 | 0.014093453 | 1 |
| Id2 | 0.251617736 | 0.02144406 | 1 |
| Tfpi | 0.261448414 | 0.02658275 | 1 |
| H19 | 0.370943384 | 0.039498989 | 1 |
| Polg | 0.250212268 | 0.05050624 | 1 |
| Rbp4 | 0.848762445 | 0.050572955 | 1 |
| H2-K1 | 0.30106861 | 0.05431406 | 1 |
| Id1 | 0.273256866 | 0.05441887 | 1 |
| Apob | 1.368224302 | 0.060494006 | 1 |
| Mt1 | 0.338350668 | 0.073631691 | 1 |
| Lgals2 | 0.416067224 | 0.084872497 | 1 |
| Nrk | 0.554052601 | 0.099212299 | 1 |
| Bst2 | 0.326230704 | 0.107049918 | 1 |
| Gsn | 0.266696355 | 0.160145861 | 1 |
| Tceal8 | 0.302903834 | 0.172674271 | 1 |
| Ass1 | 0.32203303 | 0.196641036 | 1 |
| Flt1 | 0.321275371 | 0.202645992 | 1 |
| Clu | 0.437318271 | 0.219686766 | 1 |
| Lrp2 | 0.455220586 | 0.223990401 | 1 |
| Hspb1 | 0.286221901 | 0.240157504 | 1 |
| Aqp8 | 0.461751642 | 0.401591418 | 1 |
| Selenop | 0.37912372 | 0.773637006 | 1 |

TABLE 9B

| | | | | q-value | q-value |
|---|---|---|---|---|---|
| | | | q-value | FDR | FDR |
| GO_BP_ID | Name | p-value | Bonferroni | B&H | B&Y |
| GO:0016192 | vesicle-mediated transport | 5.01E−09 | 1.22E−05 | 1.22E−05 | 1.02E−04 |
| GO:0006897 | endocytosis | 6.64E−08 | 1.61E−04 | 8.07E−05 | 6.76E−04 |
| GO:0051180 | vitamin transport | 1.23E−07 | 2.99E−04 | 8.19E−05 | 6.85E−04 |
| GO:0006898 | receptor-mediated endocytosis | 1.35E−07 | 3.27E−04 | 8.19E−05 | 6.85E−04 |
| GO:0072659 | protein localization to plasma membrane | 1.26E−06 | 3.06E−03 | 5.37E−04 | 4.49E−03 |
| GO:1990778 | protein localization to cell periphery | 1.35E−06 | 3.27E−03 | 5.37E−04 | 4.49E−03 |
| GO:0060627 | regulation of vesicle-mediated transport | 1.55E−06 | 3.76E−03 | 5.37E−04 | 4.49E−03 |
| GO:0030163 | protein catabolic process | 6.83E−06 | 1.66E−02 | 2.07E−03 | 1.74E−02 |
| GO:0007009 | plasma membrane organization | 8.45E−06 | 2.05E−02 | 2.13E−03 | 1.78E−02 |
| GO:0022604 | regulation of cell morphogenesis | 8.76E−06 | 2.13E−02 | 2.13E−03 | 1.78E−02 |
| GO:0051049 | regulation of transport | 9.71E−06 | 2.36E−02 | 2.15E−03 | 1.80E−02 |
| GO:0097067 | cellular response to thyroid hormone | 1.19E−05 | 2.88E−02 | 2.19E−03 | 1.83E−02 |
| | stimulus | | | | |
| GO:0044248 | cellular catabolic process | 1.21E−05 | 2.93E−02 | 2.19E−03 | 1.83E−02 |
| GO:0072657 | protein localization to membrane | 1.26E−05 | 3.06E−02 | 2.19E−03 | 1.83E−02 |
| GO:0030100 | regulation of endocytosis | 1.50E−05 | 3.64E−02 | 2.43E−03 | 2.03E−02 |
| GO:0046903 | secretion | 1.96E−05 | 4.77E−02 | 2.98E−03 | 2.50E−02 |
| GO:0040011 | locomotion | 2.25E−05 | 5.47E−02 | 3.09E−03 | 2.59E−02 |
| GO:0051656 | establishment of organelle localization | 2.29E−05 | 5.57E−02 | 3.09E−03 | 2.59E−02 |
| GO:0043112 | receptor metabolic process | 2.59E−05 | 6.28E−02 | 3.31E−03 | 2.77E−02 |
| GO:0070254 | mucus secretion | 2.79E−05 | 6.78E−02 | 3.33E−03 | 2.79E−02 |
| GO:0044257 | cellular protein catabolic process | 2.95E−05 | 7.17E−02 | 3.33E−03 | 2.79E−02 |
| GO:0071495 | cellular response to endogenous stimulus | 3.13E−05 | 7.61E−02 | 3.33E−03 | 2.79E−02 |
| GO:0051701 | interaction with host | 3.27E−05 | 7.94E−02 | 3.33E−03 | 2.79E−02 |
| GO:0022603 | regulation of anatomical structure | 3.29E−05 | 8.00E−02 | 3.33E−03 | 2.79E−02 |
| | morphogenesis | | | | |
| GO:0010770 | positive regulation of cell morphogenesis | 4.62E−05 | 1.12E−01 | 4.49E−03 | 3.76E−02 |
| | involved in differentiation | | | | |
| GO:0010769 | regulation of cell morphogenesis involved | 5.10E−05 | 1.24E−01 | 4.77E−03 | 4.00E−02 |
| | in differentiation | | | | |
| GO:0046718 | viral entry into host cell | 6.67E−05 | 1.62E−01 | 5.02E−03 | 4.21E−02 |
| GO:0097066 | response to thyroid hormone | 7.16E−05 | 1.74E−01 | 5.02E−03 | 4.21E−02 |
| GO:0032489 | regulation of Cdc42 protein signal | 7.39E−05 | 1.80E−01 | 5.02E−03 | 4.21E−02 |
| | transduction | | | | |
| GO:0051640 | organelle localization | 7.46E−05 | 1.81E−01 | 5.02E−03 | 4.21E−02 |
| GO:0051806 | entry into cell of other organism | 7.50E−05 | 1.82E−01 | 5.02E−03 | 4.21E−02 |
| | involved in symbiotic interaction | | | | |
| GO:0052126 | movement in host environment | 7.50E−05 | 1.82E−01 | 5.02E−03 | 4.21E−02 |
| GO:0030260 | entry into host cell | 7.50E−05 | 1.82E−01 | 5.02E−03 | 4.21E−02 |
| GO:0044409 | entry into host | 7.50E−05 | 1.82E−01 | 5.02E−03 | 4.21E−02 |
| GO:0051828 | entry into other organism involved | 7.50E−05 | 1.82E−01 | 5.02E−03 | 4.21E−02 |
| | in symbiotic interaction | | | | |
| GO:0052192 | movement in environment of other organism | 7.50E−05 | 1.82E−01 | 5.02E−03 | 4.21E−02 |
| | involved in symbiotic interaction | | | | |
| GO:0009057 | macromolecule catabolic process | 7.93E−05 | 1.93E−01 | 5.02E−03 | 4.21E−02 |
| GO:1901575 | organic substance catabolic process | 8.05E−05 | 1.96E−01 | 5.02E−03 | 4.21E−02 |
| GO:0006820 | anion transport | 8.06E−05 | 1.96E−01 | 5.02E−03 | 4.21E−02 |
| GO:0015031 | protein transport | 1.04E−04 | 2.53E−01 | 6.29E−03 | 5.27E−02 |
| GO:0051046 | regulation of secretion | 1.06E−04 | 2.58E−01 | 6.29E−03 | 5.27E−02 |
| GO:0006766 | vitamin metabolic process | 1.13E−04 | 2.73E−01 | 6.51E−03 | 5.45E−02 |
| GO:0007033 | vacuole organization | 1.16E−04 | 2.81E−01 | 6.53E−03 | 5.47E−02 |
| GO:0045055 | regulated exocytosis | 1.23E−04 | 2.98E−01 | 6.63E−03 | 5.56E−02 |
| GO:0006931 | substrate-dependent cell migration, cell | 1.23E−04 | 2.99E−01 | 6.63E−03 | 5.56E−02 |
| | attachment to substrate | | | | |
| GO:0006811 | ion transport | 1.36E−04 | 3.30E−01 | 7.18E−03 | 6.01E−02 |
| GO:0006887 | exocytosis | 1.53E−04 | 3.71E−01 | 7.90E−03 | 6.62E−02 |
| GO:0006901 | vesicle coating | 1.69E−04 | 4.10E−01 | 8.27E−03 | 6.93E−02 |
| GO:0019882 | antigen processing and presentation | 1.71E−04 | 4.15E−01 | 8.27E−03 | 6.93E−02 |
| GO:0023056 | positive regulation of signaling | 1.74E−04 | 4.23E−01 | 8.27E−03 | 6.93E−02 |

For the TE lineage, only two DEGs (Gjb2 and Arhgel6) were identified to be significantly different between the two samples (FIG. 4P and Table 10).

TABLE 10
Gene Expression TE Lineage

| Gene_symbol | log2FoldChange | p_val | p_val_adj |
|---|---|---|---|
| Gjb2 | 0.337337885 | 2.95E−14 | 5.90E−11 |
| Arhgef6 | 0.263634473 | 1.97E−08 | 3.94E−05 |
| Spp1 | 0.366202218 | 2.58E−05 | 0.051525748 |
| Dab2 | −0.52691081 | 4.59E−05 | 0.091809336 |
| Snx2 | −0.332889145 | 0.00027157 | 0.543139689 |
| Perp | −0.280401191 | 0.000639094 | 1 |
| Apela | 0.274414303 | 0.001458758 | 1 |
| Lmna | −0.294968971 | 0.00162325 | 1 |
| Cstb | −0.343481888 | 0.002450833 | 1 |
| Anxa2 | −0.360048259 | 0.002479273 | 1 |
| Ptges | −0.3594359 | 0.004531606 | 1 |
| 1700013H16Rik | 0.872254034 | 0.010140151 | 1 |
| Clic4 | −0.349487689 | 0.010448112 | 1 |
| Slc2a3 | −0.381854965 | 0.012304132 | 1 |
| Apoe | −0.256310448 | 0.015921222 | 1 |
| Cd63 | −0.423442602 | 0.01592302 | 1 |
| Sord | −0.274436526 | 0.018484153 | 1 |
| Lrpap1 | −0.26598074 | 0.022371414 | 1 |
| Ldha | 0.409787783 | 0.022449532 | 1 |
| Msn | −0.252707359 | 0.023084166 | 1 |
| Mgst3 | −0.26795715 | 0.02704497 | 1 |
| Dkk1 | 0.323232 | 0.027719109 | 1 |
| Cdkn1a | −0.259001334 | 0.028281006 | 1 |
| Igfbp2 | 0.391085331 | 0.028666025 | 1 |
| Crip2 | −0.3055985 | 0.029860743 | 1 |
| Tpm1 | −0.283819559 | 0.033478841 | 1 |
| Epop | 0.396679276 | 0.037180493 | 1 |
| Lrp2 | −0.534901872 | 0.044828403 | 1 |
| Tmem37 | 0.374877558 | 0.045124276 | 1 |
| Flna | −0.256874612 | 0.049113955 | 1 |
| Abracl | −0.325655784 | 0.064369123 | 1 |
| Ctsl | −0.267848375 | 0.064472681 | 1 |
| Peg10 | 0.376054798 | 0.070882632 | 1 |
| Atp1b1 | −0.298526728 | 0.072267246 | 1 |
| Malat1 | −0.952735671 | 0.079011484 | 1 |
| Cnn2 | −0.259283054 | 0.080353628 | 1 |
| Oat | −0.292488404 | 0.095879419 | 1 |
| Ccnd3 | 0.517107185 | 0.10339408 | 1 |
| Calm1 | −0.287114596 | 0.112788141 | 1 |
| Dstn | −0.29465755 | 0.119390051 | 1 |
| Trap1a | 0.420361357 | 0.133350017 | 1 |
| Ahnak | −0.430058076 | 0.148327705 | 1 |
| Pim3 | 0.48241429 | 0.15461847 | 1 |
| Emilin1 | −0.270746586 | 0.192245069 | 1 |
| Tfrc | 0.303678364 | 0.239482805 | 1 |
| Ascl2 | 0.32190661 | 0.246136678 | 1 |
| Wfdc2 | 0.530051238 | 0.260477468 | 1 |
| Slc38a4 | 0.621714844 | 0.26119585 | 1 |
| Gm4926 | −0.322136744 | 0.26779565 | 1 |
| Gjb3 | 0.466918943 | 0.282584794 | 1 |
| Slc29a1 | 0.250800666 | 0.31430777 | 1 |
| Phlda2 | 1.019277417 | 0.319414802 | 1 |
| Klhl13 | 0.488103597 | 0.32608919 | 1 |
| Gata3 | −0.25197904 | 0.338483794 | 1 |
| H19 | 0.882437363 | 0.343186011 | 1 |
| Xist | −0.758571059 | 0.346872781 | 1 |
| Dusp9 | 0.275468483 | 0.351562903 | 1 |
| Coil | −0.504295062 | 0.363080903 | 1 |
| Sin3b | 0.462205235 | 0.409410602 | 1 |
| Cebpb | 0.453924171 | 0.434035228 | 1 |
| Hmgn5 | 0.320043023 | 0.434060363 | 1 |
| Rhox5 | 1.107444289 | 0.440896364 | 1 |
| Maged1 | 0.264196284 | 0.463832744 | 1 |
| Gata2 | 0.436313062 | 0.562787743 | 1 |
| Utf1 | 0.337186802 | 0.577341471 | 1 |
| Rhox9 | 1.193384447 | 0.581981203 | 1 |
| Wtap | −0.251090182 | 0.598557507 | 1 |
| Myh10 | −0.274651847 | 0.623059394 | 1 |
| Pdcd4 | −0.372102293 | 0.680406499 | 1 |
| Bex3 | −0.304663993 | 0.711547095 | 1 |
| Hspb1 | 0.261792297 | 0.727505499 | 1 |
| Cdkn1c | 1.433591002 | 0.792012314 | 1 |
| Rhox6 | 1.251235238 | 0.835387097 | 1 |
| Slc2a1 | 0.476438758 | 0.8421147 | 1 |
| Peg3 | 0.308978465 | 0.864374164 | 1 |
| Uchl1 | 0.26775256 | 0.874361505 | 1 |
| Id3 | 0.341053113 | 0.875681215 | 1 |
| Nup62cl | 0.2841828 | 0.926061001 | 1 |
| Plac1 | 0.303029585 | 0.930567243 | 1 |
| Nrk | 0.505471603 | 0.95453255 | 1 |

### Example 5: In Vitro Developmental Uses of EPS-Blastoids

ESCs, TSCs, and XEN cells, which are considered the in vitro counterparts of EPI, TE, and PE lineages, respectively, could all be derived directly from blastocysts. That EPS-blastoids could also give rise to these three stem cell lines was determined. Using the ground state culture condition (Ying et al., 2008), ESC lines were successfully established from four out of five EPS-blastoids. These ESCs formed colonies with similar morphologies to those generated from natural blastocysts and expressed the pluripotency factors OCT4, NANOG, and SOX2, but not the trophoblast marker CDX2 (FIG. 5A and FIG. 5B). Injection of EPS-blastoid-derived ESCs into natural blastocysts resulted in adult chimeras (FIG. 5C). Eight TSC lines were derived from 17 EPS-blastoids. These TSCs morphologically resembled those generated from natural blastocysts and expressed the TE transcription factors CDX2 and EOMES, but not OCT4 or NANOG (FIG. 5D and FIG. 5E). Injection of EPS-blastoid-derived TSCs into blastocyst followed by embryo transfer generated chimeric placental tissues that contained CK8+ EPS-blastoid TSC-derivatives (FIG. 5F). Finally, two XEN cell lines were also established from six EPS-blastoids (FIG. 5G). These EPS-blastoid-derived XEN cells expressed the PE transcription factors GATA4 and GATA6 (FIG. 5H and FIG. 5I) and could chimerize host yolk sac in utero (FIG. 5J).

A recently developed in vitro culture (IVC) system enabled the development of mouse and human blastocysts beyond the implantation stages in vitro, and has also been successfully used to assemble postimplantation embryo-like structures (Bedzhov and Zernicka-Goetz, 2014; Bedzhov et al., 2014a; Harrison et al., 2017; Sozen et al., 2018). It was tested whether the IVC condition could support the development of EPS-blastoids beyond the implantation stage. In agreement with the reports from the Zernicka-Goetz group (Bedzhov and Zernicka-Goetz, 2014), in vitro culture of blastocysts generated an egg cylinder structure with extraembryonic ectoderm (ExE, marked by TFAP2C) and EPI (marked by SOX2) as two hemispheres enclosed by the visceral endoderm (VE, marked by GATA6) (FIG. 5K). Similar structures formed when EPS-blastoids were cultured in the IVC media (FIG. 5L). Approximately 49% of cultured blastocysts and 32% cultured EPS-blastoids generated an organized egg cylinder structure (FIG. 5M).

The cellular and molecular events that contributed to the generation of the egg cylinder structures from EPS-blastoids were examined. During the peri-implantation stage, EPI cells become polarized and form a rosette-like structure with apical domains clustered in the center. In cultured EPS-blastoids, F-actin was enriched in the center of the EPI-like compartment and the cells adopted a rosette-like configuration, reminiscent of a pen-implantation embryo at ˜E4.5-E4.75 stage (FIG. 5N). The polarity protein aPKC lined the cells that formed the cavity within the EPI-like compartment (FIG. 5O), characteristic of an E5.25-E5.5 embryo. Lumenogenesis in the postimplantation embryos depends on membrane repulsion mediated by podocalyxin (PCX) (Bedzhov and Zernicka-Goetz, 2014). As seen in natural embryos, PCX levels were highest on the apical side of the cells enclosing the lumen in both the EPI- and ExE-like compartments of EPS-blastoid-derived structures (FIG. 5P and FIG. 5Q). Lumenogenesis also depends on signaling from the VE-derived basement membrane, and it was determined that both the EPI- and ExE-like compartments were surrounded by a laminin-containing basement membrane adjacent to the GATA4+ VE-like cells (FIG. 5R and FIG. 5S).

In sum, EPS-blastoids could give rise to functional ESCs, TSCs, and XENs, and upon further cultivation, could develop into postimplantation embryo-like structures.

### Example 6: In Vivo Development of EPS-Blastoids

A more stringent functional test for blastoids is to determine whether they develop into fetuses in utero. To this end, EPS-blastoids were transferred into pseudopregnant mice at 2.5 days post coitum (dpc) and their in vivo developmental potential was analyzed. At 7.5 dpc, decidua formed in the uteri of both control mice and surrogates that had been transferred with EPS-blastoids (FIG. 6A and FIG. 6B). Vascular permeability of EPS-blastoid-derived implantation sites were confirmed by Evans blue dye (FIG. 6C and FIG. 6D). Transfer experiments were performed using EPS-blastoids generated from several different lines and overall ˜7% of transferred EPS-blastoids implanted and induced decidualization (FIG. 6E and Table 11). This efficiency is comparable to that reported for ETS-blastoids (Rivron et al., 2018). Although control decidua appeared uniform in size, the size of EPS-blastoid-induced decidua varied, with some similar to the control and others much smaller (FIG. 6F). Genomic PCR analysis using a primer pair specific for the tdTomato gene showed that the decidua tissue contained tdTomato+ cells derived from EPS-blastoids (FIG. 6G). Immunohistochemistry analysis of decidua sections also confirmed the presence of tdTomato+ cells (FIG. 6H). In addition, an embryonic axis was established in deciduae induced by both a control blastocyst and an EPS-blastoid (FIG. 6I). Some structures formed within the EPS-blastoid-derived deciduae at 6.5, 7.5, and 8.5 dpc, respectively (Table 11), which all appeared retarded or malformed when compared with control E6.5-E8.5 embryos (FIG. 6J, FIG. 6K, FIG. 6L, and FIG. 6M). Nonetheless, the presence of OCT4+, EOMES+, and GATA4+ cells was detected in sections prepared from these structures (FIG. 6N to FIG. 6S). These results show that EPS-blastoids implant, trigger decidualization, and continue to grow inside the uterus.

TABLE 11
Summary of Decidualization Efficiency
of EPS-blastoids Transfer Experiments

| | | Number of | | |
|---|---|---|---|---|
| | | EPS-blastoid | Number of | Decidua/EPS- |
| Experiment | Cell line | transferred | Decidua | blastoids (%) |
| 1st | EPS_Td_1 | 40 | 2 | 5 |
| 2nd | EPS_Td_2 | 40 | 3 | 7.5 |
| 3rd | EPS_Td_1 | 20 | 1 | 5 |
| 4th | EPS_Td_2 | 20 | 2 | 10 |
| 5th | ES-converted | 20 | 1 | 5 |
| | EPS_1 | | | |
| 6th | ES-converted | 20 | 2 | 10 |
| | EPS_2 | | | |
| 7th | EPS_Td_2 | 20 | 2 | 10 |
| 8th | EPS_Td_1 | 40 | 5 | 12.5 |
| 9th | EPS_Td_2 | 40 | 2 | 5 |
| 10th | EPS_Td_1 | 40 | 2 | 5 |
| Total | | 300 | 22 | 7.33 |

TABLE 12
Summary of the Efficiency of Recovered Embryo/Embryo-
like Structures from Deciduae

| | Control | | | EPS-blastoid-derived | | |
|---|---|---|---|---|---|---|
| | Number of | Number of | Percentage | Number of | Number of | Percentage |
| dpc | decidua | embryos | (%) | decidua | embryo-like structures | (%) |
| 6.5 | 4 | 2 | 50 | 5 | 2 | 40 |
| 7.5 | 20 | 16 | 80 | 2 | 1 | 50 |
| 8.5 | 15 | 12 | 80 | 2 | 1 | 50 |

### Example 7: Methods for Generating EPS-Blastoids from Somatic Cells

To show that EPS-blastoids form without using any cells of embryonic origin, EPS-blastoids were generated from somatic cells. Through somatic cell reprogramming, EPS cells were established from mouse ear fibroblasts (induced EPS cells, or iEPS cells), which were subsequently used for blastoid formation. Blastoids were successfully generated from iEPS cells (referred to as iEPS-blastoids) in ˜15% of aggregates (FIG. 7A, FIG. 7B, and Table 1). Similar to EPS-blastoids, iEPS-blastoids also morphologically resembled natural blastocysts and were of similar size as E3.5 blastocysts (FIG. 7A and FIG. 7C). In addition, the process of the induction of iEPS-blastoids recapitulated the compaction, polarization, and changes in subcellular YAP localization (FIG. 7D, FIG. 7E, and FIG. 7F). iEPS-blastoids displayed the correct spatial expression of markers for both embryonic and extraembryonic lineages (FIG. 7G and FIG. 7H). In addition, further culture of iEPS-blastoids in IVC media generated egg-cylinder structures containing ExE-, EPI-, and VE-like compartments (marked by TFAP2C, SOX2/OCT4, and GATA4, respectively) (FIG. 7I, FIG. 7J, and FIG. 7K). Lastly, iEPS-blastoids were also able to implant into the uterus and induced the formation of decidua (FIG. 7L). Collectively, these data demonstrated that iEPS-blastoids generated from adult somatic cells are similar to those from embryo-derived stem cells.

### Example 8: Materials and Method Useful in the Present Disclosure

### Mice

All procedures related to animals were performed following the ethical guidelines of the Salk Institute for Biological Studies. Animal protocols were reviewed and approved by the Salk Institute Institutional Animal Care and Use Committee (IACUC) before any experiments were performed. C57BL/6J (Stock No: 000664 Black 6), ICR mice (Stock No: 009122), and C57BL/6-Tg(CAG-EGFP)1Osb/J (Stock No: 003291) were obtained from The Jackson Laboratory. To prepare pseudopregnant surrogates, ICR female mice (8-12 weeks old) in the estrus were mated with vasectomized ICR male mice. Mice were housed in a 12 hr light/12 hr dark cycle in a temperature-controlled facility with free access to water and food.

### Culture of Mouse Embryos

Mouse 2-cell embryo or blastocysts were flushed out of the uterus of pregnant female C57BL/6J or ICR and cultured in drops of KSOM medium (homemade or Millipore, MR-020P-5D; see also Summers 2013) covered by a layer of mineral oil (Sigma-Aldrich, M8410) in a humidified incubator under 5% CO₂ at 37° C. Homemade KSOM was prepared according to a previously published recipe (Wu et al., 2017). The KSOM medium contains: NaCl (95 mM), KCl (2.5 mM), KH₂PO₄ (0.35 mM), MgSO₄ (0.2 mM), NaHCO₃ (25 mM), CaCl₂ (1.71 mM), Naz-EDTA (0.01 mM), L-glutamine (1.0 mM), Na lactate (10 mM), Na pyruvate (0.2 mM), glucose (5.56 mM), essential amino acid (EAA; 10.0 m/l), non-essential amino acid (NEAA; 5.0 ml/l), and BSA (4 g/l). In some experiments, KSOM-HEPES medium was used. KSOM-HEPES medium was prepared using the same amount of chemicals as KSOM with the following changes: using lower amount of NaHCO3 (5 mM), the addition of HEPES-Na (20 mM), without EAA or NEAA, and substitution of BSA by PVA (0.1 g/l). All reagents were from Sigma-Aldrich except for NEAA and EAA, which were from Thermo Fisher Scientific. The sex of the mouse embryos was not determined. Both male and female embryos were used in all experiments.

To culture blastocysts beyond the implantation stage, a protocol developed by the Zernicka-Goetz group (Bedzhov and Zernicka-Goetz, 2014; Bedzhov et al., 2014b) was used. Blastocysts were first treated in drops of Tyrode's Solution, Acidic (Sigma-Aldrich) for one to two minutes to digest the zona pellucida. The zona-free blastocysts were washed in drops of KSOM medium and transferred into u-Slide 8 well (ibidi, 80826) containing pre-equilibrated IVC-1 medium (Cell Guidance Systems, M11-25). 20-25 blastocysts were plated into each well of u-Slide. Within 2 to 3 days, blastocysts attached to the plate and medium was replaced with pre-equilibrated IVC-2 (Cell Guidance Systems, M12-25). The culture continued for an additional 4-6 days and was fixed with 4% PFA for 15 min at room temperature for immunofluorescence analysis.

### Culture of Mouse Stem Cells

All stem cell lines were cultured on a layer of irradiated CF1 mouse embryonic fibroblasts (MEF) under 20% O₂ and 5% CO₂ at 37° C. The chimera-competent naïve ES cell line (B6N-22; male) was a gift from Fumihiro Sugiyama (Tanimoto et al., 2008). ES cells were cultured in N2B27-based medium. N2B27 basal medium was composed of 1:1 mixture of DMEM/F-12 (11330-032) and Neurobasal (21103-049) supplemented with 0.5X N2 (17502-048), 0.5X B27 (17504-044), 1X NEAA (11140-050), 1X GlutaMAX (35050-061), 0.1 mM 2-mercaptoethanol (21985-023), and 0.1% BSA (15260-037, optional) or 5% KnockOut Serum Replacement (10828-028, optional) (all from Thermo Fisher Scientific). Mouse ESCs were maintained in N2B27 medium supplemented with 10 ng/ml hLIF (Peprotech, 300-05), 3 μM CHIR99021 (Reagents Direct, 27-H76), and 1 μM PD0325901 (Selleck Chemicals, S1036) (hereinafter referred to as N2B27_{2iL}) (Ying et al., 2008) on a layer of irradiated MEF and passaged every two to three days using TrypLE (Thermo Fisher Scientific, 12604-013). The B6 GFP+ naïve ES cell line was derived from C57BL/6-Tg(CAG-EGFP)10sb/J blastocyst using the 2iL protocol. Blastocysts were collected from timed-pregnant mice and transferred onto a MEF feeder layer in a 96-well plate and cultured in N2B27_{2iL} medium. The cell outgrowth was dissociated and replated on new MEF feeder cells. Cell lines were established by dissociating individual colony using 0.05% trypsin-EDTA and replating into a new well.

The two EPS cell lines (EPS 1 and EPS 2, tdTomato+; both were male) derived from 8-cell embryos were obtained from the Hongkui Deng's lab. These two cell lines EPS cells were cultured on irradiated MEF cells in N2B27 basal medium supplemented with 10 ng/ml LIF (Peprotech, 300-05), 3 μM CHIR99021 (Reagents Direct, 27-H76), 2 μM (S)-(+)-Dimethindene maleate (Tocris, 1425), and 2 μM minocycline hydrochloride (Santa Cruz Biotechnology, sc-203339) (hereinafter referred to as N2B27_{LCDM}). In some experiments, the EPSC culture protocol developed by Pentao Liu's lab was used (Yang et al., 2017b). The Liu-EPS culture medium was CDF12 basal medium supplemented with 10 ng/ml hLIF (Peprotech, 300-05), 3 μM CHIR99021 (Reagents Direct, 27-H76), 1 μM PD0325901 (Selleck Chemicals, S1036), 4 μM JNK Inhibitor VIII (Millipore, 420135), 10 μM SB203580 (Tocris, 1402), 0.3 μM A-419259 (Tocris, 3914), and 5 μM XAV939 (Sigma-Aldrich, X3004). EPS cells were routinely passaged every two days at a ratio of 1:10 to 1:20. CDF12 basal medium was composed of DMEM/F-12 (11330-032) supplemented with 20% KnockOut Serum Replacement (10828-028), 1X NEAA (11140050), 1X GlutaMAX (35050-061), and 0.1 mM 2-mercaptoethanol (21985-023) (all from Thermo Fisher Scientific). The female X-GFP mEpiSC (female) was a gift from Dr. Azim Surani (Bao et al., 2009). EpiSCs were cultured in CDF12 medium supplemented with 12.5 ng/ml bFGF (Peprotech, 100-18B). To convert naïve ESCs or EpiSCs into EPS cells, naïve ESC or EpiSCs were first seeded on MEF feeder cells with ESC or EpiSC medium, respectively. After 24 hr, the medium was removed and replaced with EPS medium. The conversion process usually completed after five passages in the EPS conditions.

TSCs were maintained in basal TSC medium supplemented with 25 ng/ml rhFGF4 (R&D, 235F4025) and 1 μg/ml Heparin (Sigma-Aldrich, H3149) on a layer of irradiated MEF (Tanaka et al., 1998). TSC basal medium was composed of RPMI 1640 (11875-093) supplemented with 20% Fetal Bovine Serum (FBS) (16000-044), 1X GlutaMAX (35050061), 1X Sodium pyruvate (11360-070), and 0.1 mM 2-mercaptoethanol (21985-023) (all from Thermo Fisher Scientific). TSCs were passaged every five to seven days at 1:5-1:10 using 0.05% trypsin (Thermo Fisher Scientific, 25300-054). In addition, XEN cells were cultured in the TSC basal medium and passaged every 4 to 5 days using TrypLE.

### Culture of Mouse Ear Fibroblasts

Mouse ear fibroblasts were derived by plating minced ear tissue in DMEM (11950-040) supplemented with 10% FBS (16000-044), 1X NEAA (11140-050), and 1X GlutaMAX (35050-061) (all from Thermo Fisher Scientific) under 20% O₂ and 5% CO₂ at 37° C. When confluent, ear fibroblasts were split using TrypLE (Thermo Fisher Scientific, 12604-013) at 1:5.

### Reprogramming of Mouse Ear Fibroblasts

Retrovirus containing the four Yamanaka factors (Takahashi and Yamanaka, 2006) were packaged in 293 cells by transfection of pMXs-c-Myc, pMXs-Klf4, pMXs-Sox2, pMXs-Oct3/4 (all from Addgene). Mouse ear fibroblasts at passage 1 to 3 were used for reprogramming into iPS cells by incubation with the mixed retrovirus for two days. Then medium was replaced with CDF12 supplemented with 10 ng/ml LIF (CDF12_{LIF}). iPS colonies were picked up, dissociated with TrypLE, and replated into a new MEF well for establishing individual iPS cell line. iPS cells were routinely cultured in either CDF12_{LIF} or N2B27_{2iL}. iPS cells were converted into EPS cells by culturing in N2B27_{LCDM} for at least five passages.

### Lentiviral Transduction of EPS Cells

Lentiviral particle encoding the puromycin resistant gene and the mCherry gene (Lenti-EFla-puromycin-mcherry) (Liao et al., 2015) were packaged in 293 cells via transfection. Medium supernatant containing the lentiviral particles was collected 48 hr after transfection and concentrated by ultracentrifugation at 25,000 r.p.m (82,700g) at 4° C. for 2 hr using a Beckman ultracentrifuge (Beckman Coulter) (Kutner et al., 2009). The lentivirus pellet was resuspended with cold DPBS. EPS cells were transduced by incubating with lentivirus-containing N2B27_{LCDM} for 48 hr. Upon passaging, puromycin (1 μg/ml; InvivoGen, ant-pr-1) was supplemented in the medium to eliminate untransduced cells.

### Generation of EPS-Blastoids

EPS colonies were dissociated into single cells by incubation with TrypLE (Thermo Fisher Scientific, 12604-013). Cell resuspension was transferred into a 0.1% gelatin-coated plate and incubated at 37° C. for 30 min to allow irradiated MEF cells attach to the plate. The supernatant containing the EPS cells were collected, filtered through a 40 μm cell strainer, and counted using the TC-10 counter (Bio-Rad, 1450001). AggreWell 400 (STEMCELL Technologies, 34415) was prepared following the manufacturer's instructions. EPS-blastoid basal medium is composed of 25% TSC basal medium (see above), 25% N2B27 basal medium (see above), and 50% KSOM (see above). In some experiments, M16 (Sigma-Aldrich, M7292) was used to replace KSOM. Approximately 6,000 cells (five cells per microwell for 1200 microwells) were resuspended in EPS-blastoid basal medium supplemented with 2 μM ROCK inhibitor Y-27632 (Reagents Direct, 53-B80-50), 12.5 ng/ml rhFGF4 (R&D, 235F4025), 0.5 μg/ml Heparin (Sigma-Aldrich, H3149), 3 μM GSK3 inhibitor CHIR99021 (Reagents Direct, 27-H76), 5 ng/ml BMP4 (Proteintech, HZ-1040), and 0.5 μM A83-01 (Axon Medchem, 1421) and seeded into one well of the 24-well AggreWell plate. The plate was centrifuged at 300g for one minute and transferred into an incubator. The day of cell seeding was counted as day 0 of the process. Medium was removed 24 h later (day 1) and replaced with fresh medium without Y-27632. Additional medium change is optional for the rest of the EPS-blastoid formation process. Starting from day 4, blastoids were manually picked up using a mouth pipette (Sigma-Aldrich, A5177) under a stereomicroscope for analysis or downstream experiments. For testing of the effect of antagonists or inhibitors on EPS-blastoid induction, chemicals were added to the medium at day 1. XAV939 (5 μM; Tocris, 3748) or IWR-1-endo (10 μM; STEMCELL Technologies, 72562) was used to inhibit Wnt signaling. Verteporfin (2 μM; Tocris, 5305) was used to inhibit YAP/TEAD interaction. For testing whether a single EPS cell forms into a blastoid, WT cells and Puromycin+/mcherry+ cells were mixed at a ratio of 10:1 and seeded onto Aggrewell 400 as stated above. Puromycin (0.25 μg/ml; InvivoGen, ant-pr-1) was added to the medium 24 hours later to eliminate helper cells gradually.

Derivation of Three Types of Stem Cells from EPS-Blastoids

To derive ES and TS cells, individual EPS-blastoid was transferred onto a MEF feeder layer in a 96-well plate and cultured with ES culture medium (N2B27_{2iL}) or TS culture medium, respectively. Within 2-3 days, EPS-blastoids attached to the plate and outgrowth was observed. Outgrowth was dissociated with 0.05% trypsin and plated into a new plate with MEF feeders. Individual colony was manually picked, dissociated, and seeded onto a new 96-cell MEF feeders for cell line derivation. The derivation of XEN cell line was performed following an established protocol (Rugg-Gunn, 2017) with modifications. EPS-blastoids were plated individually in a 24-well plate pre-coated with MEF feeders in XEN derivation medium (TSC basal medium supplemented with 25 ng/ml rhFGF4 and 1 μg/ml Heparin). The outgrowth was formed around day 3. And medium was changed every 3 days until the XEN cells are around 80% confluent. The XEN cells were dissociated into single cells using TrypLE Express for 5 min at 37° C. with gentle pipetting. Dissociated cells were transferred into a 15 ml falcon tube containing 5× volume of digestion mixture and collected by centrifugation at 300 g for 5 min. After removal of the supernatant, the XEN cells were resuspended and seeded into a 6-well plate pre-coated with MEF feeders. After 2-3 passages, the medium was switched to XEN culture medium. Chimeric assays of ES and TS cells from EPS-blastoids

ICR female mice were superovulated by intraperitoneal (i.p.) injection of 7.5 Unit of pregnant mares' serum gonadotrophin (PMSG; Prospec-Tany Technogene, HOR-272) and 46-48 hr later 7.5 Unit of human chorionic gonadotrophin (HCG; Sigma-Aldrich, CG10-1VL), and then mated with ICR male mice immediately. The blastocysts were flushed from female mice 3.5 days after detection of the vaginal plug, and cultured in KSOM under 37° C. with an atmosphere of 5% CO2 in the air. The blastoid-derived ES or TS cells were dissociated into single cells and placed into the working medium before blastocyst injection. For each chimeric blastocyst, 12-15 ES or TS cells were injected into the cavity of blastocyst assisted with a PIEZO impact drive (Primetech, Ibaraki, Japan). After injection, the chimeric blastocysts were rinsed three times and cultured in KSOM at 37° C. with an atmosphere of 5% CO2 in the air. Fifteen to twenty chimeric blastocysts, which re-expanded after injection, were transferred into the uterine horn of 2.5 dpc pseudopregnant ICR female mice. For the ES chimeric group, full-term pups were delivery naturally from the pregnant mice at 17.5 days after embryo transfer; for TS chimeric group, the E14.5 fetus was dissected from uterine of pregnant mice 12.5 days after embryo transfer. Placenta was fixed with 4% PFA overnight and embedded in OCT. Frozen sections (10 μm thick) were cut using a microtome cryostat (Leica, model #CM1900-3-1).

Chimeric Assay of XEN Cells Derived from EPS-Blastoids

ICR female mice in natural estrous cycles were mated with same strain males, and blastocysts were collected from uterine at 3.5 dpc in KSOM-HEPES. They were cultured in the KSOM under 37° C. and 5% CO2 until microinjection of XEN cells. The EPS-blastoid derived XEN cells were dissociated into single cells and placed into KSOM on ice before blastocyst injection. Fifteen cells were introduced into the blastocoel assisted with a PIEZO impact drive. Microinjected blastocysts were cultured in KSOM until embryo transfer to the surrogates. Microinjected blastocysts were surgically transferred to the uterine horn of 2.5 dpc pseudopregnant ICR females. 15-18 blastocysts were transferred to each surrogate. E11.5 fetuses were dissected from uterine for analysis.

### In Vitro Culture of EPS-Blastoids Beyond Implantation

EPS-blastoids were cultured beyond the implantation stage using a protocol as for blastocyst (see above). EPS-blastoids were manually picked up using a mouth pipette, washed twice with pre-equilibrated IVC-1 medium (Cell Guidance Systems, M11), and transferred into a μ-Slide 8-well (ibidi, 80826) containing the IVC-1 medium. Around 20-30 EPS-blastoids were plated in one well of the μ-Slide. Within one or two days, EPS-blastoids attached to the plate. Once the EPS-blastoids attached, the medium was switched to IVC-2 medium (Cell Guidance Systems, M12). In two to four days, postimplantation embryo-like structures emerged and were fixed with 4% paraformaldehyde (PFA) for 15 min at room temperature for immunofluorescence staining analysis.

### EPS-Blastoid Transfer

EPS-blastoids were manually picked up under a stereomicroscope and transferred into KSOM droplets using a mouth pipette. The surrogate at 2.5 days post coitum (dpc) was anesthetized with ketamine (Putney) and xylazine (Akorn) and the uterine horn was exposed surgically. After three washes in KSOM, EPS-blastoids were loaded to the pipette with air bubble and transferred to the uterine horn, which was previously punctured with a 27 G needle. Around 20 EPS-blastoids were transferred into each uterine horn. The process of transfer was typically performed within 20-30 min per surrogate. A C-section was performed at 6.5, 7.5, or 8.5 dpc, and the uterus was dissected out. For staining with Evans blue, the surrogate mice received a tail vein injection of 0.5% Evans Blue (MP Biomedicals, 151108) 15 min before the C-section. Deciduae were dissected out of the uterus, and embryo-like structures were dissected out of the deciduae. Tissue samples were fixed with 4% PFA overnight and embedded in OCT. Frozen sections (10 μm thick) were cut using a microtome cryostat (Leica, model #CM1900-3-1).

### Immunofluorescence Staining

Immunofluorescence for 2D cell culture, 3D cell aggregates, EPS-blastoid, early mouse embryos, and postimplantation embryo-like structures was performed following a previously established protocol (Gu et al., 2018) with small modifications. The samples were fixed with freshly prepared 4% PFA in PBS for 15 min at room temperature and permeabilized with 0.2% Triton X-100 in PBS for 15 min. Samples were then blocked with blocking buffer (PBS containing 5% normal donkey serum (NDS), 2% BSA, and 0.1% Tween 20) at room temperature for two hours or overnight (O/N) at 4° C. Primary antibodies diluted in blocking buffer were applied to samples and incubated for two hours at room temperature or O/N at 4° C. Samples were washed for three times with PBS containing 0.1% Tween 20 followed by the incubation with fluorescence-conjugated secondary antibodies diluted in blocking buffer (2-5 μg/ml) for 1 hr (2D culture) or 2 hr (3D structures and postimplantation embryo-like structures) at room temperature. Samples were washed for three times with PBS containing 0 1% Tween 20. Nuclei were counterstained with Hoechst 33342 at 1 μg/ml. In some experiments for staining with membrane-associated protein (E-cadherin, ZO1, and PARE), Saponin (0.1%; MP Biomedicals, 102855) was used for permeabilization and wash to replace Triton X-100 and Tween20. For staining of tissue cryosections, an additional step of antigen retrieval between permeabilization and blocking was performed to incubate the sections in 1X HistoVT One (Nacalai Tesque, 06380-05) at 70° C. for 20 min. When using mouse antibody on mouse tissue, Mouse on Mouse Basic Kit (Vector Laboratories, Cat# BMK-2202) was used after blocking with normal serum and BSA. Also, to reduce background, 1X TruBlack Lipofuscin Autofluorescence Quencher (Biotium, 23007) was applied to sections as the last step of the staining process. Image acquisition was performed using a Zeiss LSM 710 or 880 confocal microscope. Images were processed using Fiji (ImageJ, v2.0.0) (Rueden et al., 2017)(Schindelin et al., 2012) or Zen (Zeiss). 3D cell counting was performed using the Imaris software (Oxford Instruments). The primary antibodies and dilutions used were: anti-CDX2 (1:100; Biogenex, MU392A), anti-KRT8 (1:5; Developmental Studies Hybridoma Bank, TROMA-1), anti-EOMES (1:200; Abcam, Ab23345), anti-ECAD (1:200; Dako, M3612), anti-ZO1 (1:150, Invitrogen, 61-7300), Rabbit anti-OCT4 (1:200; Abcam, ab19857), Rabbit anti-GATA6 (1:100; Cell Signaling Technology, 5851), Mouse anti-laminin gamma 1 (1:5; DSHB, 2E8), Goat anti-GATA6 (1:200; R and D Systems, AF1700), anti-PAR6 (1:50, Santa Cruz Biotechnology, sc-166405), anti-YAP (1:100, Santa Cruz Biotechnology, sc101199), anti-active YAP (1:100, Abcam, ab205270), anti-GFP (1:1000, MBL, M048-3), anti-TFAP2C (1:50; Santa Cruz Biotechnology, sc-12762), anti-OCT4 (1:100; Santa Cruz Biotechnology, sc-5279), anti-NANOG (1:100; Invitrogen, 14-5761-80), anti-SOX2 (1:100; R&D, AF2018), anti-GATA4 (1:100; Santa Cruz Biotechnology, sc-1237), anti-aPKC (1:50; Santa Cruz Biotechnology, sc-17781), and anti-Podocalyxin (PCX) (1:200; R&D, MAB1556). Secondary antibodies used were: Alexa Fluor 488 Donkey anti-Rat IgG (H+L) (Thermo Fisher Scientific, A-21208), Alexa Fluor 488-AffiniPure Donkey Anti-Goat IgG (H+L) (Jackson ImmunoResearch Labs, 705545-147), Alexa Fluor 488-AffiniPure Donkey Anti-Mouse IgG (H+L) (Jackson ImmunoResearch Labs, 715-545-151), Alexa Fluor 488-AffiniPure Donkey Anti-Rabbit IgG (H+L) (Jackson ImmunoResearch Labs, 711-545-152), Alexa Fluor 555 Donkey Anti-Mouse IgG (H+L) (Thermo Fisher Scientific, A-31570), Alexa Fluor 555 Donkey anti-Rat IgG (H+L) (Abcam, ab150154), Alexa Fluor 647 Donkey anti-Rat IgG (H+L) (Abcam, ab150155), Alexa Fluor 647-AffiniPure Donkey Anti-Goat IgG (H+L) (Jackson ImmunoResearch Labs, 705-605-147), Alexa Fluor 647-AffiniPure Donkey Anti-Mouse IgG (H+L) (Jackson ImmunoResearch Labs, 715-605-151), Alexa Fluor 647-AffiniPure Donkey Anti-Rabbit IgG (H+L) (Jackson ImmunoResearch Labs, 711-605-152), DyLight 550 Donkey anti-Goat IgG (H+L) (Thermo Fisher Scientific, SA5-10087), DyLight 550 Donkey anti-Rabbit IgG (H+L) (Thermo Fisher Scientific, SA5-10039). F-actin was directly stained with Phalloidin CruzFluor 488 Conjugated antibody (1:1000; Santa Cruz Biotechnology, sc-363791) along with other secondary antibodies in blocking buffer.

### Immunohistochemistry

Immunohistochemistry (IHC) was performed using the ImmPRESSTM HRP Anti-Rabbit IgG (Peroxidase) Polymer Detection Kit (Vector laboratories, MP-7401) according to the manufacturer's instructions with minor modifications. The frozen sections were firstly treated with citrate-based (Vector laboratories, H-3300) for antigen unmasking. Blocking was done with 2.5% normal horse blocking serum included in the kit for 1 hr at room temperature. The primary antibody for tdTomato (1:200; Rockland, 600-401-379) was applied to the sections and incubated overnight at 4° C. All washes were performed with 0.1% Tween-20 (PBS) for 5 min. The color was developed using ImmPACT DAB Peroxidase (HRP) Substrate (Vector laboratories, SK-4105) according to the manufacturer's instructions. Lastly, the sections were counterstained with hematoxylin and went through a series of ethanol-based dehydration and xylene-based clearing and mounted with mounting medium.

### Genomic DNA PCR

Decidua tissue sample was minced and resuspended in TE buffer. Tissue was digested by treating with 0.3 mg/ml proteinase K (Thermo Fisher Scientific, AM2546) at 55° C. overnight. Genomic DNA preparations were incubated at 95° C. for 5 min to inactivate proteinase K before used for PCR. An ultraconserved noncoding element (UNCE) overlapping with the Tfap2a locus was used as an internal control (Cohen et al., 2016). The tdTomato gene was amplified by a nested PCR. The first round of PCR was done with an external primer set: 5′-GGC GAG GAG GTC ATC AAA GAG T-3′, 5′-ATG GTG TAG TCC TCG TTG TGG G-3′. PCR product of the first PCR was diluted at 1:200 and 1 μl of the diluted sample was used as the template for the second round nested PCR with the following primers: 5′-ACA TCC CCG ATT ACA AGA AGC-3′, 5′-TTG TAG ATC AGC GTG CCG TC-3′. All PCR reactions were performed with PrimeSTAR GXL DNA Polymerase (Clontech, R050B). PCR products were resolved in a 2% agarose gel with TBE buffer. Images were acquired using a Bio-Rad Gel Doc XR+ system with Image Lab software.

### Transcriptome Analysis

Total RNA was isolated from eight individual EPS-blastoids collected at day five using the TRIzol (Thermo Fisher Scientific, 15596026) method. RNA-Seq libraries were constructed using the Illumina Smart-Seq2 (Picelli et al., 2013) using Nextera XT DNA sample preparation kit (Illumina, FC-131-1096) and Nextera XT 24-index kit (Illumina, FC-131-1001), and 2×150 bp pair-end sequencing was performed on an Illumina HiSeq Xten. Sequencing reads were filtered and mapped to the mouse genome build mm10 using the HISAT2 alignment program (Kim et al., 2019). De novo transcriptome assembly and transcript and gene abundance calculations were performed using the StringTie assembler (Pertea et al., 2015). The expression values of each gene were normalized using FPKM. RNA-Seq data of morula stage and E3.5 early blastocyst stage embryos were obtained from published datasets (GSE98150 and GSE87504, respectively) (Sampath Kumar et al., 2017; Wang et al., 2018). Raw read data were downloaded and processed using the same pipeline as that used for EPS-blastoid data. Differentially expressed genes (DEGs) were calculated using the R package ballgown (Frazee et al., 2015). DEGs were deemed significant if they passed the following cutoff parameters: FPKM>1, absolute value of log 2 ratio >1, and Q-value (adjusted p-value) <0.05. Gene ontology (GO) and KEGG pathway analyses were performed using Fisher's exact test, and the false discovery rate (FDR) was controlled by the BH method. Principle components analysis was performed using the R package ade4 (Dray and Dufour, 2007). Cluster analysis was performed using the R package pvclust (Suzuki and Shimodaira, 2006). Heatmaps were generated using the R package pheatmap (Kolde, 2012).

### Single-Cell RNA-Seq Library Generation

EPS-blastoids were manually picked up using mouth pipette and washed three times in PBS containing 0.04% BSA. Around 500 EPS-blastoids were harvested and dissociated with a homemade enzyme mix composed of 0.5X versene (Lonza, 17711E), 0.5X Acumax (Innovative Cell Tech, AM105), and 0.05X Dnase (STEMCELL Technologies, 07900) at 37° C. for 30min with agitation. Dissociated cells were spun down and wash with PBS+0.04% BSA for three times and resuspended in the same buffer. Cell density was determined by a TC10 cell counter (Bio-Rad, 1450001). Blastocysts were dissociated using the same protocol. Dissociated cells (˜4800 cells for EPS-blastoids and 1000 cells for blastocysts) were loaded into the Chromium Single Cell B Chip (10X Genomics, PN-120262) and processed in the Chromium single cell controller (10X Genomics) to generate single-cell gel beads in the emulsion according to the manufacturer's protocol. The library was generated using the Chromium Single Cell 3′ Reagent Kits v3 (10X Genomics, PN-1000092) and Chromium i7 Multiplex Kit (10X Genomics, PN-120262) according to the manufacturer's manual. The two libraries were pooled and sequenced using Nextseq 500 (150 cycles, high output).

### Single-Cell RNA-Seq Data Analysis

STAR v2.5.1b1 (Dobin et al., 2013) was used to align reads to the 10x Genomics pre-built mm10 reference genome and utilized the CellRanger v3.0.2 (10X Genomics) software for blastocysts (288 cells) and EPS-blastoids (3528 cells) datasets with the default setting for de-multiplexing to generate feature-barcode matrix. The R package Seurat v3.0.12 (Stuart et al., 2019) was used to read and analyze feature-barcode matrix following the steps: First, cells were filtered that have unique feature counts over 5000 according to quality control matrix plots (184 and 2518 cells in the blastocysts and EPS-blastoids group passed the filter, respectively); Then UMI counts were normalized with NormalizeData function using the default settings. Seurat's RunUMAP function was used to perform a non-linear dimension reduction and clustering with resolution setting at 0.2. Differentially expressed genes within the clusters between blastocysts and EPS-blastoids were determined by the FindMarkers function using a bimodal likelihood ratio test. For the differentially expressed genes, whether each had enriched GO terms in biological process and molecular functions was tested using the ToppGene Suite3 (Chen et al., 2009). Unsupervised clustering analysis (UCA) was performed using the R package ComplexHeatmap v2.1.0 (Gu et al., 2016) with clustering_distance_columns=“manhattan”.

### Quantification and Statistical Analysis

The sample size was not predetermined using any statistical methods or packages before experimentation. Quantification details on the number of biological replicates (n value) and data presentation were included in figure legends. Values were shown as the mean and error bars represented SEM unless otherwise indicated. Statistical analysis details were described in figure legends or method details. No method was used to determine whether the data met assumptions of the statistical approach. Differences were considered to be significant when the P (or adjusted P) values were smaller than 0.05. Graphs were generated using Prism or R package ggplot2 (Wickham, 2016) or other R packages described in the method details.

### Data and Code Availability

R scripts used for the single-cell RNA-Seq analysis are available upon request. The sequencing data have been deposited at the NCBI Gene Expression Omnibus under the following accession number: GSE135289 (bulk RNA-Seq) and GSE135701 (single-cell RNA-Seq).

### Example 9: Culture of Human EPS or Liu-EPSC Cells

All stem cell lines were cultured on a layer of irradiated CF1 mouse embryonic fibroblasts (MEF) under 20% O₂ and 5% CO₂ at 37° C.

Human primed embryonic stem cells were cultured in CDF12 medium, which was composed of DMEM/F-12 (11330-032) supplemented with 20% KnockOut Serum Replacement (10828-028), 1X NEAA (11140-050), 1X GlutaMAX (35050-061), 0.1 mM 2-mercaptoethanol (21985-023) (all from Thermo Fisher Scientific), and 10 ng/mL FGF2 (Peprotech). To convert human primed ESCs into EPS or Liu-EPSC cells, human primed ESCs were first seeded on MEF feeder cells with CDF12 medium. After 24 h, the medium was removed and replaced with EPS or Liu-EPSC medium. After 3-4 days, cell colonies were dissociated into single cells with Accumax (Stemcell Technology, 07921), and passaged into new CF1 MEF plate at 1:5-1:10 in EPS or Liu-EPSC medium with 10 μM Rock inhibitor Y-27632 (Reagents Direct, 53-B80-50). Change medium without Rock inhibitor Y-27632 next day. The conversion process usually completed after three to five passages in the EPS conditions according to the references (Yang et al, Cell, 2017; Gao et al, Nature Cell Biology).

EPS medium is composed of N2B27 basal medium supplemented with 10 ng/mL LIF (Peprotech, 300-05), 1.5 μM CHIR99021 (Reagents Direct, 27-H76), 2 μM (S)-(+)-Dimethindene maleate (Tocris, 1425), 2 μM minocycline hydrochloride (Santa Cruz Biotechnology, sc-203339) (hereinafter referred to as N2B27-LCDM), and 2 μM IWR endo-1 (Selleck, S7086). EPSC medium is composed of N2B27 basal medium supplemented 1.0 μM CHIR99021, 0.1 μM A419259 (Tocris, cat. no. 3914), 2.5 μM XAV939 or 2.0 μM IWR-1, 65 μg/ml vitamin C, 10 ng/ml LIF (SCI), 0.25 μM SB590885 and 2.0 μM SP600125. N2B27 basal medium was composed of 1:1 mixture of DMEM/F-12 (11330-032) and Neurobasal (21103-049) supplemented with 0.5X N2 (17502-048), 0.5X B27 (17504-044), 1X NEAA (11140-050), 1X GlutaMAX (35050-061), 0.1 mM 2-mercaptoethanol (21985-023), and 0.1% BSA (15260-037, optional) or 5% KnockOut Serum Replacement (10828-028, optional) (all from Thermo Fisher Scientific).

### Example 10: Generation of Human Blastoids

EPS or EPSC colonies were dissociated into single cells by incubation with Accumax (Stemcell Technology, 07921). Cell resuspension was transferred into a 0.1% gelatin-coated plate and incubated at 37° C. for 30 min to allow irradiated MEF cells attach to the plate. The supernatant containing the EPS or EPSC cells were collected, filtered through a 40 μm cell strainer, and counted using the TC-10 counter (Bio-Rad, 1450001). AggreWell 400 (STEMCELL Technologies, 34415) was prepared following the manufacturer's instructions. EPS-blastoid basal medium is composed of 25% TSC basal medium, 25% N2B27 basal medium (see above), and 50% KSOM. In some experiments, M16 (Sigma-Aldrich, M7292) was used to replace KSOM. Approximately 24,000 cells (20 cells per microwell for 1200 microwells) were resuspended in EPS-blastoid basal medium supplemented with 2 μM ROCK inhibitor Y-27632 (Reagents Direct, 53-B80-50), 12.5 ng/mL rhFGF4 (R&D, 235F4025), 0.5 μg/mL Heparin (Sigma-Aldrich, H3149), 3 μM GSK3 inhibitor CHIR99021 (Reagents Direct, 27-H76), 5 ng/mL BMP4 (Proteintech, HZ-1040), and 0.5 μM A83-01 (Axon Medchem, 1421) and seeded into one well of the 24-well AggreWell plate. The plate was centrifuged at 300 g for one minute and transferred into an incubator. The day of cell seeding was counted as day 0 of the process. Medium was removed 24 h later (day 1) and replaced with fresh medium without Y-27632. Additional medium change is optional for the rest of the EPS-blastoid formation process. Starting from day 5 or day 6, blastoids were manually picked up using a mouth pipette (Sigma-Aldrich, A5177) under a stereomicroscope for analysis or downstream experiments. TSC basal medium was composed of RPMI 1640 (11875-093) supplemented with 20% Fetal Bovine Serum (FBS) (16000-044), 1X GlutaMAX (35050-061), 1X Sodium pyruvate (11360-070), and 0.1 mM 2-mercaptoethanol (21985-023) (all from Thermo Fisher Scientific). Homemade KSOM was prepared according to a previously published recipe (Wu et al., 2017). The KSOM medium contains: NaCl (95 mM), KC1 (2.5 mM), KH2PO4 (0.35 mM), MgSO4 (0.2 mM), NaHCO3 (25 mM), CaCl2 (1.71 mM), Na2-EDTA (0.01 mM), L-glutamine (1.0 mM), Na lactate (10 mM), Na pyruvate (0.2 mM), glucose (5.56 mM), essential amino acid (EAA; 10.0 m/l), non-essential amino acid (NEAA; 5.0 m/l), and BSA (4 g/l).

While preferred embodiments of the present invention have been shown and described herein, it will be obvious to those skilled in the art that such embodiments are provided by way of example only. Numerous variations, changes, and substitutions will now occur to those skilled in the art without departing from the invention. Various explicit examples of compositions of matter and processes/methods are described herein, the components or steps of which are optionally utilized in any composition of matter and/or process/method described herein, as applicable. It should be understood that various alternatives to the embodiments described herein may be employed. It is intended that the following claims define the scope of the invention and that methods and structures within the scope of these claims and their equivalents be covered thereby.

1. A method of producing a blastoid, the method comprising:
(a) obtaining or providing an extended pluripotent stem (EPS) cell; (b) culturing the EPS cell in a medium comprising one or more factors selected from the group consisting of a ROCK inhibitor, a FGF, Heparin, a Wnt agonist, BMP, and a TGF-β signaling inhibitor; and (c) isolating the resulting blastoid.

2. The method of claim 1, wherein the medium comprises:
two or more factors selected from the group consisting of the ROCK inhibitor, the FGF, Heparin, the Wnt agonist, the BMP, and the TGF-β signaling inhibitor;
three or more factors selected from the group consisting of the ROCK inhibitor, the FGF, Heparin, the Wnt agonist, the BMP, and the TGF-β signaling inhibitor;
four or more factors selected from the group consisting of the ROCK inhibitor, the FGF, Heparin, the Wnt agonist, the BMP, and the TGF-β signaling inhibitor;
five or more factors selected from the group consisting of the ROCK inhibitor, the FGF, Heparin, the Wnt agonist, the BMP, and a TGF-β signaling inhibitor; or
the ROCK inhibitor, the FGF, Heparin, the Wnt agonist, the BMP, and the TGF-β signaling inhibitor.

3.-6. (canceled)

7. The method of claim 1, wherein the ROCK inhibitor is Y-27632, the FGF is FGF4, the Wnt agonist is Wnt-3a or CHIR99021, the BMP is BMP4, and/or the TGF-β signaling inhibitor is A83-01, SB431543, OR REPSOX.

8. The method of claim 1, wherein the EPS cell is cultured in a v-bottomed microwell plate.

9. The method of claim 8, wherein:
(i) the v-bottomed microwell plate is an AggreWell plate;
(ii) the v-bottomed plate is centrifuged at about 300×g after the cell and medium is added to the plate; or
(iii) both (i) and (ii).

10. (canceled)

11. The method of claim 1, wherein:
(i) after about 24 hours, the medium is replaced with a medium without the ROCK inhibitor;
(ii) the culturing is conducted for about 5 days;
(iii) the EPS cell is cultured with a trophectoderm (TE) cell at step (b);
(iv) the EPS cell is cultured in a medium comprising a KSOM or comprising an M16 medium; or
(v) any combination of (i)-(iv).

12.-13. (canceled)

14. A method of assisted reproduction of an individual, the method comprising: (a) obtaining or providing an extended pluripotent stem (EPS) cell derived from the individual; (b) culturing the EPS cell in a medium comprising one or more of factors selected from the group consisting of a ROCK inhibitor, a FGF, Heparin, a Wnt agonist, a BMP, and a TGF-β signaling inhibitor; (c) isolating a resulting blastoid; (d) transferring the resulting blastoid to a uterus.

15. The method of claim 14, wherein the medium comprises:
two or more factors selected from the group consisting of the ROCK inhibitor, the FGF, Heparin, the Wnt agonist, the BMP, and the TGF-β signaling inhibitor;
three or more factors selected from the group consisting of the ROCK inhibitor, the FGF, Heparin, the Wnt agonist, the BMP, and the TGF-β signaling inhibitor;
four or more factors selected from the group consisting of the ROCK inhibitor, the FGF, Heparin, the Wnt agonist, the BMP, and the TGF-β signaling inhibitor;
five or more factors selected from the group consisting of the ROCK inhibitor, the FGF, Heparin, the Wnt agonist, the BMP, and the TGF-β signaling inhibitor; or
the ROCK inhibitor, the FGF, Heparin, the Wnt agonist, the BMP, and the TGF-β signaling inhibitor.

16.-19. (canceled)

20. The method of claim 14, wherein the ROCK inhibitor is Y-27632, the FGF is FGF4, the Wnt agonist is Wnt-3a or CHIR99021, the BMP is BMP4, and/or the TGF-β signaling inhibitor is A83-01, SB431543, or REPSOX.

21. The method of claim 14, wherein:
(i) the EPS cell is cultured in a v-bottomed microwell plate;
(ii) EPS cell is an induced EPS cell derived from a somatic cell; or
(iii) both (i) and (ii).

22. The method of claim 21, wherein:
(i) the v-bottomed microwell plate is an AggreWell plate;
(ii) the v-bottomed plate is centrifuged at about 300×g after the cell and medium is added to the plate; or
(iii) both (i) and (ii).

23. (canceled)

24. The method of claim 14, wherein:
(i) after about 24 hours, the medium is replaced with a medium without Y-27632;
(ii) the culturing is conducted for about 5 days;
(iii) the EPS cell is cultured with a trophectoderm (TE) cell at step (b);
(iv) the EPS cell is cultured in a medium comprising a KSOM or comprising an M16 medium; or
(v) any combination of (i)-(iv).

25. (canceled)

26. The method of claim 14, wherein:
(i) the individual is a mammal selected from a mouse, a rat, a rabbit, a horse, a sheep, a cow, a dog, a cat, an elephant, a whale, a rhinoceros, a non-human primate, or a human;
(ii) the uterus is receptive to implantation; or
(iii) both (i) and (ii).

27.-29. (canceled)

30. A method of determining a drug toxicity, the method comprising: (a) obtaining or providing a blastoid produced by a method according to claim 1; (b) contacting the blastoid to the drug; and (c) detecting signs of toxicity.

31. The method of claim 30, wherein the signs of toxicity comprise cell death, loss of blastoid cell organization, arrest in blastoid growth or development.

32. (canceled)

33. A blastoid, e.g., produced or producible by a method comprising: (a) obtaining an extended pluripotent stem (EPS) cell; (b) culturing the EPS cell in a medium comprising one or more of factors selected from the group consisting of a ROCK inhibitor, a FGF, Heparin, a Wnt agonist, a BMP, and a TGF-β signaling inhibitor; and (c) isolating the resulting blastoid.

34. The blastoid of claim 33, wherein the medium comprises:
two or more factors selected from the group consisting of the ROCK inhibitor, the FGF, Heparin, the Wnt agonist, the BMP, and the TGF-β signaling inhibitor;
three or more factors selected from the group consisting of the ROCK inhibitor, the FGF, Heparin, the Wnt agonist, the BMP, and the TGF-β signaling inhibitor;
four or more factors selected from the group consisting of the ROCK inhibitor, the FGF, Heparin, the Wnt agonist, the BMP, and the TGF-β signaling inhibitor;
five or more factors selected from the group consisting of the ROCK inhibitor, the FGF, Heparin, the Wnt agonist, the BMP, and the TGF-β signaling inhibitor; or
the ROCK inhibitor, the FGF, Heparin, the Wnt agonist, the BMP, and the TGF-β signaling inhibitor.

35.-38. (canceled)

39. The blastoid of claim 33, wherein the ROCK inhibitor is Y-27632, the FGF is FGF4, the Wnt agonist is Wnt-3a or CHIR99021, the BMP is BMP4, and/or the TGF-β signaling inhibitor is A83-01, SB431543, or REPSOX.

40. The blastoid of claim 33, wherein the EPS cell is cultured in a v-bottomed microwell plate.

41. The blastoid of claim 40, wherein:
(i) the v-bottomed microwell plate is an AggreWell plate;
(ii) the v-bottomed plate is centrifuged at about 300×g after the cell and medium is added to the plate; or
(iii) both (i) and (ii).

42. (canceled)

43. The blastoid of claim 33, wherein:
(i) after about 24 hours, the medium is replaced with a medium without the ROCK inhibitor
(ii) the culturing is conducted for about 5 days;
(iii) the EPS cell is cultured with a trophectoderm (TE) cell at step (b); or
(iv) any combination of (i)-(iii).

44. (canceled)

45. The blastoid of claim 33, wherein:
(i) the EPS cell is cultured in a medium comprising a KSOM or comprising an M16 medium;
(ii) the EPS cell is an induced EPS cell derived from a somatic cell;
(iii) the EPS cell is derived from a mammal selected from a mouse, a rat, a rabbit, a horse, a sheep, a cow, a dog, a cat, an elephant, a whale, a rhinoceros, a non-human primate, or a human; or
(iv) any combination of (i)-(iii).

46. The blastoid of claim 33, wherein:
(i) the Wnt agonist is CHIR99021;
(ii) the TGF-β signaling inhibitor comprises A83-01, SB431543, or REPSOX; or
(iii) both (i) and (ii).

47.-50. (canceled)
